(19) United States
(12) Patent Application Publication
(10) Pub. No.: US 2026/0242367 A1
(43) Pub. Date: Aug. 20, 2026

(54) Substituted Amine Compounds, Compositions and Methods of Use

(71) Applicant: Deep Apple Therapeutics, Inc., South San Francisco, CA (US)

(72) Inventors: Stefan GAHBAUER, South San Francisco, CA (US); Craig Stivala, San Mateo, CA (US); Jason R. Zbieg, Montara, CA (US); Liang Zhao, San Mateo, CA (US)

(21) Appl. No.: 19/539,414

(22) Filed: Feb. 13, 2026

Related U.S. Application Data
(60) Provisional application No. 63/760,918, filed on Feb. 20, 2025.
(60) Provisional application No. 63/966,774, filed on Jan. 23, 2026.

(51) Int. Cl. C07D 413/14; C07D 401/14; C07D 487/04

(52) U.S. Cl. CPC C07D 413/14; C07D 401/14; C07D 487/04

(57) ABSTRACT

The present disclosure provides compounds that are useful for the treatment of conditions mediated by MRGPRX2. Also provided are pharmaceutical compositions containing such compounds, and methods of treatment using such compounds.

### CROSS REFERENCE TO RELATED APPLICATIONS

This application claims priority to U.S. Provisional Application No. 63/760,918, filed on Feb. 20, 2025, and U.S. Provisional Application No. 63/966,774, filed on Jan. 23, 2026, the disclosures of each of which are hereby incorporated by reference in their entireties.

### FIELD OF INVENTION

The present disclosure provides compounds that modulate MRGPRX2 and are therefore useful for the treatment of conditions, diseases and/or disorders mediated by MRGPRX2. Also provided are pharmaceutical compositions containing such compounds and processes for preparing such.

### BACKGROUND

Mas-related G-protein coupled receptor member X2 is a protein that, in humans, is encoded by the MRGPRX2 gene and is most abundant on cutaneous mast cells.

Agonists are gyrase inhibitors like ciprofloxacin and non-depolarizing neuromuscular blocking agents like atracurium as well as vancomycin. Activation of MRGPRX2 leads to mast cell degranulation with subsequent pseudo-allergic reactions.

Mature mammalian mast cells ordinarily reside: Near blood vessels or nerves; beneath or within epithelia; within airways, gastrointestinal, and genitourinary tracts; and near smooth muscle and mucus-producing glands. Classically, mast cells are activated by IgE antibodies, secreting a wide range of substances with local and systemic effects including histamine, serotonin, proteases, chemokines, and cytokines. Indeed, mast cells are implicated in the progression and/or maintenance of many diseases (Nature Immunology, 6, 135-142 (2005)).

Recent work has emphasized the role of the Mas-related G protein-coupled receptor (MRGPR) family, specifically, Mrgprb2, in mast cell activation (Nature, 519, 237-241 (2015)). Mrgprb2 is the mouse receptor for several cationic molecules, collectively called basic secretagogues, and the ortholog of the human receptor MRGPRX2 (Adv. Immunol., 136, 123-62 (2017)). To date, Mrgprb2 and MRGPRX2 have been reported to be expressed only on certain populations of mast cells (Nature Immunology, 25 17, 878-887 (2016); Annu. Rev. Immunol., 38, 49-77 (2020)). This knowledge provides the opportunity to target mast cell degranulation in a very precise manner.

WO 2022/073904 discloses MrgX2 amide antagonists useful for treating MrgX2 mediated diseases such as urticaria and atopic dermatitis. WO2022/152852, WO2022/152853 and WO2022/073904/WO2022/073905 also disclose MrgX2 amide antagonists useful for treating MrgX2 mediated diseases. WO2021/092262 discloses MRGPRX2 antagonists, typically amides, useful for the treatment of inflammatory conditions such as atopic dermatitis. More recently WO2024/226914 also discloses MRGPRX2 antagonists useful for treating such disorders.

In summary, a potent, selective antagonist of MRGPRX2 that blocks IgE-independent mast cell de-granulation is expected to provide therapeutic benefit in mast-cell driven pathologies including skin disorders such as urticaria, atopic dermatitis and rosacea as well as additional indications like inflammatory disease conditions and autoimmune disease conditions. Thus, there is a need and a continuing search in this field of art for new therapies.

### SUMMARY

The present invention relates to compounds of Formula (I), as described herein including stereochemical isomeric forms thereof and pharmaceutically acceptable salts thereof, which may be useful for mediating MRGPRX2 and/or treating or preventing MRGPRX2 mediated conditions and diseases. More specifically, in some embodiments, provided herein are compounds of Formula (I) or a pharmaceutically acceptable salt thereof:

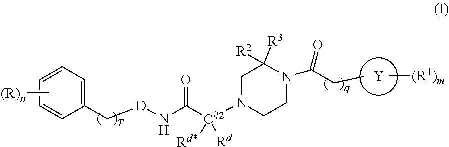

wherein
T is 1 or 0; provided that when T is 1, D is a 5-membered heteroaryl group having one or two heteroatoms selected from nitrogen and oxygen, and D is unsubstituted or is mono-substituted with (C₁-C₄)alkyl, mono-, di-, or tri-fluoromethyl; and
when T is 0, D is

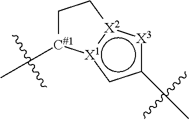

and;
X¹ and X² are each independently N or C;
X³ is N or CH;
C^{#1} is a first stereocenter;
C^{#2} is a second stereocenter;
n is 1, 2 or 3;
each R is independently H or halo;
either R^{d*} is H and R^{d} is methyl or R^{d*} is methyl and R^{d} is H;
Y is absent or Y is selected from
   a 5- or 6-membered heteroaryl group having one to three heteroatoms independently selected from nitrogen, oxygen and sulfur;
   a 4- to 6-membered heterocycloalkyl group having 1 to 2 heteroatoms independently selected from nitrogen, oxygen and sulfur;
   a phenyl ring fused to a 5- or 6-membered heteroaryl group to form a bicyclic group having one to three heteroatoms independently selected from nitrogen, oxygen and sulfur;
   a 5- or 6-membered heteroaryl group fused to a 5- or 6-membered heteroaryl group to form a bicyclic group having one to five heteroatoms independently selected from nitrogen, oxygen and sulfur;
   a 5- to 7-membered heterocycloalkyl group fused to a 5- or 6-membered heteroaryl group to form a bicyclic group having one to four heteroatoms independently selected from nitrogen, oxygen and sulfur;
   a (C₅-C₇)cycloalkyl group fused to a 5- or 6-membered heteroaryl group to form a bicyclic group, said 5- or 6-membered heteroaryl group having one to three heteroatoms independently selected from nitrogen, oxygen and sulfur;
   a phenyl group;
   a (C₄-C₇)cycloalkyl group; or
   a phenyl group fused to a 5- to 7-membered heterocycloalkyl group to form a bicyclic group, said heterocycloalkyl group having one or two heteroatoms independently selected from nitrogen, oxygen and sulfur;
m is 0, 1 or 2;
each R¹ is independently H, oxo, amino, cyano, halo, hydroxy, (C₁-C₄)alkyl, (C₃-C₄)cycloalkyl, (C₂-C₄)alkenyl, (C₁-C₄)alkoxy, mono-, di- or tri-fluoromethyl, hydroxy(C₁-C₄)alkyl, aminosulfonyl, methylsulfonylamino, (C₁-C₄)alkylsulfonyl, mono-N, or di-N,N—(C₁-C₄)amino, piperazinylcarbonyl, pyridinyl, pyrazinylcarbonyl, or R¹ forms an N-oxide when bonded to a ring nitrogen;
R² and R³ are H or R² and R³ are methyl; and
q is 0, 1 or 2.

Also provided herein, in some embodiments, are pharmaceutical compositions comprising a compound of Formula (I) or a pharmaceutically acceptable salt thereof and a pharmaceutically acceptable excipient.

Further provided herein, in some embodiments, are methods of modulating MRGPRX2 activity; and/or methods of modulating mast cell degranulation, and/or methods of treating, preventing or ameliorating an MRGPRX2-mediated disease or disorder in a subject in need thereof, wherein each of these methods independently comprises administering an effective amount of a compound of Formula (I) or a pharmaceutically acceptable salt thereof, or administering an effective amount of a pharmaceutical composition comprising a compound of Formula (I) or a pharmaceutically acceptable salt thereof and a pharmaceutically acceptable excipient.

Further provided herein, in some embodiments, are methods for treating, preventing or ameliorating a disease or conditions chosen from chronic spontaneous urticaria, mastocytosis, cold urticaria, atopic dermatitis, Asian atopic dermatitis, European atopic dermatitis, rosacea, autoimmune diseases, Crohn's disease, ulcerative colitis, irritable bowel syndrome, rheumatoid arthritis, fibromyalgia, endometriosis, nasal polyps, neuropathic pain, inflammatory pain, pseudo-allergic drug reactions, chronic itch, drug-induced anaphylactoid reactions, metabolic syndrome, esophagus reflux, asthma, cough, migraine, sinusitis, urticaria, chronic inducible urticaria, chronic pruritus, acute pruritus, prurigo nodularis, osteoarthritis, pseudo anaphylaxis, contact urticaria, lupus erythematosus (SLE), psoriasis, psoriatic arthritis, bronchial asthma, systemic mastocytosis, cutaneous mastocytosis, mastocytic enterocolitis, mast cell activation syndrome (MCAS), interstitial cystitis, food allergy, allergic rhinitis, microbial infection, eosinophilic esophagitis (EOE) and chronic pain, preferably wherein the condition is atopic dermatitis.

Also provided herein are compounds of Formula (I) or a pharmaceutically acceptable salt thereof for use as a medicament. In some embodiment the compound is for use in treating disease or disorder mediated by MRGPRX2.

Also provided herein is the use of a compound of Formula (I) or a pharmaceutically acceptable salt thereof in the manufacture of a medicament for treating a MRGPRX2-mediated disease or disorder.

### BRIEF DESCRIPTION OF THE DRAWINGS

FIG. 1A shows the crystal structure of Compound 3.

FIG. 1B is a table of crystal data and structure refinement for Compound 3.

FIG. 2A is the ¹H-NMR spectra of Compound 3 Form I.

FIG. 2B is the ¹H-NMR spectra of Compound 3 Form V.

FIG. 3A is the XRPD spectra of Compound 3 Form I.

FIG. 3B is the XRPD spectra of Compound 3 Form V.

FIG. 4A is the DSC spectra of Compound 3 Form I.

FIG. 4B is the DSC spectra of Compound 3 Form V.

FIG. 5A is the TGA spectra of Compound 3 Form I.

FIG. 5B is the TGA spectra of Compound 3 Form V.

FIG. 6 is the XRPD spectra of Compound 1 L-Tartrate salt.

FIG. 7 is the DSC spectra of Compound 1 L-Tartrate salt.

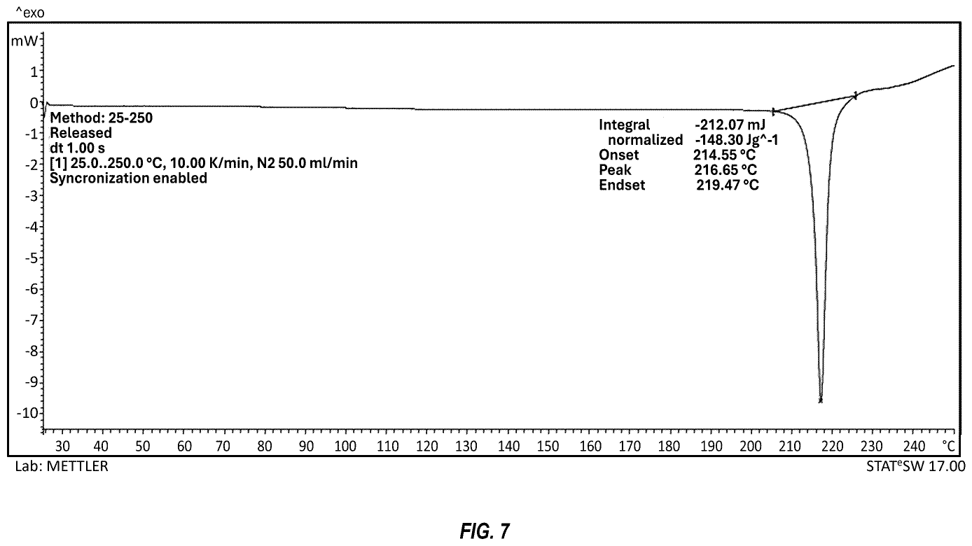

FIG. 8 is the TGA spectra of Compound 1 L-Tartrate salt.

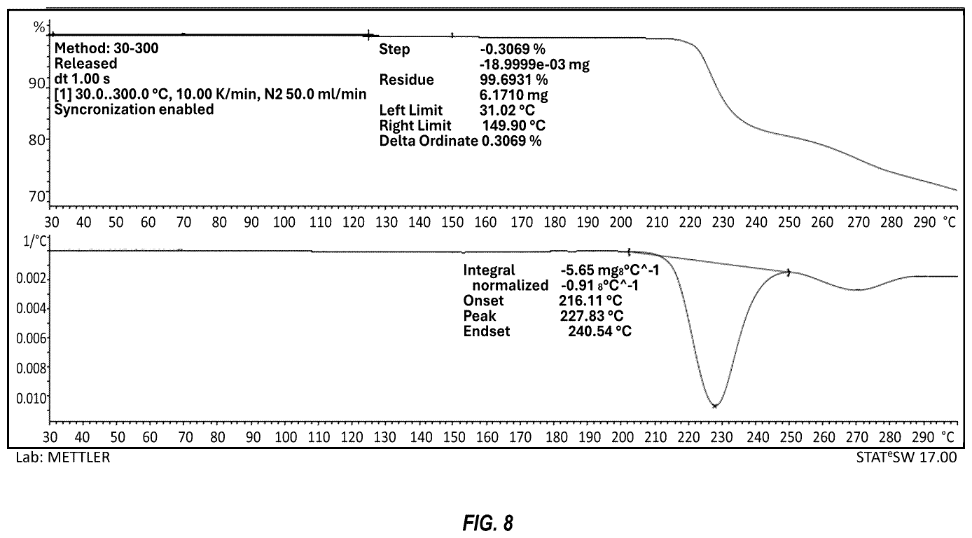

FIG. 9 shows that humanMRGPRX2 is expressed in X2-KI mice in the representative histogram showing peritoneal mast cells from wild type (WT) and X2-KI. Mast cells were identified as CD117+ FcεR1a+ and stained with anti-human MRGPRX2 (WT and X2-KI mice) or isotype control (X2-KI mice). Human MRGPRX2 is expressed in X2-KI mice (black histogram) compared to WT mice (grey histogram).

FIG. 10 shows that oral treatment of X2-KI mice with Compound 1 (3, 6, and 15 mg/kg (mpk)) inhibited mast cell degranulation and vascular permeability induced by the MRGPRX2 agonist C-14 (cortistatin-14). Data shown is Mean+/−SEM from n=10-12. Stats: One way-ANOVA with Dunnet's Multiple Comparisons. *P<0.05, ***P<0.0005, ****P<0.0001.

### DETAILED DESCRIPTION

Unless otherwise stated, the following terms used in the specification and claims are defined for the purposes of this disclosure and have the following meanings.

“Alkyl” means a saturated, straight, or branched hydrocarbon moiety having the specified number of carbon atoms. The term “(C₁-C₆)alkyl” refers to an alkyl moiety containing from 1 to 6 carbon atoms. Exemplary alkyls include, but are not limited to, methyl, ethyl, n-propyl, isopropyl, n-butyl, isobutyl, s-butyl, t-butyl, pentyl, and hexyl. and the like.

“Alkenyl” means a saturated, straight, or branched hydrocarbon moiety having the specified number of carbon atoms and at least one carbon-carbon double bond. The term “(C₁-C₆)alkenyl” refers to an alkenyl moiety containing from 1 to 6 carbon atoms. Exemplary alkenyls include, but are not limited to, ethenyl, n-propenyl, isopropenyl, n-butenyl, isobutenyl, s-butenyl, t-butenyl, pentenyl, and hexenyl. and the like.

“Alkoxy” means a —OR radical where R is alkyl as defined above, e.g., methoxy, ethoxy, propoxy, or 2-propoxy, n-, iso-, or tert-butoxy, and the like. The term “(C₁-C₄)alkoxy” refers to a straight- or branched-chain hydrocarbon radical having at least 1 and up to 4 carbon atoms attached through an oxygen linking atom.

When the term “alkyl” is used in combination with other substituent groups, such as “halo(C₁-C₄)alkyl”, “aryl(C₁-C₄)alkyl-”, or “(C₁-C₄)alkoxy(C₁-C₄)alkyl-”, the term “alkyl” is intended to encompass a divalent straight or branched-chain hydrocarbon radical, wherein the point of attachment is through the alkyl moiety. The term “halo(C₁-C₄)alkyl” is intended to mean a radical having one or more halogen atoms, which may be the same or different, at one or more carbon atoms of an alkyl moiety containing from 1 to 4 carbon atoms, which is a straight or branched-chain carbon radical. Examples of “halo(C₁-C₄)alkyl” groups useful in the present invention include, but are not limited to, —CF₃ (trifluoromethyl), —CCI₃ (trichloromethyl), 1,1-difluoroethyl, 2-fluoro-2-methylpropyl, 2,2-difluoropropyl, 2,2,2-trifluoroethyl, and hexafluoroisopropyl. Examples of “aryl(C₁-C₄)alkyl” or “phenyl(C₁-C₄)alkyl” groups useful in the present invention include, but are not limited to, benzyl and phenethyl. Examples of “(C₁-C₄)alkoxy(C₁-C₄)alkyl-” groups useful in the present invention include, but are not limited to, methoxymethyl, methoxyethyl, methoxyisopropyl, ethoxymethyl, ethoxyethyl, ethoxyisopropyl, isopropoxymethyl, isopropoxyethyl, isopropoxyisopropyl, t-butoxymethyl, t-butoxy ethyl, and t-butoxyisopropyl. As used herein, “(C₁-C₄)Alkylsulfonyl” useful in the present invention include, but are not limited to, —(O)₂S-methyl, —(O)₂S-ethyl, —(O)₂S-propyl, —(O)₂S-isopropyl, and the like.

As used herein, the term “cycloalkyl” refers to a non-aromatic, saturated, cyclic hydrocarbon ring containing the specified number of carbon atoms. For example, the term (C₃-C₈)cycloalkyl” refers to a non-aromatic cyclic hydrocarbon ring having from three to eight ring carbon atoms. Exemplary “(C₃-C₈)cycloalkyl” groups include cyclopropyl, cyclobutyl, cyclopentyl, cyclohexyl, cycloheptyl, and cyclooctyl.

As used herein, “heterocycloalkyl” represents a group or moiety comprising a nonaromatic, monovalent monocyclic radical, which is saturated or partially unsaturated and includes for example, one to three heteroatoms, selected independently from oxygen, sulfur, and nitrogen. For example, the term “5 to 7 membered heterocycloalkyl” refers to a heterocycloalkyl group containing 5, 6 or 7 ring atoms, which includes for example one to three heteroatoms selected independently from oxygen, sulfur, and nitrogen. Illustrative examples of 4 to 6 membered heterocycloalkyl groups useful in the present invention include, but are not limited to azetidinyl, oxetanyl, pyrrolidinyl, pyrazolidinyl, pyrazolinyl, imidazolidinyl, imidazolinyl, oxazolinyl, thiazolinyl, tetrahydrofuranyl, dihydrofuranyl, 1,3-dioxolanyl, piperidinyl, piperazinyl, morpholinyl, thiomorpholinyl, tetrahydropyranyl, dihydropyranyl, 1,3-dioxanyl, 1,4-dioxanyl, 1,3-oxathiol any 1,1,3-oxathianyl, 1,3-dithianyl, 1,4-oxathiolanyl, 1,4-oxathianyl, and 1,4-dithianyl.

As used herein, “amino” refers to primary, secondary, and tertiary nitrogen containing groups.

As used herein “halo” means fluoro, chloro, bromo, or iodo; in one embodiment halo is fluoro or chloro.

As used herein “hydroxy” means an —OH group.

As used herein “cyano” means a —CN group.

As used herein, unless otherwise stated, “aryl” refers to monovalent monocyclic, fused bicyclic, or fused tricyclic groups having 6 to 16 carbon atoms and having at least one aromatic ring that complies with Huckel's Rule. Examples of “aryl” groups are phenyl, naphthyl, indenyl, dihydroindenyl, anthracenyl, phenanthrenyl, and the like. As used herein, “arylene” refers to a bivalent aryl group.

As used herein, unless otherwise stated, “heteroaryl” represents a group or moiety comprising an aromatic monovalent monocyclic or bicyclic radical, or tricyclic radical containing 5 to 16 ring atoms, including 1 to 6 heteroatoms independently selected from nitrogen, oxygen, and sulfur. Illustrative examples of heteroaryl groups useful in the present invention include, but are not limited to, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, triazinyl, benzofuranyl, indolizinyl, indolyl, isoindolyl, dihydroindolyl, benzimidazolyl, benzoxazolyl, benzthiazolyl, benzoisothiazolyl, indazolyl, imidazopyridinyl, pyrazolopyridinyl, benzotriazolyl, triazolopyridinyl, purinyl, quinolinyl, isoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl.

As used herein “Oxo” means an ═(O) group where the oxygen is bound to any atom and “carbonyl” means a >C(O) or >C═O or C═O group.

As used herein “sulfonyl” refers to an S(O)₂ group.

As used herein “aminosulfonyl” refers to a H₂NS(O)₂— group.

“Mammal” as used herein means domesticated animals (such as dogs, cats, and horses), and humans. In one embodiment, mammal is a human.

It is to be understood that if a carbocyclic or heterocyclic moiety may be bonded or otherwise attached to a designated substrate through differing ring atoms without denoting a specific point of attachment, then all possible points are intended, whether through a carbon atom or, for example, a trivalent nitrogen atom. For example, the term “pyridyl” means 2-, 3-, or 4-pyridyl, the term “thienyl” means 2-, or 3-thienyl, and so forth.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

It is understood that the pharmaceutically acceptable salts are non-toxic. For a review on suitable salts, see Handbook of Pharmaceutical Salts. Properties, Selection, and Use by Stahl and Wermuth (Wiley-VCH, 2002). Additional information on suitable pharmaceutically acceptable salts can be found in Remington The Science and Practice of Pharmacy, 23rd ed., Elsevier Science, 2020, which is incorporated herein by reference.

As used herein “optional” or “optionally” means that the subsequently described event or circumstance may but need not occur, and that the description includes instances where the event or circumstance occurs and instances in which it does not. For example, “heterocycloalkyl group optionally substituted with an alkyl group” means that the alkyl may but need not be present, and the description includes situations where the heterocycloalkyl group is substituted with an alkyl group and situations where the heterocycloalkyl group is not substituted with alkyl.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” (includes pharmaceutically acceptable carriers, stabilizers, and the like) as described herein.

“Treating” or “treatment” of a disease includes:
(1) preventing the disease, e.g., causing the clinical symptoms of the disease not to develop in a mammal that may be exposed to or predisposed to the disease but does not yet experience or display symptoms of the disease;
(2) inhibiting the disease, e.g., arresting or reducing the development of the disease or its clinical symptoms; or
(3) relieving the disease, e.g., causing regression of the disease or its clinical symptoms. Thus, prevent, preventing, and the like can refer to the prevention of the disease or condition in the patient. For example, if an individual at risk of contracting a condition/disease is treated with the methods of the present disclosure and does not later contract the condition/disease, then the disease has been prevented, at least over a period of time, in that individual.

A “therapeutically effective amount” means the amount of a compound of Formula (I) (or any of the embodiments thereof described herein), that, when administered to a mammal for treating a disease, is sufficient to treat the disease. The “therapeutically effective amount” will vary depending on the compound, the disease and its severity and the age, weight, etc., of the mammal to be treated as further described herein.

The compounds described herein may in some cases exist as diastereomers, enantiomers, or other stereoisomeric forms. A “stereoisomer” refers to a compound made up of the same atoms bonded by the same bonds but having different three-dimensional structures, which are not interchangeable. The present disclosure contemplates various stereoisomers and mixtures thereof and includes “enantiomers,” which refers to two stereoisomers whose molecules are nonsuperimposable mirror images of one another. or “diastereomers” are stereoisomers that have at least two asymmetric atoms, but which are not mirror-images of each other. Thus, the compounds of the present invention can exist in the form of various stereoisomers, R and S isomers, depending upon the presence of asymmetric carbon atoms. As used herein, they may be referred to as the “R configuration’ or “S configuration” or the like. The present invention encompasses both the individual isomers and mixtures thereof, including racemic and diastereomeric mixtures. All chiral, diastereomeric, racemic forms, as individual forms, and mixtures thereof, are within the scope of this disclosure, unless the specific stereochemistry or isomeric form is specifically indicated.

The term “stereoisomer” compound as used herein refers to one stereoisomer (for example, geometric isomer, enantiomer, diastereomer and atropoisomer) of a compound that is substantially free of other stereoisomers of that compound. For example, a stereoisomerically pure compound having one chiral center will be substantially free of the mirror image enantiomer of the compound and a stereoisomerically pure compound having two chiral centers will be substantially free of the other enantiomer and diastereomers of the compound. A typical stereoisomerically pure compound comprises greater than about 80% by weight of one stereoisomer of the compound and equal or less than about 20% by weight of other stereoisomers of the compound, greater than about 90% by weight of one stereoisomer of the compound and equal or less than about 10% by weight of the other stereo isomers of the compound, greater than about 95% by weight of one stereoisomer of the compound and equal or less than about 5% by weight of the other stereoisomers of the compound, or greater than about 97% by weight of one stereoisomer of the compound and equal or less than about 3% by weight of the other stereoisomers of the compound.

Compounds of the present disclosure containing an asymmetrically substituted atom may be isolated in optically active, optically enriched, optically pure, or racemic forms. It is well known in the art how to prepare optically active forms, such as by resolution of materials. Separation of stereoisomers may be performed by chromatography or by forming diastereomers and separating by recrystallization, or chromatography, or any combination thereof (Jean Jacques, Andre Collet, Samuel H. Wilen, “Enantiomers, Racemates and Resolutions,” John Wiley and Sons, Inc., 1981, herein incorporated by reference for this disclosure). Stereoisomers may also be obtained by stereoselective synthesis. Those skilled in the art are familiar with methods for determining absolute stereochemistry e.g., X-Ray powder diffraction. Stereoisomers may also be obtained by stereoselective synthesis into their compounding pure enantiomers by forming a diastereomeric salt with an optically pure chiral base or acid (e.g., 1-phenyl-ethylamine or tartaric acid) and separating the diastereomers by fractional crystallization followed by neutralization to break the salt, thus providing the corresponding pure enantiomers.

Certain compounds of Formula (I) (or any of the embodiments thereof described herein) and/or a pharmaceutically acceptable salt thereof may exist as tautomers and/or geometric isomers. All possible tautomers and cis and trans isomers, as individual forms and mixtures thereof, are within the scope of this disclosure. For example, pyrazole tautomers as shown below are equivalent structures. The depiction of one such structure is intended to encompass both structures.

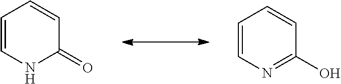 [SMILES: O=c1cccc[nH]1 ; Oc1ccccn1]

Additionally, as used herein the term alkyl includes all the possible isomeric forms of said alkyl group albeit only a few examples are set forth. Furthermore, when the cyclic groups such as heteroaryl, heterocyclyl are substituted, they include all the positional isomers.

The compounds described herein include hydrates and solvates of the compounds or pharmaceutically acceptable salts thereof. The term solvate is used herein to describe a molecular complex comprising the compound of the invention and one or more pharmaceutically acceptable solvent molecules, for example, ethanol. Such solvent molecules are those commonly used in the pharmaceutical art, which are known to be innocuous to the recipient, e.g., water, ethanol, and the like. Other solvents may be used as intermediate solvates in the preparation of more desirable solvates, such as methanol, methyl t-butyl ether, ethyl acetate, methyl acetate, (S)-propylene glycol, (R)-propylene glycol, 1,4-butyne-diol, and the like.

The term hydrate is employed when said solvent is water. Pharmaceutically acceptable solvates include hydrates and other solvates wherein the solvent of crystallization may be isotopically substituted, e.g., D₂O. d-acetone, d-DMSO. The solvates and/or hydrates preferably exist in crystalline form. A classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

The present disclosure also includes the prodrugs of compounds of Formula (I) (or any of the embodiments thereof described herein) and/or a pharmaceutically acceptable salt thereof. The term prodrug is intended to represent covalently bonded carriers, which are capable of releasing the active ingredient of Formula (I) (or any of the embodiments thereof described herein) when the prodrug is administered to a mammalian subject. Release of the active ingredient occurs in vivo. Prodrugs can be prepared by techniques known to one skilled in the art. These techniques generally modify appropriate functional groups in a given compound. These modified functional groups, however, regenerate original functional groups in vivo or by routine manipulation. Prodrugs of compounds of Formula (I) (or any of the embodiments thereof described herein) include compounds wherein a hydroxy, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups in compounds of Formula (I)), amides (e.g., trifluoroacetylamino, acetylamino, and the like), and the like. Prodrugs of compounds of Formula (I) (or any of the embodiments thereof described herein) and/or a pharmaceutically acceptable salt thereof are also within the scope of this disclosure.

Also included within the scope of the invention are multi-component complexes (other than salts and solvates) wherein the drug and at least one other component are present in stoichiometric or non-stoichiometric amounts. The compounds of the invention may also exist as complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionized, partially ionized, or non-ionized. For a review of such complexes, see J Pharm Sci, 64 (8), 1269-1288 by Haleblian (August 1975).

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration. Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e., as a carrier, diluent, or solubilizer. Most commonly used for these purposes are alpha-, beta- and gamma-cyclodextrins.

The present disclosure also includes polymorphic forms (amorphous as well as crystalline). Thus, the compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

Certain compounds of the present invention or combination agents may exist in more than one crystal form (generally referred to as “polymorphs”). Polymorphs may be prepared by crystallization under various conditions, for example, using different solvents or different solvent mixtures for recrystallization; crystallization at different temperatures; and/or various modes of cooling, ranging from very fast to very slow cooling during crystallization. Polymorphs may also be obtained by heating or melting the compound of the present invention followed by gradual or fast colling. The presence of polymorphs may be determined by solid probe NMR spectroscopy, IR spectroscopy, differential scanning calorimetry, powder X-ray diffraction or such other techniques.

The compounds disclosed herein, in some embodiments, are used in different enriched isotopic forms. Thus, the compounds of the present disclosure optionally contain unnatural proportions of atomic isotopes at one or more atoms that constitute such compounds. For example, the compounds may be labeled with isotopes, such as for example, deuterium (2H), tritium (3H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds of the present invention,

For example, compounds having the present structures except for the replacement of a hydrogen by a deuterium or tritium, or the replacement of a carbon by ¹³C- or ¹⁴C-enriched carbon are within the scope of the present disclosure. In certain embodiments, the compounds disclosed herein have some or all of the ¹H atoms replaced with ²H atoms. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs. Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32. Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Also included within the scope of the invention are metabolites of compounds of Formula I, that is, compounds formed in vivo upon administration of the drug. Some examples of metabolites in accordance with the invention include
(i) where the compound of Formula I contains a methyl group, an hydroxymethyl derivative thereof (—CH₃->—CH₂OH);
(ii) where the compound of Formula I contains an alkoxy group, an hydroxy derivative thereof (—OR->—OH);
(iii) where the compound of Formula I contains a tertiary amino group, a secondary amino derivative thereof (—NRR->—NHR or —NHR);
(iv) where the compound of Formula I contains a secondary amino group, a primary derivative thereof (—NHR->—NH₂);
(v) where the compound of Formula I contains a phenyl moiety, a phenol derivative thereof (-Ph->-PhOH); and
(vi) where the compound of Formula I contains an amide group, a carboxylic acid derivative thereof (—CONH₂->COOH).

Provided herein is a compound of Formula (I) or a pharmaceutically acceptable salt thereof:

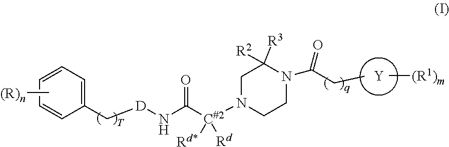

wherein
T is 1 or 0; provided that when T is 1, D is a 5-membered heteroaryl group having one or two heteroatoms selected from nitrogen and oxygen, and D is unsubstituted or is mono-substituted with (C₁-C₄)alkyl, mono-, di-, or tri-fluoromethyl; and
when T is 0, D is

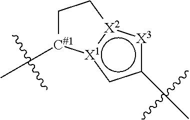

and;
X¹ and X² are each independently N or C;
X³ is N or CH;
C^{#1} is a first stereocenter;
C^{#2} is a second stereocenter;
n is 1, 2 or 3;
each R is independently H or halo;
either R^{d*} is H and R^{d} is methyl or R^{d*} is methyl and R^{d} is H;
Y is absent or Y is selected from
   a 5- or 6-membered heteroaryl group having one to three heteroatoms independently selected from nitrogen, oxygen and sulfur;
   a 4- to 6-membered heterocycloalkyl group having 1 to 2 heteroatoms independently selected from nitrogen, oxygen and sulfur;
   a phenyl ring fused to a 5- or 6-membered heteroaryl group to form a bicyclic group having one to three heteroatoms independently selected from nitrogen, oxygen and sulfur;
   a 5- or 6-membered heteroaryl group fused to a 5- or 6-membered heteroaryl group to form a bicyclic group having one to five heteroatoms independently selected from nitrogen, oxygen and sulfur;
   a 5- to 7-membered heterocycloalkyl group fused to a 5- or 6-membered heteroaryl group to form a bicyclic group having one to four heteroatoms independently selected from nitrogen, oxygen and sulfur;
   a (C₅-C₇)cycloalkyl group fused to a 5- or 6-membered heteroaryl group to form a bicyclic group, said 5- or 6-membered heteroaryl group having one to three heteroatoms independently selected from nitrogen, oxygen and sulfur;
   a phenyl group;
   a (C₄-C₇)cycloalkyl group; or
   a phenyl group fused to a 5- to 7-membered heterocycloalkyl group to form a bicyclic group, said heterocycloalkyl group having one or two heteroatoms independently selected from nitrogen, oxygen and sulfur;
m is 0, 1 or 2;
each R¹ is independently H, oxo, amino, cyano, halo, hydroxy, (C₁-C₄)alkyl, (C₃-C₄)cycloalkyl, (C₂-C₄)alkenyl, (C₁-C₄)alkoxy, mono-, di- or tri-fluoromethyl, hydroxy(C₁-C₄)alkyl, aminosulfonyl, methylsulfonylamino, (C₁-C₄)alkylsulfonyl, mono-N, or di-N,N—(C₁-C₄)amino, piperazinylcarbonyl, pyridinyl, pyrazinylcarbonyl, or R¹ forms an N-oxide when bonded to a ring nitrogen; R² and R³ are H or R² and R³ are methyl; and
q is 0, 1 or 2.

In some embodiments, D is a 5-membered heteroaryl group having one to two heteroatoms independently selected from nitrogen and oxygen and D is unsubstituted or mono-substituted with (C₁-C₄)alkyl or mono-, di-, or tri-fluoromethyl. In some embodiments, D is a

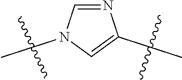 [SMILES: *c1cn(*)cn1]

group, unsubstituted or mono-substituted with (C₁-C₄)alkyl or mono-, di-, or tri-fluoromethyl. In some embodiments, D is a

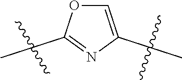 [SMILES: *c1coc(*)n1]

group, unsubstituted or mono-substituted with (C₁-C₄)alkyl or mono-, di-, or tri-fluoromethyl. In some embodiments, D is substituted with methyl, or di-fluoromethyl, or tri-fluoromethyl. In some embodiments, D is substituted with methyl. In some embodiments, D is substituted with di-, or tri-fluoromethyl. In

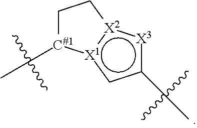

some embodiments, D is.

In some embodiments, R is halo. In some embodiments, R is fluoro.

In some embodiments, n is 2.

In some embodiments, X¹ and X³ are N and X² is C. In some embodiments, X² and X³ are N and X¹ is C.

In some embodiments, the first stereocenter is in an R configuration. In some embodiments, the first stereocenter is in an S configuration. In some embodiments, the second stereocenter is in an R configuration. In some embodiments, the second stereocenter is in an S configuration.

In some embodiments, R² and R³ are H. In some embodiments, R² and R³ are methyl.

In some embodiments, q is 0. In some embodiments, q is 1. In some embodiments, q is 2.

In some embodiments, Y is a 5- or 6-membered heteroaryl group having one to three heteroatoms independently selected from nitrogen, oxygen, and sulfur. In some embodiments, Y is a 4- to 6-membered heterocycloalkyl group having one or two heteroatoms independently selected from nitrogen, oxygen, and sulfur. In some embodiments, Y is a phenyl group fused to a 5- or 6-membered heteroaryl group to form a bicyclic group having one to three heteroatoms independently selected from nitrogen, oxygen, and sulfur. In some embodiments, Y is a 5- or 6-membered heteroaryl group fused to a 5- or 6-membered heteroaryl group to form a bicyclic group having one to five heteroatoms independently selected from nitrogen, oxygen, and sulfur. In some embodiments, Y is a 5- to 7-membered heterocycloalkyl group fused to a 5- or 6-membered heteroaryl group to form a bicyclic group having one to four heteroatoms independently selected from nitrogen, oxygen, and sulfur. In some embodiments, Y is a (C₅-C₇)cycloalkyl group fused to a 5- or 6-membered heteroaryl group, said 5- or 6-membered heteroaryl group having one to three heteroatoms independently selected from nitrogen, oxygen, and sulfur. In some embodiments, Y is a phenyl group. In some embodiments, Y is a (C₅-C₇)cycloalkyl group. In some embodiments, Y is a phenyl group fused to a 5- to 7-membered heterocycloalkyl group having one or two heteroatoms independently selected from nitrogen, oxygen, and sulfur.

In some embodiments, R¹ is oxo or R¹ forms an N-oxide when bonded to a ring nitrogen. In some embodiments, R¹ is oxo or R¹ forms an N-oxide when bonded to a ring nitrogen; and m is 1.

In some embodiments, R¹ is fluoro, (C₁-C₃)alkyl, (C₃-C₄)cycloalkyl, amino, cyano, hydroxy or hydroxy(C₁-C₂)alkyl; and m is 1 or 2.

In some embodiments, Y is

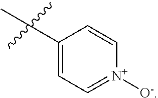

In some embodiments, Y is

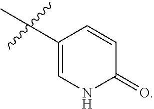 [SMILES: *c1ccc(=O)[nH]c1]

In some embodiments, Y is

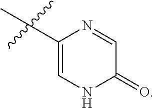 [SMILES: *c1c[nH]c(=O)cn1]

In some embodiments, Y is

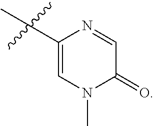 [SMILES: *c1cn(C)c(=O)cn1]

In some embodiments, Y is

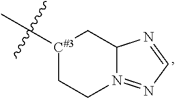

wherein C^{#3} is a third stereocenter. In some embodiments, third stereocenter is in an R configuration. In some embodiments, third stereocenter is in an S configuration.

In some embodiments, Y is

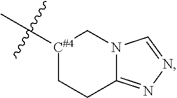

wherein C^{#4} is a fourth stereocenter. In some embodiments, the fourth stereocenter is in an R configuration. In some embodiments, the fourth stereocenter is in an S configuration.

In some embodiments, Y is

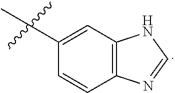 [SMILES: *c1ccc2nc[nH]c2c1]

In some embodiments, D is

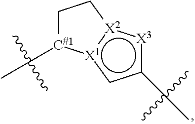

is halo, n is 2, X¹ and X³ are N, X² is C, and q is 0 or 1. In some embodiments, R² and R³ are each methyl and q is 0.

In some embodiments, D is

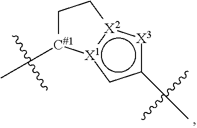

R is halo, n is 2, X² and X³ are N, X¹ is C, and q is 0 or 1. In some embodiments, wherein R² and R³ are each methyl and q is 0.

In some embodiments, Y is a 5- or 6-membered heteroaryl group having one or two nitrogen, R¹ is oxo or R¹ forms an N-oxide when bonded to a ring nitrogen, and m is 1.

In some embodiments, Y is a 5-6 membered heteroaryl ring having one to two nitrogen, R¹ is (C₁-C₄)alkyl and R¹ is oxo or R¹ forms an N-oxide when bonded to a ring nitrogen and m is 2.

In some embodiments, the compound of Formula (I) is a compound as recited in the Examples.

### General Methods

Certain processes for the manufacture of the compounds of this invention are provided as further features of the invention and are illustrated by the following exemplary reaction schemes. Those skilled in the art will appreciate that other synthetic routes may be used to synthesize the inventive compounds. For a more detailed description of the individual reaction steps, see the Examples section below. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art. In particular, it is noted that the compounds prepared according to these Schemes may be modified further to provide new Examples within the scope of this invention. In addition, it will be evident from the detailed descriptions given in the Experimental section that the modes of preparation employed extend further than the general procedures described herein. Additionally, general principles of organic chemistry are described in “Organic Chemistry”, Thomas Sorrell, University Science Books, Sausalito: 2005, and “March's Advanced Organic Chemistry: Reactions Mechanisms and Structure”, 8^{th} Ed., Ed.: Smith, M. B., John Wiley & Sons, New York: 2019, the entire contents of which are hereby incorporated by reference.

The starting materials are generally available from commercial sources such as Merck Sigma-Aldrich Inc. and Enamine Ltd. Aldrich Chemicals (Milwaukee, Wis.) or are readily prepared using methods known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including Supplements (also available via the Beilstein online database).

As an initial note, in the preparation of compounds of the present invention, it is noted that some of the preparation methods useful for the preparation of the compounds described herein may require protection of remote functionality (e.g., primary amine, secondary amine, carboxyl in intermediates). The need for such protection will vary depending on the nature of the remote functionality and the conditions of the preparative methods and can be readily determined by one of ordinary skill in the art. The use of such protection/deprotection methods is also within the ordinary skill in the art. For a general description of protecting groups and their use, see T. W. Greene, Protective Groups in Organic Synthesis, John Wiley & Sons, New York, 1991 and Greene's Protective Groups inorganic Synthesis, John Wiley &Sons, New York 2006.

For example, certain compounds contain primary amines or carboxylic acid functionalities which may interfere with reactions at other sites of the molecule if left unprotected. Accordingly, such functionalities may be protected by an appropriate protecting group which may be removed in a subsequent step. Suitable protecting groups for amine and carboxylic acid protection include those protecting groups commonly used in peptide synthesis (such as N-t-butoxycarbonyl, benzyloxycarbonyl, and 9-fluorenylmethylenoxycarbonyl for amines and lower alkyl or benzyl esters for carboxylic acids) which are generally not chemically reactive under the reaction conditions described and can typically be removed without chemically altering other functionality in the Formula I compound.

As can be appreciated by the skilled artisan, the above synthetic Schemes and representative examples (below) are not intended to comprise a comprehensive list of all means by which the compounds described and claimed in this application may be synthesized. Further methods will be evident to those of ordinary skill in the art. Additionally, the various synthetic steps described above may be performed in an alternate sequence or order to give the desired compounds. The disclosure further encompasses “intermediate” compounds, including structures produced from the synthetic procedures described, whether isolated or generated in-situ and not isolated, prior to obtaining the finally desired compound. These intermediates are included in the scope of this disclosure. Exemplary embodiments of such intermediate compounds are set forth in the Examples below.

Unless stated otherwise, the variables introduced in the General Methods schemes are as defined herein.

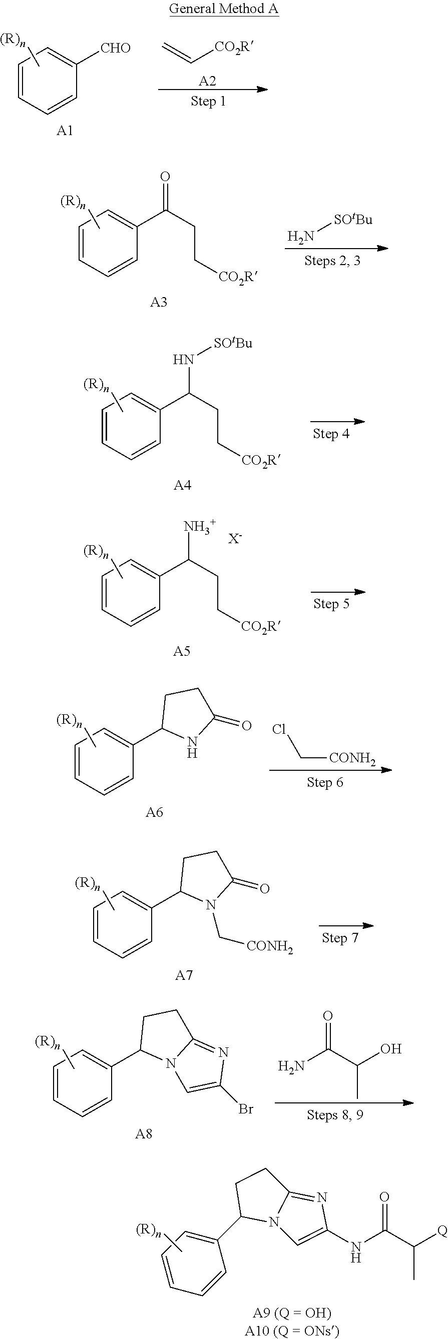

General Method A describes a method for the synthesis of 6,7-dihydro-5H-pyrrolo[1,2-a]imidazole intermediates represented by structural formula A10. In the scheme, R′ is alkyl; X⁻ is the counterion of a strong acid, such as Cl⁻; ONs' is a sulfonate leaving group such as 2-nitrobenzenesulfonyl (ONs), OTs, OMs, etc.; and all other variables are as defined herein.

Step 1 is a Stetter reaction (Stetter, H. et al., Chem. Ber. 1976, 109, 541-545) wherein benzaldehyde or a substituted variant thereof (A1) is treated with sodium cyanide and an acrylic ester (A2). The reaction can be conducted in a polar aprotic solvent such as N, N-dimethylformamide at a temperature of about 0-80° C., typically around 0-65° C. Preferably, the reaction is maintained at around 25-65° C. by means of a water bath to control any exothermic rise in temperature. The product of the reaction is a γ-keto ester, A3.

In Step 2, A3 is condensed with 2-methylpropane-2-sulfinamide in neat titanium (IV) ethoxide at a temperature of around 55-110° C., typically at about 65-80° C., and preferably at around 70° C. When conducted at the preferred temperature, the reaction requires about 2.5-3.5 hours. The product of the reaction, an iminosulfinamide (structure not shown), can be isolated if desired or can be reduced in situ.

Step 3 describes a process for the in-situ reduction of the iminosulfinamide whereby the reaction mixture from Step 2 is diluted with an ethereal solvent and treated with a reducing agent. Suitable reductants for iminosulfinamides include sodium borohydride (WO/2024/250985), diethyl zinc (Liu, T. et al., J. Org. Chem. 2015, 80, 11441-11446), diisobutylaluminum hydride (DIBAL-H) (WO/2013/030138), and lithium tri-sec-butylborohydride (L-Selectride) (Colyer, J. T. et al., J. Org. Chem. 2006, 71, 6859-6862). In a preferred example, the Step 2 product mixture is diluted with tetrahydrofuran and treated with L-Selectride at −78° C. for 1-2 hours to afford the A4 sulfinamide.

In Step 4, A4 is treated with a strong acid such as hydrogen chloride in an alcoholic solvent such as methanol. This reaction can be conducted between about 0-80° C. and is typically run at about room temperature for around 8-16 hours. The product of the reaction is an amine salt, A5.

In Step 5, base and heat are applied to A5 to induce lactamization. For example, the amine salt can be suspended in a polar aprotic solvent such as 1,4-dioxane and treated with an organic base such as DBU at a temperature of about 65-110° C., preferably around 85° C. When conducted at the preferred temperature, the reaction typically requires about 12-16 hours, and the product of the reaction is a pyrrolidinone, A6.

In Step 6, A6 is alkylated with 2-chloroacetamide or a functional equivalent. In this reaction, A6 is first treated with a strong base such as sodium hydride to effect deprotonation of the pyrrolidinone nitrogen atom. This type of reaction is common in the art and is usually conducted in an ethereal solvent such as tetrahydrofuran at a temperature of about 0° C. to about room temperature for about 5-30 min. Upon deprotonation, 2-chloroacetamide is added followed by stirring for around 2-24 hours. When conducted at room temperature, the reaction is expected to take around 2-8 hours, although certain sterically hindered substrates may require more reaction time or heating to about 60-85° C. The product of the reaction is A7.

Step 7 describes a dehydration/cyclization reaction whereby A7 is converted into A8. In this transformation, A7 is dissolved in an inert polar aprotic solvent such as acetonitrile and treated with a dehydrating reagent such as phosphoryl tribromide at about 0° C. The mixture is subsequently heated to about 65-110° C., preferably around 80° C., for around 2 hours to afford A8.

Step 8 is a copper-mediated N-arylation reaction between A8 and 2-hydroxypropanamide. In practice, A8 is mixed with copper (I) iodide, a chelating diamine such as N, N′-dimethylethylenediamine (DMEDA) or N¹,N²-dimethylcyclohexane-1,2-diamine, 2-hydroxypropanamide, and an inorganic base such as potassium carbonate, in a polar aprotic solvent at around 70-120° C. In a typical example, the reaction mixture is prepared in acetonitrile at room temperature and subsequently heated at reflux overnight to afford A9.

In Step 9, A9 is converted into a sulfonate by treating with a sulfonating agent. Such agents include p-toluene sulfonyl chloride, p-toluene sulfonic anhydride, and similar reagents including 2-nitrobenzenesulfonyl chloride (NsCl). In a preferred example, a solution of A9 in a polar aprotic solvent such as dichloromethane in the presence of 4-dimethylaminopyridine (DMAP) and an organic base such as triethylamine is treated with NsCl. The reaction may be conducted from about −10-60° C., preferably between about 0° C. and about room temperature. When conducted at the preferred temperature, the reaction is typically complete within about 2-6 hours, and the product of the reaction is the sulfonate represented by A10.

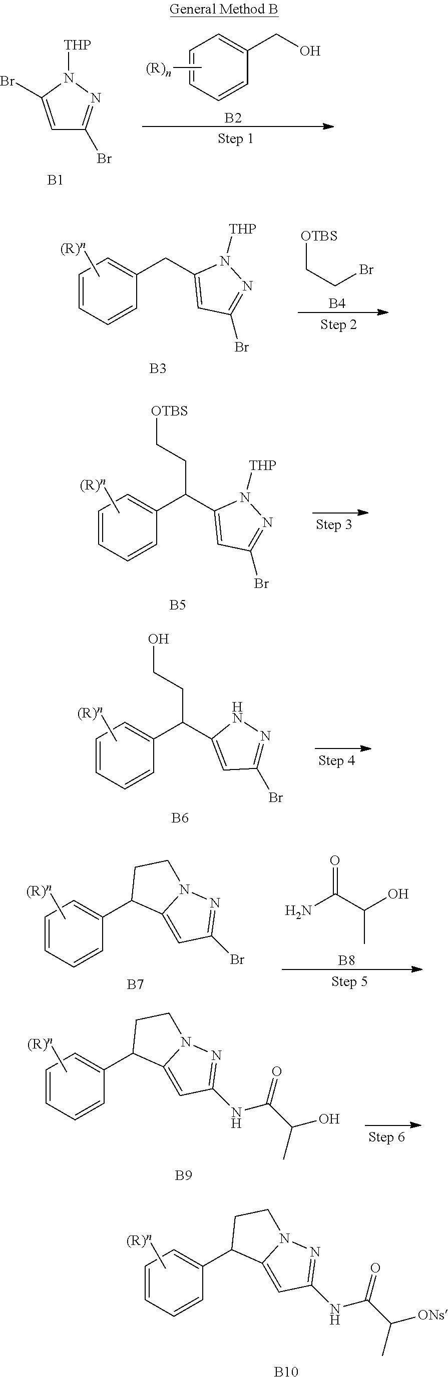

General Method B describes a method for the synthesis of 5,6-dihydro-4H-pyrrolo[1,2-b]pyrazole derivatives represented by structural formula B10. In the scheme, THP represents a pyrazole protecting group such as tetrahydropyranyl or Boc; OTBS represents a silyl ether hydroxyl protecting group such as a tert-butyldimethyl silyl ether; ONs' is a sulfonate leaving group such as 2-nitrobenzenesulfonyl (ONs), OTs, OMs, etc.; and all other variables are as defined herein.

In Step 1, commercially sourced, N-protected pyrazole B1 undergoes iridium catalyzed SP²-SP³ cross coupling reaction, also known as deoxygenative alkylation (Plunkett, S. et al., Org. lett. 2025, 11689-11694; Dong, Z. and MacMillan, D. W. C., Nature 2021, 598, 451-456). In this reaction, a degassed solution of the alcohol B2 is mixed with 5,7-di-tert-butyl-3-phenylbenzo[d]oxazol-3-ium tetrafluoroborate and pyridine in an ethereal solvent such as methyl-tert-butyl ether at a temperature of around 0-25° C. for 10-20 min. The mixture is then added to a solution of an iridium catalyst such as [Ir(ppy)₂(dtbbpy)]PF₆, [4,4′-Bis(tert-butyl)-2,2′-bipyridine]nickel dibromide, 1-azabicyclo[2.2.2]octane, and the dibromopyrazole B1 in a polar aprotic solvent such as N,N-dimethylacetamide, followed by irradiation under 450 nm LED modules for around 12-24 hours, typically around 16-18 hours, to afford B3.

In Step 2, B3 is lithiated by treatment with lithium diisopropylamide (LDA) in an ethereal solvent such as THF at a temperature of about −60° C. to −90° C., typically around −78 ° C., for 2-20 min, typically 5-10 min. The mixture is then treated with B4 or a functional equivalent and held for about 1-6 hours, typically around 2 hours, prior to warming to around 0° C. where it is quenched with a solution of ammonium chloride. The product of the reaction is a silyl ether, B5.

Step 3 is a bis-deprotection reaction involving cleavage of both the THP and silyl ether groups. Each of these transformations is well documented in the literature and can be accomplished in one step through treatment with a strong acid in any of a variety of different polar protic and polar aprotic solvents. For example, treatment of B5 with HCl in dioxane at around 0-80° C. for around 1-8 hours affords cleavage of both groups. When conducted at the preferred temperature of around 25° C., the reaction is expected to take around 2 hours.

Step 4 is known by those skilled in the art as an intramolecular Mitsunobu reaction. In this transformation, B6 is treated with triphenylphosphine and a dialkyl azodicarboxylate such as diisopropyl azodicarboxylate in a polar aprotic solvent such as THF. The reaction is typically initiated at around ice bath temperature where it is held for 10-15 min prior to warming to around 25-60° C., preferably room temperature, for about 1-12 hours. When conducted at the preferred temperature, the reaction is expected to require around 2-4 hours. The product of the reaction is a pyrrolopyrazole derivative, represented by B7.

The transformations depicted in Steps 5 and 6 include a copper-mediated N-arylation reaction to afford B9 and subsequent conversion into a sulfonate, B10. These reactions were described previously herein.

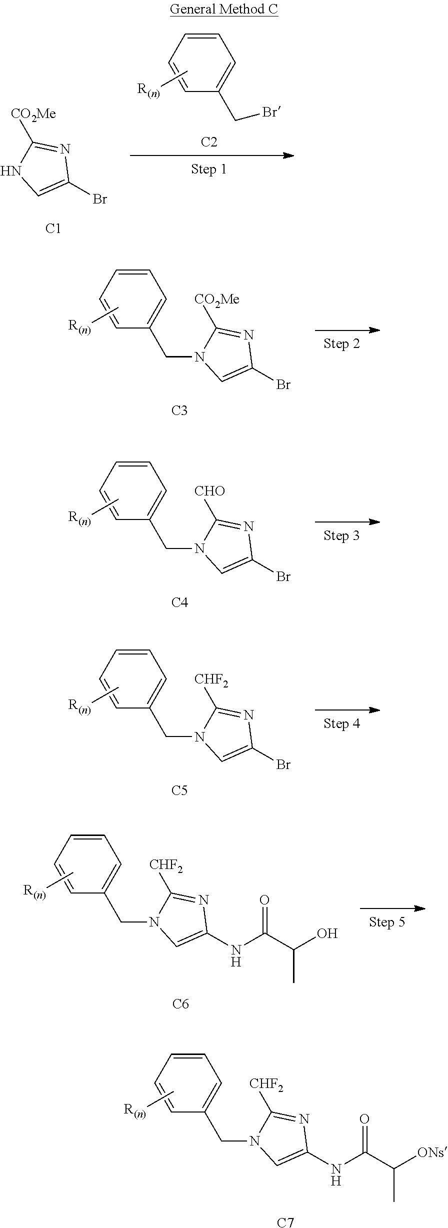

General Method C describes a synthetic sequence for the preparation of 2-difluoromethyl imidazole derivatives represented by structural formula C7. In the scheme, Br′ is a leaving group such as Br, Cl, I, or a sulfonate; ONs' is a sulfonate leaving group such as 2-nitrobenzenesulfonyl (ONs), OTs, OMs, etc.; and all other variables are as described herein.

In Step 1, commercially available methyl 4-bromo-1H-imidazole-2-carboxylate (C1) undergoes S_{N}² alkylation with a benzylic electrophile, represented by C2. This reaction, which is common in the art, is typically performed in a polar aprotic solvent in the presence of an organic or inorganic base. For example, the reaction may be performed in acetonitrile or N, N-dimethylformamide in the presence of N, N-diisopropylethylamine or potassium carbonate. This reaction can be conducted from around 0-160° C., with the effective temperature and required time dependent on the nature of C2. Most commonly, the reaction is run between room temperature and 70° C. For example, a stirred mixture of C1, C2, and potassium carbonate in acetonitrile affords the N-benzylimidazole C3 within around 18 hours at room temperature.

In Step 2, the ester C3 is reduced to the aldehyde C4. One skilled in the art will appreciate that this transformation can be realized in two steps. For example, the ester may be reduced to the corresponding alcohol with a reducting agent, such as a borohydride or an aluminum hydride, in an ethereal solvent, and subsequently oxidized to the aldehyde via the well-known Swern or Dess-Martin oxidation. One skilled in the art will recognize that C4 may also be prepared in one step upon treatment of C3 with diisobutylaluminum hydride in a polar aprotic solvent such as dichloromethane or tetrahydrofuran at −78° C. to −100° C. For the one step method, the reaction should not be maintained at around −65° C. to −78° C., and should preferrably be held and quenched at around −70° C. to −78° C.

In Step 3, the aldehyde C4 is treated with diethylaminosulfur trifluoride (DAST) in dichloromethane. This reaction is typically begun at around ice bath temperature, and is then warmed to room temperature for around 12-24 hours. The product of the reaction is the difluoromethylimidazole C5.

The transformations depicted in Steps 4 and 5 include a copper-mediated N-arylation reaction to afford C6 and subsequent conversion into a sulfonate, C7. Each of these transformations were described previously herein.

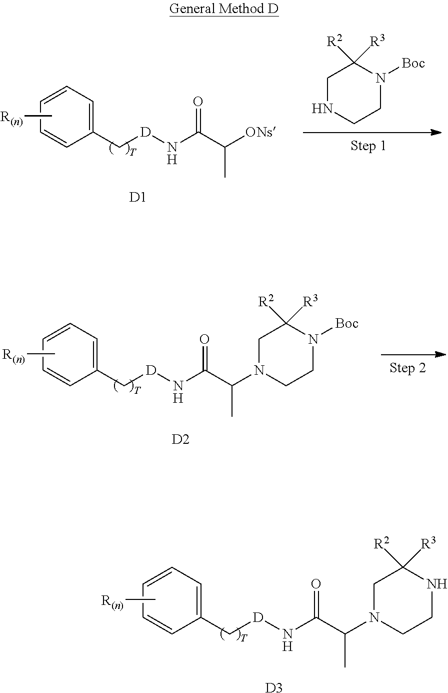

General Method D describes a method for the preparation of piperazine derivatives represented by structural formula D3. In the scheme, ONs' is a sulfonate leaving group such as 2-nitrobenzenesulfonyl (ONs), OTs, OMs, etc.; and all variables are as defined herein.

Step 1 is an S_{N}² reaction, described previously herein. In this embodiment of the reaction, D1 is converted into D2 by treatment with with tert-butyl 2,2-dimethylpiperazine-1-carboxylate in N, N-dimethylformamide in the presence of lithium triflate and an organic base such as N, N-diisopropylethylamine. The reaction may be run at 0-60° C., typically from 0° C. to room temperature, and preferrably at around 0° C.

In Step 2, the N-Boc group of D2 is cleaved by treatment with a strong acid. The skilled practitioner will appreciate that there are many sets of conditions that will accomplish this transformation. For example, group may be cleaved by treatment with trifluoroactic acid in dichloromethane, by treatment with hydrogen chloride in dioxane, or by treatment with sulfuric acid in ethyl acetate. The reaction is often run at temperatures of about 0-70° C., and is preferrably run at room temperature. The product of the reaction is the piperazine structure D3.

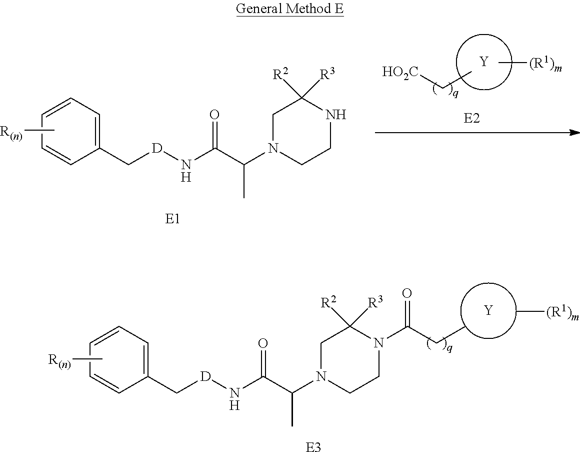

General Method E describes a method for the formation of amide structures represented by structural formula E3. In the scheme, all variables are as described herein.

Amidation reactions are well known in the art (Valeur, E and Bradley, M. Chem. Soc. Rev. 2009, 38, 606; Humphrey, J. M. and Chamberlin, A. R. Chem. Rev. 1997, 97, 2243) and are commonly but not exclusively mediated through conversion of the carboxylic acid component E2 into an electrophilic species such as an acid chloride or an active ester. In the former case, the acid chloride may be prepared upon treatment of the carboxylic acid (i.e. E2) with thionyl chloride or oxalyl chloride in a nonreactive polar aprotic solvent such as dichloromethane, which may optionally be performed in the presence of an amine base such as DIEA. These reactions are typically run at about 0° C. to around room temperature, although certain hindered substrates or weakly nucleophilic amines may require heating to around 60° C. or more to energize the reaction. Once the acid chloride is prepared, it may be used to acylate E1 by mixing with a polar aprotic inert organic solvent such as DCM in the presence of a base such as triethylamine according to well documented methods. Alternatively, a wide variety of coupling reagents have been developed to mediate amidation reactions through in situ formation of an intermediate active ester. These reagents include carbodiimides such as DCC or EDCI, and uranium salts such as HATU or TCFH, and are usually conducted in the presence of an amine base such as DIEA, triethylamine, N-methylimidazole, or DBU among others. Additionally, reactions mediated in this way are often conducted in the presence of an additive such as HOAt or HOBt. These additives can be useful in catalyzing the reaction, reducing side products, and reducing racemization of chiral acid components. Amidation reactions are typically conducted in polar aprotic solvents such as dichloromethane or DMF. For the immediate method, the amine component E1 is prepared according to methods described herein. The carboxylic acid component E2 may be purchased if commercially available or may be synthesized by one of skill in the art using published methods and methods described herein. In one example of implementation, E1 is mixed with E2 and stoichiometric HATU in dichloromethane at room temperature for around 1-24 hours, typically for around 2-8 hours, and the product of the reaction is an amide, represented by structure E3.

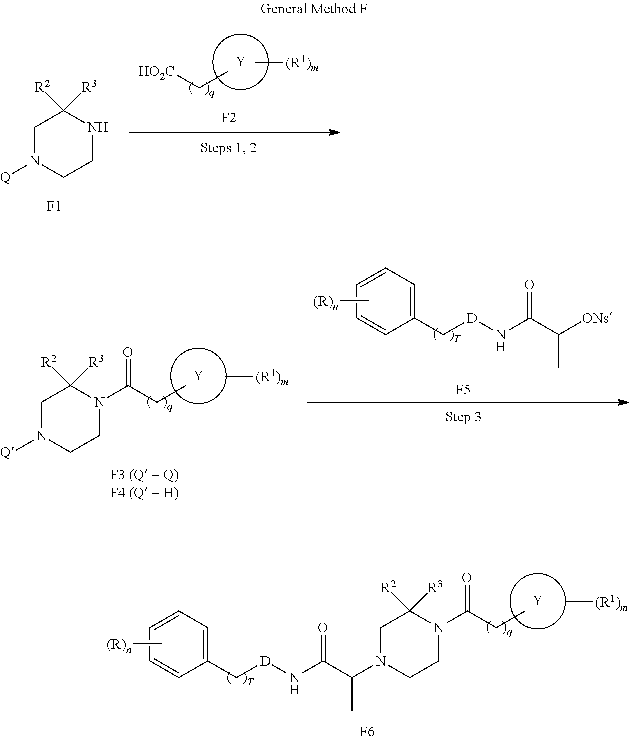

General method F describes an alternative method for the preparation of compounds represented by structural formula F6. In the scheme, Q is a urethane protecting group such as Boc; Q′ is Q or H; ONs' is a sulfonate leaving group such as 2-nitrobenzenesulfonyl (ONs), OTs, OMs, etc.; and all other variables are as described herein.

Steps 1, 2, and 3 include an amidation reaction, a carbamate deprotection, and an SN2 alkylation reaction, respectively. Each of these transformations was previously described, and the product of the reaction is represented by F6.

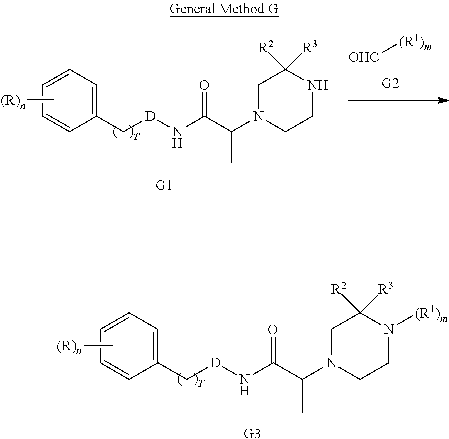

General Method G describes a method for the reductive amination of precursors G1 and G2 to afford substituted piperazine compounds represented by structural formula G3. In the scheme, all variables are as defined herein.

Reductive amination is common in organic synthesis and involves treating an amine with an aldehyde or ketone, typically in the presence of an acid catalyst, to form an imine or iminium ion intermediate, also known as a Schiff base. The Schiff base is typically reduced in situ with a reducing agent such as sodium cyanoborohydride or sodium triacetoxyborohydride. When the reducing agent is sodium cyanoborohydride, the solvent is typically an alcohol such as methanol; when the reducing agent is sodium triacetoxyborohydride, the solvent is often a polar aprotic solvent such as dichloromethane. The reaction is typically run between 0° C. and room temperature but can also be heated if necessary to energize the reaction in the case of weakly nucleophilic amines or sterically hindered substrates. The product of this reaction is an alkylated amine, G3.

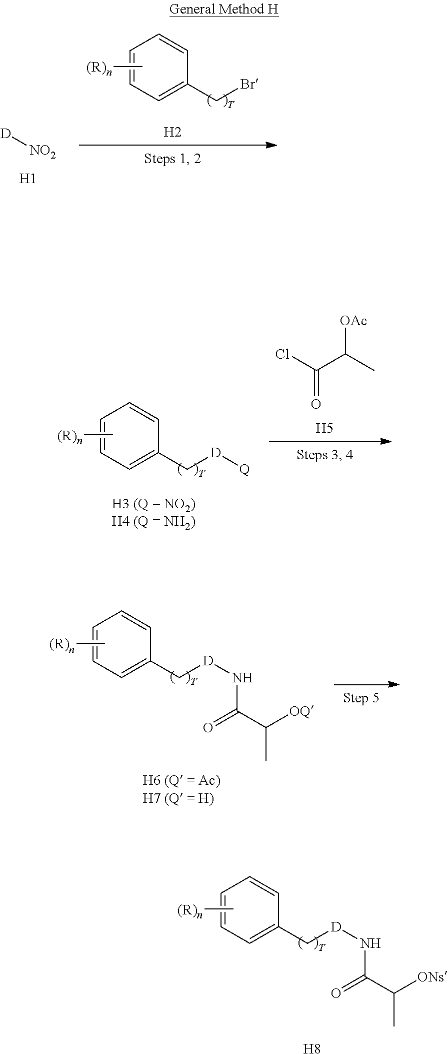

General Method H describes an alternative sequence for the synthesis of intermediates represented by structural formula H7. In the scheme, Br′ represents bromine or another leaving group such as Cl, I, or a sulfonate such as p-toluenesulphonyl; ONs' represents a sulfonate leaving group such as 2-nitrobenzenesulfonyl (ONs), OTs, OMs, etc.; and all other variables are as described herein.

Step 1 can be accomplished in different ways depending on the structure of the reactants. When T=1, an aromatic nitrogenous heterocycle, represented by H1, can be N-akylated with an electrophile such as H2. This is known in the industry as an S_{N}² reaction, which was previously described herein. When T=0, this reaction is best performed through the well-known Buchwald-Hartwig coupling, which enables the formation of carbon-nitrogen bonds between nitrogenous heterocycles and aryl halides such as bromobenzene derivatives (i.e. H2, where T=0). The Buchwald-Harwig reaction, which is mediated by a palladium catalyst/ligand/base system, can be run under a diverse conditions which are well documented in the chemical literature (Ruiz-Castillo, P.; Buchwald, S. L. Applications of Palladium-Catalyzed C—N Cross-Coupling Reactions. Chemical Reviews 2016, 116 (19), 12564-12649. DOI: 10.1021/acs.chemrev.6b00512. (2) Heravi, M. M.; Kheilkordi, Z.; Zadsirjan, V.; Heydari, M.; Malmir, M. Buchwald-Hartwig reaction: An overview. Journal of Organometallic Chemistry 2018, 861, 17-104). The product of Step 1 is an N-alkyl aromatic heterocycle or an N-aryl aromatic heterocycle, represented by H3.

In Step 2, the H3 nitro group is reduced to afford the aromatic amine H4. This type of reduction can be accomplished through a number of methods including catalytic hydrogenation, Raney nickel reduction, lithium aluminum hydride reduction, and iron or zinc-mediated reductions. These methods are commonly employed by skilled chemistry practitioners under reaction conditions readily available in the chemical literature. For example, if one wishes to utilize catalytic hydrogenation, one could expose a mixture of H3 and a palladium catalyst such as 10% palladium on carbon in an organic solvent such as ethanol, tetrahydrofuran, or dichloromethane, to an atmosphere of hydrogen gas for around 1-24 hours. This reaction may be run from around 0° C. to around 100° C., typically from about room temperature to around 65° C., and preferrably at room temperature. When run at room temperature, the reaction is expected to require around 2-12 hours.

In Step 3, the amine H4 is undergoes an amidation reaction, also known as an acylation, which was described previously herein. For example, a solution of the amine H4 in a polar aprotic solvent such as dichloromethane may be treated with an acid chloride, represented by H5, and an organic base such as triethylamine. The reaction can be run at around 0-80° C., and is typically run at around 0° C. to room temperature. When conducted at the preferred temperature of about 0° C., the reaction may require around 24 hours. The product of the reaction is represented by H6.

Step 4 is a saponification reaction whereby the acetate H6 is converted into the alcohol H7. Saponification is well known to those skilled in the art, and is typically conducted in an aqueous environment and mediated by a hydroxide base. For example, a mixture of H6 in a solution of aqueous THF may be treated with lithium hydroxide at about 0-80° C., and is typically run at about 0-65° C. When run at the preferred temperature of about room temperature, the reaction is expected to require around 4-12 hours.

In Step 5, the alcohol H7 is sulfonated upon treatment with a sulfonating agent such as 2-nitrobenzenesulfonyl chloride. This reaction was described previously herein. The product of the reaction is represented by H8.

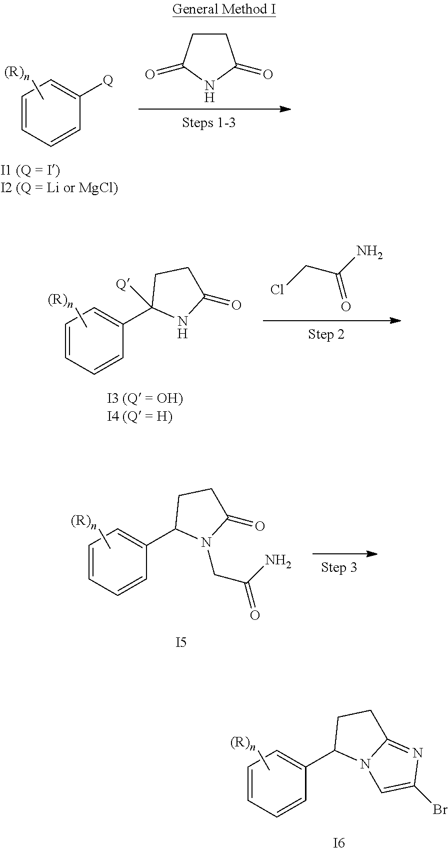

General Method I describes an alternative method for the formation of 2-bromo-6,7-dihydro-5H-pyrrolo[1,2-a]imidazole derivatives represented by structural formula I6. In the Scheme, I′ represents a halogen such as I, Cl or Br; and all other variables are as defined herein. In Step 1, a halobenzene derivative, represented by I1, undergoes metal-halogen exchange and subsequent addition to succinimide to afford a metalated species, I2. The metal-halogen exchange is well known in the art and is accomplished by treatment of an aryl halide with an active metal species such as an alkyllithium reagent or a turbo-Grignard reagent in an ethereal solvent. For example, treatment of a THF solution of I1 with the turbo-Grignard reagent isopropyl magnesium chloride-LiCl at an initial temperature of around −30° C. followed by warming to room temperature affords an arylmagnesium species, represented by I2.

In Step 2, The solution containing I2 is sequentially cooled to around −78° C., mixed with a THF solution of succinimide, and brought to room temperature for about two hours to afford the amidal I3.

In Step 3, the solution of I3 is cooled to around ice bath temperature and treated with a reducing agent such as sodium cyanoborohydride. The mixture is then warmed to around room temperature for about 1-2 hours and quenched with aqueous hydrochloric acid. The product of the reaction is a pyrrolidinone, I4.

Steps 4 and 5 are an S_{N}² alkylation to afford I5 and a cyclization/dehydration reaction to afford I6, respectively. Each of these reactions was discussed previously.

### Methods of Treatment

In one aspect, provided herein are methods of modulating MRGPRX2 activity comprising administering an effective amount of the compound of Formula (I) and/or a pharmaceutically acceptable salt thereof, or a pharmaceutical composition as described herein.

Also provided, in some embodiments, is a method of modulating mast cell degranulation comprising administering an effective amount of the compound of Formula (I) and/or a pharmaceutically acceptable salt thereof, or a pharmaceutical composition as described herein.

Also provided, in some embodiments, is a method of treating, preventing or ameliorating an MRGPRX2-mediated disease or disorder in a subject in need thereof comprising administering to the subject an effective amount of the compound of Formula (I) and/or a pharmaceutically acceptable salt thereof, or a pharmaceutical composition as described herein. In some embodiments, the disease is chosen from chronic spontaneous urticaria, mastocytosis, cold urticaria, atopic dermatitis, Asian atopic dermatitis, European atopic dermatitis, rosacea, autoimmune diseases, Crohn's disease, ulcerative colitis, irritable bowel syndrome, rheumatoid arthritis, fibromyalgia, endometriosis, nasal polyps, neuropathic pain, inflammatory pain, pseudo-allergic drug reactions, chronic itch, drug-induced anaphylactoid reactions, metabolic syndrome, esophagus reflux, asthma, cough, migraine, sinusitis, urticaria, chronic inducible urticaria, chronic pruritus, acute pruritus, prurigo nodularis, osteoarthritis, pseudo anaphylaxis, contact urticaria, lupus erythematosus (SLE), psoriasis, psoriatic arthritis, bronchial asthma, systemic mastocytosis, cutaneous mastocytosis, mastocytic enterocolitis, mast cell activation syndrome (MCAS), interstitial cystitis, food allergy, allergic rhinitis, microbial infection, eosinophilic esophagitis (EOE) and chronic pain.

Also provided, in some embodiments, is a method of treating a condition chosen from chronic spontaneous urticaria, mastocytosis, cold urticaria, atopic dermatitis, Asian atopic dermatitis, European atopic dermatitis, rosacea, autoimmune diseases, Crohn's disease, ulcerative colitis, irritable bowel syndrome, rheumatoid arthritis, fibromyalgia, endometriosis, nasal polyps, neuropathic pain, inflammatory pain, pseudo-allergic drug reactions, chronic itch, drug-induced anaphylactoid reactions, metabolic syndrome, esophagus reflux, asthma, cough, migraine, sinusitis, urticaria, chronic inducible urticaria, chronic pruritus, acute pruritus, prurigo nodularis, osteoarthritis, pseudo anaphylaxis, contact urticaria, lupus erythematosus (SLE), psoriasis, psoriatic arthritis, bronchial asthma, systemic mastocytosis, cutaneous mastocytosis, mastocytic enterocolitis, mast cell activation syndrome (MCAS), interstitial cystitis, food allergy, allergic rhinitis, microbial infection, eosinophilic esophagitis (EOE) and chronic pain comprising administering to the subject an effective amount of the compound of Formula (I) and/or a pharmaceutically acceptable salt thereof, or a pharmaceutical composition as described herein.

In some embodiments, the condition is atopic dermatitis.

Also provided, in some embodiments, is the compound of Formula (I) and/or a pharmaceutically acceptable salt thereof for use as a medicament. In some embodiments, the compound is for use in treating disease or disorder mediated by MRGPRX2.

Also provided, in some embodiments, is the use of the compound of Formula (I) and/or a pharmaceutically acceptable salt thereof in the manufacture of a medicament for treating a MRGPRX2-mediated disease or disorder.

### Dosages, Administration and Pharmaceutical Compositions/Formulations

The compounds or pharmaceutical compositions described herein that can be used in therapy can be formulated and dosages established in a fashion consistent with good medical practice taking into account the disorder to be treated, the condition of the individual patient, the site of delivery of the compound or pharmaceutical composition, the method of administration and other factors known to practitioners. The compounds or pharmaceutical compositions can be prepared according to the description of preparation described herein.

A therapeutically effective amount can be the amount of a compound or pharmaceutical composition or an active component thereof sufficient to provide a beneficial effect or to otherwise reduce a detrimental non-beneficial event to the individual to whom the composition is administered. A therapeutically effective dose can be a dose that produces one or more desired or desirable (e.g., beneficial) effects for which it is administered, such administration occurring one or more times over a given period of time. An exact dose can depend on the purpose of the treatment and can be ascertainable by one skilled in the art using known techniques.

One of ordinary skill in the art would understand that the amount, duration, and frequency of administration of a pharmaceutical composition or compound described herein to a subject in need thereof depends on several factors including, for example but not limited to, the health of the subject, the specific disease or condition of the patient, the grade or level of a specific disease or condition of the patient, the additional therapeutics the subject is being or has been administered, and the like.

Pharmaceutical compositions or compounds of the present disclosure can be administered to a subject in need thereof in a first administration, and in one or more additional administrations. The one or more additional administrations can be administered to the subject in need thereof minutes, hours, days, weeks, or months following the first administration. Any one of the additional administrations can be administered to the subject in need thereof less than 21 days, or less than 14 days, less than 10 days, less than 7 days, less than 4 days or less than 1 day after the first administration. The one or more administrations can occur more than once per day, more than once per week, or more than once per month. The compounds or pharmaceutical compositions can be administered to the subject in need thereof in cycles of 21 days, 14 days, 10 days, 7 days, 4 days, or daily over a period of one to seven days.

In general, the compounds of this disclosure will be administered in a therapeutically effective amount by any of the accepted modes of administration for agents that serve similar utilities. Therapeutically effective amounts of compounds of Formula (I) may range from about 0.01 to about 500 mg/kg patient body weight per day, which can be administered in single or multiple doses. In one embodiment, the dosage level will be about 0.01 to about 250 mg/kg per day, about 0.05 to about 100 mg/kg per day, about 0.1 to about 250 mg/kg per day, about 0.1 to about 50 mg/kg per day or about 0.5 to about 100 mg/kg per day. In addition, the dosage can be about 0.05 to about 0.5, about 0.5 to about 5 or about 5 to about 50 mg/kg per day. For oral administration, the compositions may be provided in the form of tablets containing about 1.0 to about 1000 milligrams of the active ingredient, particularly about 1.0, 5.0, 10, 15, 20, 25, 50, 75, 100, 150, 200, 250, 300, 400, 500, 600, 750, 800, 900, and 1000 milligrams of the active ingredient. The actual amount of the compound of this disclosure, i.e., the active ingredient, will depend upon numerous factors such as the severity of the disease to be treated, the age and relative health of the subject, the potency of the compound being utilized, the route and form of administration, and other factors.

These dosages are based on an average human subject having a weight of about 60 kg to 70 kg. The physician will readily be able to determine doses for subjects whose weight falls outside this range, such as infants and the elderly. Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the mammalian Subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the chemotherapeutic agent and the particular therapeutic or prophylactic effect to be achieved, and (b) the limitations inherent in the art of compounding Such an active compound for the treatment of sensitivity in individuals.

Thus, one of skill in the art would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present invention.

In general, compounds of this disclosure will be administered as pharmaceutical compositions by any one of the following routes: oral, systemic (e.g., transdermal, intranasal or by suppository), or parenteral (e.g., intramuscular, intravenous, or subcutaneous) administration. The preferred manner of administration is oral using a convenient daily dosage regimen, which can be adjusted according to the degree of affliction. Compositions can take the form of tablets, pills, capsules, semisolids, powders, sustained release formulations, solutions, suspensions, elixirs, aerosols, or any other appropriate compositions. The compounds of the invention may also be administered topically, (intra)dermally, or transdermally to the skin or mucosa. Parenteral administration and administered parenterally as used herein includes modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Also provided, in some embodiments, is a pharmaceutical composition comprising the compound of Formula (I) and/or a pharmaceutically acceptable salt thereof and a pharmaceutically acceptable excipient.

A pharmaceutically acceptable excipient includes pharmaceutically acceptable material, composition, or vehicle, such as a liquid or solid filler, diluent, solvent, or encapsulating material. Each excipient must be “acceptable” in the sense of being compatible with the other ingredients of the formulation and not injurious to the patient. Some examples of materials which can serve as pharmaceutically acceptable excipients include: (1) monosaccharides, disaccharides, and other carbohydrates including glucose, sucrose; lactose; mannose, trehalose, sorbitol or dextrins and (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide, aluminum hydroxide phosphate, citrate, and other organic acids; (15) alginic acid; (16) pyrogen-free water; (17) isotonic saline; (18) Ringer's solution; (19) ethyl alcohol; (20) antioxidants including ascorbic acid and methionine; preservatives, polypeptides; proteins, such as serum albumin; hydrophilic polymers; amino acids; chelating agents such as EDTA; salt-forming counter-ions such as sodium; metal complexes; and/or non-ionic surfactants or polyethylene glycol and other non-toxic compatible substances employed in pharmaceutical formulations.

Examples of excipients and their use may be found in Remington's Pharmaceutical Sciences, 20th Edition (Lippincott Williams & Wilkins, 2000). The choice of excipient will to a large extent depend on factors such as the particular mode of administration, the effect of the excipient on solubility and stability, and the nature of the dosage form.

Methods for formulation of the pharmaceutical compositions can include formulating any of the compounds described herein with one or more inert, pharmaceutically acceptable excipients (including carriers) to form a solid, semi-solid, or liquid composition. Solid compositions can include, for example, powders, tablets, dispersible granules, and capsules, and in some aspects, the solid compositions further contain nontoxic, auxiliary substances, for example wetting or emulsifying agents, pH buffering agents, and other pharmaceutically acceptable additives. This formulation can be an aqueous solution. Alternatively, the compositions described herein can be lyophilized or in powder form for re-constitution with a suitable vehicle, e.g., sterile pyrogen-free water, before use. The active ingredients can be entrapped in microcapsules prepared, for example, by coacervation techniques or by interfacial polymerization (e.g., hydroxymethylcellulose or gelatin microcapsules and poly-(methylmethacylate) microcapsules, respectively), in colloidal drug-delivery systems (e.g., liposomes, albumin microspheres, microemulsions, nano-particles and nanocapsules) or in macroemulsions.

The pharmaceutical compositions and formulations can be sterilized. Sterilization can be accomplished by filtration through sterile filtration.

The pharmaceutical compositions described herein can be formulated for administration as an injection. Non-limiting examples of formulations for injection can include a sterile suspension, solution, or emulsion in oily or aqueous vehicles. Suitable oily vehicles can include, but are not limited to, lipophilic solvents or vehicles such as fatty oils, synthetic fatty acid esters, or liposomes. Aqueous injection suspensions can contain substances which increase the viscosity of the suspension. The suspension can also contain suitable stabilizers. Injections can be formulated for bolus injection or continuous infusion.

For parenteral administration, the compounds can be formulated in a unit dosage injectable form (e.g., solution, suspension, emulsion) in association with a pharmaceutically acceptable parenteral vehicle. Such vehicles can be inherently nontoxic, and non-therapeutic. A vehicle can be water, saline, Ringer's solution, dextrose solution, and 5% human serum albumin. Nonaqueous vehicles such as fixed oils and ethyl oleate can also be used. Liposomes can be used as carriers. The vehicle can contain minor amounts of additives such as substances that enhance isotonicity and chemical stability (e.g., buffers and preservatives).

The pharmaceutical compositions provided herein may be formulated as immediate or modified release dosage forms, including delayed-, sustained, pulsed-, controlled, targeted-, and programmed-release forms. Thus, in some preferred embodiments, the active ingredient(s) is administered in a pharmaceutical composition which is an immediate release oral dosage form, preferably but not necessarily including an enteric coating. In some preferred embodiments, the active ingredients(s) are administered in a pharmaceutical composition which is an extended-release oral dosage form, preferably but not necessarily including an enteric coating. In further preferred embodiments, the active ingredients are administered in a pharmaceutical composition which contains both an immediate release dose and an extended-release dose or pulsed release dose of the first agent preferably but not necessarily also including an enteric coating. Such dual release dosage forms achieve release of an initial dose of active ingredient, followed late in time by another pulsed release, or by a sustained release dose. Methodologies for preparing such dual release dosage forms are well known in the art.

In some embodiments, the active ingredients are formulated into a controlled release matrix tablet, which contains one or more polymeric matrix materials that promote the sustained, delayed, or pulsed release profile. Non-limiting examples of such polymeric matrix materials include cellulosic materials as described above, and carbomers, for example those sold by Lubrizol Corporation under the name Carbopol®, for example Carbopol® 71G NF, Carbopol® 971P NF and Carbopol® 974P NF polymers.

Some preferred examples of extended-release compositions suitable for use in the methods and compositions of the invention include, for example and not limitation, extended-release compositions found in nifedipine formulations such as Adalat CC®, Procardia® XL, Afeditab® CR and Nifedical® XL; and in diltiazem formulations such as Cardizem® CD, Cardizem® LA, Cardizem® SR, Cartia® XT and Dilacor® XR.

### Combination Therapy

Compounds of the present disclosure may be used in methods of treating in combination with one or more other combination agents (e.g., one, two, or three other drugs) that are used in the prevention, treatment, control, amelioration, or reduction of risk of the diseases or conditions for which compounds of the present disclosure are useful. In some embodiments, the combination of the drugs together are safer or more effective than either drug alone. In some embodiments the compound disclosed herein and the one or more combination agents have complementary activities that do not adversely affect each other. Such molecules can be present in combination in amounts that are effective for the purpose intended. Such other drug(s) may be administered, by a route and in an amount commonly used therefore, contemporaneously or sequentially with a compound of the present disclosure. When a compound of the present disclosure is used contemporaneously with one or more other drugs, in some embodiments, the agents are administered together in a single pharmaceutical composition in unit dosage form.

Accordingly, the pharmaceutical compositions of the present disclosure also include those that contain one or more other active ingredients, in addition to a compound of the present disclosure. The weight ratio of the compound of the present disclosure to the second active agent may be varied and will depend upon the effective dose of each ingredient. Generally, an effective dose of each will be used. In some embodiments, combination therapy includes therapies in which the compound of the present disclosure and one or more other drugs are administered separately, and in some cases, the two or more agents are administered on different, overlapping schedules. It is also contemplated that when used in combination with one or more other active ingredients, the compounds of the present disclosure and the other active ingredients may be used in lower doses than when each is used singly. In some embodiments, the combination agent is a drug for reduction of symptoms of ALS. In some embodiments, the combination agent is selected from an NAD supplement (such as nicotinamide riboside, offered under the trade names Basis® or Tru Niagen®), vitamin B₁₂ (oral or injection), glycopyrrolate, atropine, scopolamine, baclofen, tizanidine, mexiletine, an SSRI, a benzodiazepine, Neudexta, riluzole, and edaravone, and combinations thereof.

Since the present invention has an aspect that relates to the treatment of the disease/conditions described herein with a combination of active ingredients which may be administered separately, the invention also relates to combining separate pharmaceutical compositions in kit form. The kit comprises two separate pharmaceutical compositions: a compound of Formula I a prodrug thereof or a salt of Such compound or prodrug and a second compound as described above. The kit comprises a means for containing the separate compositions such as a container, a divided bottle or a divided foil packet. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. Second Week, Monday, Tuesday, . . . etc. Other variations of memory aids will be readily apparent. A “daily dose” can be a single tablet or capsule or several pills or capsules to be taken on a given day. Also, a daily dose of Formula I compound can consist of one tablet or capsule while a daily dose of the second compound can consist of several tablets or capsules and vice versa. The memory aid should reflect this. In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter which indicates the number of daily doses that has been dispensed. Another example of such a memory-aid is a battery-powered microchip memory coupled with a liquid crystal readout, or audible reminder signal which, for example, reads out the date that the last daily dose has been taken and/or reminds one when the next dose is to be taken. Also, as the present invention has an aspect that relates to the treatment of the disease/conditions described herein with a combination of active ingredients which may be administered jointly, the invention also relates to combining separate pharmaceutical compositions in a single dosage form, Such as (but not limited to) a single tablet or capsule, a bilayer or multilayer tablet or capsule, or through the use of segregated components or compartments within a tablet or capsule. The compounds, pharmaceutical compositions, and methods of the present disclosure can be useful for treating a subject such as, but not limited to, a mammal, a human, a non-human mammal, a domesticated animal (e.g., laboratory animals, household pets, or livestock), a non-domesticated animal (e.g., wildlife), a dog, a cat, a rodent, a mouse, a hamster, a cow, a bird, a chicken, a fish, a pig, a horse, a goat, a sheep, or a rabbit. In preferred embodiments, compounds, pharmaceutical compositions, and methods of the present disclosure are used for treating a human.

The present invention further comprises use of a compound of Formula I for use as a medicament (Such as a unit dosage tablet or unit dosage capsule). In another embodiment, the present invention comprises the use of a compound of Formula I for the manufacture of a medicament (such as a unit dosage tablet or unit dosage capsule) to treat one or more of the conditions discussed herein.

### Therapeutic Utility

Natural endogenous ligands of Mrgprb2/MRGPRX2 have been reported and are mostly neuropeptides, including substance P (SP), vasoactive intestinal polypeptide (VIP), Cortistatin-14, and pituitary adenylate cyclase activating polypeptide (PACAP). Others include B-defensin, cathelicidin (LL-37), and proadrenomedullin N-terminal 20 peptide (PAMP9-20) (Journal of Allergy and Clinical Immunology, 138, 700-710 (2016); J. Immunol., 191, 345-352 (2013); BBRC, 349, 1322-28 (2006)). Given the close proximity between mast cells and sensory nerves in various pathological conditions, it follows that neuropeptide-activated MRGPRX2 could contribute to symptoms of neurogenic inflammation including pain, swelling and pruritus. Various observations using knock-out (KO) mice are consistent with the Mrgprb2/MRGPRX2 receptors playing a role in mast cell-mediated neurogenic inflammation. For instance, Mrgprb2/MRGPRX2 agonists induce various symptoms such as flushing, swelling and itch in wild type mice, but not in Mrgprb2-deficient mice (Nature, 519, 237-241 (2015); Immunity, 50, 1163-1171 (2019)). Mrgprb2-deficient mice have also demonstrated significant reductions in inflammation (leukocyte infiltration, including mast cells), swelling, pain and overall clinical score in various disease models (Neuron, 101, 412-420 (2019); Immunity, 50, 1163-1171 (2019); Nature Immunology, 20, 1435-1443 (2019)). An important and relevant observation was the demonstration that Substance P injection could stimulate the infiltration of leukocytes in wild type and NKR1 (canonical Substance Preceptor) KO mice whereas the response was substantially blunted in Mrgprb2 null mice (Neuron, 101, 412-420 (2019)). This observation extends the role of Mrgprb2/MRGPRX2 as a key receptor in mediating Substance P-induced inflammatory responses, including pain (Neuron, 101, 353-355, (2019)). Indeed, a Substance P/Mrgprb2 sensory cluster was demonstrated to be critical in driving the clinical score of a severe preclinical model of atopic dermatitis (Nature Immunology, 20, 1435-1443 (2019)).

In addition to the various reports using Mrgprb2-deficient mice, further evidence suggests a role for various ligands of MRGPRX2 in human disease. For example, in addition to the number of MRGPRX2-expressing mast cells being significantly increased in severe chronic urticaria (Clinical and Molecular Allergy 16, 24 (2018)), PACAP nerve fibers were demonstrated to be in close proximity to tryptase-positive mast cells, providing the morphological basis for increased mast cell-sensory interactions (J. Allergy Clin. Immunol., 134, 622-633 (2014)). In support of this, patients with urticaria exhibit enhanced wheal reactions vs healthy individuals to MRGPRX2 agonists (e.g., Substance P) when injected intradermally (Allergy, 54, 46-56 (1999)). In addition, PACAP and the antimicrobial peptide, LL-37, which is implicated in cutaneous inflammation, were both demonstrated to be upregulated in rosacea (J. Invest. Dermatol., 15, 53-62, (2011)). Indeed, mast cell-deficient mice do not develop inflammation/flushing following L.L-37 injection (J. Inv. Derm., 134, 2728 (2014)) thus inferring a role for Mrgprb2.

In addition to skin disorders, mast cell involvement has been highlighted for inflammatory bowel disease (IBD) and arthritis (Immunol. Rev., 217, 38-52 (2007); Pharmacology and Therapeutics, 116, 207-235 (2007)) as well as asthma (Respiratory Research, 19, 1 (2018)) and migraine. In patients with rheumatoid arthritis (RA), the number of degranulated mast cells is increased in synovial tissue and is correlated with disease activity, as it is for patients with IBD. A positive correlation between serum Substance P levels and chronic pain intensity has been noted in both osteoarthritic and RA patients (PLOS ONE, 10, e0139206 ((2015)) and a recent article suggested that the SP-MRGPRX2 axis may play a role in the pathogenesis of RA, especially in the regulation of inflammation and pain (Allerg. Intern., 26, S9-S20 (2017)). Finally, there is a growing body of evidence for a role of PACAP in migraine pathogenesis and that it is mediated via activation of mast cells (Frontiers in Cellular Neuroscience, 13, 1-11 (2019)).

The Formula I compounds of this invention, their prodrugs and the salts of such compounds and prodrugs are all adapted to therapeutic use as agents that mediate the Mas-related G protein-coupled receptor (MRGPRX2) in mammals, particularly humans. For example, these compounds act as MRGPRX2 receptor antagonists and thus are useful for the treatment of the various conditions (e.g., those described herein) in which such action is implicated.

Given the positive correlation between activation of the MRGPRX2 receptor with the development of itch associated conditions, a pain associated condition, a pseudo-allergic reaction, an autoimmune or inflammatory disorder, or cancer-associated condition, Formula I compounds of this invention, their prodrugs and the salts of such compounds and prodrugs, by virtue of their pharmacologic action, are useful for the prevention, arrestment and/or regression of an itch associated condition, a pain associated condition, a pseudo-allergic reaction, an autoimmune or inflammatory disorder, or cancer-associated condition.

As used herein the phrase “pseudo-allergic reaction” refers to an IgE-independent allergic reaction, characterized by histamine release, inflammation, airway contraction, or any combination thereof. A pseudo-allergic reaction may be an anaphylactic reaction. A pseudo-allergic reaction may be caused by a range of cationic substances, collectively called basic secretagogues, including inflammatory peptides and drugs associated with allergic-type reactions. Thus, in one embodiment, the method of present invention is provided to treat a pseudo-allergic reaction, such as pseudo-allergic reactions caused by secretagogues, cationic peptidergic drugs, anionic peptidergic drugs, neutral peptidergic drugs, non-steroidal antagonist drugs, neuropeptides, and antimicrobial peptides. In one embodiment, the pseudo-allergic reaction is caused by MCD peptide, Substance P, VIP, PACAP, dynorphin, somatostatin, Compound 48/80, cortistatin-14, mastoparan, melettin, cathelicidin peptides, ciprofloxacin, vancomycin, leuprolide, goserelin, histrelin, triptorelin, cetrorelix, ganirelix, degarelix, octreotide, lanreotide, pasireotide, sermorelin, tesamorelin, icatibant, glatiramer acetate, teriparatide, pramlintide, bleomycin, exenatide, glucagon, liraglutide, enfuvirtide, colistimethate, succinylcholine, tubocurarine, atracurium, mivacurium, and rocuronium.

As used herein, the phrase “itch associated condition” means pruritus (including acute and chronic pruritus) associated with any condition. The itch sensation can originate, e.g., from the peripheral nervous system (e.g., dermal or neuropathic itch) or from the central nervous system (e.g., neuropathic, neurogenic or psychogenic itch). Thus, in one embodiment, the method of present invention is provided to treat an itch associated condition, such as chronic itch; contact dermatitis; allergic blepharitis; anaphylaxis; anaphylactoid drug reactions; Anaphylactic shock; Anemia; Atopic dermatitis; Bullous pemphigoid; Candidiasis; Chicken pox; end-stage renal failure; hemodialysis; Cholestatic pruritis; Chronic urticaria; Contact dermatitis, Dermatitis herpetiformis; Diabetes; Drug allergy, Dry skin; Dyshidrotic dermatitis; Ectopic eczema; Eosinophilic fasciitis; Epidermolysis bullosa; Erythrasma; Food allergy; Folliculitis; Fungal skin infection; Hemorrhoids; Herpes; HIV infection; Hodgkin's disease; Hyperthyroidism; Iodinated contrast dye allergy; Iron deficiency anemia; Kidney disease; Leukemia, porphyria; Lymphoma; Mast cell activation syndrome, Malignancy; Mastocystosis; Multiple myeloma; Neurodermatitis; Onchocerciasis; Paget's disease; Pediculosis; Polycythemia rubra vera; Prurigo nodularis; Lichen Planus; Lichen Sclerosis; Pruritus ani; Pseudo-allergic reactions; Pseudorabies; Psoriasis; Rectal prolapse; Sarcoidosis granulomas; Scabies; Schistosomiasis; Scleroderma, Severe stress, Stasia dermatitis; Swimmer's itch; Thyroid disease; Tinea cruris; Uremic Pruritus; Rosacea; Cutaneous amyloidosis; Scleroderma; Acne; wound healing; burn healing; ocular itch; and Urticaria.

As used herein, the term “autoimmune disorder,” or “inflammatory disorder” means a disease or disorder arising from and/or directed against an individual's own tissues or organs, or a co-segregate or manifestation thereof, or resulting condition therefrom. Typically, various clinical and laboratory markers of autoimmune diseases may exist including, but not limited to, hypergammaglobulinemia, high levels of autoantibodies, antigen-antibody complex deposits in tissues, clinical benefit from corticosteroid or immunosuppressive treatments, and lymphoidcell aggregates in affected tissues. Thus, in one embodiment, the method of present invention is provided to treat an autoimmune disorder, such as chronic inflammation, mast cell activation syndrome, Multiple Sclerosis, Steven Johnson's Syndrome, Toxic Epidermal Necrolysis, appendicitis, bursitis, cutaneous lupus, colitis, cystitis, dermatitis, phlebitis, reflex sympathetic dystrophy/complex regional pain syndrome (rsd/crps), rhinitis, tendonitis, tonsillitis, acne vulgaris, sinusitis, rosacea, psoriasis, graft-versus-host disease, reactive airway disorder, asthma, airway infection, allergic rhinitis, autoinflammatory disease, celiac disease, chronic prostatitis, diverticulitis, glomerulonephritis, hidradenitis suppurativa, hypersensitivities, intestinal disorder, epithelial intestinal disorder, inflammatory bowel disease, irritable bowel syndrome, Crohn's Disease, ulcerative colitis, lupus erythematous, interstitial cystitis, otitis pelvic inflammatory disease, endometrial pain, reperfusion injury, rheumatic fever, rheumatoid arthritis, sarcoidosis, transplant rejection, psoriasis, lung inflammation, chronic obstructive pulmonary disease, permanent sputum eosinophilia, eosinophilic leukemia, eosinophilic esophagitis, eosinophilic gastritis, mast cell gastrointestinal disease, hypereosinophilic syndrome, aspirin-exacerbated respiratory disease, nasal polyposis, chronic rhinosinusitis, antibody-dependent cell-mediated cytotoxicity, neurofibromatosis, swannamatoisis, tubulointerstitial nephritis, glomerulonephritis, diabetic nephropathy, allograft rejection, amyloidosis, renovascular ischemia, reflux nephropathy, polycystic kidney disease, liver fibrosis/cirrhosis, autoimmune liver disease, Biliary atresia, acute and chronic Hepatitis Band C virus, Liver tumors and cancer, Alcoholic liver disease, Polycystic liver disease, Liver cholangiocarcinoma, neuromyelitis optica spectrum disorder, cardiovascular disease, and vasculitis.

As used herein, the phrase “pain associated condition” means any pain due to a medical condition. Thus, in one embodiment, the method of present invention is provided to treat a pain associated condition, such as Acute Pain, Advanced Prostate Cancer, AIDS-Related Pain, Ankylosing Spondylitis, Arachnoiditis, Arthritis, Arthrofibrosis, Ataxic Cerebral Palsy, Autoimmune Atrophic Gastritis, Avascular Necrosis, Back Pain, Behcet's Disease (Syndrome), Burning Mouth Syndrome, Bursitis, Cancer Pain, Carpal Tunnel, Cauda Equina Syndrome, Central Pain Syndrome, Cerebral Palsy, Cervical Stenosis, Charcot-Marie-Tooth (CMT) Disease, Chronic Fatigue Syndrome (CFS), Chronic Functional Abdominal Pain (CFAP), Chronic Pain, Chronic Pancreatitis, Chronic Pelvic Pain Syndrome, Collapsed Lung (Pneumothorax), Complex Regional Pain Syndrome (RSD), Corneal Neuropathic Pain, Crohn's Disease, Degenerative Disc Disease, Dental Pain, Dercum's Disease, Dermatomyositis, Diabetic Peripheral Neuropathy (DPN), Dystonia, Ehlers-Danlos Syndrome (EDS), Endometriosis, Eosinophilia-Myalgia Syndrome (EMS), Erythromelalgia, Fibromyalgia, Gout, Headaches, Herniated disc, Hydrocephalus, Intercostal Neuraligia, Interstitial Cystitis, Irritable Bowel syndrome (IBS), Juvenile Dermatositis (Dermatomyositis), Knee Injury, Leg Pain, Loin Pain-Haematuria Syndrome, Lupus, Lyme Disease, Medullary Sponge Kidney (MSK), Meralgia Paresthetica, Mesothelioma, Migraine, Musculoskeletal pain, Myofascial Pain, Myositis, Neck Pain, Neuropathic Pain, Occipital Neuralgia, Osteoarthritis, Paget's Disease, Parsonage Turner Syndrome, Pelvic Pain, Periodontitis Pain, Peripheral Neuropathy, Phantom Limb Pain, Pinched Nerve, Polycystic Kidney Disease, Polymyalgia Rhuematica, Polymyositis, Porphyria, Post Herniorrhaphy Pain Syndrome, Post Mastectomy, Postoperative Pain, Pain Syndrome, Post Stroke Pain, Post Thoracotomy Pain Syndrome, Postherpetic Neuralgia (Shingles), Post-Polio Syndrome, Primary Lateral Sclerosis, Psoriatic Arthritis, Pudenda! Neuralgia, Radiculopathy, Raynaud's Disease, Rheumatoid Arthritis (RA), Sacroiliac Joint Dysfunction, Sarcoidosis, Scheuermann's Kyphosis Disease, Sciatica, Scoliosis, Shingles (Herpes Zoster), Sjogren's Syndrome, Spasmodic Torticollis, Sphincter of Oddi Dysfunction, Spinal Cerebellum Ataxia (SCA Ataxia), Spinal Cord Injury, Spinal Stenosis, Syringomyelia, Tarlov Cysts, Transverse Myelitis, Trigeminal Neuralgia, Neuropathic Pain, Ulcerative Colitis, Vascular Pain and Vulvodynia.

As used herein the phrase “cancer associated condition” means any disease arising from the proliferation of malignant cancerous cells. Thus, in one embodiment, the method of present invention is provided to treat a cancer/tumor associated condition, such as adenoid cystic carcinoma, adrenal gland tumor, amyloidosis, anal cancer, appendix cancer, astrocytoma, ataxia-telangiectasia, Beckwith-Wiedemann syndrome, cholangiocarcinoma, birt-hogg-dube syndrome, bone cancer, brain stem glioma, brain tumor, breast cancer (inflammatory, metastatic, male), prostrate, basal cell, melanoma, colon, colorectal, bladder, kidney cancer, lacrimal gland cancer, laryngeal and hypopharyngeal cancer, lung cancer (non-small cell, small cell), leukemia (acute lymphoblastic, acute lymphocytic, acute myeloid, B cell prolymphocytic, chronic lymphocytic, chronic myeloid, chronic T cell lymphocytic, eosinophilic), Liver Cancer, Li-Fraumeni syndrome, lymphoma (Hodgkin and non-Hodgkin), lynch syndrome, mastocytosis, medulloblastoma, meningioma, mesothelioma, multiple endocrine neoplasia, multiple myeloma, MUTYH-associated polyposis, myelodysplastic syndrome, nasal cavity and paranasal sinus cancer, neuroblastoma, neuroendocrine tumors, neurofibromatosis, penile cancer, parathyroid cancer, ovarian fallopian tube and peritoneal cancer, osteosarcoma, pituitary gland tumor, pleuropulmonary blastoma, oral and oropharyngeal, thyroid, uterine, pancreatic, carney complex, brain and spinal cord cancer, cervical cancer, Cowden syndrome, craniopharyngioma, desmoid tumor, desmoplastic infantile ganglioglioma, ependymoma, esophageal cancer, Ewing sarcoma, eye cancer, eyelid cancer, familial adenomatous polyposis, familial GIST, familial malignant melanoma, familial pancreatic cancer, gallbladder cancer, gastrointestinal stromal tumor, germ cell tumor, gestational trophoblastic disease, head and neck cancer, hereditary breast and ovarian cancer, hereditary diffuse gastric cancer, hereditary, leiomyomastosis and renal cell cancer, hereditary pancreatitis, hereditary papillary renal carcinoma, hereditary mixed polyposis syndrome, HIV/AIDS related cancers, retinoblastoma, rhabdomyosarcoma, salivary gland cancer, Kaposi sarcoma, small bowel cancer, stomach cancer, testicular cancer, thymoma and thymic carcinoma, thyroid cancer, vaginal cancer, culver cancer, Werner syndrome and Xeroderma pigmentosum.

### EXAMPLES

The following preparations of compounds of Formula (I) and intermediates are given to enable those skilled in the art to more clearly understand and to practice the present disclosure. They should not be considered as limiting the scope of the disclosure, but merely as being illustrative and representative thereof.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this disclosure can be synthesized, and various modifications to these schemes can be made and will be suggested to one skilled in the art having referred to this disclosure. The starting materials and the intermediates, and the final products of the reaction may be isolated and purified if desired using conventional techniques, including but not limited to filtration, distillation, crystallization, chromatography, and the like. Such materials may be characterized using conventional means, including physical constants and spectral data.

Unless specified to the contrary, the reactions described herein take place at atmospheric pressure over a temperature range from about −78° C. to about 150° C., or from about 0° C. to about 125° C. or at about room (or ambient) temperature, e.g., about 20° C.

Compounds of Formula (I) and sub formulae and species described herein, including those where the substituent groups as defined herein, can be prepared as illustrated and described below.

Unless otherwise noted, all reagents were used without further purification. ¹H NMR spectra were obtained in CDCl₃, DMSO-d₆, or CD₃OD, unless stated otherwise, at room temperature on a Bruker AVANCE III HD 300 MHz, Bruker AVANCE III HD 400 MHz, or AVANCE NEO 400 MHz instrument or an NMR spectrometer of similar caliber. When more than one conformer was detected, the chemical shifts for the most abundant one is reported. Chemical shifts of ¹H NMR spectra were recorded in parts per million (ppm) on the 6 scale from an internal standard of residual solvent. Splitting patterns are designed as s, singlet; d, doublet; t, triplet; q, quartet; m, multiplet; br, broad.

The following abbreviations are used in the text: LCMS=liquid chromatography-mass spectrometry, ESI=Electrospray Ionization, M+H=a unit higher than the monoisotopic mass of the uncharged molecule, HPLC=High pressure liquid chromatography, Prep-HPLC=preparatory scale HPLC, AcOH=acetic acid, ACN or MeCN=acetonitrile, Boc=tert-butyloxycarbonyl, PE=petroleum ether, EA or EtOAc=ethyl acetate, BSA=Bovine Serum Albumin, CPME=cyclopentyl methyl ether, DAST=Diethylaminosulfur trifluoride, DBU=1,8-Diazabicyclo[5.4.0]undec-7-ene, DIEA=N,N-diisopropylethylamine, DEA=diethanolamine, DMA=Dimethylacetamide, DMEDA=N,N′-Dimethylethylenediamine, DMAC=Dimethylacetamide, DMAP=4-dimethylaminopyridine, DMSO=dimethyl sulfoxide, DMF=N, N-dimethylacetamide, DCC=N,N′-Dicyclohexylcarbodiimide, DCM=dichloromethane, DMEM=Dulbecco's Modified Eagle Medium, DIBAL=Diisobutylaluminium hydride, dppf=1,1′-Bis(diphenylphosphino)ferrocene, dtbbpy=4,4′-Di-tert-butyl-2,2′-dipyridyl, EtOH=ethanol, EGTA=(ethylene glycol-bis(β-aminoethyl ethnmier)-N,N,N′,N′-tetraacetic acid), FA=formic acid, MEK=methyl ethyl ketone, MIBK=methyl isobutyl ketone, MeOH=methanol, MtBE=methyl tert-butyl ether, NBS=n-bromosuccinimide, NCS=n-chrlorosuccinimide, Otf=Trifluoromethanesulfonate, TEA or Et₃N=triethylamine, GOI=genes of interest, TsCl=p-toluenesulfonyl chloride, HATU=Hexafluorophosphate Azabenzotriazole Tetramethyl Uronium, hERG=human ether-a-go-go-related gene, HBSS=Hanks' Balanced Salt Solution, HEPES=4-(2-hydroxyethyl)-1-piperazineethane sulfonic acid, HOAt=1-Hydroxy-7-azabenzotriazole, HOBT=Hydroxybenzotriazole, IPA=isopropyl alcohol, IPAc=isopropyl acetate, LDA=Lithium diisopropylamide, L-Selectride=Lithium tri-secbutylborohydride, MtBE=methyl tert-butyl ether, NMP=N-methyl-2-pyrrolidone, SFC=Supercritical fluid chromatography, THF=tetrahydrofuran, THP=tetrahydropyranyl, TLC=thin layer chromatography, TBAF=tetra-n-butylammonium fluoride, TCFH=N,N,N′,N′-tetramethylchloroformamidinium hexafluorophosphate, TMSCl=trimethylsilyl chloride, TMSCN=trimethylsilyl cyanide, t-BuOH=tert-butyl alcohol, t-BuONa=sodium tert-butyloxide, OⁱPr=isopropoxide, Oac=acetate, OTBS=O-(tert-Butyldimethylsilyl), OMs=methanesulfonate, NMI=1-Methylimidazole, POCl₃=Phosphoryl chloride, POBr₃=Phosphoryl chloride, py=pyridine, Pd/C=palladium on carbon, HTRF—Homogeneous Time-Resolved Fluorescence, RT=retention time, h=hour, hrs=hours, aq.=aqueous, min.=minute, sat.=saturated, equiv=equivalent, e.e.=enantiomeric excess, UV=ultraviolet, XPhos=2-Dicyclohexylphosphino-2′,4′,6′-triisopropylbiphenyl, Ns=2-nitrobenzene-1-sulfonyl, ONs=2-nitrobenzenesulfonyl, OTs=p-toluenesulfonate.

Unless otherwise indicated, the chemical names used herein are generated using the Chemdraw Professional Version 23.0.1.11 software naming programs.

### Synthesis and Characterization Examples

For certain compounds disclosed herein the absolute stereochemistry has not been independently confirmed. At least one or more of the stereocenters' absolute stereochemistry are believed to be accurately represented in the name and structure of the disclosed compounds. For some compounds the stereochemistry is assigned based on analogy to known literature stereoisomers. The stereochemistry of the substituted methylene moiety (—C^{#2}(R^{d*})(R^{d})—) is believed to be accurate based on the absolute stereochemical assignment from commercially available vendors. (R)-2-hydroxypropanamide, also referred to as (R)-(+)-Lactamide, was either purchased from Bide Pharmatech Ltd. (CAS #598-81-2; Catalog #BD47136) or BBChem Co., Ltd (CAS #598-81-2; Catalog #BB211065; Batch #BB211065A23082301).

But, in some instances, the absolute stereo configurations of one or more chiral centers are arbitrarily assigned (e.g., stereochemistry of one chiral center is known and remaining chiral centers arbitrarily assigned). Accordingly, the enantiomers or diastereomers are identified by their respective properties, for example, retention times on a prep chiral HPLC, chiral SFC, NMR shift or optical rotation or its biological activities (e.g., as described further in the Examples). Thus, should the stereochemistry assigned to any compound or compounds ultimately be proven incorrect, then the analytical data (e.g., prep chiral HPLC, chiral SFC, NMR shift or optical rotation or biological activity) associated with each compound is determinative of the actual identity of the compound. In addition, in light of such corrected stereochemical designation appropriate adjustments to the stereochemistry identification contained in the description, examples, tables and claims should be adjusted as needed by one skilled in the art.

Chiral analytical separation methods (e.g., supercritical fluid chromatography (SFC), and high performance liquid chromatography (HPLC)) used in the following synthetic examples are summarized in the table below. These methods were used to identify a single compound/stereoisomer from a mixture of chiral compounds based on a peak retention time.

TABLE A
Chiral analytical separation methods

| | | | | | Column | Back |
|---|---|---|---|---|---|---|
| Method | Column | Mobile Phase | Flow Type | Flow Rate | Temp. | Pressure |
| A | CHIRAL | A: MtBE (0.1% | Isocratic: 30% B | 1.67 | Ambient | |
| | Cellulose-SB, | DEA) B: EtOH | | mL/min | | |
| | 0.46 × 5 cm, 3 μM | | | | | |
| B | CHIRALPAK IE-3, | A: MtBE (0.1% | Isocratic: 30% B | 1.67 | Ambient | |
| | 0.46 × 5 cm, 3 μM | DEA) B: MeOH | | mL/min | | |
| C | Cellulose SZ, | A: MtBE (0.1% | Isocratic: 50% B | 1.67 | Ambient | |
| | 0.46 × 5 cm, 3 μM | DEA) B: MeOH | | mL/min | | |
| D | Lux 3μ Cellulose-2, | A: Hex (0.1% | Isocratic: 40% B | 1.67 | Ambient | |
| | 4.6 × 5 cm, 3 μM | DEA) B: | | mL/min | | |
| | | EtOH:MeCN | | | | |
| | | (5:1) | | | | |
| E | CHIRALPAK IE-3, | A: MtBE (0.1% | Isocratic: 10% B | 1.67 | Ambient | |
| | 0.46 × 5 cm, 3 μM | TFA) B: MeOH | | mL/min | | |
| F | Enantiocel C4-3, | A: Hex (0.1% | Isocratic: 50% B | 1.67 | Ambient | |
| | 0.46 × 5 cm, 3 μM | DEA) B: | | mL/min | | |
| | | EtOH:MeCN | | | | |
| | | (5:1) | | | | |
| G | CHIRAL | A: MtBE (0.1% | Isocratic: 50% B | 1.67 | Ambient | |
| | Cellulose-SZ, | DEA) B: MeOH | | mL/min | | |
| | 0.46 × 5 cm, 3 μM | | | | | |
| H | CHIRALPAK ID-3, | A: MtBE (0.1% | Isocratic: 30% B | 1.67 | Ambient | |
| | 0.46 × 5 cm, 3 μM | DEA) B: MeOH | | mL/min | | |
| I | CHIRALPAK IE-3, | A: MtBE (0.1% | Isocratic: 20% B | 1.67 | Ambient | |
| | 0.46 × 5 cm, 3 μM | TFA) B: MeOH | | mL/min | | |
| J | CHIRALART | A: CO2 B: | Gradient: 10% to | 4.0 | 35° C. | 1500 psi |
| | Cellulose-SB, | MeOH (0.1% | 50% B in 2 min; | mL/min | | |
| | 0.46 × 5 cm, 3 μM | DEA) | hold at 50% B for | | | |
| | | | 1 min | | | |
| K | CHIRALPAK IH-3, | A: CO2 B: | Gradient: 5% to | 2.0 | 35° C. | 1500 psi |
| | 0.3 × 10 cm, 3 μM | MeOH (0.1% | 20% B in 6 min; | mL/min | | |
| | | DEA) | hold at 20% B for | | | |
| | | | 2 min | | | |
| L | CHIRALPAK IH-3, | A: CO2 B: | Gradient: 10% to | 2.0 | 35° C. | 1500 psi |
| | 0.3 × 10 cm, 3 μM | MeOH (0.1% | 50% B in 2 min; | mL/min | | |
| | | DEA) | hold at 50% B for | | | |
| | | | 1 min | | | |
| M | CHIRAL | A: MtBE (0.1% | Isocratic: 30% B | 1.67 | Ambient | |
| | Cellulose-SB, | DEA) B: MeOH | | mL/min | | |
| | 0.46 × 5 cm, 3 μM | | | | | |
| N | CHIRALART | A: CO2 B: | Gradient: 10% to | 2.0 | 35° C. | 1500 psi |
| | Cellulose-SZ, | MeOH (0.1% | 50% B in 2 min; | mL/min | | |
| | 0.3 × 5 cm, 3 μM | DEA) | hold at 50% B for | | | |
| | | | 1 min | | | |

### Synthesis of Intermediate A: (R)-1-(((R)-5-(3,5-difluorophenyl)-6,7-dihydro-5H-pyrrolo[1,2-a]imidazol-2-yl)amino)-1-oxopropan-2-yl 2-nitrobenzenesulfonate

### Step 1: ethyl 4-(3,5-difluorophenyl)-4-oxobutanoate

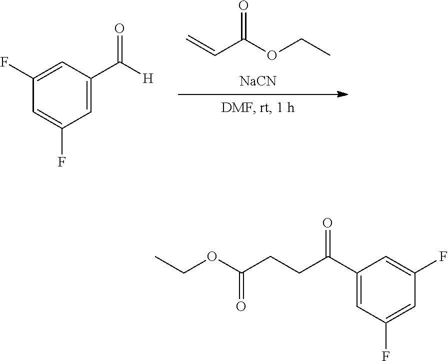

(5 batches)—To a suspension of NaCN (7.38 g, 150.6 mmol) in DMF (1070 mL) was added 3,5-difluorobenzaldehyde (214 g, 1.51 mol) and ethyl acrylate (153.8 g, 1.54 mol) at room temperature. After addition, the reaction mixture was stirred at room temperature. A significant exotherm was observed after 3 minutes (25° C. to 85° C. within 2 minutes). The mixture was stirred until it had cooled back down to 25° C. Upon completion, EtOAc (2140 mL) and water (2140 mL) were then added. Then a NaOCl solution (contains more than 10% available chlorine, 1300 mL) was added to quench the cyanide ions, while maintaining the internal temperature below 35° C. The 5 batches were combined and worked up together. The resulting mixture was phase-separated, and the aqueous phase was extracted with EtOAc (9.6 L). The combined organic layers were washed with brine (5% NaCl aq., 9.6 L) three times, dried over anhydrous Na₂SO₄, filtered and concentrated under reduced pressure. The crude residue was purified by column silica gel chromatography, eluted with PE/EtOAc (100:1 to 10:1) to give ethyl 4-(3,5-difluorophenyl)-4-oxobutanoate as a brown solid (1160 g, 69.2% yield). LCMS (ESI) [M+H]⁺: 243.1.

### Step 2: ethyl (R)-4-(((S)-tert-butylsulfinyl)amino)-4-(3,5-difluorophenyl)butanoate

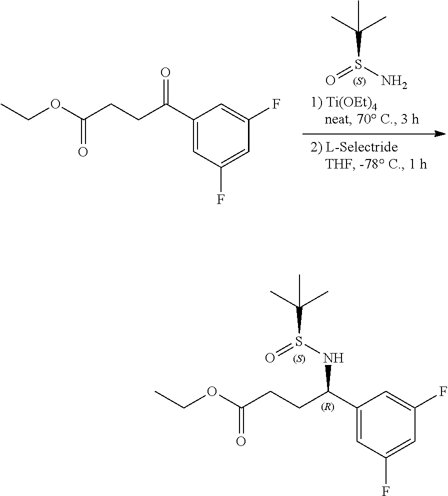

(4 batches)—(S)-tert-Butylsulfinamide (137.6 g, 1.14 mol) was added to molten Ti(OEt)₄ (518.0 g, 2.27 mol, contains approximately 20% Ti(OⁱPr)₄ and ethyl 4-(3,5-difluorophenyl)-4-oxobutanoate (275.0 g, 1.14 mol) at 70° C. The reaction mixture was stirred for 3 hours at 70±5° C. Upon completion, the reaction was cooled to 25° C. and THF (2.75 L) was added at 25±5° C. The reaction mixture was cooled to −78° C. and L-Selectride (1.37 L, 1.0 mol/L in THF) was added while maintaining the internal temperature<−70° C. The resulting mixture was stirred for at −75±5° C. for 1 hour. Upon completion, a NaHCO₃ solution (5% w/w aq., 10 V) was added. The 4 batches were combined and worked up together. EtOAc (20.0 V) was added and the mixture stirred for 30 min at 20±5° C. The resulting mixture was filtered over Celite. The filtrate was phase-separated. The organic layer was washed with brine (5% w/w aq., 5.0 V×2), and concentrated under reduced pressure to afford the crude product as a yellow oil. The crude product was recrystallized with Petroleum ether (10.0 V) at −10° C. The resulting mixture was filtered, and the wet filter cake was dried in under vacuum at 50° C. to afford ethyl (R)-4-(((S)-tert-butylsulfinyl)amino)-4-(3,5-difluorophenyl)butanoate (440.0 g—first fraction). The filtrate was purified by silica gel column chromatography, eluted with PE/EtOAc (50:1 to 3:1) to afford the crude product (440 g) as a yellow oil, which was then recrystallized with Petroleum ether (10.0 V) at −10° C. to afford ethyl (R)-4-(((S)-tert-butylsulfinyl)amino)-4-(3,5-difluorophenyl)butanoate (228.0 g—second fraction). LCMS (ESI) [M+H]⁺: 348.0.

### Step 3: (R)-5-(3,5-difluorophenyl)pyrrolidin-2-one

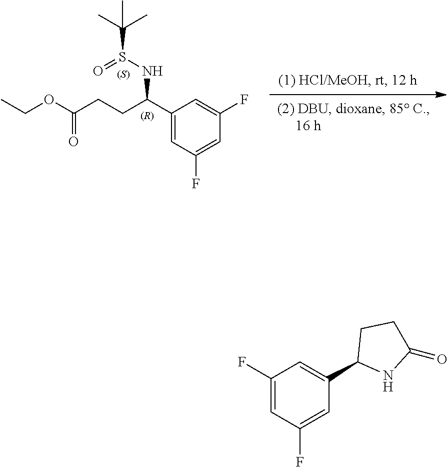

To the solution of ethyl (R)-4-(((S)-tert-butylsulfinyl)amino)-4-(3,5-difluorophenyl)butanoate (628.0 g, 1.8 mol) in MeOH (1.9 L) was added HCl/MeOH (2M, 4.0 V) at room temperature. After addition, the reaction mixture was stirred for 12 hours. Upon completion, the reaction mixture was concentrated under reduced pressure to remove EtOH. Then MeOH (1.9 L, 3.0 V) and HCl/MeOH (2M, 4.0 V) were added to the residue at room temperature. The reaction mixture was stirred for additional 12 hours. Upon completion, the reaction mixture was concentrated under reduced pressure. 1,4-Dioxane (6.3 L, 10.0 V) and DBU (1.5 eq.), were added to the crude residue and the resulting mixture was warmed to 85° C. After 16 hours, the reaction was cooled to room temperature and concentrated under reduced pressure. The crude residue was purified by silica gel column chromatography, eluted with DCM/MeOH (100:1 to 20:1) to afford (R)-5-(3,5-difluorophenyl)pyrrolidin-2-one as a brown solid (318 g, 73% yield, 99.5% ee, chiral HPLC conditions: DAICEL CHIRALPAK, OC-3, 3 μm, 4.6 mm*100 mm, 210 nm, flow rate: 0.8 mL/min, EtOH/hexanes=1/5, tₘᵢₙₒᵣ=5.89 min, tₘₐⱼₒᵣ=6.99 min). LCMS (ESI) [M+H]⁺: 198.0.

### Step 4: (R)-2-(2-(3,5-difluorophenyl)-5-oxopyrrolidin-1-yl)acetamide

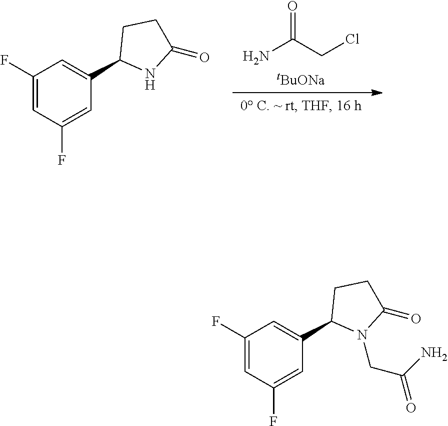

(3 batches)—To a solution of (R)-5-(3,5-difluorophenyl)pyrrolidin-2-one (100 g, 0.507 mol) in THF (4 L) was added tBuONa (58.5 g, 0.610 mol) at 0° C. After 10 minutes, chloroacetamide (52.2 g, 0.558 mol) was added in portions. The resulting mixture was allowed to warm to room temperature and stirred for 12 hours. Upon completion, AcOH (20 mL, 0.2 V) was added to adjust the pH of the reaction mixture to 5~6. The 3 batches were combined and concentrated under reduced pressure. The crude residue was purified by silica gel chromatography (DCM/MeOH: 30:1) to give (R)-2-(2-(3,5-difluorophenyl)-5-oxopyrrolidin-1-yl)acetamide as a pale yellow solid (328 g, 81% yield). LCMS (ESI) [M+H]⁺: 254.9.

### Step 5: (R)-2-bromo-5-(3,5-difluorophenyl)-6,7-dihydro-5H-pyrrolo[1,2-a]imidazole

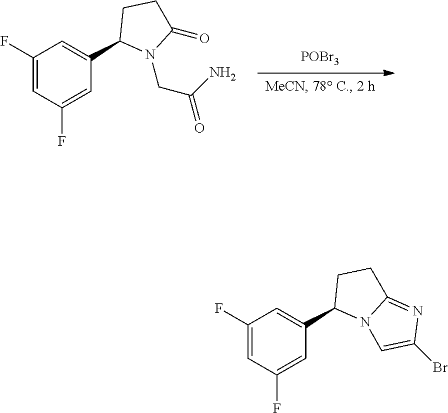 [SMILES: NC(=O)CN1C(=O)CC[C@@H]1c1cc(F)cc(F)c1 ; "POBr3" ; "MeCN, 78° C., 2 h" ; Fc1cc(F)cc([C@H]2CCc3nc(Br)cn32)c1]

(2 batches)—To a solution of (R)-2-(2-(3,5-difluorophenyl)-5-oxopyrrolidin-1-yl)acetamide (154 g, 0.606 mol) in MeCN (3.1 L, 20.0 V) was added POBr₃ (1146.8 g, 2.424 mol) dropwise at room temperature. The resulting mixture was warmed to 78° C. and stirred for 2 hours. Upon completion, the mixture was cooled to room temperature and quenched by addition of water (1.54 L, 10.0 V) while maintaining internal temperature at 30~40° C. A saturated solution of NaOH as added to the mixture to adjust the pH to 5~7. The 2 batches combined and extracted with EtOAc (1.54 L, 10.0 V). The organic layer was washed with brine (0.77 L, 5.0 V), dried over anhydrous Na₂SO₄, filtered and concentrated under reduced pressure. The crude residue was purified by silica gel chromatography (PE/EA: 1:1) to give (R)-2-bromo-5-(3,5-difluorophenyl)-6,7-dihydro-5H-pyrrolo[1,2-a]imidazole as a pale yellow solid (358 g, 90% yield). LCMS (ESI) [M+H]⁺: 298.8.

### Step 6: (R)—N—((R)-5-(3,5-difluorophenyl)-6,7-dihydro-5H-pyrrolo[1,2-a]imidazol-2-yl)-2-hydroxypropanamide

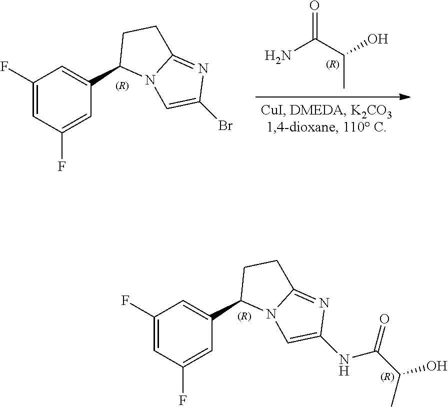

To a mixture of (R)-2-bromo-5-(3,5-difluorophenyl)-6,7-dihydro-5H-pyrrolo[1,2-a]imidazole (500 mg, 1.7 mmol) in 1,4-dioxane (12 mL) were added (R)-2-hydroxypropanamide (298 mg, 3.3 mmol), CuI (64 mg, 0.3 mmol), N,N-dimethyl-1,2-ethanediamine (59 mg, 0.4 mmol) and K₂CO₃ (463 mg, 3.4 mmol). The resulting mixture was stirred at 110° C. overnight under nitrogen atmosphere. Upon completion, the mixture was allowed to cool down to room temperature. The reaction was quenched with water at 0° C. The resulting mixture was extracted with EtOAc (3×20 mL). The combined organic layers were dried over anhydrous Na₂SO₄, filtered and concentrated under reduced pressure. The crude residue was purified by silica gel column chromatography, eluted with DCM/MeOH (19:1) to afford (R)—N—((R)-5-(3,5-difluorophenyl)-6,7-dihydro-5H-pyrrolo[1,2-a]imidazol-2-yl)-2-hydroxypropanamide (240 mg, 47% yield) as a yellow solid. LCMS (ESI) [M+H]⁺: 307.9.

### Step 7: (R)-1-(((R)-5-(3,5-difluorophenyl)-6,7-dihydro-5H-pyrrolo[1,2-a]imidazol-2-yl)amino)-1-oxopropan-2-yl 2-nitrobenzenesulfonate

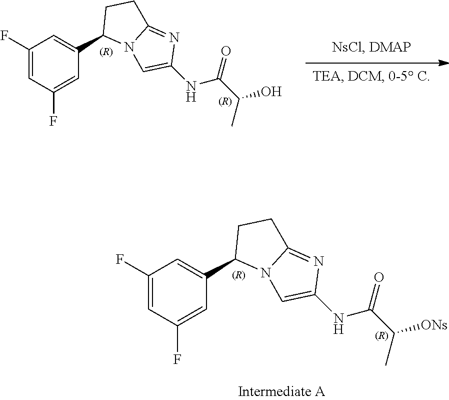

To a mixture of (R)—N—((R)-5-(3,5-difluorophenyl)-6,7-dihydro-5H-pyrrolo[1,2-a]imidazol-2-yl)-2-hydroxypropanamide (240 mg, 0.8 mmol) in DCM (15 mL) were added 2 -nitrobenzenesulfonyl chloride (208 mg, 0.9 mmol), DMAP (19 mg, 0.160 mmol) and TEA (237 mg, 2.4 mmol) at 0° C. under nitrogen atmosphere. The resulting mixture was stirred at 0° C. for 5 hours. Upon completion, the reaction was quenched with water at 0° C. The resulting mixture was extracted with EtOAc (3×30 mL). The combined organic layers were dried over anhydrous Na₂SO₄, filtered concentrated and concentrated under reduced pressure. The crude residue was purified by silica gel column chromatography, eluted with DCM/EA (1:1) to afford (R)-1-(((R)-5-(3,5-difluorophenyl)-6,7-dihydro-5H-pyrrolo[1,2-a]imidazol-2-yl)amino)-1-oxopropan-2-yl 2-nitrobenzenesulfonate (Intermediate A) (162 mg, 42% yield) as a yellow solid. LCMS (ESI) [M+H]⁺: 493.0.

### Synthesis of Intermediate B and Intermediate C: (2R)-1-((4-(3,5-difluorophenyl)-5,6-dihydro-4H-pyrrolo[1,2-b]pyrazol-2-yl)amino)-1-oxopropan-2-yl 2-nitrobenzenesulfonate & (2S)—N-(4-(3,5-difluorophenyl)-5,6-dihydro-4H-pyrrolo[1,2-b]pyrazol-2-yl)-2-(3,3-dimethylpiperazin-1-yl)propanamide

### Step 1: 3,5-dibromo-1-(tetrahydro-2H-pyran-2-yl)-1H-pyrazole

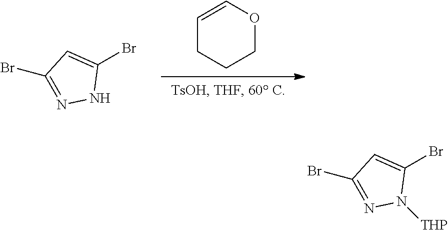

A solution of 3,5-dibromo-1H-pyrazole (5.00 g, 22.1 mmol), 3,4-dihydro-2H-pyran (2.23 g, 26.6 mmol) and TsOH monohydrate (381 mg, 2.2 mmol) in THF (60 mL) was stirred at 60° C. for 2 hours under nitrogen atmosphere. Upon completion, the mixture was allowed to cool down to room temperature. The resulting mixture was concentrated under reduced pressure. The crude residue was purified by silica gel column chromatography, eluted with PE/EA (1:1) to afford 3,5-dibromo-1-(tetrahydro-2H-pyran-2-yl)-1H-pyrazole (5.30 g, 77% yield) as a white solid. LCMS (ESI) [M+H]⁺: 309.

### Step 2: 3-bromo-5-(3,5-difluorobenzyl)-1-(tetrahydro-2H-pyran-2-yl)-1H-pyrazole

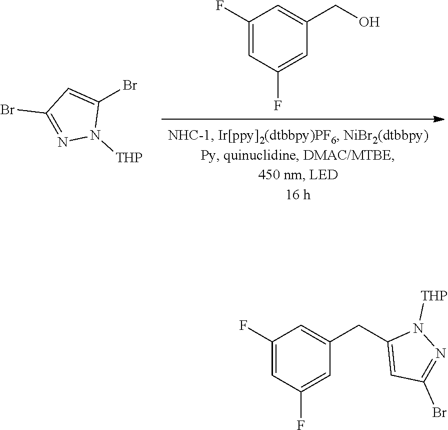

An oven-dried 50 mL vial was charged with (3,5-difluorophenyl)methanol (1.22 g, 8.5 mmol), 5,7-di-tert-butyl-3-phenylbenzo[d]oxazol-3-ium tetrafluoroborate (NHC-1; 3.06 g, 7.7 mmol) and a magnetic stir bar. After, the vial was vacuumed and refilled with nitrogen gas twice. 2-Methoxy-2-methylpropane (37 mL) was added and the reaction stirred at 0° C. for 10 minutes. Then a pyridine solution (0.61 g, 7.7 mmol in 0.5 mL 2-methoxy-2-methylpropane) was added dropwise at 0° C. over the course of 2 minutes. The resulting solution stirred at room temperature for 10 minutes. A white solid precipitated out during this time. In a separate oven-dried 250 mL flask N,N-Dimethylacetamide (37.5 mL) was added to [Ir(ppy)₂(dtbbpy)]PF₆ (0.07 g, 73.0 μmol), [4,4′-Bis(tert-butyl)-2,2′-bipyridine]nickel dibromide (0.18 g, 0.4 mmol), 1-azabicyclo[2.2.2]octane (0.94 g, 8.5 mmol), 3,5-dibromo-1-(oxan-2-yl)pyrazole (1.50 g, 4.8 mmol) and a magnetic stir bar were added under nitrogen atmosphere. The flask containing the methyl tert-butyl ether suspension was transferred to a 50 mL syringe under air. Then, a syringe filter and new needle were installed on the syringe before the methyl tert-butyl ether solution was injected through the syringe filter into the dimethylacetamide solution. The reaction mixture was sparged with nitrogen for 15 minutes before being sealed with parafilm. The flask was then stirred and irradiated under 450 nm LED modules for 16 hours. Upon completion, the reaction was quenched with water. The resulting mixture was extracted with EtOAc (3×90 mL). The combined organic layers were washed with water (3×95 mL), dried over anhydrous Na₂SO₄, filtered and concentrated under reduced pressure. The crude residue was purified by reversed-phase flash chromatography (conditions: column: C18 silica gel; mobile phase A: water (0.1% formic acid), Mobile Phase B: MeCN; Gradient (B %): 0% to 100% in 25 minutes; detector: UV 254 nm) to afford 3-bromo-5-(3,5-difluorobenzyl)-1-(tetrahydro-2H-pyran-2-yl)-1H-pyrazole (800 mg, 46% yield) as a light yellow oil. LCMS (ESI) [M+H]⁺: 357.

### Step 3: 3-bromo-5-(3-((tert-butyldimethylsilyl)oxy)-1-(3,5-difluorophenyl)propyl)-1-(tetrahydro-2H-pyran-2-yl)-1H-pyrazole

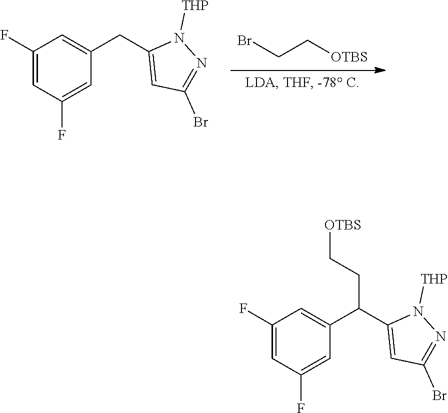

LDA (1.5 M in THF, 1.6 mL, 2.4 mmol) was added dropwise to a solution of 3-bromo-5-(3,5-difluorobenzyl)-1-(tetrahydro-2H-pyran-2-yl)-1H-pyrazole (700 mg, 2.0 mmol) in THF (30 mL) dropwise over 10 minutes at −78° C. under nitrogen atmosphere. After 15 minutes (2-bromoethoxy)(tert-butyl)dimethylsilane (562 mg, 2.4 mmol) was added dropwise over 10 min at −78° C. The resulting mixture was stirred at −78° C. for an additional 2 hours. Upon completion, the reaction mixture was quenched with saturated aqueous NH₄Cl solution (15 mL) at 0° C. The aqueous layer was extracted with EtOAc (3×60 mL). The combined organic layers were dried over anhydrous Na₂SO₄, filtered and concentrated under reduced pressure. The crude residue was purified by silica gel column chromatography, eluted with PE/EA (2:1) to afford 3-bromo-5-(3-((tert-butyldimethylsilyl)oxy)-1-(3,5-difluorophenyl)propyl)-1-(tetrahydro-2H-pyran-2-yl)-1H-pyrazole (600 mg, 59% yield) as a colorless oil. LCMS (ESI) [M+H]⁺: 515.

### Step 4: 3-(3-bromo-1H-pyrazol-5-yl)-3-(3,5-difluorophenyl)propan-1-ol

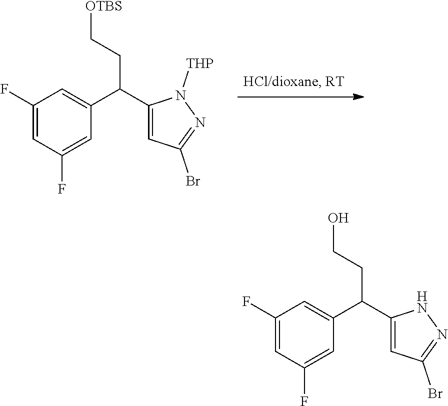

A solution of 3-bromo-5-(3-((tert-butyldimethylsilyl)oxy)-1-(3,5-difluorophenyl)propyl)-1-(tetrahydro-2H-pyran-2-yl)-1H-pyrazole (600 mg, 1.2 mmol) in 4M HCl in 1,4-dioxane (8 mL) was stirred at room temperature for 2 hours. Upon completion, the resulting mixture was concentrated under reduced pressure. The crude residue was purified by reversed-phase flash chromatography (conditions: column: C18 silica gel; mobile phase A: water (0.1% formic acid), Mobile Phase B: MeCN; Gradient (B %): 0% to 100% in 20 minutes; detector: UV 254 nm) to afford 3-(3-bromo-1H-pyrazol-5-yl)-3-(3,5-difluorophenyl)propan-1-ol (210 mg, 57% yield) as a colorless oil. LCMS (ESI) [M+H]⁺: 317.

### Step 5: 2-bromo-4-(3,5-difluorophenyl)-5,6-dihydro-4H-pyrrolo[1,2-b]pyrazole

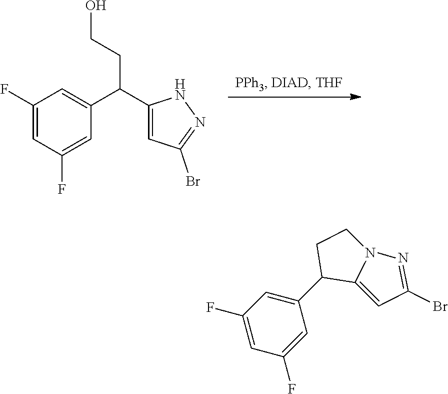

A solution of 3-(3-bromo-1H-pyrazol-5-yl)-3-(3,5-difluorophenyl)propan-1-ol (200 mg, 0.6 mmol) and PPh₃ (198 mg, 0.8 mmol) in THF (10 mL) was stirred at 0° C. under nitrogen atmosphere. After 10 minutes, DIAD (153 mg, 0.8 mmol) was added dropwise over 5 minutes. The resulting mixture was warmed to room temperature and stirred for an additional 2 hours. Upon completion, the reaction mixture was quenched with water (10 mL) at 0° C. The resulting mixture was extracted with EtOAc (3×20 mL). The combined organic layers were washed with brine, dried over anhydrous Na₂SO₄, filtered and concentrated under reduced pressure. The crude residue was purified by reversed-phase flash chromatography (conditions: column: C18 silica gel; mobile phase A: water (0.1% formic acid), Mobile Phase B: MeCN; Gradient (B %): 0% to 100% in 20 minutes; detector: UV 254 nm) to afford 2-bromo-4-(3,5-difluorophenyl)-5,6 -dihydro-4H-pyrrolo[1,2-b]pyrazole (120 mg, 64% yield) as a light yellow oil. LCMS (ESI) [M+H]⁺: 299.

### Step 6: (2R)—N-(4-(3,5-difluorophenyl)-5,6-dihydro-4H-pyrrolo[1,2-b]pyrazol-2-yl)-2-hydroxypropanamide

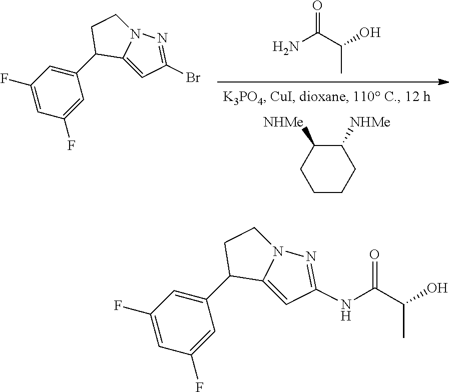

A solution of 2-bromo-4-(3,5-difluorophenyl)-5,6-dihydro-4H-pyrrolo[1,2-b]pyrazole (120 mg, 0.4 mmol), (R)-2-hydroxypropanamide (42 mg, 0.5 mmol), (1R,2R)—N¹,N²-dimethylcyclohexane-1,2-diamine (22 mg, 0.2 mmol), CuI (15 mg, 80 μmol) and K₃PO₄ (170 mg, 0.8 mmol) in 1,4-dioxane (10 mL) was stirred at 110° C. for 12 hours under nitrogen atmosphere. Upon completion, the mixture was allowed to cool down to room temperature and was diluted with water (10 mL) at 0° C. The resulting mixture was extracted with EtOAc (3×20 mL). The combined organic layers were dried over anhydrous Na₂SO₄, filtered and concentrated under reduced pressure. The crude residue was purified by reversed-phase flash chromatography (conditions: column: C18 silica gel; mobile phase A: water (0.1% formic acid), Mobile Phase B: MeCN; Gradient (B %): 0% to 100% in 20 minutes; detector: UV 254 nm) to afford (2R)—N-(4-(3,5-difluorophenyl)-5,6-dihydro-4H-pyrrolo[1,2-b]pyrazol-2-yl)-2-hydroxypropanamide (100 mg, 81% yield) as a light yellow solid. LCMS (ESI) [M+H]⁺: 308.

### Step 7: (2R)-1-((4-(3,5-difluorophenyl)-5,6-dihydro-4H-pyrrolo[1,2-b]pyrazol-2-yl)amino)-1-oxopropan-2-yl 2-nitrobenzenesulfonate

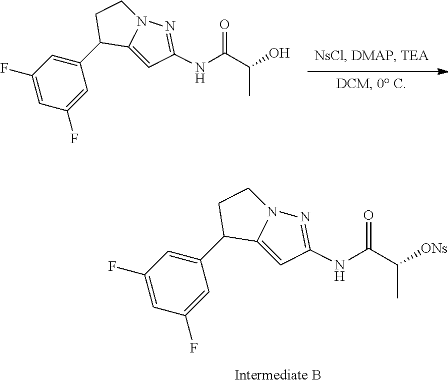

A solution of (2R)—N-(4-(3,5-difluorophenyl)-5,6-dihydro-4H-pyrrolo[1,2-b]pyrazol-2-yl)-2-hydroxypropanamide (100 mg, 0.3 mmol), DMAP (8 mg, 60 μmol) and 2-nitrobenzenesulfonyl chloride (28 mg, 0.1 mmol) in DCM (6 mL) was stirred at 0° C. under nitrogen atmosphere. After 3 minutes, TEA (98 mg, 1.0 mmol) was added dropwise over 5 minutes at 0° C. The resulting mixture was stirred at 0° C. for an additional 5 hours. Upon completion, the reaction mixture was diluted with ice water (10 mL) at 0° C. The resulting mixture was extracted with CH₂Cl₂ (3×12 mL). The combined organic layers were dried over anhydrous Na₂SO₄, filtered and concentrated under reduced pressure. The crude residue was purified by silica gel column chromatography, eluted with CH₂Cl₂/EA (1:1) to afford (2R)-1-((4-(3,5-difluorophenyl)-5,6-dihydro-4H-pyrrolo[1,2-b]pyrazol-2-yl)amino)-1-oxopropan-2-yl 2-nitrobenzenesulfonate (Intermediate B) (70 mg, 44% yield) as a yellow solid. LCMS (ESI) [M+H]⁺: 493.

### Step 8: tert-butyl 4-((2S)-1-((4-(3,5-difluorophenyl)-5,6-dihydro-4H-pyrrolo[1,2-b]pyrazol-2-yl)amino)-1-oxopropan-2-yl)-2,2-dimethylpiperazine-1-carboxylate

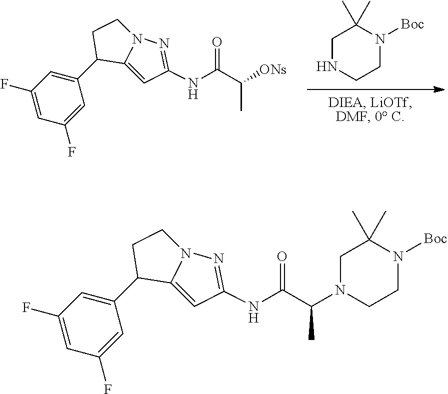

To a mixture of tert-butyl 2,2-dimethylpiperazine-1-carboxylate (818 mg, 3.8 mmol) in DMF (20 mL) were added DIEA (740 mg, 5.7 mmol), lithium trifluoromethanesulfonate (893 mg, 5.7 mmol) and (2R)-1-((4-(3,5-difluorophenyl)-5,6-dihydro-4H-pyrrolo[1,2-b]pyrazol-2-yl)amino)-1-oxopropan-2-yl 2-nitrobenzenesulfonate (940 mg, 1.9 mmol) dropwise at 0° C. under nitrogen atmosphere. The resulting mixture was stirred at 0° C. for 2 days. Upon completion, the reaction mixture was directly purified by reversed-phase flash chromatography (conditions: column: C18 silica gel; mobile phase A: water (10 mmol/L NH₄HCO₃), Mobile Phase B: MeCN; Gradient (B %): 0% to 100% in 20 minutes; detector: UV 254 nm) to afford tert-butyl 4-((2S)-1-((4-(3,5-difluorophenyl)-5,6-dihydro-4H-pyrrolo[1,2-b]pyrazol-2-yl)amino)-1-oxopropan-2-yl)-2,2-dimethylpiperazine-1-carboxylate (868 mg, 90% yield) as a yellow solid. LCMS (ESI) [M+H]⁺: 504.

### Step 9: (2S)—N-(4-(3,5-difluorophenyl)-5,6-dihydro-4H-pyrrolo[1,2-b]pyrazol-2-yl)-2-(3,3-dimethylpiperazin-1-yl)propanamide

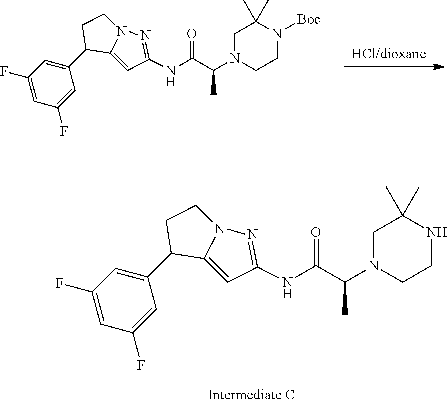 [SMILES: C[C@@H](C(=O)Nc1cc2n(n1)CCC2c1cc(F)cc(F)c1)N1CCN(C(=O)OC(C)(C)C)C(C)(C)C1 ; "HCl/dioxane" ; C[C@@H](C(=O)Nc1cc2n(n1)CCC2c1cc(F)cc(F)c1)N1CCNC(C)(C)C1]

To a mixture of tert-butyl 4-((2S)-1-((4-(3,5-difluorophenyl)-5,6-dihydro-4H-pyrrolo[1,2-b]pyrazol-2-yl)amino)-1-oxopropan-2-yl)-2,2-dimethylpiperazine-1-carboxylate (868 mg, 1.7 mmol) in 1,4-dioxane (2 mL) was added 4M HCl in 1,4-dioxane (10 mL) dropwise at 0° C. The resulting mixture was warmed to room temperature and stirred for 2 hours. Upon completion, the resulting mixture was concentrated under reduced pressure to afford (2S)—N-(4-(3,5-difluorophenyl)-5,6-dihydro-4H-pyrrolo[1,2-b]pyrazol-2-yl)-2-(3,3-dimethylpiperazin-1-yl)propanamide (Intermediate C) (780 mg, crude) as a white solid, which was used in the next step without further purification. LCMS (ESI) [M+H]⁺: 404.

### Example 1: 4-(4-((S)-1-(((R)-5-(3,5-difluorophenyl)-6,7-dihydro-5H-pyrrolo[1,2-a]imidazol-2-yl)amino)-1-oxopropan-2-yl)-2,2-dimethylpiperazine-1-carbonyl)pyridine 1-oxide

### Step 1: tert-butyl 4-((S)-1-(((R)-5-(3,5-difluorophenyl)-6,7-dihydro-5H-pyrrolo[1,2-a]imidazol-2-yl)amino)-1-oxopropan-2-yl)-2,2-dimethylpiperazine-1-carboxylate

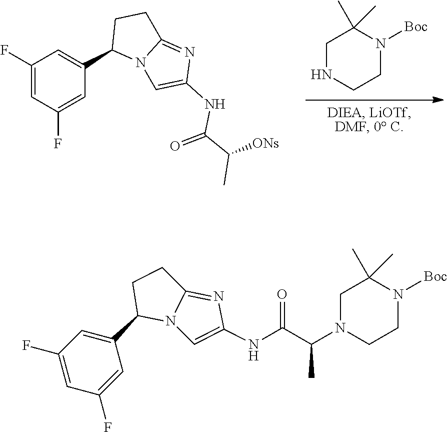

To a mixture of tert-butyl 2,2-dimethylpiperazine-1-carboxylate (783 mg, 3.7 mmol) in DMF (10 mL) were added DIEA (708 mg, 5.5 mmol), lithium trifluoromethanesulfonate (855 mg, 5.5 mmol) and (R)-1-(((R)-5-(3,5-difluorophenyl)-6,7-dihydro-5H-pyrrolo[1,2-a]imidazol-2-yl)amino)-1-oxopropan-2-yl 2-nitrobenzenesulfonate (900 mg, 1.8 mmol) in DMF (10 mL) dropwise at 0° C. under nitrogen atmosphere. The resulting mixture was stirred at 0° C. for 2 days. Upon completion, the reaction mixture was directly purified by reversed-phase flash chromatography (conditions: column: C18 silica gel; mobile phase A: water (10 mmol/L NH₄HCO₃), Mobile Phase B: MeCN; Gradient (B %): 0% to 100% in 20 minutes; detector: UV 254 nm) to afford tert-butyl 4-((S)-1-(((R)-5-(3,5-difluorophenyl)-6,7-dihydro-5H-pyrrolo[1,2-a]imidazol-2-yl)amino)-1-oxopropan-2-yl)-2,2-dimethylpiperazine-1-carboxylate (750 mg, 81% yield) as a yellow solid. LCMS (ESI) [M+H]⁺: 504.

### Step 2: (S)—N—((R)-5-(3,5-difluorophenyl)-6,7-dihydro-5H-pyrrolo[1,2-a]imidazol-2-yl)-2-(3,3-dimethylpiperazin-1-yl)propanamide

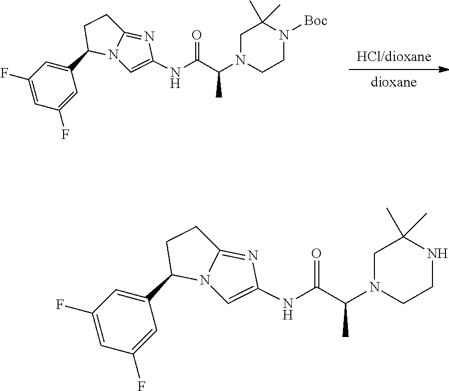 [SMILES: C[C@@H](C(=O)Nc1cn2c(n1)CC[C@@H]2c1cc(F)cc(F)c1)N1CCN(C(=O)OC(C)(C)C)C(C)(C)C1 ; "HCl/dioxane" ; "dioxane" ; C[C@@H](C(=O)Nc1cn2c(n1)CC[C@@H]2c1cc(F)cc(F)c1)N1CCNC(C)(C)C1]

To a mixture of tert-butyl 4-((S)-1-(((R)-5-(3,5-difluorophenyl)-6,7-dihydro-5H-pyrrolo[1,2-a]imidazol-2-yl)amino)-1-oxopropan-2-yl)-2,2-dimethylpiperazine-1-carboxylate (730 mg, 1.5 mmol) in 1,4-dioxane (2 mL) was added 4M HCl in 1,4-dioxane (10 mL) dropwise at 0° C. The resulting mixture was warmed to room temperature and stirred for 1 hour. Upon completion, the reaction mixture was concentrated under reduced pressure. The crude residue was purified by reversed-phase flash chromatography (conditions: column: C18 silica gel; mobile phase A: water (0.1% formic acid), Mobile Phase B: MeCN; Gradient (B %): 0% to 100% in 20 minutes; detector: UV 254 nm) to afford (S)—N—((R)-5-(3,5-difluorophenyl)-6,7-dihydro-5H-pyrrolo[1,2-a]imidazol-2-yl)-2-(3,3-dimethylpiperazin-1-yl)propanamide (500 mg, 85% yield) as a white solid. LCMS (ESI) [M+H]⁺: 404

### Step 3: 4-(4-((S)-1-(((R)-5-(3,5-difluorophenyl)-6,7-dihydro-5H-pyrrolo[1,2-a]imidazol-2-yl)amino)-1-oxopropan-2-yl)-2,2-dimethylpiperazine-1-carbonyl)pyridine 1-oxide

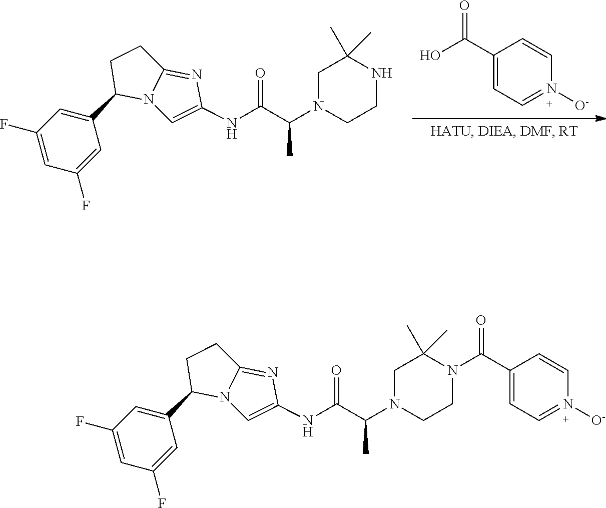

A solution of (S)—N—((R)-5-(3,5-difluorophenyl)-6,7-dihydro-5H-pyrrolo[1,2-a]imidazol-2-yl)-2-(3,3-dimethylpiperazin-1-yl)propanamide (50 mg, 0.1 mmol), 4-carboxypyridine 1-oxide (25 mg, 0.2 mmol), HATU (70 mg, 0.2 mmol) and DIEA (48 mg, 0.4 mmol) in DMF (3 mL) were stirred at room temperature for 1 hour under nitrogen atmosphere. Upon completion, the resulting mixture was directly purified by Prep-HPLC (conditions: Column: Xbridge Phenyl OBD Column, 30*150 mm 5 μm; Mobile Phase A: Water (10 mmol/L NH₄HCO₃), Mobile Phase B: ACN; Flow rate: 60 mL/min; Gradient (B %): 27% to 42% in 17 minutes; Wave Length: 254 nm/220 nm; RT1 (min): 9.72) to afford 4-(4-((S)-1-(((R)-5-(3,5-difluorophenyl)-6,7-dihydro-5H-pyrrolo[1,2-a]imidazol-2-yl)amino)-1-oxopropan-2-yl)-2,2-dimethylpiperazine-1-carbonyl)pyridine 1-oxide (23.5 mg, Compound 1) as a white solid.

| Compound# | Characterization Data |
|---|---|
| 1 | LCMS (ESI) [M + H]⁺: 525.3 |
| | ¹H NMR (400 MHz, DMSO-d₆) δ 9.96 (s, 1H), |
| | 8.20 (d, J = 7.2 Hz, 2H), 7.42 (d, J = 7.2 Hz, |
| | 2H), 7.23-7.19 (m, 1H), 6.97-6.95 |
| | (m, 3H), 5.39-5.37 (m, 1H), 3.38-3.36 |
| | (m, 1H), 3.31-3.26 (m, 2H), 2.97-2.86 |
| | (m, 2H), 2.84-2.74 (m, 1H), |
| | 2.65-2.61 (m, 1H), 2.56-2.50 (m, 1H), |
| | 2.45-2.38 (m, 3H), 1.44 (d, J = 4.8 |
| | Hz, 6H), 1.15 (d, J = 6.8 Hz, 3H). |

### Compound 1 Absolute Crystal Structure Determination

The crystal of Compound 1 was obtained by recrystallization in acetone via slow solvent evaporation.

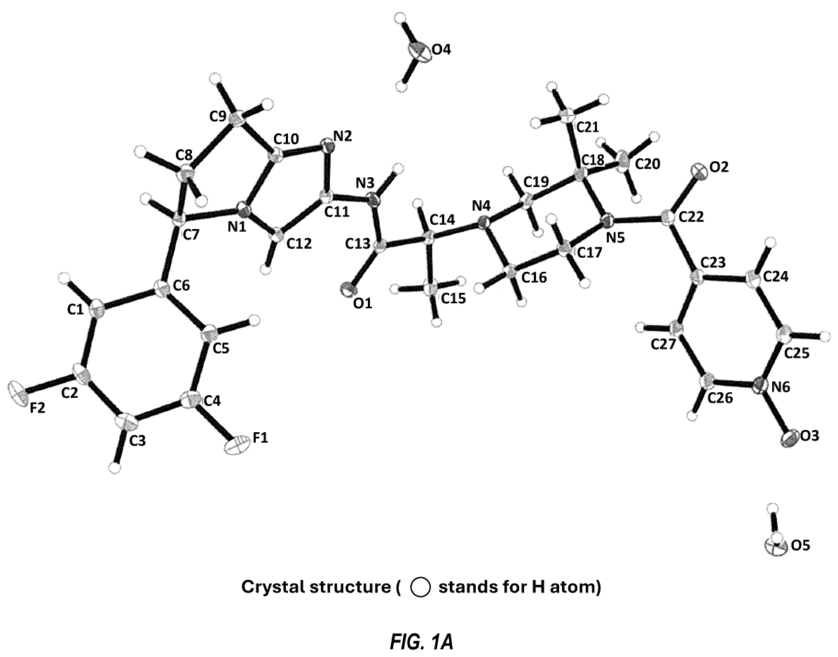

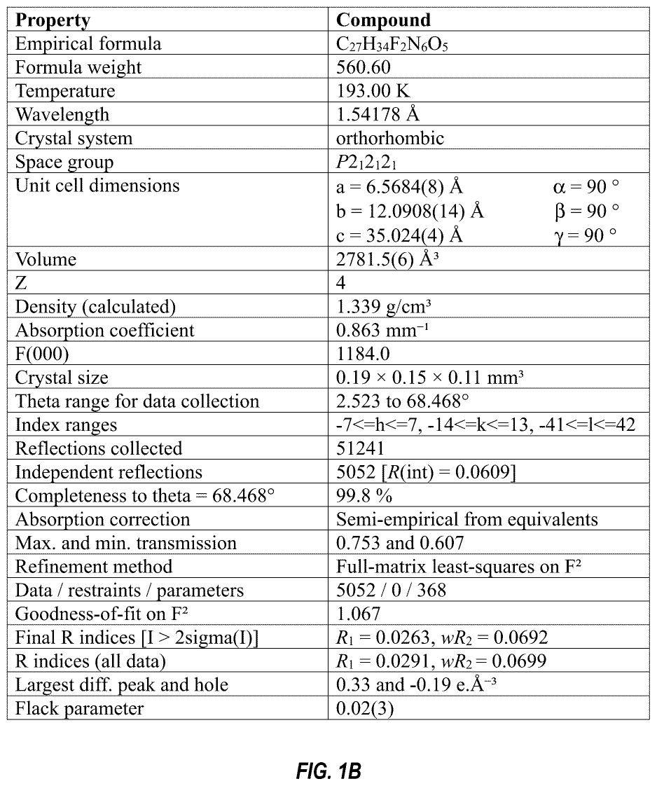

The single crystal X-Ray diffraction results using the crystalline sample of Compound 1 is shown in FIG. 1 (the crystal structure is shown in FIG. 1A, and crystal data are shown in FIGS. 1B-1E).

Compound 1 belongs to the orthorhombic system with the space group P2₁2₁2₁, and the molecular formula is C₂₇H₃₄F₂N₆O₅. The smallest asymmetric unit of the single crystal structure contains one organic molecule of the Compound 1 sample and two molecules of solvent water. The absolute configuration of the compound was successfully determined by single-crystal diffraction data, with the chiral carbons C7 and C14 identified as “R” and “S” configurations, respectively. The structure of the selected crystal sample, as determined by single-crystal X-ray data, is presented in the figure below.

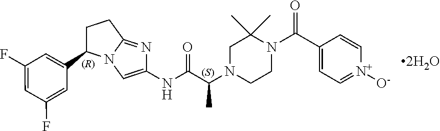

### Example 2: (S)—N—((R)-5-(3,5-difluorophenyl)-6,7-dihydro-5H-pyrrolo[1,2-a]imidazol-2-yl)-2-(3,3-dimethyl-4-(6-oxo-1,6-dihydropyridine-3-carbonyl)piperazin-1-yl)propanamide

### Step 1: (S)—N—((R)-5-(3,5-difluorophenyl)-6,7-dihydro-5H-pyrrolo[1,2-a]imidazol-2-yl)-2-(3,3-dimethyl-4-(6-oxo-1,6-dihydropyridine-3-carbonyl)piperazin-1-yl)propanamide

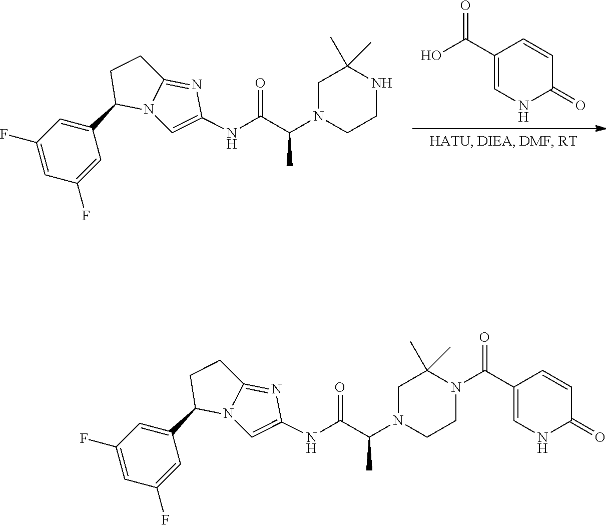

A solution of (S)—N—((R)-5-(3,5-difluorophenyl)-6,7-dihydro-5H-pyrrolo[1,2-a]imidazol-2-yl)-2-(3,3-dimethylpiperazin-1-yl)propanamide (100 mg, 0.2 mmol), 6-oxo-1,6-dihydropyridine-3-carboxylic acid (69 mg, 0.5 mmol), HATU (141 mg, 0.4 mmol) and DIEA (96 mg, 0.7 mmol) in DMF (3.5 mL) were stirred at room temperature for 4 hours under nitrogen atmosphere. Upon completion, the resulting mixture was directly purified by Prep-HPLC (conditions: Column: XBridge Shield RP18 OBD Column 30*150 mm, 5 μm; Mobile Phase A: Water (10 mmol/L NH₄HCO₃), Mobile Phase B: ACN; Flow rate: 60 mL/min; Gradient (B %): 24% to 39% in 17 minutes; Wave Length: 254 nm/220 nm; RT1 (min): 9.94) to afford (S)—N—((R)-5-(3,5-difluorophenyl)-6,7-dihydro-5H-pyrrolo[1,2-a]imidazol-2-yl)-2-(3,3-dimethyl-4-(6-oxo-1,6-dihydropyridine-3-carbonyl)piperazin-1-yl)propanamide (16.5 mg) as a white solid.

| Compound# | Characterization Data |
|---|---|
| 2 | LCMS (ESI) [M + H]⁺: 525.2 |
| | ¹H NMR (400 MHz, DMSO-d₆) δ 11.80 (bs, 1H), 9.92 |
| | (s, 1H), 7.56 (s, 1H), 7.54 (d, J = 2.6 Hz, 1H), 7.46-7.43 |
| | (m, 1H), 6.97-6.95 (m, 3H), 6.31 (d, J = |
| | 9.2 Hz, 1H), 5.40-5.67 (m, 1H), 3.42-3.40 |
| | (m, 1H), 3.32-3.29 (m, 2H), 2.98-2.94 (m, 2H), 2.92-2.89 |
| | (m, 1H), 2.82-2.79 (m, 1H), 2.51-2.49 (m, 1H), 2.42-2.34 |
| | (m, 3H), 1.38 (d, J = 3.6 Hz, 6H), 1.15 (d, J = 6.9 Hz, 3H). |

### Example 3: (S)—N—((R)-5-(3,5-difluorophenyl)-6,7-dihydro-5H-pyrrolo[1,2-a]imidazol-2-yl)-2-(3,3-dimethyl-4-(5-oxo-4,5-dihydropyrazine-2-carbonyl)piperazin-1-yl)propanamide

### Step 1: (S)—N—((R)-5-(3,5-difluorophenyl)-6,7-dihydro-5H-pyrrolo[1,2-a]imidazol-2-yl)-2-(3,3-dimethyl-4-(5-oxo-4,5-dihydropyrazine-2-carbonyl)piperazin-1-yl)propanamide

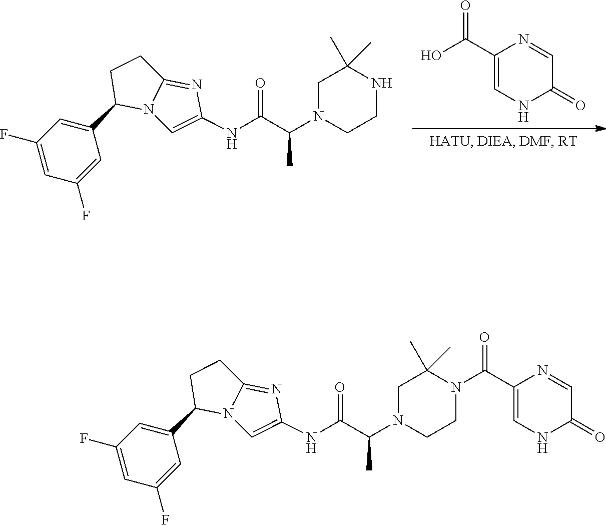

A solution of (S)—N—((R)-5-(3,5-difluorophenyl)-6,7-dihydro-5H-pyrrolo[1,2-a]imidazol-2-yl)-2-(3,3-dimethylpiperazin-1-yl)propanamide (50 mg, 0.1 mmol), 5-oxo-4,5-dihydropyrazine-2-carboxylic acid (26 mg, 0.2 mmol), HATU (70 mg, 0.2 mmol) and DIEA (48 mg, 0.4 mmol) in DMF (2 mL) were stirred at room temperature for 16 hours under nitrogen atmosphere. Upon, completion, the resulting mixture was directly purified by Prep-HPLC (conditions: Column: XBridge Shield RP18 OBD Column 30*150 mm, 5 μm; Mobile Phase A: Water (10 mmol/L NH₄HCO₃), Mobile Phase B: MeOH; Flow rate: 60 mL/min; Gradient (B %): 43% to 58% in 17 minutes; Wave Length: 254 nm/220 nm; RT1 (min): 13.47) to afford (S)—N—((R)-5-(3,5-difluorophenyl)-6,7-dihydro-5H-pyrrolo[1,2-a]imidazol-2-yl)-2-(3,3-dimethyl-4-(5-oxo-4,5-dihydropyrazine-2-carbonyl)piperazin-1-yl)propanamide (18.2 mg, Compound 3) as a yellow solid.

| Compound# | Characterization Data |
|---|---|
| 3 | LCMS (ESI) [M + H]⁺: 526.3 |
| | ¹H NMR (400 MHz, DMSO-d₆) δ 9.92 (s, 1H), |
| | 7.84 (s, 1H), 7.75 (s, 1H), 7.27-7.18 (m, 1H), 6.97-6.95 |
| | (m, 3H), 5.42-5.35 (m, 1H), 3.45-3.36 (m, 3H), |
| | 3.33-2.98 (m, 1H), 3.00-2.88 (m, 2H), 2.83-2.75 |
| | (m, 1H), 2.69-2.65 (m, 1H), 2.40-2.31 |
| | (m, 3H), 1.41 (d, J = 2.8 Hz, 6H), |
| | 1.15 (d, J = 6.9 Hz, 3H). |

### Example 4: (S)—N—((R)-5-(3,5-difluorophenyl)-6,7-dihydro-5H-pyrrolo[1,2-a]imidazol-2-yl)-2-(4-(4-(2-hydroxyethyl)-5-oxo-4,5-dihydropyrazine-2-carbonyl)-3,3-dimethylpiperazin-1-yl)propanamide

### Step 1: (S)-2-(4-(4-(2-((tert-butyldimethylsilyl)oxy)ethyl)-5-oxo-4,5-dihydropyrazine-2-carbonyl)-3,3-dimethylpiperazin-1-yl)-N—((R)-5-(3,5-difluorophenyl)-6,7-dihydro-5H-pyrrolo[1,2-a]imidazol-2-yl)propanamide

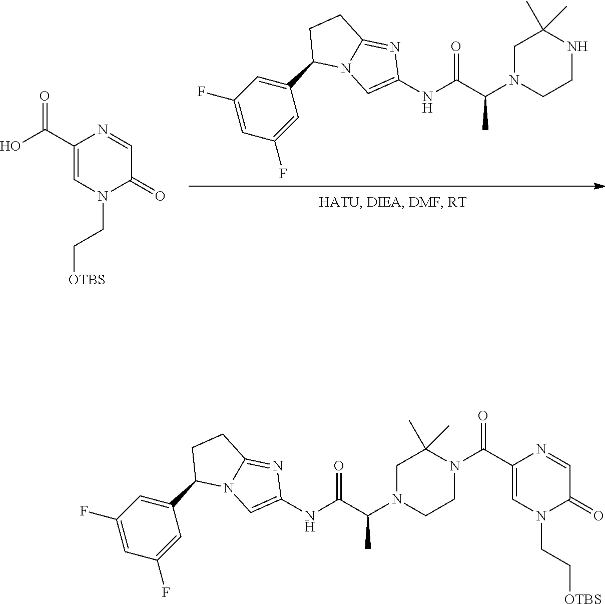 [SMILES: CC(C)(C)[Si](C)(C)OCCn1cc(C(=O)O)ncc1=O ; C[C@@H](C(=O)Nc1cn2c(n1)CC[C@@H]2c1cc(F)cc(F)c1)N1CCNC(C)(C)C1 ; C[C@@H](C(=O)Nc1cn2c(n1)CC[C@@H]2c1cc(F)cc(F)c1)N1CCN(C(=O)c2cn(CCO[Si](C)(C)C(C)(C)C)c(=O)cn2)C(C)(C)C1]

A solution of 4-(2-((tert-butyldimethylsilyl)oxy)ethyl)-5-oxo-4,5-dihydropyrazine-2-carboxylic acid (100 mg, 0.3 mmol), (S)—N—((R)-5-(3,5-difluorophenyl)-6,7-dihydro-5H-pyrrolo[1,2-a]imidazol-2-yl)-2-(3,3-dimethylpiperazin-1-yl)propanamide (108 mg, 0.3 mmol), HATU (191 mg, 0.5 mmol) and DIEA (129 mg, 1.0 mmol) in DMF (5 mL) were stirred at room temperature for 4 hours under nitrogen atmosphere. Upon completion, the resulting mixture was directly purified by reversed-phase flash chromatography (conditions: column: C18 silica gel; mobile phase A: water (10 mmol/L NH₄HCO₃), Mobile Phase B: MeCN; Gradient (B %): 0% to 100% in 20 minutes; detector: UV 254 nm) to afford (S)-2-(4-(4-(2-((tert-butyldimethylsilyl)oxy)ethyl)-5-oxo-4,5-dihydropyrazine-2-carbonyl)-3,3-dimethylpiperazin-1-yl)-N—((R)-5-(3,5-difluorophenyl)-6,7-dihydro-5H-pyrrolo[1,2-a]imidazol-2-yl)propanamide (45 mg, 19% yield) as a yellow solid. LCMS (ESI) [M+H]⁺: 684.

### Step 2: (S)—N—((R)-5-(3,5-difluorophenyl)-6,7-dihydro-5H-pyrrolo[1,2-a]imidazol-2-yl)-2-(4-(4-(2-hydroxyethyl)-5-oxo-4,5-dihydropyrazine-2-carbonyl)-3,3-dimethylpiperazin-1-yl)propanamide

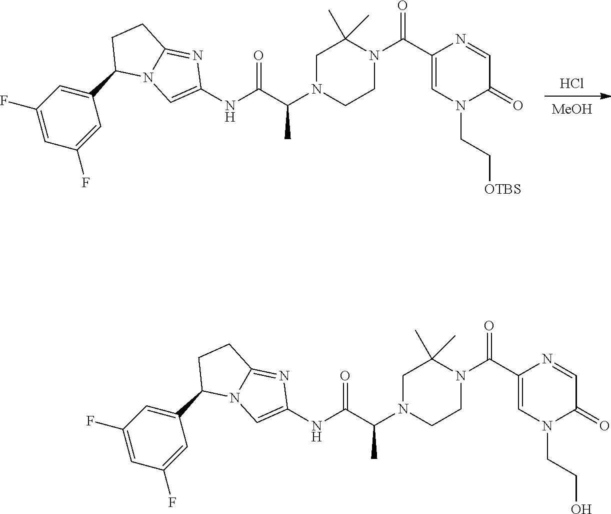 [SMILES: "MeOH" ; C[C@@H](C(=O)Nc1cn2c(n1)CC[C@@H]2c1cc(F)cc(F)c1)N1CCN(C(=O)c2cn(CCO[Si](C)(C)C(C)(C)C)c(=O)cn2)C(C)(C)C1 ; Cl ; C[C@@H](C(=O)Nc1cn2c(n1)CC[C@@H]2c1cc(F)cc(F)c1)N1CCN(C(=O)c2cn(CCO)c(=O)cn2)C(C)(C)C1]

To a mixture of (S)-2-(4-(4-(2-((tert-butyldimethylsilyl)oxy)ethyl)-5-oxo-4,5-dihydropyrazine-2-carbonyl)-3,3-dimethylpiperazin-1-yl)-N—((R)-5-(3,5-difluorophenyl)-6,7-dihydro-5H-pyrrolo[1,2-a]imidazol-2-yl)propanamide (30 mg, 40.0 μmol) in MeOH (5 mL) was added HCl (0.1 mL, 12 M) at 0° C. under nitrogen atmosphere. The resulting mixture was warmed to room temperature and stirred for 4 hours. Upon completion, the resulting mixture was concentrated under reduced pressure. The crude residue was purified by reversed-phase flash chromatography (conditions: column: C18 silica gel; mobile phase A: water (10 mmol/L NH₄HCO₃), Mobile Phase B: MeCN; Gradient (B %): 0% to 100% in 20 minutes; detector: UV 254 nm) to afford (S)—N—((R)-5-(3,5-difluorophenyl)-6,7-dihydro-5H-pyrrolo[1,2-a]imidazol-2-yl)-2-(4-(4-(2-hydroxyethyl)-5-oxo-4,5-dihydropyrazine-2-carbonyl)-3,3-dimethylpiperazin-1-yl)propanamide (5.1 mg, Compound 4) as a white solid.

| Compound# | Characterization Data |
|---|---|
| 4 | LCMS (ESI) [M + H]⁺: 570.4 |
| | ¹H NMR (400 MHz, DMSO-d₆) δ 9.94 (s, 1H), 8.39 |
| | (s, 1H), 7.96 (s, 1H), 7.25-7.20 (m, 1H), 6.97-6.95 |
| | (m, 3H), 5.40-5.37 (m, 1H), 4.95 (bs, 1H), |
| | 3.97-3.95 (m, 2H), 3.66-3.60 (m, 2H), |
| | 3.47-3.36 (m, 2H), 3.00-2.92 (m, 2H), |
| | 2.88-2.83 (m, 1H), 2.82-2.79 (m, 1H), |
| | 2.69-2.67 (m, 1H), 2.41-2.32 (m, 3H), |
| | 1.42 (d, J = 3.2 Hz, 6H), 1.14 (d, J = 6.9 Hz, 3H). |

### Example 5: (S)—N—((R)-4-(3,5-difluorophenyl)-5,6-dihydro-4H-pyrrolo[1,2-b]pyrazol-2-yl)-2-(3,3-dimethyl-4-(6-oxo-1,6-dihydropyridine-3-carbonyl)piperazin-1-yl)propanamide (5a) and (S)—N—((S)-4-(3,5-difluorophenyl)-5,6-dihydro-4H-pyrrolo[1,2-b]pyrazol-2-yl)-2-(3,3-dimethyl-4-(6-oxo-1,6-dihydropyridine-3-carbonyl)piperazin-1-yl)propanamide (5b)

### Step 1: (2S)—N-(4-(3,5-difluorophenyl)-5,6-dihydro-4H-pyrrolo[1,2-b]pyrazol-2-yl)-2-(3,3-dimethyl-4-(6-oxo-1,6-dihydropyridine-3-carbonyl)piperazin-1-yl)propanamide

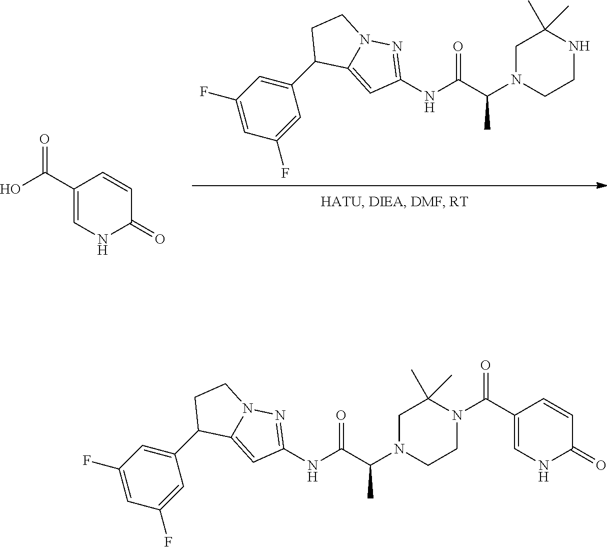 [SMILES: O=C(O)c1ccc(=O)[nH]c1 ; C[C@@H](C(=O)Nc1cc2n(n1)CCC2c1cc(F)cc(F)c1)N1CCNC(C)(C)C1 ; C[C@@H](C(=O)Nc1cc2n(n1)CCC2c1cc(F)cc(F)c1)N1CCN(C(=O)c2ccc(=O)[nH]c2)C(C)(C)C1]

A solution of 6-oxo-1,6-dihydropyridine-3-carboxylic acid (52 mg, 0.4 mmol), (2S)—N-(4-(3,5-difluorophenyl)-5,6-dihydro-4H-pyrrolo[1,2-b]pyrazol-2-yl)-2-(3,3-dimethylpiperazin-1-yl)propanamide (100 mg, 0.2 mmol), DIEA (128 mg, 1.0 mmol) and HATU (141 mg, 0.4 mmol) in DMF (5 mL) were stirred at room temperature for 2 hours under nitrogen atmosphere. The resulting mixture was directly purified by reversed-phase flash chromatography (conditions: column: C18 silica gel; mobile phase A: water (10 mmol/L NH₄HCO₃), Mobile Phase B: MeCN; Gradient (B %): 0% to 100% in 20 minutes; detector: UV 254 nm) to afford (2S)—N-(4-(3,5-difluorophenyl)-5,6-dihydro-4H-pyrrolo[1,2-b]pyrazol-2-yl)-2-(3,3-dimethyl-4-(6-oxo-1,6-dihydropyridine-3-carbonyl)piperazin-1-yl)propanamide (61 mg, 47% yield) as a light yellow solid. LCMS (ESI) [M+H]⁺: 525.

### Step 2: (S)—N—((R)-4-(3,5-difluorophenyl)-5,6-dihydro-4H-pyrrolo[1,2-b]pyrazol-2-yl)-2-(3,3-dimethyl-4-(6-oxo-1,6-dihydropyridine-3-carbonyl)piperazin-1-yl)propanamide and (S)—N—((S)-4-(3,5-difluorophenyl)-5,6-dihydro-4H-pyrrolo[1,2-b]pyrazol-2-yl)-2-(3,3-dimethyl-4-(6-oxo-1,6-dihydropyridine-3-carbonyl)piperazin-1-yl)propanamide

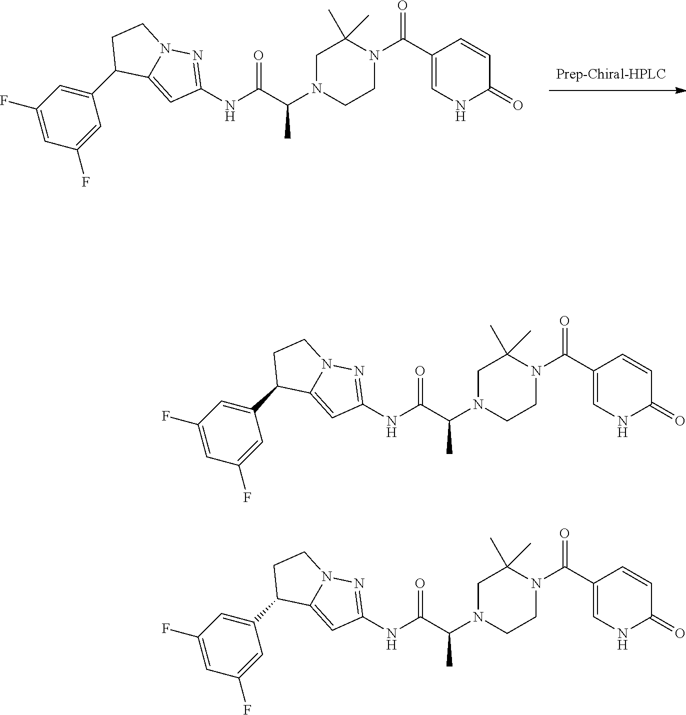 [SMILES: C[C@@H](C(=O)Nc1cc2n(n1)CCC2c1cc(F)cc(F)c1)N1CCN(C(=O)c2ccc(=O)[nH]c2)C(C)(C)C1 ; C[C@@H](C(=O)Nc1cc2n(n1)CC[C@@H]2c1cc(F)cc(F)c1)N1CCN(C(=O)c2ccc(=O)[nH]c2)C(C)(C)C1 ; C[C@@H](C(=O)Nc1cc2n(n1)CC[C@H]2c1cc(F)cc(F)c1)N1CCN(C(=O)c2ccc(=O)[nH]c2)C(C)(C)C1]

(2S)—N-(4-(3,5-difluorophenyl)-5,6-dihydro-4H-pyrrolo[1,2-b]pyrazol-2-yl)-2-(3,3-dimethyl-4-(6-oxo-1,6-dihydropyridine-3-carbonyl)piperazin-1-yl)propanamide (61 mg) was resolved by Prep-Chiral HPLC (conditions: Column: CHIRAL ART Cellulose-SB, 3*25 cm, 5 m; Mobile Phase A: MTBE (10 mM NH₃-MeOH), Mobile Phase B: EtOH; Flow rate: 40 mL/min; Gradient (B %): isocratic 30% B; Wave Length: 242/214 nm; RT1 (min): 7.84; RT2 (min): 11.68) to afford (S)—N—((R)-4-(3,5-difluorophenyl)-5,6-dihydro-4H-pyrrolo[1,2-b]pyrazol-2-yl)-2-(3,3-dimethyl-4-(6-oxo-1,6-dihydropyridine-3-carbonyl)piperazin-1-yl)propanamide (Compound 5a, peak 1, 18.3 mg; Method A, 0.88 min, Peak 1) as a white solid and the second peak (S)—N—((S)-4-(3,5-difluorophenyl)-5,6-dihydro-4H-pyrrolo[1,2-b]pyrazol-2-yl)-2-(3,3-dimethyl-4-(6-oxo-1,6-dihydropyridine-3-carbonyl)piperazin-1-yl)propanamide (Compound 5b, peak 2, 18.5 mg; Method A, 1.25 min, Peak 2) as a white solid.

| Compound# | Characterization Data |
|---|---|
| 5a | LCMS (ESI) [M + H]⁺: 525.3 |
| | ¹H NMR (400 MHz, DMSO-d₆) δ 11.80 (bs, 1H), |
| | 10.08 (s, 1H), 7.55 (s, 1H), 7.54-7.46 (m, 1H), 7.17-7.14 |
| | (m, 1H), 7.04-6.98 (m, 2H), 6.32-6.25 |
| | (m, 2H), 4.55 (t, J = 7.6 Hz, 1H), 4.23-4.19 |
| | (m, 1H), 4.05-4.03 (m, 1H), 3.38-3.33 (m, 3H), 3.02-2.99 |
| | (m, 1H), 2.68-2.59 (m, 1H), 2.57-2.51 |
| | (m, 1H), 2.49-2.45 (m, 1H), |
| | 2.41 (s, 2H), 1.39 (d, J = 4.0 Hz, 6H), 1.16 (d, J = 6.8 Hz, 3H). |
| 5b | LCMS (ESI) [M + H]⁺: 525.3 |
| | ¹H NMR (400 MHz, DMSO-d₆) δ 11.81 (bs, 1H), |
| | 10.10 (s, 1H), 7.54 (s, 1H), 7.44 (d, J = 6.8 Hz, 1H), 7.14 |
| | (t, J = 8.8 Hz, 1H), 7.01-6.99 (m, 2H), |
| | 6.32-6.26 (m, 2H), 4.55 (t, J = 7.6 Hz, 1H), |
| | 4.21-4.19 (m, 1H), 4.05-4.02 (m, 1H), 3.37-3.32 (m, 3H), |
| | 3.02-2.99 (m, 1H), 2.68-2.66 (m, 1H), 2.59-2.57 (m, 1H), |
| | 2.50-2.36 (m, 3H), 1.38 (d, J = 2.4 Hz, |
| | 6H), 1.16 (d, J = 6.8 Hz, 3H). |

### Example 6: 4-(4-((S)-1-(((R)-4-(3,5-difluorophenyl)-5,6-dihydro-4H-pyrrolo[1,2-b]pyrazol-2-yl)amino)-1-oxopropan-2-yl)-2,2-dimethylpiperazine-1-carbonyl)pyridine 1-oxide (6a) and 4-(4-((S)-1-(((S)-4-(3,5-difluorophenyl)-5,6-dihydro-4H-pyrrolo[1,2-b]pyrazol-2-yl)amino)-1-oxopropan-2-yl)-2,2-dimethylpiperazine-1-carbonyl)pyridine 1-oxide (6b)

### Step 1: 4-(4-((2S)-1-((4-(3,5-difluorophenyl)-5,6-dihydro-4H-pyrrolo[1,2-b]pyrazol-2-yl)amino)-1-oxopropan-2-yl)-2,2-dimethylpiperazine-1-carbonyl)pyridine 1-oxide

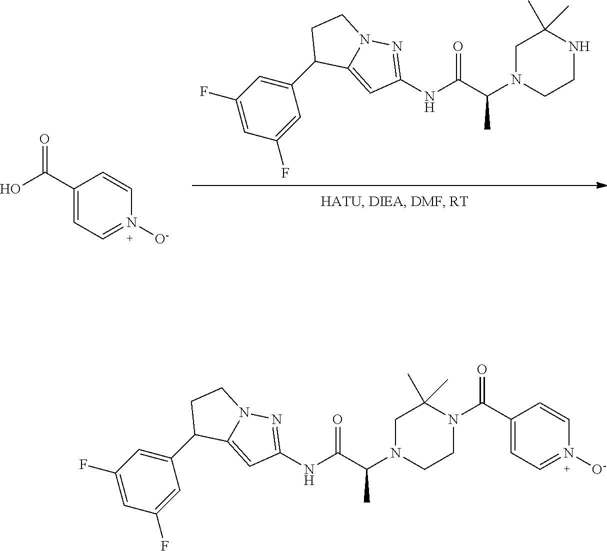

A solution of (2S)—N-(4-(3,5-difluorophenyl)-5,6-dihydro-4H-pyrrolo[1,2-b]pyrazol-2-yl)-2-(3,3-dimethylpiperazin-1-yl)propanamide (139 mg, 0.3 mmol), 4-carboxypyridine 1-oxide (72 mg, 0.5 mmol), DIEA (156 mg, 1.2 mmol) and HATU (197 mg, 0.5 mmol) in DMF (4.5 mL) were stirred at room temperature for 2 hours under nitrogen atmosphere. Upon completion, the reaction was quenched with water (10 mL) at 0° C. The resulting mixture was extracted with EtOAc (3×6 mL). The combined organic layers were washed with brine (3×8 mL), dried over anhydrous Na₂SO₄, filtered and concentrated under reduced pressure. The crude residue was purified by silica gel column chromatography, eluted with DCM/MeOH (9:1) to afford 4-(4-((2S)-1-((4-(3,5-difluorophenyl)-5,6-dihydro-4H-pyrrolo[1,2-b]pyrazol-2-yl)amino)-1-oxopropan-2-yl)-2,2-dimethylpiperazine-1-carbonyl)pyridine 1-oxide (140 mg, 77% yield) as a yellow solid. LCMS (ESI) [M+H]⁺: 525.

### Step 2: 4-(4-((S)-1-(((R)-4-(3,5-difluorophenyl)-5,6-dihydro-4H-pyrrolo[1,2-b]pyrazol-2-yl)amino)-1-oxopropan-2-yl)-2,2-dimethylpiperazine-1-carbonyl)pyridine 1-oxide and 4-(4-((S)-1-(((S)-4-(3,5-difluorophenyl)-5,6-dihydro-4H-pyrrolo[1,2-b]pyrazol-2-yl)amino)-1-oxopropan-2-yl)-2,2-dimethylpiperazine-1-carbonyl)pyridine 1-oxide

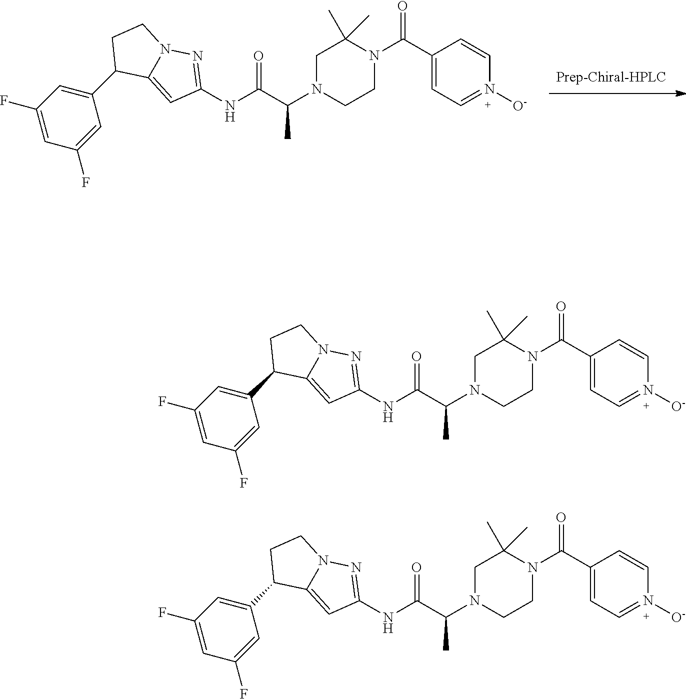

4-(4-((2S)-1-((4-(3,5-difluorophenyl)-5,6-dihydro-4H-pyrrolo[1,2-b]pyrazol-2-yl)amino)-1-oxopropan-2-yl)-2,2-dimethylpiperazine-1-carbonyl)pyridine 1-oxide (140 mg) was resolved by Prep-Chiral-HPLC (conditions: Column: CHIRALPAK IE, 3*25 cm, 5 μm; Mobile Phase A: MTBE (10 mM NH₃), Mobile Phase B: MeOH; Flow rate: 40 mL/min; Gradient (B %): isocratic 30% B; Wave Length: 216/286 nm; RT1 (min): 10.24; RT2 (min): 14.01) to afford the 4-(4-((S)-1-(((R)-4-(3,5-difluorophenyl)-5,6-dihydro-4H-pyrrolo[1,2-b]pyrazol-2-yl)amino)-1-oxopropan-2-yl)-2,2-dimethylpiperazine-1-carbonyl)pyridine 1-oxide (Compound 6a, peak 1, 48.9 mg; Method B, 1.17 min, Peak 1) as an off-white solid and 4-(4-((S)-1-(((S)-4-(3,5-difluorophenyl)-5,6-dihydro-4H-pyrrolo[1,2-b]pyrazol-2-yl)amino)-1-oxopropan-2-yl)-2,2-dimethylpiperazine-1-carbonyl)pyridine 1-oxide (Compound 6b, peak 2, 48.2 mg; Method B, 1.64 min, Peak 2) as a light yellow solid.

| Compound# | Characterization Data |
|---|---|
| 6a | LCMS (ESI) [M + H]⁺: 525.2 |
| | ¹H NMR (400 MHz, DMSO-d₆) δ 10.12 (s, 1H), |
| | 8.24-8.19 (m, 2H), 7.42-7.40 (m, 2H), |
| | 7.17-7.12 (m, 1H), 7.04-7.01 (m, 2H), 6.26 (s, 1H), |
| | 4.55-4.53 (m, 1H), 4.22-4.19 (m, 1H), 4.05-4.03 (m, 1H), |
| | 3.39-3.37 (m, 1H), 3.32-3.28 (m, 2H), 3.02-2.98 (m, 1H), |
| | 2.69-2.66 (m, 1H), 2.58-2.52 (m, 1H), 2.49-2.45 (m, 1H), |
| | 2.42 (s, 2H), 1.43 (d, J = 4.8 Hz, 6H), 1.16 (d, J = 6.8 Hz, 3H). |
| 6b | LCMS (ESI) [M + H]⁺: 525.3 |
| | ¹H NMR (400 MHz, DMSO-d₆) δ 10.13 (s, 1H), |
| | 8.21 (d, J = 7.2 Hz, 2H), 7.41 (d, J = 7.2 Hz, |
| | 2H), 7.16-7.14 (m, 1H), 7.01-6.99 (m, 2H), 6.26 |
| | (s, 1H), 4.53 (t, J = 7.6 Hz, 1H), 4.20-4.17 (m, 1H), 4.07-4.05 |
| | (m, 1H), 3.38-3.35 (m, 1H), 3.31-3.28 (m, 2H), 3.02-2.98 (m, |
| | 1H), 2.68-2.64 (m, 1H), 2.59-2.53 (m, 1H), 2.49-2.38 (m, 3H), |
| | 1.43 (d, J = 3.2 Hz, 6H), 1.16 (d, J = 6.8 Hz, 3H). |

### Example 7: (S)—N—((R)-5-(3,5-difluorophenyl)-6,7-dihydro-5H-pyrrolo[1,2-a]imidazol-2-yl)-2-(3,3-dimethyl-4-((R)-5,6,7,8-tetrahydro-[1,2,4]triazolo[4,3-a]pyridine-6-carbonyl)piperazin-1-yl)propanamide (7a) and (S)—N—((R)-5-(3,5-difluorophenyl)-6,7-dihydro-5H-pyrrolo[1,2-a]imidazol-2-yl)-2-(3,3-dimethyl-4-((S)-5,6,7,8-tetrahydro-[1,2,4]triazolo[4,3-a]pyridine-6-carbonyl)piperazin-1-yl)propanamide (7b)

### Step 1: (2S)—N—((R)-5-(3,5-difluorophenyl)-6,7-dihydro-5H-pyrrolo[1,2-a]imidazol-2-yl)-2-(3,3-dimethyl-4-(5,6,7,8-tetrahydro-[1,2,4]triazolo[4,3-a]pyridine-6-carbonyl)piperazin-1-yl)propanamide

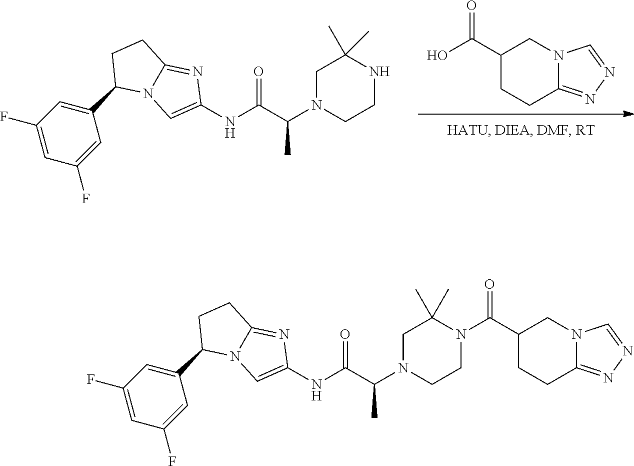 [SMILES: C[C@@H](C(=O)Nc1cn2c(n1)CC[C@@H]2c1cc(F)cc(F)c1)N1CCNC(C)(C)C1 ; O=C(O)C1CCc2nncn2C1 ; C[C@@H](C(=O)Nc1cn2c(n1)CC[C@@H]2c1cc(F)cc(F)c1)N1CCN(C(=O)C2CCc3nncn3C2)C(C)(C)C1]

A solution of (S)—N—((R)-5-(3,5-difluorophenyl)-6,7-dihydro-5H-pyrrolo[1,2-a]imidazol-2-yl)-2-(3,3-dimethylpiperazin-1-yl)propanamide (100 mg, 0.2 mmol), 5,6,7,8-tetrahydro-[1,2,4]triazolo[4,3-a]pyridine-6-carboxylic acid (62 mg, 0.4 mmol), DIEA (96 mg, 0.7 mmol) and HATU (141 mg, 0.4 mmol) in DMF (3.5 mL) were stirred at room temperature for 4 hours under nitrogen atmosphere. Upon completion, the resulting mixture was purified by reversed-phase flash chromatography (conditions: column: C18 silica gel; mobile phase A: water (0.1% NH₃·H₂O), Mobile Phase B: MeCN; Gradient (B %): 0% to 100% B in 20 minutes; detector: UV 254 nm) to afford (2S)—N—((R)-5-(3,5-difluorophenyl)-6,7-dihydro-5H-pyrrolo[1,2-a]imidazol-2-yl)-2-(3,3-dimethyl-4-(5,6,7,8-tetrahydro-[1,2,4]triazolo[4,3-a]pyridine-6-carbonyl)piperazin-1-yl)propanamide (35 mg, 25% yield) as a white solid. LCMS (ESI) [M+H]⁺: 553.

### Step 2: (S)—N—((R)-5-(3,5-difluorophenyl)-6,7-dihydro-5H-pyrrolo[1,2-a]imidazol-2-yl)-2-(3,3-dimethyl-4-((R)-5,6,7,8-tetrahydro-[1,2,4]triazolo[4,3-a]pyridine-6-carbonyl)piperazin-1-yl)propanamide and (S)—N—((R)-5-(3,5-difluorophenyl)-6,7-dihydro-5H-pyrrolo[1,2-a]imidazol-2-yl)-2-(3,3-dimethyl-4-((S)-5,6,7,8-tetrahydro-[1,2,4]triazolo[4,3-a]pyridine-6-carbonyl)piperazin-1-yl)propanamide

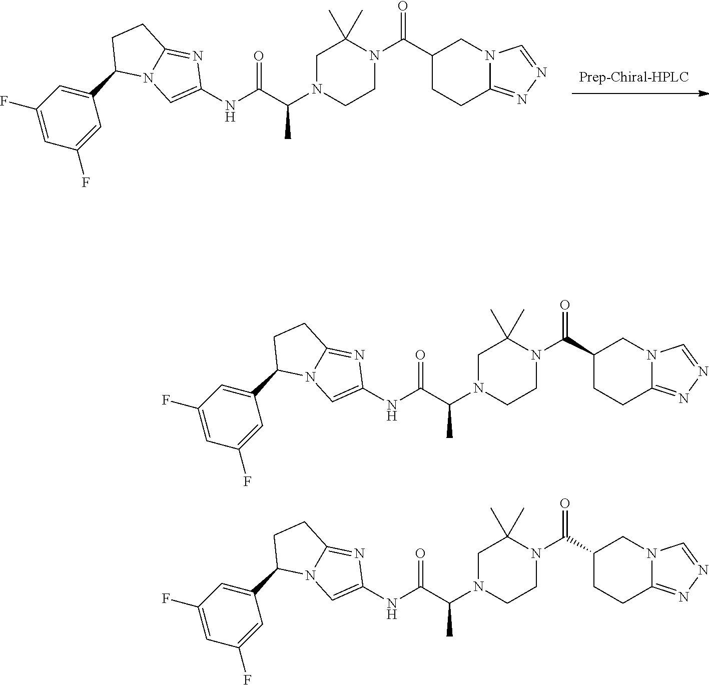 [SMILES: C[C@@H](C(=O)Nc1cn2c(n1)CC[C@@H]2c1cc(F)cc(F)c1)N1CCN(C(=O)C2CCc3nncn3C2)C(C)(C)C1 ; C[C@@H](C(=O)Nc1cn2c(n1)CC[C@@H]2c1cc(F)cc(F)c1)N1CCN(C(=O)[C@@H]2CCc3nncn3C2)C(C)(C)C1 ; C[C@@H](C(=O)Nc1cn2c(n1)CC[C@@H]2c1cc(F)cc(F)c1)N1CCN(C(=O)[C@H]2CCc3nncn3C2)C(C)(C)C1]

(2S)—N—((R)-5-(3,5-difluorophenyl)-6,7-dihydro-5H-pyrrolo[1,2-a]imidazol-2-yl)-2-(3,3-dimethyl-4-(5,6,7,8-tetrahydro-[1,2,4]triazolo[4,3-a]pyridine-6-carbonyl)piperazin-1-yl)propanamide (35 mg) was resolved by Prep-Chiral HPLC (conditions: Column: CHIRALPAK SZ 3*25 cm, 5 μm; Mobile Phase A: MTBE (10 mM NH₃), Mobile Phase B: MeOH; Flow rate: 40 mL/min; Gradient (B %): isocratic 50% B; Wave Length: 238/202 nm; RT1 (min): 7.84; RT2 (min): 10.44) to afford (S)—N—((R)-5-(3,5-difluorophenyl)-6,7-dihydro-5H-pyrrolo[1,2-a]imidazol-2-yl)-2-(3,3-dimethyl-4-((R)-5,6,7,8-tetrahydro-[1,2,4]triazolo[4,3-a]pyridine-6-carbonyl)piperazin-1-yl)propanamide (Compound 7a) (peak 1, 10.6 mg; Method C, 0.75 min, Peak 1) as a white solid and (S)—N—((R)-5-(3,5-difluorophenyl)-6,7-dihydro-5H-pyrrolo[1,2-a]imidazol-2-yl)-2-(3,3-dimethyl-4-((S)-5,6,7,8-tetrahydro-[1,2,4]triazolo[4,3-a]pyridine-6-carbonyl)piperazin-1-yl)propanamide (Compound 7b) (peak 2, 12.8 mg; Method C, 0.92 min, Peak 2) as a white solid.

| Compound# | Characterization Data |
|---|---|
| 7a | LCMS (ESI) [M + H]⁺: 553.2 |
| | ¹H NMR (400 MHz, DMSO-d₆) δ 9.95 (s, 1H), 8.33 (s, 1H), 7.23 (t, J = 9.4 Hz, 1H), 6.96-6.94 |
| | (m, 3H), 5.40-5.37 (m, 1H), 4.12-4.07 (m, 1H), 3.96-3.93 (m, 1H), 3.52-3.48 (m, 2H), |
| | 3.36-3.31 (m, 1H), 3.30-3.26 (m, 1H), 3.01-2.90 (m, 2H), 2.85-2.76 (m, 3H), 2.67-2.51 |
| | (m, 2H), 2.39-2.27 (m, 3H), 2.06-1.98 (m, 1H), 1.90-1.76 (m, 1H), 1.35 (d, J = 6.8 Hz, 6H), |
| | 1.14 (d, J = 6.8 Hz, 3H). |
| 7b | LCMS (ESI) [M + H]⁺: 553.2 |
| | ¹H NMR (400 MHz, DMSO-d₆) δ 9.94 (s, 1H), 8.33 (s, 1H), 7.22 (t, J = 9.4 Hz, 1H), 6.96-6.94 |
| | (m, 3H), 5.39-5.37 (m, 1H), 4.12-4.08 (m, 1H), 3.96-3.91 (m, 1H), 3.53-3.46 (m, 2H), |
| | 3.35-3.32 (m, 1H), 3.30-3.27 (m, 1H), 3.02-2.83 (m, 2H), 2.81-2.76 (m, 3H), 2.74-2.70 |
| | (m, 1H), 2.51-2.49 (m, 1H), 2.39-2.37 (m, 3H), 2.03-1.93 (m, 1H), 1.90-1.78 (m, 1H), |
| | 1.35 (d, J = 6.8 Hz, 6H), 1.14 (d, J = 6.8 Hz, 3H). |

### Example 8: (S)—N—((R)-5-(3,5-difluorophenyl)-6,7-dihydro-5H-pyrrolo[1,2-a]imidazol-2-yl)-2-(3,3-dimethyl-4-((R)-5,6,7,8-tetrahydro-[1,2,4]triazolo[1,5-a]pyridine-6-carbonyl)piperazin-1-yl)propanamide (8a) and (S)—N—((R)-5-(3,5-difluorophenyl)-6,7-dihydro-5H-pyrrolo[1,2-a]imidazol-2-yl)-2-(3,3-dimethyl-4-((S)-5,6,7,8-tetrahydro-[1,2,4]triazolo[1,5-a]pyridine-6-carbonyl)piperazin-1-yl)propanamide (8b)

### Step 1: 5,6,7,8-tetrahydro-[1,2,4]triazolo[1,5-a]pyridine-6-carboxylic acid

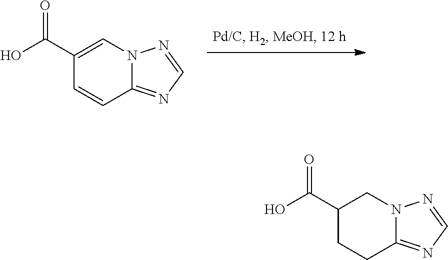 [SMILES: O=C(O)c1ccc2ncnn2c1 ; O=C(O)C1CCc2ncnn2C1]

A mixture of [1,2,4]triazolo[1,5-a]pyridine-6-carboxylic acid (150 mg, 0.9 mmol) and palladium (10 mg, 10% on charcoal) in methanol (10 ml) was stirred for 12 hours at room temperature under hydrogen atmosphere (30 atm) in a pressure reactor. Upon completion, the resulting mixture was filtered through a celite pad using methanol. The filtrate was concentrated under reduced pressure to afford 5,6,7,8-tetrahydro-[1,2,4]triazolo[1,5-a]pyridine-6-carboxylic acid (70 mg, crude) as a white solid, which was used in the next step without further purification. LCMS (ESI) [M+H]⁺: 168.

### Step 2: (2S)—N—((R)-5-(3,5-difluorophenyl)-6,7-dihydro-5H-pyrrolo[1,2-a]imidazol-2-yl)-2-(3,3-dimethyl-4-(5,6,7,8-tetrahydro-[1,2,4]triazolo[1,5-a]pyridine-6-carbonyl)piperazin-1-yl)propanamide

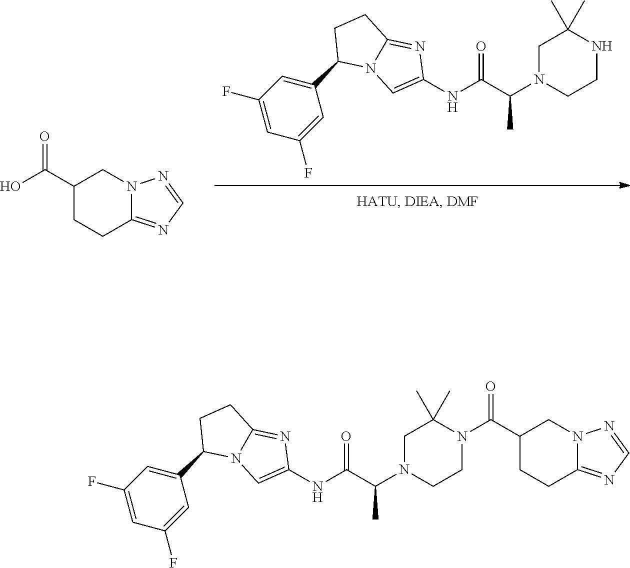 [SMILES: O=C(O)C1CCc2ncnn2C1 ; C[C@@H](C(=O)Nc1cn2c(n1)CC[C@@H]2c1cc(F)cc(F)c1)N1CCNC(C)(C)C1 ; C[C@@H](C(=O)Nc1cn2c(n1)CC[C@@H]2c1cc(F)cc(F)c1)N1CCN(C(=O)C2CCc3ncnn3C2)C(C)(C)C1]

A solution of (S)—N—((R)-5-(3,5-difluorophenyl)-6,7-dihydro-5H-pyrrolo[1,2-a]imidazol-2-yl)-2-(3,3-dimethylpiperazin-1-yl)propanamide (110 mg, 0.3 mmol), 5,6,7,8-tetrahydro-[1,2,4]triazolo[1,5-a]pyridine-6-carboxylic acid (68 mg, 0.4 mmol), DIEA (105 mg, 0.8 mmol) and HATU (155 mg, 0.4 mmol) in DMF (6 mL) were stirred at room temperature for 4 hours under nitrogen atmosphere. Upon completion, the resulting mixture was directly purified by reversed-phase flash chromatography (conditions: column: C18 silica gel; mobile phase A: water (0.1% NH₃H₂O), Mobile Phase B: MeCN; Gradient (B %): 0% to 100% in 20 minutes; detector: UV 254 nm) to afford (2S)—N—((R)-5-(3,5-difluorophenyl)-6,7-dihydro-5H-pyrrolo[1,2 -a]imidazol-2-yl)-2-(3,3-dimethyl-4-(5,6,7,8-tetrahydro-[1,2,4]triazolo[1,5-a]pyridine-6-carbonyl)piperazin-1-yl)propanamide (25 mg, 16% yield) as a white solid. LCMS (ESI) [M+H]⁺: 553

### Step 3: (S)—N—((R)-5-(3,5-difluorophenyl)-6,7-dihydro-5H-pyrrolo[1,2-a]imidazol-2-yl)-2-(3,3-dimethyl-4-((R)-5,6,7,8-tetrahydro-[1,2,4]triazolo[1,5-a]pyridine-6-carbonyl)piperazin-1-yl)propanamide and (S)—N—((R)-5-(3,5-difluorophenyl)-6,7-dihydro-5H-pyrrolo[1,2-a]imidazol-2-yl)-2-(3,3-dimethyl-4-((S)-5,6,7,8-tetrahydro-[1,2,4]triazolo[1,5-a]pyridine-6-carbonyl)piperazin-1-yl)propanamide

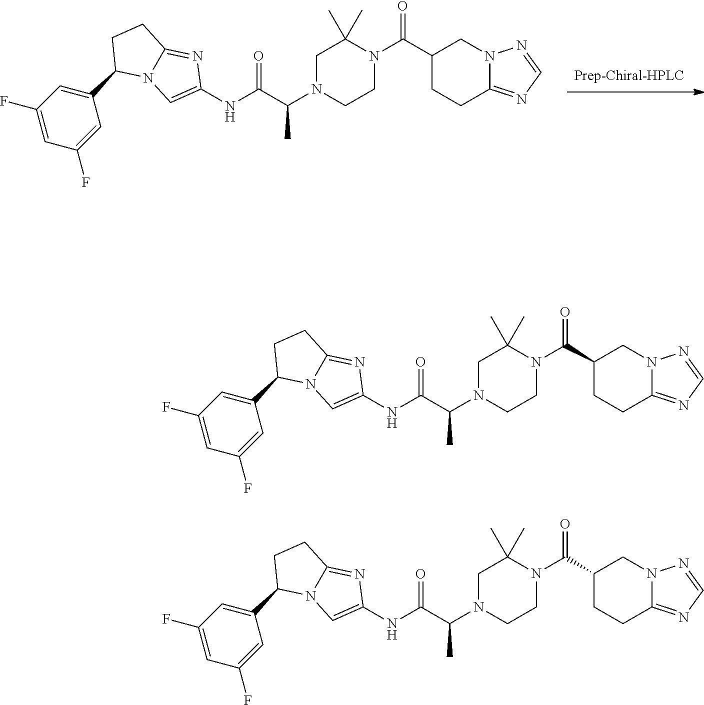 [SMILES: C[C@@H](C(=O)Nc1cn2c(n1)CC[C@@H]2c1cc(F)cc(F)c1)N1CCN(C(=O)C2CCc3ncnn3C2)C(C)(C)C1 ; C[C@@H](C(=O)Nc1cn2c(n1)CC[C@@H]2c1cc(F)cc(F)c1)N1CCN(C(=O)[C@@H]2CCc3ncnn3C2)C(C)(C)C1 ; C[C@@H](C(=O)Nc1cn2c(n1)CC[C@@H]2c1cc(F)cc(F)c1)N1CCN(C(=O)[C@H]2CCc3ncnn3C2)C(C)(C)C1]

(2S)—N—((R)-5-(3,5-difluorophenyl)-6,7-dihydro-5H-pyrrolo[1,2-a]imidazol-2-yl)-2-(3,3-dimethyl-4-(5,6,7,8-tetrahydro-[1,2,4]triazolo[1,5-a]pyridine-6-carbonyl)piperazin-1-yl)propanamide (23 mg) was resolved by Prep-Chiral HPLC (conditions: Column: Lux 5 μm Cellulose-2 3*25 cm, 5 μm; Mobile Phase A: Hex (0.5% 2M NH₃-MeOH), Mobile Phase B: EtOH:ACN=5:1; Flow rate: 40 mL/min; Gradient (B %): isocratic 40% B; Wave Length: 240/250 nm; RT1 (min): 16.5; RT2 (min): 20.2) to afford (S)—N—((R)-5-(3,5-difluorophenyl)-6,7-dihydro-5H-pyrrolo[1,2-a]imidazol-2-yl)-2-(3,3-dimethyl-4-((R)-5,6,7,8-tetrahydro-[1,2,4]triazolo[1,5-a]pyridine-6-carbonyl)piperazin-1-yl)propanamide (Compound 8a, peak 1, 9.3 mg; Method D, 1.73 min, Peak 1) as a white solid and (S)—N—((R)-5-(3,5-difluorophenyl)-6,7-dihydro-5H-pyrrolo[1,2-a]imidazol-2-yl)-2-(3,3-dimethyl-4-((S)-5,6,7,8-tetrahydro-[1,2,4]triazolo[1,5-a]pyridine-6-carbonyl)piperazin-1-yl)propanamide (Compound 8b, peak 2, 8.2 mg; Method D, 2.04 min, Peak 2) as a white solid.

| Compound# | Characterization Data |
|---|---|
| 8a | LCMS (ESI) [M + H]⁺: 553.4 |
| | ¹H NMR (400 MHz, DMSO-d₆) δ 9.94 (s, 1H), 7.82 (s, 1H), 7.24 (t, J = 5.6 Hz, 1H), 6.96-6.94 |
| | (m, 3H), 5.40-5.37 (m, 1H), 4.13-4.08 (m, 2H), 3.53-3.44 (m, 3H), 3.37-3.32 (m, 1H), |
| | 2.96-2.92 (m, 2H), 2.90-2.82 (m, 3H), 2.80-2.76 (m, 2H), 2.39-2.36 (m, 1H), 2.33-2.29 |
| | (m, 2H), 2.13-2.03 (m, 1H), 1.88-1.83 (m, 1H), 1.36 (d, J = 6.0 Hz, 6H), 1.14 (d, J = 6.8 Hz, |
| | 3H). |
| 8b | LCMS (ESI) [M + H]⁺: 553.4 |
| | ¹H NMR (400 MHz, DMSO-d₆) δ 9.94 (s, 1H), 7.82 (s, 1H), 7.24 (t, J = 5.6 Hz, 1H), 6.97-6.94 |
| | (m, 3H), 5.40-5.37 (m, 1H), 4.13-4.08 (m, 2H), 3.53-3.44 (m, 3H), 3.37-3.32 (m, 1H), |
| | 2.96-2.92 (m, 2H), 2.90-2.82 (m, 3H), 2.80-2.76 (m, 2H), 2.39-2.36 (m, 1H), 2.33-2.29 |
| | (m, 2H), 2.13-2.03 (m, 1H), 1.89-1.83 (m, 1H), 1.36 (d, J = 6.0 Hz, 6H), 1.14 (d, J = 6.8 Hz, |
| | 3H). |

### Example 9: (S)—N—((R)-4-(3,5-difluorophenyl)-5,6-dihydro-4H-pyrrolo[1,2-b]pyrazol-2-yl)-2-(4-(4-(2-hydroxyethyl)-5-oxo-4,5-dihydropyrazine-2-carbonyl)-3,3-dimethylpiperazin-1-yl)propanamide (9a) and (S)—N—((S)-4-(3,5-difluorophenyl)-5,6-dihydro-4H-pyrrolo[1,2-b]pyrazol-2-yl)-2-(4-(4-(2-hydroxyethyl)-5-oxo-4,5-dihydropyrazine-2-carbonyl)-3,3-dimethylpiperazin-1-yl)propanamide (9b)

### Step 1: methyl 4-(2-((tert-butyldimethylsilyl)oxy)ethyl)-5-oxo-4,5-dihydropyrazine-2-carboxylate

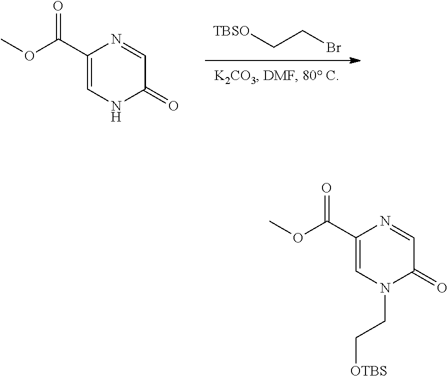 [SMILES: COC(=O)c1c[nH]c(=O)cn1 ; CC(C)(C)[Si](C)(C)OCCBr ; COC(=O)c1cn(CCO[Si](C)(C)C(C)(C)C)c(=O)cn1]

A solution of methyl 5-oxo-4H-pyrazine-2-carboxylate (2.00 g, 13.0 mmol), (2-bromoethoxy)(tert-butyl)dimethylsilane (6.20 g, 26.0 mmol) and K₂CO₃ (5.30 g, 38.9 mmol) in DMF (50 mL) was stirred at 80° C. for 8 hours under nitrogen atmosphere. Upon completion, water (500 mL) was added. The reaction mixture was extracted with EtOAc (3×500 mL). The combined organic layers were washed with brine (500 mL), dried over anhydrous MgSO₄, filtered and concentrated under reduced pressure. The crude residue was purified by silica gel column chromatography, eluted with CH₂Cl₂/PE (1:1) to afford methyl 4-(2-((tert-butyldimethylsilyl)oxy)ethyl)-5-oxo-4,5-dihydropyrazine-2-carboxylate (2.00 g, 49% yield) as a yellow oil. LCMS (ESI) [M+H]⁺: 313

### Step 2: 4-(2-((tert-butyldimethylsilyl)oxy)ethyl)-5-oxo-4,5-dihydropyrazine-2-carboxylic acid

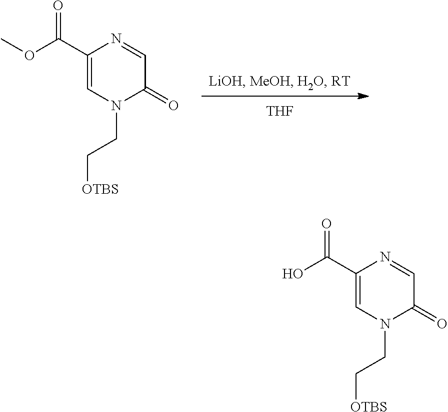 [SMILES: COC(=O)c1cn(CCO[Si](C)(C)C(C)(C)C)c(=O)cn1 ; CC(C)(C)[Si](C)(C)OCCn1cc(C(=O)O)ncc1=O ; "THF"]

A solution of methyl 4-(2-((tert-butyldimethylsilyl)oxy)ethyl)-5-oxo-4,5-dihydropyrazine-2-carboxylate (2.00 g, 6.4 mmol) and LiOH monohydrate (0.77 g, 32.0 mmol) in THF (30 mL) and H₂O (30 mL) was stirred at room temperature for 1 hour under nitrogen atmosphere. Upon completion, the resulting mixture was concentrated under reduced pressure. The crude residue was purified by reversed-phase flash chromatography (conditions: column: C18 silica gel; mobile phase A: water (0.1% FA), Mobile Phase B: MeCN; Gradient (B %): 10% to 50% in 10 minutes; detector: UV 254 nm) to afford 4-(2-((tert-butyldimethylsilyl)oxy)ethyl)-5-oxo-4,5-dihydropyrazine-2-carboxylic acid (1.4 g, 73% yield) as a yellow oil. LCMS (ESI) [M+H]⁺: 299

### Step 3: (2S)-2-(4-(4-(2-((tert-butyldimethylsilyl)oxy)ethyl)-5-oxo-4,5-dihydropyrazine-2-carbonyl)-3,3-dimethylpiperazin-1-yl)-N-(4-(3,5-difluorophenyl)-5,6-dihydro-4H-pyrrolo[1,2-b]pyrazol-2-yl)propanamide

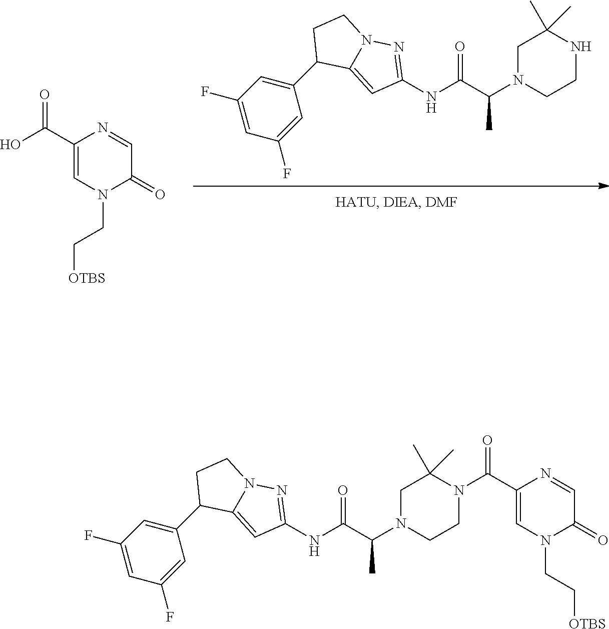 [SMILES: CC(C)(C)[Si](C)(C)OCCn1cc(C(=O)O)ncc1=O ; C[C@@H](C(=O)Nc1cc2n(n1)CCC2c1cc(F)cc(F)c1)N1CCNC(C)(C)C1 ; C[C@@H](C(=O)Nc1cc2n(n1)CCC2c1cc(F)cc(F)c1)N1CCN(C(=O)c2cn(CCO[Si](C)(C)C(C)(C)C)c(=O)cn2)C(C)(C)C1]

A solution of 4-(2-((tert-butyldimethylsilyl)oxy)ethyl)-5-oxo-4,5-dihydropyrazine-2-carboxylic acid (111 mg, 0.4 mmol), (2S)—N-(4-(3,5-difluorophenyl)-5,6-dihydro-4H-pyrrolo[1,2-b]pyrazol-2-yl)-2-(3,3-dimethylpiperazin-1-yl)propanamide (100 mg, 0.2 mmol), DIEA (128 mg, 1.0 mmol) and HATU (141 mg, 0.4 mmol) in DMF (5 mL) were stirred at room temperature for 2 hours under nitrogen atmosphere. Upon completion, the mixture was directly purified by reversed-phase flash chromatography (conditions: column: C18 silica gel; mobile phase A: water (0.1% formic acid), Mobile Phase B: MeCN; Gradient (B %): 0% to 100% in 20 minutes; detector: UV 254 nm) to afford (2S)-2-(4-(4-(2-((tert-butyldimethylsilyl)oxy)ethyl)-5-oxo-4,5-dihydropyrazine-2-carbonyl)-3,3-dimethylpiperazin-1-yl)-N-(4-(3,5-difluorophenyl)-5,6-dihydro-4H-pyrrolo[1,2-b]pyrazol-2-yl)propanamide (90 mg, 53% yield) as a light yellow solid. LCMS (ESI) [M+H]⁺: 684

### Step 4: (2S)—N-(4-(3,5-difluorophenyl)-5,6-dihydro-4H-pyrrolo[1,2-b]pyrazol-2-yl)-2-(4-(4-(2-hydroxyethyl)-5-oxo-4,5-dihydropyrazine-2-carbonyl)-3,3-dimethylpiperazin-1-yl)propanamide

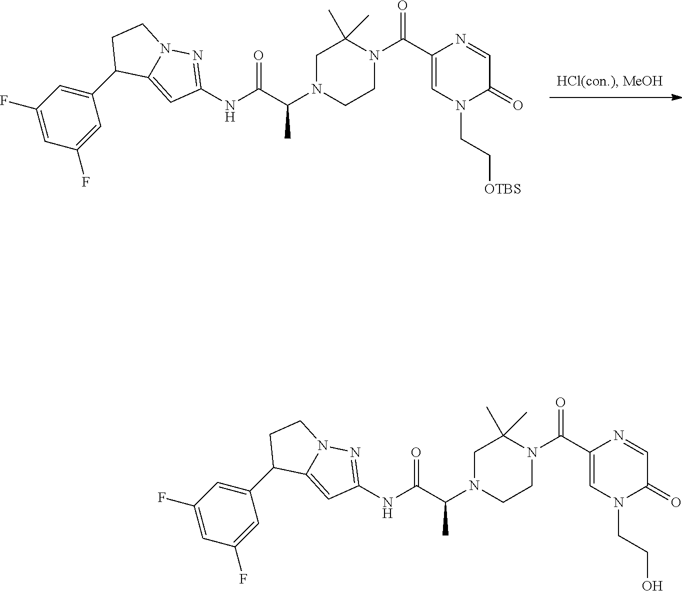 [SMILES: C[C@@H](C(=O)Nc1cc2n(n1)CCC2c1cc(F)cc(F)c1)N1CCN(C(=O)c2cn(CCO[Si](C)(C)C(C)(C)C)c(=O)cn2)C(C)(C)C1 ; C[C@@H](C(=O)Nc1cc2n(n1)CCC2c1cc(F)cc(F)c1)N1CCN(C(=O)c2cn(CCO)c(=O)cn2)C(C)(C)C1]

To a mixture of (2S)-2-(4-(4-(2-((tert-butyldimethylsilyl)oxy)ethyl)-5-oxo-4,5-dihydropyrazine-2-carbonyl)-3,3-dimethylpiperazin-1-yl)-N-(4-(3,5-difluorophenyl)-5,6-dihydro-4H-pyrrolo[1,2-b]pyrazol-2-yl)propanamide (86 mg, 0.1 mmol) in MeOH (2 mL) was added HCl (0.1 mL, 12M) at 0° C. under nitrogen atmosphere. The resulting mixture was warmed to room temperature and stirred for 2 hours. Upon completion, the reaction mixture was concentrated under reduced pressure. The crude residue was purified by silica gel column chromatography, eluted with CH₂Cl₂/MeOH (19:1) to afford (2S)—N-(4-(3,5-difluorophenyl)-5,6-dihydro-4H-pyrrolo[1,2-b]pyrazol-2-yl)-2-(4-(4-(2-hydroxyethyl)-5-oxo-4,5-dihydropyrazine-2-carbonyl)-3,3-dimethylpiperazin-1-yl)propanamide (71 mg, 99% yield) as a white solid. LCMS (ESI) [M+H]⁺: 570

### Step 5: (S)—N—((R)-4-(3,5-difluorophenyl)-5,6-dihydro-4H-pyrrolo[1,2-b]pyrazol-2-yl)-2-(4-(4-(2-hydroxyethyl)-5-oxo-4,5-dihydropyrazine-2-carbonyl)-3,3-dimethylpiperazin-1-yl)propanamide and (S)—N—((S)-4-(3,5-difluorophenyl)-5,6-dihydro-4H-pyrrolo[1,2-b]pyrazol-2-yl)-2-(4-(4-(2-hydroxyethyl)-5-oxo-4,5-dihydropyrazine-2-carbonyl)-3,3-dimethylpiperazin-1-yl)propanamide

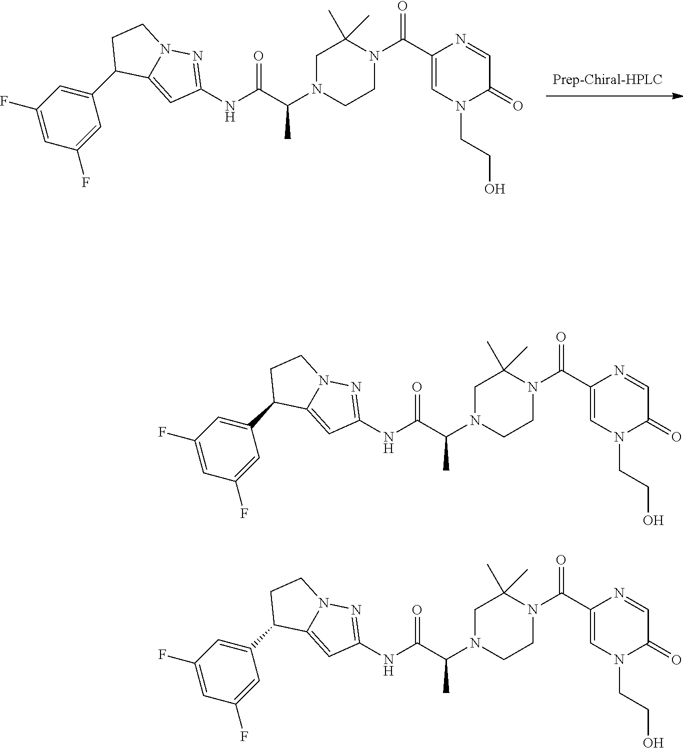 [SMILES: C[C@@H](C(=O)Nc1cc2n(n1)CCC2c1cc(F)cc(F)c1)N1CCN(C(=O)c2cn(CCO)c(=O)cn2)C(C)(C)C1 ; C[C@@H](C(=O)Nc1cc2n(n1)CC[C@@H]2c1cc(F)cc(F)c1)N1CCN(C(=O)c2cn(CCO)c(=O)cn2)C(C)(C)C1 ; C[C@@H](C(=O)Nc1cc2n(n1)CC[C@H]2c1cc(F)cc(F)c1)N1CCN(C(=O)c2cn(CCO)c(=O)cn2)C(C)(C)C1]

(2S)—N-(4-(3,5-difluorophenyl)-5,6-dihydro-4H-pyrrolo[1,2-b]pyrazol-2-yl)-2-(4-(4-(2-hydroxyethyl)-5-oxo-4,5-dihydropyrazine-2-carbonyl)-3,3-dimethylpiperazin-1-yl)propanamide (53 mg) was resolved by Prep-Chiral HPLC (conditions: Column: CHIRALPAK IE, 3*25 cm, 5 m; Mobile Phase A: MTBE (0.1% FA), Mobile Phase B: MeOH; Flow rate: 40 mL/min; Gradient (B %): isocratic 10% B; Wave Length: 242/202 nm; RT1 (min): 16.0; RT2 (min): 24.0) to afford (S)—N—((R)-4-(3,5-difluorophenyl)-5,6-dihydro-4H-pyrrolo[1,2-b]pyrazol-2-yl)-2-(4-(4-(2-hydroxyethyl)-5-oxo-4,5-dihydropyrazine-2-carbonyl)-3,3-dimethylpiperazin-1-yl)propanamide (Compound 9a, peak 1, 21.3 mg; Method E, 1.90 min, Peak 1) as a white solid and (S)—N—((S)-4-(3,5-difluorophenyl)-5,6-dihydro-4H-pyrrolo[1,2-b]pyrazol-2-yl)-2-(4-(4-(2-hydroxyethyl)-5-oxo-4,5-dihydropyrazine-2-carbonyl)-3,3-dimethylpiperazin-1-yl)propanamide (Compound 9b, peak 2, 22.5 mg; Method E, 2.94 min, Peak 2) as a white solid.

| Compound# | Characterization Data |
|---|---|
| 9a | LCMS (ESI) [M + H]⁺: 570.3 |
| | ¹H NMR (400 MHz, DMSO-d₆) δ 10.08 (s, 1H), 7.96 (s, 1H), 7.93 (s, 1H), 7.16-7.13 (m, 1H), |
| | 7.03-7.01 (m, 2H), 6.25 (s, 1H), 4.94 (t, J = 5.6 Hz, 1H), 4.55-4.51 (m, 1H), 4.22-4.17 (m, |
| | 1H), 4.05-4.02 (m, 1H), 3.96 (t, J = 5.0 Hz, 2H), 3.65-3.61 (m, 2H), 3.49-3.45 (m, 2H), 3.36- |
| | 3.31 (m, 1H), 3.01-2.99 (m, 1H), 2.69-2.67 (m, 1H), 2.56-2.51 (m, 1H), 2.49-2.41 (m, |
| | 1H), 2.38 (s, 2H), 1.42 (d, J = 3.2 Hz, 6H), 1.16 (d, J = 7.2 Hz, 3H). |
| 9b | LCMS (ESI) [M + H]⁺: 570.3 |
| | ¹H NMR (400 MHz, DMSO-d₆) δ 10.11 (s, 1H), 7.96 (s, 1H), 7.93 (s, 1H), 7.16 (t, J = 4.6 Hz, |
| | 1H), 7.01-6.99 (m, 2H), 6.26 (s, 1H), 4.94 (t, J = 5.4 Hz, 1H), 4.54 (t, J = 7.6 Hz, 1H), 4.21- |
| | 4.17 (m, 1H), 4.05-4.02 (m, 1H), 3.96 (t, J = 5.2 Hz, 2H), 3.65-3.61 (m, 2H), 3.47-3.45 (m, |
| | 2H), 3.36-3.31 (m, 1H), 3.01-2.99 (m, 1H), 2.69-2.67 (m, 1H), 2.56-2.52 (m, 1H), 2.49- |
| | 2.43 (m, 1H), 2.40-2.37 (m, 2H), 1.42 (d, J = 2.4 Hz, 6H), 1.16 (d, J = 6.8 Hz, 3H). |

### Example 10: (S)—N—((R)-5-(3,5-difluorophenyl)-6,7-dihydro-5H-pyrrolo[1,2-a]imidazol-2-yl)-2-(3,3-dimethyl-4-((R)-5,6,7,8-tetrahydro-[1,2,4]triazolo[4,3-a]pyridine-7-carbonyl)piperazin-1-yl)propanamide (10a) and (S)—N—((R)-5-(3,5-difluorophenyl)-6,7-dihydro-5H-pyrrolo[1,2-a]imidazol-2-yl)-2-(3,3-dimethyl-4-((S)-5,6,7,8-tetrahydro-[1,2,4]triazolo[4,3-a]pyridine-7-carbonyl)piperazin-1-yl)propanamide (10b)

### Step 1: 5,6,7,8-tetrahydro-[1,2,4]triazolo[4,3-a]pyridine-7-carboxylic acid

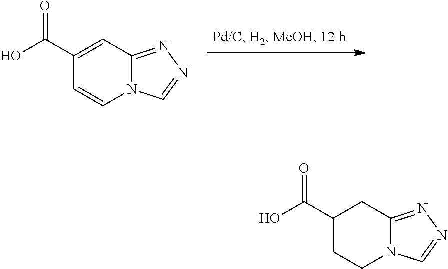 [SMILES: O=C(O)c1ccn2cnnc2c1 ; O=C(O)C1CCn2cnnc2C1]

A mixture of [1,2,4]triazolo[4,3-a]pyridine-7-carboxylic acid (150 mg, 0.9 mmol) and palladium (10 mg, 10% on charcoal) in methanol (10 ml) was stirred for 12 hours at room temperature under hydrogen atmosphere (30 atm) in a pressure reactor. Upon completion, the resulting mixture was filtered through a celite pad with methanol. The filtrate was concentrated under reduced pressure to afford 5,6,7,8-tetrahydro-[1,2,4]triazolo[4,3-a]pyridine-7-carboxylic acid (100 mg, crude) as a colorless oil, which was used in the next step without further purification. LCMS (ESI) [M+H]⁺: 168

### Step 2: (2S)—N—((R)-5-(3,5-difluorophenyl)-6,7-dihydro-5H-pyrrolo[1,2-a]imidazol-2-yl)-2-(3,3-dimethyl-4-(5,6,7,8-tetrahydro-[1,2,4]triazolo[4,3-a]pyridine-7-carbonyl)piperazin-1-yl)propanamide

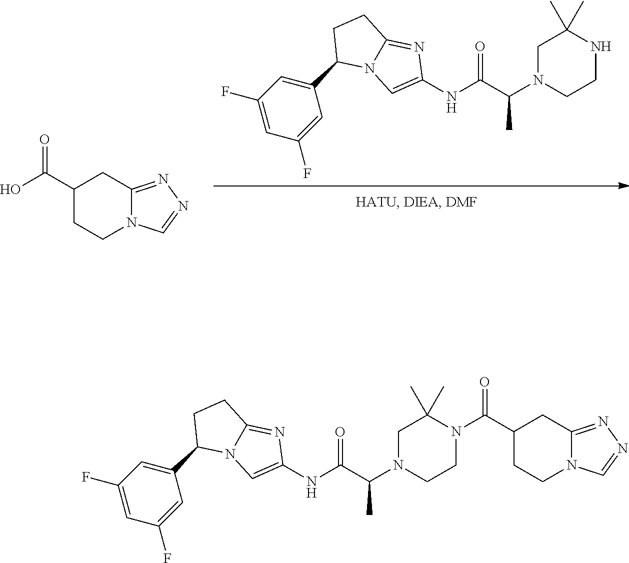 [SMILES: O=C(O)C1CCn2cnnc2C1 ; C[C@@H](C(=O)Nc1cn2c(n1)CC[C@@H]2c1cc(F)cc(F)c1)N1CCNC(C)(C)C1 ; C[C@@H](C(=O)Nc1cn2c(n1)CC[C@@H]2c1cc(F)cc(F)c1)N1CCN(C(=O)C2CCn3cnnc3C2)C(C)(C)C1]

A solution of 5,6,7,8-tetrahydro-[1,2,4]triazolo[4,3-a]pyridine-7-carboxylic acid (30 mg, 0.2 mmol), (S)—N—((R)-5-(3,5-difluorophenyl)-6,7-dihydro-5H-pyrrolo[1,2-a]imidazol-2-yl)-2-(3,3-dimethylpiperazin-1-yl)propanamide (108 mg, 0.3 mmol), DIEA (69 mg, 0.5 mmol) and HATU (102 mg, 0.3 mmol) in DMF (3.5 mL) were stirred at room temperature for 4 hours under nitrogen atmosphere. Upon completion, the resulting mixture was directly purified by reversed-phase flash chromatography (conditions: column: C18 silica gel; mobile phase A: water (0.1% formic acid), Mobile Phase B: MeCN; Gradient (B %): 5% to 95% in 25 minutes; detector: UV 254 nm) to afford (2S)—N—((R)-5-(3,5-difluorophenyl)-6,7-dihydro-5H-pyrrolo[1,2-a]imidazol-2-yl)-2-(3,3-dimethyl-4-(5,6,7,8-tetrahydro-[1,2,4]triazolo[4,3-a]pyridine-7-carbonyl)piperazin-1-yl)propanamide (30 mg, 30% yield) as a white solid. LCMS (ESI) [M+H]⁺: 553

### Step 3: (S)—N—((R)-5-(3,5-difluorophenyl)-6,7-dihydro-5H-pyrrolo[1,2-a]imidazol-2-yl)-2-(3,3-dimethyl-4-((R)-5,6,7,8-tetrahydro-[1,2,4]triazolo[4,3-a]pyridine-7-carbonyl)piperazin-1-yl)propanamide and (S)—N—((R)-5-(3,5-difluorophenyl)-6,7-dihydro-5H-pyrrolo[1,2-a]imidazol-2-yl)-2-(3,3-dimethyl-4-((S)-5,6,7,8-tetrahydro-[1,2,4]triazolo[4,3-a]pyridine-7-carbonyl)piperazin-1-yl)propanamide

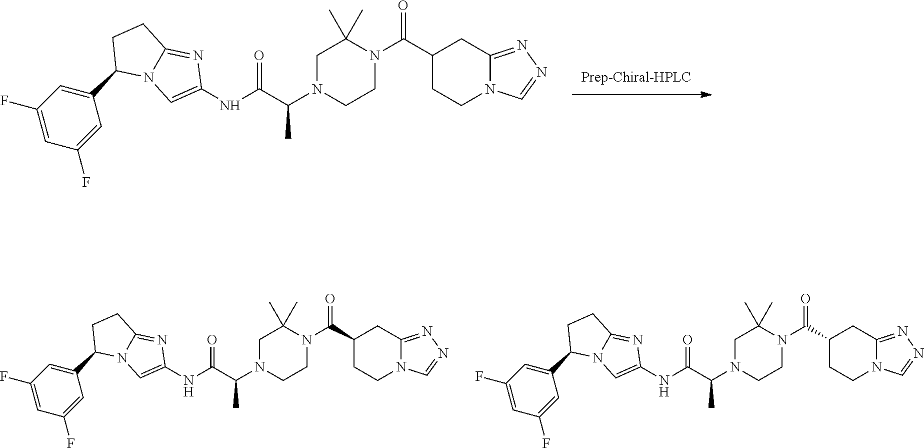 [SMILES: C[C@@H](C(=O)Nc1cn2c(n1)CC[C@@H]2c1cc(F)cc(F)c1)N1CCN(C(=O)C2CCn3cnnc3C2)C(C)(C)C1 ; C[C@@H](C(=O)Nc1cn2c(n1)CC[C@@H]2c1cc(F)cc(F)c1)N1CCN(C(=O)[C@@H]2CCn3cnnc3C2)C(C)(C)C1 ; C[C@@H](C(=O)Nc1cn2c(n1)CC[C@@H]2c1cc(F)cc(F)c1)N1CCN(C(=O)[C@H]2CCn3cnnc3C2)C(C)(C)C1]

(2S)—N—((R)-5-(3,5-difluorophenyl)-6,7-dihydro-5H-pyrrolo[1,2-a]imidazol-2-yl)-2-(3,3-dimethyl-4-(5,6,7,8-tetrahydro-[1,2,4]triazolo[4,3-a]pyridine-7-carbonyl)piperazin-1-yl)propanamide (21 mg) was resolved by Prep-Chiral HPLC (conditions: Column: Lux 5 μm Cellulose-4, 3*25 cm, 5 μm; Mobile Phase A: Hex (0.5% 2M NH₃-MeOH), Mobile Phase B: EtOH:ACN=5:1; Flow rate: 40 mL/min; Gradient (B %): isocratic 50% B; Wave Length: 210/240 nm; RT1 (min): 13.18; RT2 (min): 16.23) to afford (S)—N—((R)-5-(3,5-difluorophenyl)-6,7-dihydro-5H-pyrrolo[1,2-a]imidazol-2-yl)-2-(3,3-dimethyl-4-((R)-5,6,7,8-tetrahydro-[1,2,4]triazolo[4,3-a]pyridine-7-carbonyl)piperazin-1-yl)propanamide (Compound 10a, peak 1, 10.9 mg; Method F, 1.41 min, Peak 1) as a white solid and (S)—N—((R)-5-(3,5-difluorophenyl)-6,7-dihydro-5H-pyrrolo[1,2-a]imidazol-2-yl)-2-(3,3-dimethyl-4-((S)-5,6,7,8-tetrahydro-[1,2,4]triazolo[4,3-a]pyridine-7-carbonyl)piperazin-1-yl)propanamide (Compound 10b, peak 2, 10.4 mg; Method F, 1.99 min, Peak 2) as a white solid.

| Compound# | Characterization Data |
|---|---|
| 10a | LCMS (ESI) [M + H]⁺: 553.4 |
| | ¹H NMR (400 MHz, DMSO-d₆) δ 9.94 (s, 1H), 8.34 (s, 1H), 7.23 (t, J = 5.6 Hz, 1H), 6.96- |
| | 6.94 (m, 3H), 5.40-5.37 (m, 1H), 4.01-3.94 (m, 2H), 3.53-3.48 (m, 2H), 3.37-3.32 (m, |
| | 1H), 3.30-3.22 (m, 2H), 3.00-2.88 (m, 3H), 2.78-2.76 (m, 2H), 2.70-2.67 (m, 1H), 2.49- |
| | 2.39 (m, 1H), 2.37-2.32 (m, 2H), 2.01- 1.99 (m, 1H), 1.86-1.84 (m, 1H), 1.36 (d, J = 1.2 |
| | Hz, 6H), 1.14 (d, J = 6.8 Hz, 3H). |
| 10b | LCMS (ESI) [M + H]⁺: 553.4 |
| | ¹H NMR (400 MHz, DMSO-d₆) δ 9.93 (s, 1H), 8.34 (s, 1H), 7.23 (t, J = 9.2, Hz, 1H), 6.96- |
| | 6.94 (m, 3H), 5.40-5.37 (m, 1H), 4.01-3.94 (m, 2H), 3.51-3.49 (m, 2H), 3.28-3.25 (m, |
| | 2H), 2.97-2.86 (m, 3H), 2.80-2.76 (m, 2H), 2.66-2.55 (m, 1H), 2.50-2.39 (m, 1H), 2.39- |
| | 2.36 (m, 1H), 2.35-2.27 (m, 2H), 2.00-1.92 (m, 1H), 1.86-1.84 (m, 1H), 1.37 (d, J = 6.4 |
| | Hz, 6H), 1.14 (d, J = 6.8 Hz, 3H). |

### Example 11: 4-(4-((S)-1-(((R)-4-(3,5-difluorophenyl)-5,6-dihydro-4H-pyrrolo[1,2-b]pyrazol-2-yl)amino)-1-oxopropan-2-yl)piperazine-1-carbonyl)pyridine 1-oxide (11a) and 4-(4-((S)-1-(((S)-4-(3,5-difluorophenyl)-5,6-dihydro-4H-pyrrolo[1,2-b]pyrazol-2-yl)amino)-1-oxopropan-2-yl)piperazine-1-carbonyl)pyridine 1-oxide (11b)

### Step 1: 4-(4-(tert-butoxycarbonyl)piperazine-1-carbonyl)pyridine 1-oxide

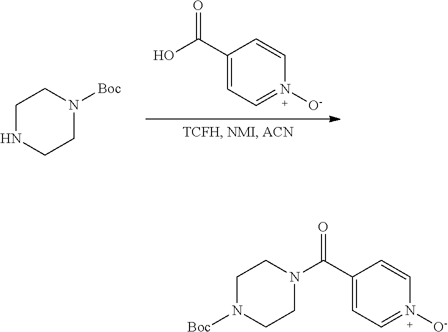

A solution of 4-carboxypyridine 1-oxide (1.00 g, 7.2 mmol), tert-butyl piperazine-1-carboxylate (1.61 g, 8.6 mmol), N-(chloro(dimethylamino)methylene)-N-methylmethanaminium hexafluorophosphate(V) (4.03 g, 14.4 mmol) and 1-methyl-1H-imidazole (2.36 g, 28.7 mmol) in ACN (20 mL) was stirred for 4 hours at room temperature. Upon completion, the reaction was quenched with ice water at 0° C. The resulting mixture was extracted with EtOAc (3×25 mL). The combined organic layers were dried over anhydrous Na₂SO₄, filtered and concentrated under reduced pressure. The crude residue was purified by silica gel column chromatography, eluted with CH₂Cl₂/MeOH (20:1) to afford 4-(4-(tert-butoxycarbonyl)piperazine-1-carbonyl)pyridine 1-oxide (1.80 g, 81% yield) as a light yellow oil. LCMS (ESI) [M+H]⁺: 308

### Step 2: 4-(piperazine-1-carbonyl)pyridine 1-oxide

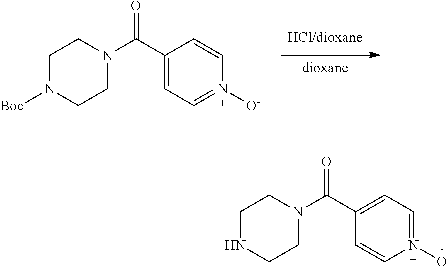

To a mixture of 4-(4-(tert-butoxycarbonyl)piperazine-1-carbonyl)pyridine 1-oxide (1.60 g, 5.2 mmol) in 1,4-dioxane (4 mL) was added 4M HCl in 1,4-dioxane (20 mL) dropwise at 0° C. The resulting mixture was warmed to room temperature and stirred for 2 hours. Upon completion, the reaction mixture was concentrated under reduced pressure to afford 4-(piperazine-1-carbonyl)pyridine 1-oxide (1.00 g, crude) as an off-white solid, which was used in the next step without further purification. LCMS (ESI) [M+H]⁺: 208

### Step 3: 4-(4-((2S)-1-((4-(3,5-difluorophenyl)-5,6-dihydro-4H-pyrrolo[1,2-b]pyrazol-2-yl)amino)-1-oxopropan-2-yl)piperazine-1-carbonyl)pyridine 1-oxide

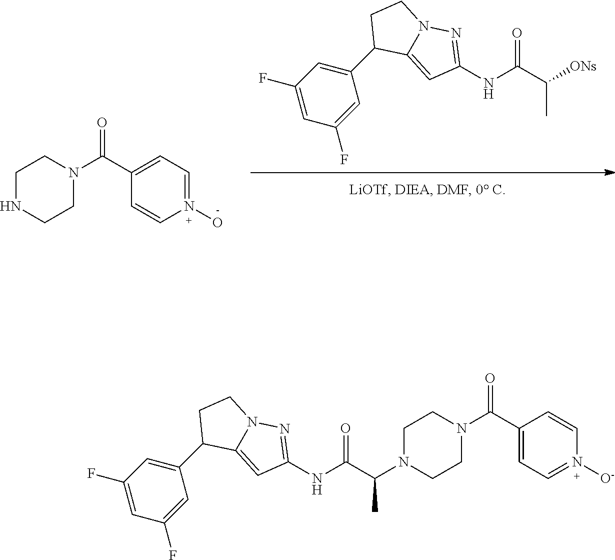

A solution of 4-(piperazine-1-carbonyl)pyridine 1-oxide (80 mg, 0.4 mmol), DIEA (175 mg, 1.4 mmol), (2R)-1-((4-(3,5-difluorophenyl)-5,6-dihydro-4H-pyrrolo[1,2-b]pyrazol-2-yl)amino)-1-oxopropan-2-yl 2-nitrobenzenesulfonate (228 mg, 0.5 mmol) and lithium trifluoromethanesulfonate (211 mg, 1.4 mmol) in DMF (8 mL) was stirred at 0° C. for 15 hours under nitrogen atmosphere. The mixture was purified by reversed-phase flash chromatography with the following conditions: column: C18 silica gel; mobile phase, MeCN in water (0.1% formic acid), 0% to 100% gradient in 20 minutes; detector: UV 254 nm) to afford 4-(4-((2S)-1-((4-(3,5-difluorophenyl)-5,6-dihydro-4H-pyrrolo[1,2-b]pyrazol-2-yl)amino)-1-oxopropan-2-yl)piperazine-1-carbonyl)pyridine 1-oxide (87 mg, 45% yield) as a light yellow solid. LCMS (ESI) [M+H]⁺: 497

### Step 4: 4-(4-((S)-1-(((R)-4-(3,5-difluorophenyl)-5,6-dihydro-4H-pyrrolo[1,2-b]pyrazol-2-yl)amino)-1-oxopropan-2-yl)piperazine-1-carbonyl)pyridine 1-oxide and 4-(4-((S)-1-(((S)-4-(3,5-difluorophenyl)-5,6-dihydro-4H-pyrrolo[1,2-b]pyrazol-2-yl)amino)-1-oxopropan-2-yl)piperazine-1-carbonyl)pyridine 1-oxide

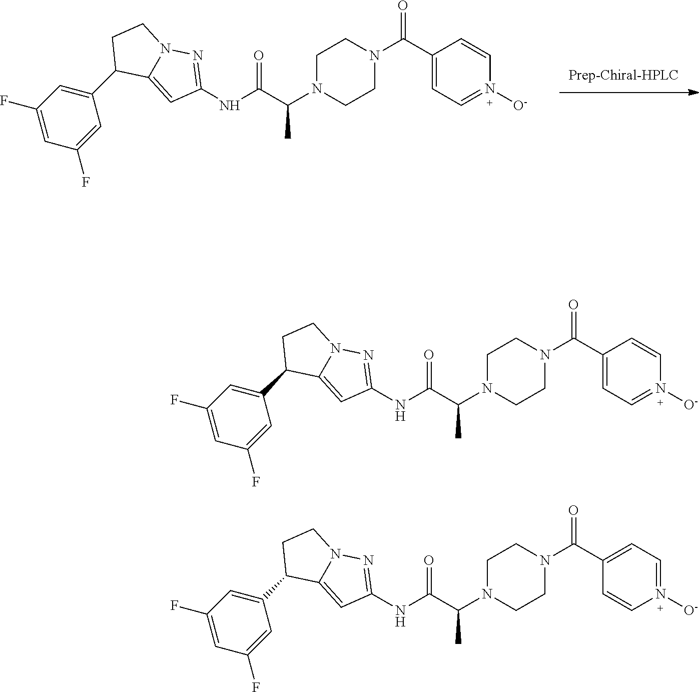

4-(4-((2S)-1-((4-(3,5-difluorophenyl)-5,6-dihydro-4H-pyrrolo[1,2-b]pyrazol-2-yl)amino)-1-oxopropan-2-yl)piperazine-1-carbonyl)pyridine 1-oxide (54 mg) was resolved by Prep-Chiral-HPLC (conditions: Column: CHIRAL ART Cellulose-SZ, 3*25 cm, 5 μm; Mobile Phase A: MTBE (10 mM NH₃-MeOH), Mobile Phase B: MeOH; Flow rate: 40 mL/min; Gradient (B %): isocratic 50% B; Wave Length: 212/282 nm; RT1 (min): 8.34; RT2 (min): 11.60) to afford 4-(4-((S)-1-(((R)-4-(3,5-difluorophenyl)-5,6-dihydro-4H-pyrrolo[1,2-b]pyrazol-2-yl)amino)-1-oxopropan-2-yl)piperazine-1-carbonyl)pyridine 1-oxide (Compound 11a, peak 1, 19.7 mg; Method G, 1.46 min, Peak 1) as a light pink solid and 4-(4-((S)-1-(((S)-4-(3,5 -difluorophenyl)-5,6-dihydro-4H-pyrrolo[1,2-b]pyrazol-2-yl)amino)-1-oxopropan-2-yl)piperazine-1-carbonyl)pyridine 1-oxide (Compound 11b, peak 2, 23.6 mg; Method G, 2.14 min, Peak 2) as a white solid.

| Compound# | Characterization Data |
|---|---|
| 11a | LCMS (ESI) [M + H]⁺: 497.2 |
| | ¹H NMR (400 MHz, DMSO-d₆) δ 10.19 (s, 1H), 8.23 (d, J = 7.2 Hz, 2H), 7.43 (d, J = 6.8 Hz, |
| | 2H), 7.16 (t, J = 5.8 Hz, 1H), 7.03-6.99 (m, 2H), 6.24 (s, 1H), 4.53 (t, J = 3.6 Hz, 1H), 4.21-4.18 |
| | (m, 1H), 4.06-4.00 (m, 1H), 3.69-3.46 (m, 2H), 3.41-3.38 (m, 2H), 3.03-2.98 (m, 1H), |
| | 2.71-2.50 (m, 5H), 2.47-2.42 (m, 1H), 1.15 (d, J = 6.8 Hz, 3H). |
| 11b | LCMS (ESI) [M + H]⁺: 497.2 |
| | ¹H NMR (400 MHz, DMSO-d₆) δ 10.19 (s, 1H), 8.23 (d, J = 6.8 Hz, 2H), 7.43 (d, J = 7.2 Hz, 2H), |
| | 7.16 (t, J = 4.6 Hz, 1H), 7.00-6.98 (m, 2H), 6.25 (s, 1H), 4.53 (t, J = 7.6 Hz, 1H), 4.23-4.18 |
| | (m, 1H), 4.06-4.00 (m, 1H), 3.67-3.45 (m, 2H), 3.48-3.39 (m, 2H), 3.03-2.98 (m, 1H), |
| | 2.67-2.49 (m, 5H), 2.46-2.43 (m, 1H), 1.15 (d, J = 6.8 Hz, 3H). |

### Example 12: 4-(4-((S)-1-(((R)-5-(3,5-difluorophenyl)-6,7-dihydro-5H-pyrrolo[1,2-a]imidazol-2-yl)amino)-1-oxopropan-2-yl)piperazine-1-carbonyl)pyridine 1-oxide

### Step 1: (R)-1-(((R)-5-(3,5-difluorophenyl)-6,7-dihydro-5H-pyrrolo[1,2-a]imidazol-2-yl)amino)-1-oxopropan-2-yl 2-nitrobenzenesulfonate

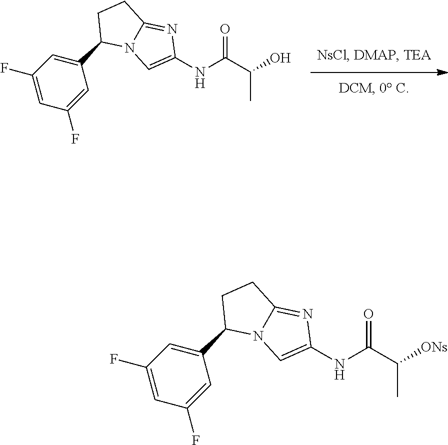

A solution of (R)—N—((R)-5-(3,5-difluorophenyl)-6,7-dihydro-5H-pyrrolo[1,2-a]imidazol-2-yl)-2-hydroxypropanamide (1.60 g, 5.2 mmol), DMAP (254 mg, 2.1 mmol), TEA (3.16 g, 31.2 mmol) and 2-nitrobenzene sulfonyl chloride (1.73 g, 7.8 mmol) in DCM (30 mL) were stirred at 0° C. for 4 hours under nitrogen atmosphere. Upon completion, the reaction was quenched with water (20 mL) at 0° C. The aqueous layer was extracted with DCM (3×60 mL). The combined organic layers were dried over anhydrous Na₂SO₄, filtered and concentrated under reduced pressure. The crude residue was purified by silica gel column chromatography, eluted with DCM/EA (1:1) to afford (R)-1-(((R)-5-(3,5-difluorophenyl)-6,7-dihydro-5H-pyrrolo[1,2-a]imidazol-2-yl)amino)-1-oxopropan-2-yl 2-nitrobenzenesulfonate (1.40 g, 54% yield) as a yellow solid. LCMS (ESI) [M+H]⁺: 493

### Step 2: 4-(4-((S)-1-(((R)-5-(3,5-difluorophenyl)-6,7-dihydro-5H-pyrrolo[1,2-a]imidazol-2-yl)amino)-1-oxopropan-2-yl)piperazine-1-carbonyl)pyridine 1-oxide

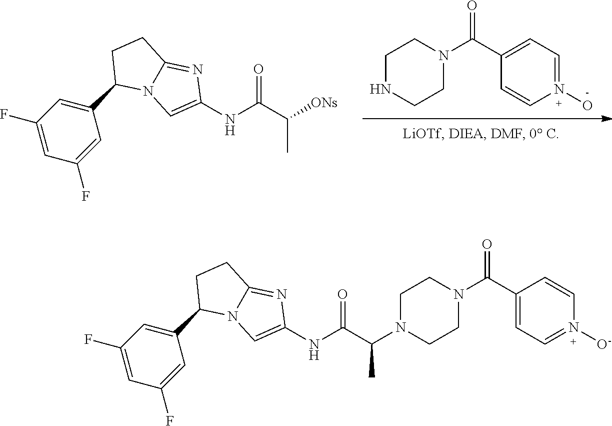

A solution of 4-(piperazine-1-carbonyl)pyridine 1-oxide (84 mg, 0.4 mmol), DIEA (78 mg, 0.6 mmol), (R)-1-(((R)-5-(3,5-difluorophenyl)-6,7-dihydro-5H-pyrrolo[1,2-a]imidazol-2-yl)amino)-1-oxopropan-2-yl 2-nitrobenzenesulfonate (100 mg, 0.2 mmol) and lithium trifluoromethanesulfonate (95 mg, 0.6 mmol) in DMF (3.5 mL) was stirred at 0° C. for 2 days under nitrogen atmosphere. Upon completion, the resulting mixture was directly purified by Prep-HPLC (conditions: Column: XBridge Shield RP18 OBD Column 30*150 mm, 5 μm; Mobile Phase A: Water (10 mmol/L NH₄HCO₃), Mobile Phase B: ACN; Flow rate: 60 mL/min; Gradient (B %): 16% to 30% in 17 minutes; Wave Length: 254 nm/220 nm; RT1 (min): 15.54) to afford 4-(4-((S)-1-(((R)-5-(3,5-difluorophenyl)-6,7-dihydro-5H-pyrrolo[1,2-a]imidazol-2-yl)amino)-1-oxopropan-2-yl)piperazine-1-carbonyl)pyridine 1-oxide (Compound 12, 20.3 mg) as a white solid.

| Compound# | Characterization Data |
|---|---|
| 12 | LCMS (ESI) [M + H]⁺: 497.1 |
| | ¹H NMR (400 MHz, DMSO-d₆) δ 10.03 (s, 1H), 8.22 (d, J = 7.2, 2H), 7.43 (d, J = 7.2, 2H), |
| | 7.24 (t, J = 9.2 Hz, 1H), 6.97-6.93 (m, 3H), 5.39-5.36 (m, 1H), 3.58-3.41 (m, 2H), 3.39-3.36 |
| | (m, 3H), 2.97-2.91 (m, 2H), 2.79-2.76 (m, 1H), 2.67-2.51 (m, 2H), 2.49-2.42 (m, 2H), |
| | 2.38-2.35 (m, 1H), 1.13 (d, J = 6.8 Hz, 3H). |

### Example 13: (S)—N—((R)-5-(3,5-difluorophenyl)-6,7-dihydro-5H-pyrrolo[1,2-a]imidazol-2-yl)-2-(3,3-dimethyl-4-(4-methyl-5-oxo-4,5-dihydropyrazine-2-carbonyl)piperazin-1-yl)propanamide

### Step 1: 4-methyl-5-oxo-4,5-dihydropyrazine-2-carboxylic acid

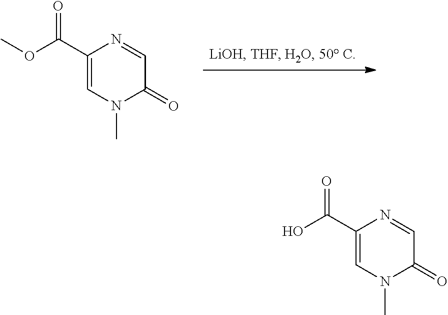

To a mixture of methyl 4-methyl-5-oxo-4,5-dihydropyrazine-2-carboxylate (100 mg, 0.6 mmol) in THF (2 mL) were added LiOH monohydrate (71 mg, 3.0 mmol) and H₂O (2 mL) at room temperature under nitrogen atmosphere. The resulting mixture was stirred at 50° C. overnight. Upon completion, the mixture was allowed to cool down to room temperature and was then acidified to pH 3 with HCl (1M). The resulting mixture was concentrated under reduced pressure. The crude residue was purified by reversed-phase flash chromatography (conditions: column: C18 silica gel; mobile phase A: water (0.1% formic acid), Mobile Phase B: MeCN; Gradient (B %): 0% to 100% in 20 minutes; detector: UV 254 nm and UV 220 nm) to afford 4-methyl-5-oxo-4,5-dihydropyrazine-2-carboxylic acid (60 mg, 65% yield) as a white solid. LCMS (ESI) [M+H]⁺: 155

### Step 2: (S)—N—((R)-5-(3,5-difluorophenyl)-6,7-dihydro-5H-pyrrolo[1,2-a]imidazol-2-yl)-2-(3,3-dimethyl-4-(4-methyl-5-oxo-4,5-dihydropyrazine-2-carbonyl)piperazin-1-yl)propanamide

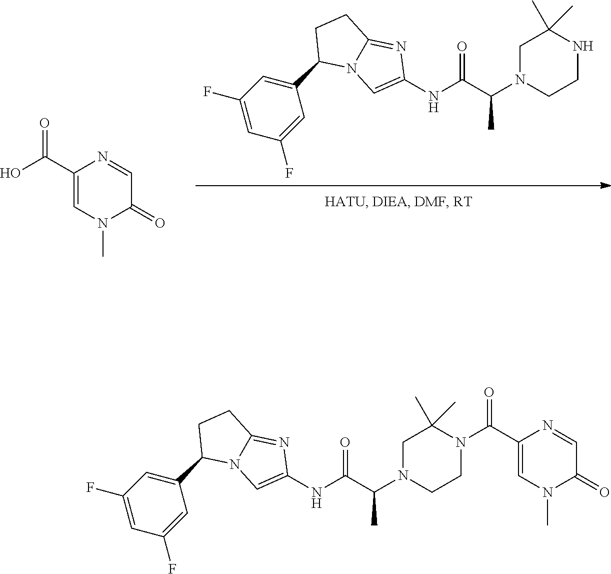 [SMILES: Cn1cc(C(=O)O)ncc1=O ; C[C@@H](C(=O)Nc1cn2c(n1)CC[C@@H]2c1cc(F)cc(F)c1)N1CCNC(C)(C)C1 ; C[C@@H](C(=O)Nc1cn2c(n1)CC[C@@H]2c1cc(F)cc(F)c1)N1CCN(C(=O)c2cn(C)c(=O)cn2)C(C)(C)C1]

A solution of 4-methyl-5-oxo-4,5-dihydropyrazine-2-carboxylic acid (40 mg, 0.2 mmol), (S)—N—((R)-5-(3,5-difluorophenyl)-6,7-dihydro-5H-pyrrolo[1,2-a]imidazol-2-yl)-2-(3,3-dimethylpiperazin-1-yl)propanamide (52 mg, 0.1 mmol), DIEA (50 mg, 0.4 mmol) and HATU (74 mg, 0.2 mmol) in DMF (5 mL) were stirred at room temperature for 4 hours under nitrogen atmosphere. Upon completion, the resulting mixture was directly purified by Prep-HPLC (conditions: Column: XBridge Prep OBD C18 Column, 30*150 mm, 5 μm; Mobile Phase A: Water (10 mmol/L NH₄HCO₃), Mobile Phase B: MeOH; Flow rate: 60 mL/min; Gradient (B %): 51% to 59% in 10 minutes; Wave Length: 254 nm/220 nm; RT1 (min): 10.13) to afford (S)—N—((R)-5-(3,5-difluorophenyl)-6,7-dihydro-5H-pyrrolo[1,2-a]imidazol-2-yl)-2-(3,3-dimethyl-4-(4-methyl-5-oxo-4,5-dihydropyrazine-2-carbonyl)piperazin-1-yl)propanamide (Compound 13, 19.5 mg) as a white solid.

| Compound# | Characterization Data |
|---|---|
| 13 | LCMS (ESI) [M + H]⁺: 540.2 |
| | ¹H NMR (400 MHz, DMSO-d₆) δ 9.93 (s, 1H), 8.10 (s, 1H), 7.90 (d, J = 1.0 Hz, 1H), |
| | 7.24-7.22 (m, 1H), 6.96-6.94 (m, 3H), 5.40-5.36 (m, 1H), 3.48-3.40 (m, 5H), 3.39-3.34 |
| | (m, 1H), 2.99-2.86 (m, 2H), 2.78-2.76 (m, 1H), 2.68-2.66 (m, 1H), 2.55-2.50 (m, 1H), |
| | 2.41-2.35 (m, 3H), 1.42 (d, J = 3.6 Hz, 6H), 1.15 (d, J = 6.8 Hz, 3H). |

### Example 14: (S)—N—((R)-5-(3,5-difluorophenyl)-6,7-dihydro-5H-pyrrolo[1,2-a]imidazol-2-yl)-2-(3,3-dimethyl-4-((R)-5,6,7,8-tetrahydro-[1,2,4]triazolo[1,5-a]pyridine-7-carbonyl)piperazin-1-yl)propanamide (14a) and (S)—N—((R)-5-(3,5-difluorophenyl)-6,7-dihydro-5H-pyrrolo[1,2-a]imidazol-2-yl)-2-(3,3-dimethyl-4-((S)-5,6,7,8-tetrahydro-[1,2,4]triazolo[1,5-a]pyridine-7-carbonyl)piperazin-1-yl)propanamide (14b)

### Step 1: 5,6,7,8-tetrahydro-[1,2,4]triazolo[1,5-a]pyridine-7-carboxylic acid

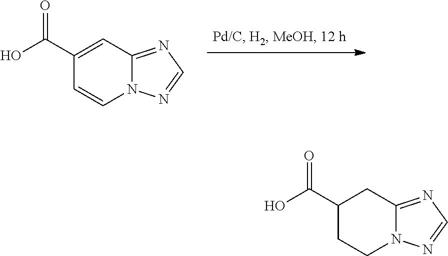 [SMILES: O=C(O)c1ccn2ncnc2c1 ; O=C(O)C1CCn2ncnc2C1]

A mixture of [1,2,4]triazolo[1,5-a]pyridine-7-carboxylic acid (150 mg, 0.9 mmol) and palladium (10 mg, 10% on charcoal) in methanol (10 ml) was stirred for 12 hours at room temperature under hydrogen atmosphere (30 atm) in a pressure reactor. Upon completion, the resulting mixture was filtered through a celite pad with methanol. The filtrate was concentrated under reduced pressure to afford 5,6,7,8-tetrahydro-[1,2,4]triazolo[1,5-a]pyridine-7-carboxylic acid (100 mg, crude) as a colorless oil, which was used in the next step without further purification. LCMS (ESI) [M+H]⁺: 168.

### Step 2: (2S)—N—((R)-5-(3,5-difluorophenyl)-6,7-dihydro-5H-pyrrolo[1,2-a]imidazol-2-yl)-2-(3,3-dimethyl-4-(5,6,7,8-tetrahydro-[1,2,4]triazolo[1,5-a]pyridine-7-carbonyl)piperazin-1-yl)propanamide

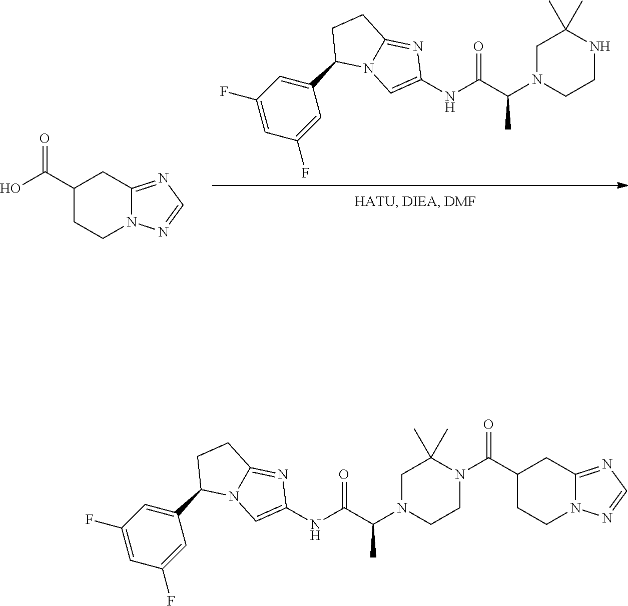 [SMILES: O=C(O)C1CCn2ncnc2C1 ; C[C@@H](C(=O)Nc1cn2c(n1)CC[C@@H]2c1cc(F)cc(F)c1)N1CCNC(C)(C)C1 ; C[C@@H](C(=O)Nc1cn2c(n1)CC[C@@H]2c1cc(F)cc(F)c1)N1CCN(C(=O)C2CCn3ncnc3C2)C(C)(C)C1]

A solution of (S)—N—((R)-5-(3,5-difluorophenyl)-6,7-dihydro-5H-pyrrolo[1,2-a]imidazol-2-yl)-2-(3,3-dimethylpiperazin-1-yl)propanamide (70 mg, 0.2 mmol), 5,6,7,8-tetrahydro-[1,2,4]triazolo[1,5-a]pyridine-7-carboxylic acid (43 mg, 0.3 mmol), DIEA (67 mg, 0.5 mmol) and HATU (98 mg, 0.3 mmol) in DMF (3.5 mL) were stirred at room temperature for 4 hours under nitrogen atmosphere. Upon completion, the resulting mixture was directly purified by reversed-phase flash chromatography (conditions: column: C18 silica gel; mobile phase A: water (0.1% NH₃·H₂O), Mobile Phase B: MeCN; Gradient (B %): 0% to 100% in 20 minutes; detector: UV 254 nm) to afford (2S)—N—((R)-5-(3,5-difluorophenyl)-6,7-dihydro-5H-pyrrolo[1,2-a]imidazol-2-yl)-2-(3,3-dimethyl-4-(5,6,7,8-tetrahydro-[1,2,4]triazolo[1,5-a]pyridine-7-carbonyl)piperazin-1-yl)propanamide (30 mg, 31% yield) as a white solid. LCMS (ESI) [M+H]⁺: 553

### Step 3: (S)—N—((R)-5-(3,5-difluorophenyl)-6,7-dihydro-5H-pyrrolo[1,2-a]imidazol-2-yl)-2-(3,3-dimethyl-4-((R)-5,6,7,8-tetrahydro-[1,2,4]triazolo[1,5-a]pyridine-7-carbonyl)piperazin-1-yl)propanamide and (S)—N—((R)-5-(3,5-difluorophenyl)-6,7-dihydro-5H-pyrrolo[1,2-a]imidazol-2-yl)-2-(3,3-dimethyl-4-((S)-5,6,7,8-tetrahydro-[1,2,4]triazolo[1,5-a]pyridine-7-carbonyl)piperazin-1-yl)propanamide

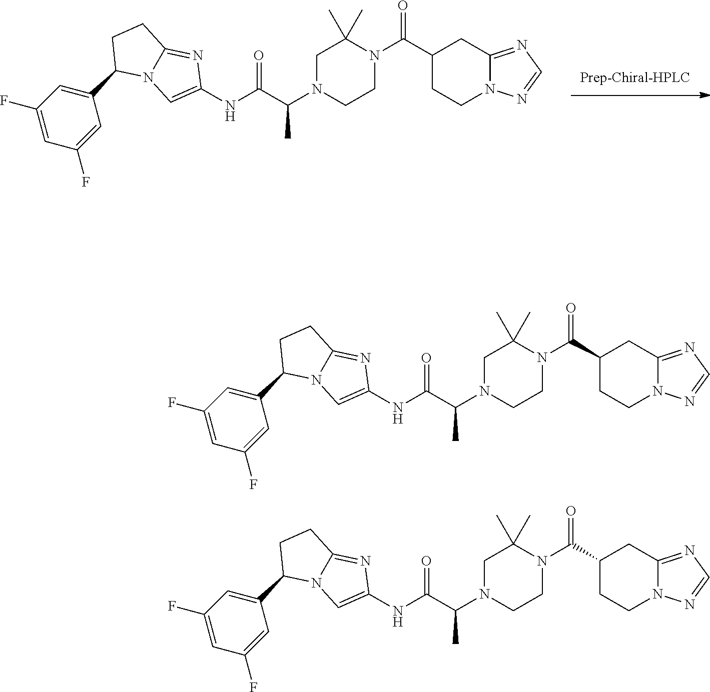 [SMILES: C[C@@H](C(=O)Nc1cn2c(n1)CC[C@@H]2c1cc(F)cc(F)c1)N1CCN(C(=O)C2CCn3ncnc3C2)C(C)(C)C1 ; C[C@@H](C(=O)Nc1cn2c(n1)CC[C@@H]2c1cc(F)cc(F)c1)N1CCN(C(=O)[C@@H]2CCn3ncnc3C2)C(C)(C)C1 ; C[C@@H](C(=O)Nc1cn2c(n1)CC[C@@H]2c1cc(F)cc(F)c1)N1CCN(C(=O)[C@H]2CCn3ncnc3C2)C(C)(C)C1]

(2S)—N—((R)-5-(3,5-difluorophenyl)-6,7-dihydro-5H-pyrrolo[1,2-a]imidazol-2-yl)-2-(3,3-dimethyl-4-(5,6,7,8-tetrahydro-[1,2,4]triazolo[1,5-a]pyridine-7-carbonyl)piperazin-1-yl)propanamide (19 mg) was resolved by Prep-Chiral HPLC (conditions: Column: CHIRALPAK ID, 2*25 cm, 5 μm; Mobile Phase A: MTBE (0.5% 2M NH₃-MeOH), Mobile Phase B: MeOH; Flow rate: 20 mL/minutes; Gradient (B %): isocratic 30% B; Wave Length: 254/220 nm; RT1 (min): 8.9; RT2 (min): 11.1) to afford (S)—N—((R)-5-(3,5-difluorophenyl)-6,7-dihydro-5H-pyrrolo[1,2-a]imidazol-2-yl)-2-(3,3-dimethyl-4-((R)-5,6,7,8-tetrahydro-[1,2,4]triazolo[1,5-a]pyridine-7-carbonyl)piperazin-1-yl)propanamide (Compound 14a, peak 1, 7.7 mg; Method H, 1.18 min, Peak 1) as a white solid and (S)—N—((R)-5-(3,5-difluorophenyl)-6,7-dihydro-5H-pyrrolo[1,2-a]imidazol-2-yl)-2-(3,3-dimethyl-4-((S)-5,6,7,8-tetrahydro-[1,2,4]triazolo[1,5-a]pyridine-7-carbonyl)piperazin-1-yl)propanamide (Compound 14b, peak 2, 8.1 mg; Method H, 1.62 min, Peak 2) as a white solid.

| Compound# | Characterization Data |
|---|---|
| 14a | LCMS (ESI) [M + H]⁺: 553.3 |
| | ¹H NMR (400 MHz, DMSO-d₆) δ 9.93 (s, 1H), 7.82 (s, 1H), 7.22 (t, J = 5.8 Hz, 1H), |
| | 7.00-6.95 (m, 3H), 5.39-5.37 (m, 1H), 4.15-4.04 (m, 2H), 3.56-3.50 (m, 2H), 3.36-3.34 |
| | (m, 1H), 3.31-3.29 (m, 1H), 3.01-2.90 (m, 2H), 2.88-2.76 (m, 3H), 2.68-2.64 (m, 1H), |
| | 2.61-2.51 (m, 1H), 2.38-2.27 (m, 3H), 2.12-2.07 (m, 1H), 1.99-1.96 (m, 1H), 1.36 |
| | (d, J = 5.2 Hz, 6H), 1.14 (d, J = 7.2 Hz, 3H). |
| 14b | LCMS (ESI) [M + H]⁺: 553.3 |
| | ¹H NMR (400 MHz, DMSO-d₆) δ 9.93 (s, 1H), 7.82 (s, 1H), 7.22 (t, J = 5.8 Hz, 1H), |
| | 6.96-6.89 (m, 3H), 5.40-5.37 (m, 1H), 4.09-4.04 (m, 2H), 3.52-3.48 (m, 2H), 3.35-3.33 |
| | (m, 1H), 3.31-3.29 (m, 1H), 2.97-2.92 (m, 2H), 2.90-2.51 (m, 5H), 2.40-2.27 (m, 3H), |
| | 2.13-2.08 (m, 1H), 1.99-1.95 (m, 1H), 1.36 (d, J = 3.2 Hz, 6H), 1.14 (d, J = 6.8 Hz, 3H). |

### Example 15: (S)—N—((R)-4-(3,5-difluorophenyl)-5,6-dihydro-4H-pyrrolo[1,2-b]pyrazol-2-yl)-2-(3,3-dimethyl-4-(5-oxo-4,5-dihydropyrazine-2-carbonyl)piperazin-1-yl)propanamide (15a) and (S)—N—((S)-4-(3,5-difluorophenyl)-5,6-dihydro-4H-pyrrolo[1,2-b]pyrazol-2-yl)-2-(3,3-dimethyl-4-(5-oxo-4,5-dihydropyrazine-2-carbonyl)piperazin-1-yl)propanamide (15b)

### Step 1: (2S)—N-(4-(3,5-difluorophenyl)-5,6-dihydro-4H-pyrrolo[1,2-b]pyrazol-2-yl)-2-(3,3-dimethyl-4-(5-oxo-4,5-dihydropyrazine-2-carbonyl)piperazin-1-yl)propanamide

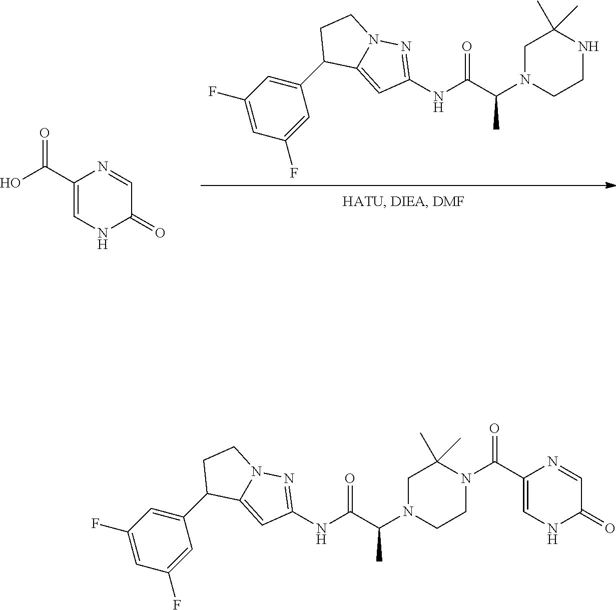 [SMILES: O=C(O)c1c[nH]c(=O)cn1 ; C[C@@H](C(=O)Nc1cc2n(n1)CCC2c1cc(F)cc(F)c1)N1CCNC(C)(C)C1 ; C[C@@H](C(=O)Nc1cc2n(n1)CCC2c1cc(F)cc(F)c1)N1CCN(C(=O)c2c[nH]c(=O)cn2)C(C)(C)C1]

A solution of 5-oxo-4,5-dihydropyrazine-2-carboxylic acid (52 mg, 0.4 mmol), (2S)—N-(4-(3,5-difluorophenyl)-5,6-dihydro-4H-pyrrolo[1,2-b]pyrazol-2-yl)-2-(3,3-dimethylpiperazin-1-yl)propanamide (100 mg, 0.2 mmol), DIEA (128 mg, 1.0 mmol) and HATU (141 mg, 0.4 mmol) in DMF (4.5 mL) were stirred at room temperature for 2 hours under nitrogen atmosphere. Upon completion, the mixture was directly purified by reversed-phase flash chromatography (conditions: column: C18 silica gel; mobile phase A: water (0.1% formic acid), Mobile Phase B: MeCN; Gradient (B %): 0% to 100% in 20 minutes; detector: UV 254 nm) to afford (2S)—N-(4-(3,5-difluorophenyl)-5,6-dihydro-4H-pyrrolo[1,2-b]pyrazol-2-yl)-2-(3,3-dimethyl-4-(5-oxo-4,5-dihydropyrazine-2-carbonyl)piperazin-1-yl)propanamide (102 mg, 78% yield) as a light yellow solid. LCMS (ESI) [M+H]⁺: 526

### Step 2: (S)—N—((R)-4-(3,5-difluorophenyl)-5,6-dihydro-4H-pyrrolo[1,2-b]pyrazol-2-yl)-2-(3,3-dimethyl-4-(5-oxo-4,5-dihydropyrazine-2-carbonyl)piperazin-1-yl)propanamide and (S)—N—((S)-4-(3,5-difluorophenyl)-5,6-dihydro-4H-pyrrolo[1,2-b]pyrazol-2-yl)-2-(3,3-dimethyl-4-(5-oxo-4,5-dihydropyrazine-2-carbonyl)piperazin-1-yl)propanamide

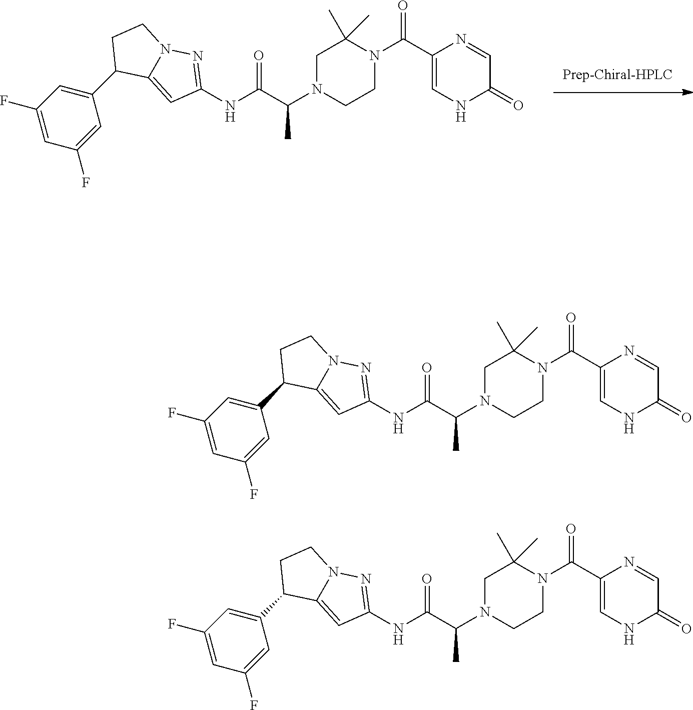 [SMILES: C[C@@H](C(=O)Nc1cc2n(n1)CCC2c1cc(F)cc(F)c1)N1CCN(C(=O)c2c[nH]c(=O)cn2)C(C)(C)C1 ; C[C@@H](C(=O)Nc1cc2n(n1)CC[C@@H]2c1cc(F)cc(F)c1)N1CCN(C(=O)c2c[nH]c(=O)cn2)C(C)(C)C1 ; C[C@@H](C(=O)Nc1cc2n(n1)CC[C@H]2c1cc(F)cc(F)c1)N1CCN(C(=O)c2c[nH]c(=O)cn2)C(C)(C)C1]

(2S)—N-(4-(3,5-difluorophenyl)-5,6-dihydro-4H-pyrrolo[1,2-b]pyrazol-2-yl)-2-(3,3-dimethyl-4-(5-oxo-4,5-dihydropyrazine-2-carbonyl)piperazin-1-yl)propanamide (95 mg) was resolved by Prep-Chiral HPLC (conditions: Column: CHIRALPAK IE, 3*25 cm, 5 μm; Mobile Phase A: MTBE (0.1% FA), Mobile Phase B: MeOH; Flow rate: 40 mL/min; Gradient (B %): isocratic 20% B; Wave Length: 242/204 nm; RT1 (min): 6.21; RT2 (min): 8.06) to afford (S)—N—((R)-4-(3,5-difluorophenyl)-5,6-dihydro-4H-pyrrolo[1,2-b]pyrazol-2-yl)-2-(3,3-dimethyl-4-(5-oxo-4,5-dihydropyrazine-2-carbonyl)piperazin-1-yl)propanamide (Compound 15a, peak 1, 30.2 mg; Method I, 0.82 min, Peak 1) as a white solid and (S)—N—((S)-4-(3,5-difluorophenyl)-5,6-dihydro-4H-pyrrolo[1,2-b]pyrazol-2-yl)-2-(3,3-dimethyl-4-(5-oxo-4,5-dihydropyrazine-2-carbonyl)piperazin-1-yl)propanamide (Compound 15b, peak 2, 32.0 mg; Method I, 1.09 min, Peak 2) as a white solid.

| Compound# | Characterization Data |
|---|---|
| 15a | LCMS (ESI) [M + H]⁺: 526.3 |
| | ¹H NMR (400 MHz, DMSO-d₆) δ 12.56 (bs, 1H), 10.07 (s, 1H), 7.90 (s, 1H), 7.72 (s, 1H), |
| | 7.15 (t, J = 3.6 Hz, 1H), 7.03-7.00 (m, 2H), 6.25 (s, 1H), 4.53 (t, J = 7.4 Hz, 1H), 4.23-4.19 |
| | (m, 1H), 4.06-4.03 (m, 1H), 3.47-3.43 (m, 2H), 3.37-3.34 (m, 1H), 3.02-2.98 (m, 1H), |
| | 2.69-2.67 (m, 1H), 2.55-2.50 (m, 1H), 2.49-2.43 (m, 1H), 2.41-2.37 (m, 2H), 1.41 |
| | (d, J = 3.2 Hz, 6H), 1.15 (d, J = 6.8 Hz, 3H). |
| 15b | LCMS (ESI) [M + H]⁺: 526.3 |
| | ¹H NMR (400 MHz, DMSO-d₆) δ 12.56 (bs, 1H), 10.11 (s, 1H), 7.91 (s, 1H), 7.72 (s, 1H), |
| | 7.16 (t, J = 4.6 Hz, 1H), 7.02-6.99 (m, 2H), 6.26 (s, 1H), 4.53 (t, J = 7.6 Hz, 1H), 4.20-4.17 |
| | (m, 1H), 4.05-4.02 (m, 1H), 3.45-3.41 (m, 2H), 3.36-3.32 (m, 1H), 3.02-2.99 (m1H), |
| | 2.68-2.66 (m, 1H), 2.57-2.51 (m, 1H), 2.46-2.39 (m, 1H), 2.39-2.31 (m, 2H), 1.40 |
| | (d, J = 2.0 Hz, 6H), 1.15 (d, J = 6.8 Hz, 3H). |

### Example 16: (S)-2-(4-acetyl-3,3-dimethylpiperazin-1-yl)-N—((R)-5-(3,5-difluorophenyl)-6,7-dihydro-5H-pyrrolo[1,2-a]imidazol-2-yl)propanamide

### Step 1: (S)-2-(4-acetyl-3,3-dimethylpiperazin-1-yl)-N—((R)-5-(3,5-difluorophenyl)-6,7-dihydro-5H-pyrrolo[1,2-a]imidazol-2-yl)propanamide

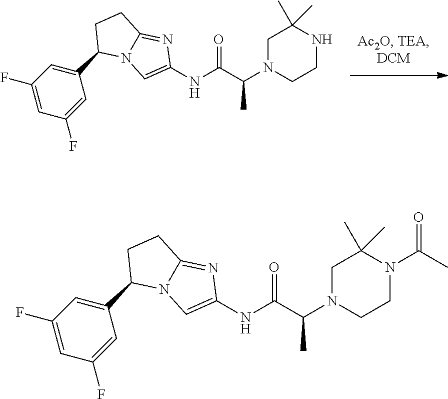 [SMILES: C[C@@H](C(=O)Nc1cn2c(n1)CC[C@@H]2c1cc(F)cc(F)c1)N1CCNC(C)(C)C1 ; CC(=O)N1CCN([C@@H](C)C(=O)Nc2cn3c(n2)CC[C@@H]3c2cc(F)cc(F)c2)CC1(C)C]

To a mixture of (S)—N—((R)-5-(3,5-difluorophenyl)-6,7-dihydro-5H-pyrrolo[1,2-a]imidazol-2-yl)-2-(3,3-dimethylpiperazin-1-yl)propanamide (40 mg, 0.1 mmol) in DCM (3 mL) were added acetic anhydride (20 mg, 0.2 mmol) and TEA (50 mg, 0.5 mmol) at room temperature. After 1 hour, the reaction mixture was concentrated under reduced pressure. The crude residue was purified by Prep-HPLC (conditions: Column: XBridge Prep OBD C18 Column 30*150 mm, 5 μm; Mobile Phase A: Water (10 mmol/L NH₄HCO₃), Mobile Phase B: ACN; Flow rate: 60 mL/min; Gradient (B %): 28% to 42% in 10 minutes; Wave Length: 254 nm/220 nm; RT1(min): 10.12) to afford (S)-2-(4-acetyl-3,3-dimethylpiperazin-1-yl)-N—((R)-5-(3,5-difluorophenyl)-6,7-dihydro-5H-pyrrolo[1,2-a]imidazol-2-yl)propanamide (Compound 16, 11.5 mg) as a white solid.

| Compound# | Characterization Data |
|---|---|
| 16 | LCMS (ESI) [M + H]⁺: 446.2 |
| | ¹H NMR (400 MHz, DMSO-d₆) δ 9.88 (s, 1H), 7.22 (t, J = 2.2 Hz, 1H), 6.96-6.93 (m, 3H), |
| | 5.39-5.37 (m, 1H), 3.37-3.34 (m, 2H), 3.33-3.31 (m, 1H), 3.03-2.87 (m, 2H), 2.81-2.78 |
| | (m, 1H), 2.68-2.62 (m, 1H), 2.54-2.52 (m, 1H), 2.39-2.37 (m, 1H), 2.35-2.30 (m, 2H), |
| | 1.94 (s, 3H), 1.35 (d, J = 4.8 Hz, 6H), 1.13 (d, J = 6.8 Hz, 3H). |

### Example 17: (S)-2-(4-acetylpiperazin-1-yl)-N—((R)-5-(3,5-difluorophenyl)-6,7-dihydro-5H-pyrrolo[1,2-a]imidazol-2-yl)propanamide

### Step 1: (S)-2-(4-acetylpiperazin-1-yl)-N—((R)-5-(3,5-difluorophenyl)-6,7-dihydro-5H-pyrrolo[1,2-a]imidazol-2-yl)propanamide

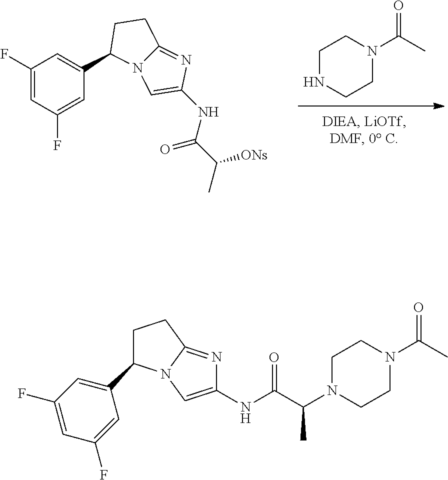

To a mixture of 1-(piperazin-1-yl)ethan-1-one (15 mg, 0.1 mmol) in DMF (2 mL) were added DIEA (31 mg, 0.2 mmol), lithium trifluoromethanesulfonate (38 mg, 0.2 mmol) and (R)-1-(((R)-5-(3,5-difluorophenyl)-6,7-dihydro-5H-pyrrolo[1,2-a]imidazol-2-yl)amino)-1-oxopropan-2-yl 2-nitrobenzenesulfonate (40 mg, 0.1 mmol) at 0° C. under nitrogen atmosphere. The resulting mixture was stirred at 0° C. overnight. Upon completion, the resulting mixture was directly purified by Prep-HPLC (conditions: Column: XBridge Prep OBD C18 Column 30*150 mm, 5 μm; Mobile Phase A: Water (10 mmol/L NH₄HCO₃), Mobile Phase B: ACN; Flow rate: 60 mL/min; Gradient (B %): 20% to 33% in 10 minutes; Wave Length: 254 nm/220 nm; RT1 (min): 10.9) to afford (S)-2-(4-acetylpiperazin-1-yl)-N—((R)-5-(3,5-difluorophenyl)-6,7-dihydro-5H-pyrrolo[1,2-a]imidazol-2-yl)propanamide (Compound 17, 17.7 mg) as a white solid.

| Compound# | Characterization Data |
|---|---|
| 17 | LCMS (ESI) [M + H]⁺: 418.2 |
| | ¹H NMR (400 MHz, DMSO-d₆) δ 9.99 (s, 1H), 7.22 (t, J = 5.8 Hz, 1H), 6.98-6.90 (m, 3H), |
| | 5.41-5.36 (m, 1H), 3.48-3.39 (m, 4H), 3.36 (d, J = 7.0 Hz, 1H), 3.04-2.86 (m, 2H), 2.83-2.71 |
| | (m, 1H), 2.53-2.50 (m, 1H), 2.49-2.46 (m, 2H), 2.39-2.32 (m, 2H), 1.96 (s, 3H), 1.12 |
| | (d, J = 7.2 Hz, 3H). |

### Example 18: (S)—N—((R)-5-(3,5-difluorophenyl)-6,7-dihydro-5H-pyrrolo[1,2-a]imidazol-2-yl)-2-(3,3,4-trimethylpiperazin-1-yl)propanamide

### Step 1: (S)—N—((R)-5-(3,5-difluorophenyl)-6,7-dihydro-5H-pyrrolo[1,2-a]imidazol-2-yl)-2-(3,3,4-trimethylpiperazin-1-yl)propanamide

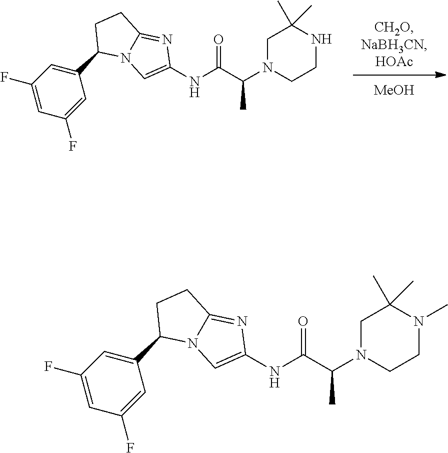 [SMILES: "MeOH" ; C[C@@H](C(=O)Nc1cn2c(n1)CC[C@@H]2c1cc(F)cc(F)c1)N1CCNC(C)(C)C1 ; C[C@@H](C(=O)Nc1cn2c(n1)CC[C@@H]2c1cc(F)cc(F)c1)N1CCN(C)C(C)(C)C1]

A solution of (S)—N—((R)-5-(3,5-difluorophenyl)-6,7-dihydro-5H-pyrrolo[1,2-a]imidazol-2-yl)-2-(3,3-dimethylpiperazin-1-yl)propanamide (40 mg, 0.1 mmol), sodium cyanoborohydride (18 mg, 0.3 mmol), AcOH (1 mg, 10 μmol) and formaldehyde solution (0.05 mL, 37% in water) in MeOH (2.5 mL) was stirred at room temperature for 2 hours under nitrogen atmosphere. Upon completion, the resulting mixture was concentrated under reduced pressure. The crude residue was purified by Prep-HPLC (conditions: Column: XBridge Prep OBD C18 Column 30*150 mm, 5 μm; Mobile Phase A: Water (10 mmol/L NH₄HCO₃), Mobile Phase B: ACN; Flow rate: 60 mL/min; Gradient (B %): 24% to 38% in 10 minutes; Wave Length: 254 nm/220 nm; RT1 (min): 9.48) to afford (S)—N—((R)-5-(3,5-difluorophenyl)-6,7-dihydro-5H-pyrrolo[1,2-a]imidazol-2-yl)-2-(3,3,4-trimethylpiperazin-1-yl)propanamide (Compound 18, 15.7 mg) as a white solid.

| Compound# | Characterization Data |
|---|---|
| 18 | LCMS (ESI) [M + H]⁺: 418.2 |
| | ¹H NMR (400 MHz, DMSO-d₆) δ 9.67 (s, 1H), 7.24 (t, J = 4.6 Hz, 1H), 6.95-6.93 (m, 3H), |
| | 5.40-5.37 (m, 1H), 3.22-3.18 (m, 1H), 2.98-2.91 (m, 2H), 2.80-2.76 (m, 1H), 2.46-2.36 |
| | (m, 5H), 2.18 (s, 2H), 2.09 (s, 3H), 1.09 (d, J = 6.8 Hz, 3H), 0.97 (d, J = 2.8 Hz, 6H). |

### Example 19: (S)—N—((R)-5-(3,5-difluorophenyl)-6,7-dihydro-5H-pyrrolo[1,2-a]imidazol-2-yl)-2-(3,3-dimethyl-4-(tetrahydro-2H-pyran-4-carbonyl)piperazin-1-yl)propanamide

### Step 1: (S)—N—((R)-5-(3,5-difluorophenyl)-6,7-dihydro-5H-pyrrolo[1,2-a]imidazol-2-yl)-2-(3,3-dimethyl-4-(tetrahydro-2H-pyran-4-carbonyl)piperazin-1-yl)propanamide

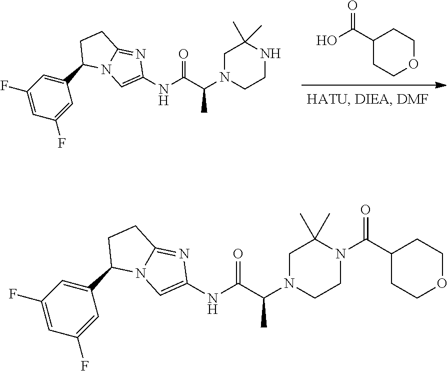

A solution of (S)—N—((R)-5-(3,5-difluorophenyl)-6,7-dihydro-5H-pyrrolo[1,2-a]imidazol-2-yl)-2-(3,3-dimethylpiperazin-1-yl)propanamide (40 mg, 90 μmol), tetrahydro-2H-pyran-4-carboxylic acid (25 mg, 0.2 mmol), HATU (56 mg, 0.1 mmol) and DIEA (38 mg, 0.3 mmol) in DMF (1.5 mL) was stirred at room temperature for 4 hours under nitrogen atmosphere. Upon completion, the resulting mixture was directly purified by Prep-HPLC (conditions: Column: Xselect CSH Prep C18, 30*150 mm 5 μm; Mobile Phase A: Water (0.1% FA), Mobile Phase B: ACN; Flow rate: 60 mL/min; Gradient (B %): gradient 11% to 32% in 10 minutes; Wave Length: 254 nm/220 nm; RT1 (min): 12.02) to afford (S)—N—((R)-5-(3,5-difluorophenyl)-6,7-dihydro-5H-pyrrolo[1,2-a]imidazol-2-yl)-2-(3,3-dimethyl-4-(tetrahydro-2H-pyran-4-carbonyl)piperazin-1-yl)propanamide (16.6 mg) as a white solid.

| Compound# | Characterization Data |
|---|---|
| 19 | LCMS (ESI) [M + H]⁺: 516.2 |
| | ¹H NMR (400 MHz, DMSO-d₆) δ 9.89 (s, 1H), 7.22 (t, J = 5.8 Hz, 1H), 7.00-6.92 (m, 3H), |
| | 5.40-5.37 (m, 1H), 3.82-3.78 (m, 2H), 3.48-3.40 (m, 2H), 3.38-3.34 (m, 3H), 3.05-2.86 |
| | (m, 2H), 2.82-2.73 (m, 2H), 2.66-2.61 (m, 1H), 2.54-2.51 (m, 1H), 2.38-2.31 (m, 1H), |
| | 2.30-2.22 (m, 2H), 1.54-1.50 (m, 4H), 1.34 (d, J = 4.0 Hz, 6H), 1.13 (d, J = 6.8 Hz, 3H). |

### Example 20: (S)-2-(4-(cyclohexanecarbonyl)-3,3-dimethylpiperazin-1-yl)-N—((R)-5-(3,5-difluorophenyl)-6,7-dihydro-5H-pyrrolo[1,2-a]imidazol-2-yl)propanamide

### Step 1: (S)-2-(4-(cyclohexanecarbonyl)-3,3-dimethylpiperazin-1-yl)-N—((R)-5-(3,5-difluorophenyl)-6,7-dihydro-5H-pyrrolo[1,2-a]imidazol-2-yl)propanamide

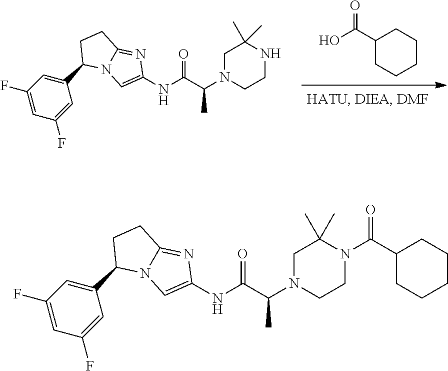

A solution of (S)—N—((R)-5-(3,5-difluorophenyl)-6,7-dihydro-5H-pyrrolo[1,2-a]imidazol-2-yl)-2-(3,3-dimethylpiperazin-1-yl)propanamide (40 mg, 90 μmol), cyclohexanecarboxylic acid (25 mg, 0.2 mmol), HATU (56 mg, 0.1 mmol) and DIEA (38 mg, 0.3 mmol) in DMF (2 mL) was stirred at room temperature for 4 hours under nitrogen atmosphere. Upon completion, the resulting mixture was directly purified by Prep-HPLC (conditions: Column: Xselect CSH Prep C18, 30*150 mm 5 μm; Mobile Phase A: Water (0.1% FA), Mobile Phase B: ACN; Flow rate: 60 mL/min; Gradient (B %): gradient 26% to 47% in 10 minutes; Wave Length: 254 nm/220 nm; RT1 (min): 10.67) to afford (S)-2-(4-(cyclohexanecarbonyl)-3,3-dimethylpiperazin-1-yl)-N—((R)-5-(3,5-difluorophenyl)-6,7-dihydro-5H-pyrrolo[1,2-a]imidazol-2-yl)propanamide (Compound 20, 20.4 mg) as a white solid.

| Compound# | Characterization Data |
|---|---|
| 20 | LCMS (ESI) [M + H]⁺: 514.3 |
| | ¹H NMR (400 MHz, DMSO-d₆) δ 11.18 (bs, 1H), 7.24 (t, J = 9.2, 1H), 7.07-6.90 (m, 3H), |
| | 5.49-5.41 (m, 1H), 4.08-3.82 (m, 2H), 3.72-3.45 (m, 4H), 3.28-3.08 (m, 2H), 3.07-2.96 |
| | (m, 2H), 2.95-2.85 (m, 1H), 2.55-2.51 (m, 1H), 2.44-2.29 (m, 1H), 1.76-1.56 (m, 5H), |
| | 1.55-1.34 (m, 7H), 1.33-1.07 (m, 6H). |

### Example 21: (S)—N—((R)-5-(3,5-difluorophenyl)-6,7-dihydro-5H-pyrrolo[1,2-a]imidazol-2-yl)-2-(2,2-dimethylmorpholino)propanamide

### Step 1: (S)—N—((R)-5-(3,5-difluorophenyl)-6,7-dihydro-5H-pyrrolo[1,2-a]imidazol-2-yl)-2-(2,2-dimethylmorpholino)propanamide

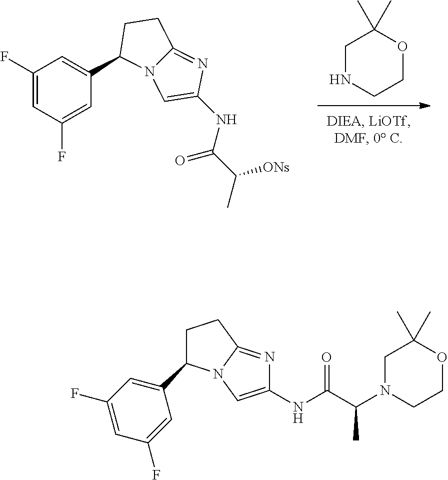

To a mixture of 2,2-dimethylmorpholine (14 mg, 0.1 mmol) in DMF (2 mL) were added DIEA (31 mg, 0.2 mmol), lithium trifluoromethanesulfonate (38 mg, 0.2 mmol) and (R)-1-(((R)-5-(3,5-difluorophenyl)-6,7-dihydro-5H-pyrrolo[1,2-a]imidazol-2-yl)amino)-1-oxopropan-2-yl 2-nitrobenzenesulfonate (40 mg, 80 μmol) in portions at 0° C. under nitrogen atmosphere. The resulting mixture was stirred at 0° C. overnight. Upon completion, the resulting mixture was purified by Prep-HPLC (conditions: Column: XBridge Prep OBD C18 Column 30*150 mm, 5 m; Mobile Phase A: Water (10 mmol/L NH₄HCO₃), Mobile Phase B: ACN; Flow rate: 60 mL/min; Gradient (B %): 30% to 44% in 10 minutes; Wave Length: 254 nm/220 nm; RT1 (min): 10.95) to afford (S)—N—((R)-5-(3,5-difluorophenyl)-6,7-dihydro-5H-pyrrolo[1,2-a]imidazol-2-yl)-2-(2,2-dimethylmorpholino)propanamide (17.5 mg) as a white solid.

| Compound# | Characterization Data |
|---|---|
| 21 | LCMS (ESI) [M + H]⁺: 405.1 |
| | ¹H NMR (400 MHz, DMSO-d₆) δ 9.82 (s, 1H), 7.22 (t, J = 8.2 Hz, 1H), 7.00-6.91 (m, 3H), |
| | 5.41-5.37 (m, 1H), 3.61 (t, J = 4.8 Hz, 2H), 3.25-3.22 (m, 1H), 3.03-2.91 (m, 2H), 2.82-2.76 |
| | (m, 1H), 2.47-2.42 (m, 1H), 2.39-2.33 (m, 2H), 2.29-2.26 (m, 2H), 1.16 (s, 6H), 1.11 |
| | (d, J = 7.2 Hz, 3H). |

### Synthesis of Intermediate D: (S)—N-(1-(3,5-difluorobenzyl)-2-(difluoromethyl)-1H-imidazol-4-yl)-2-(3,3-dimethylpiperazin-1-yl)propanamide

### Step 1: methyl 4-bromo-1-(3,5-difluorobenzyl)-1H-imidazole-2-carboxylate

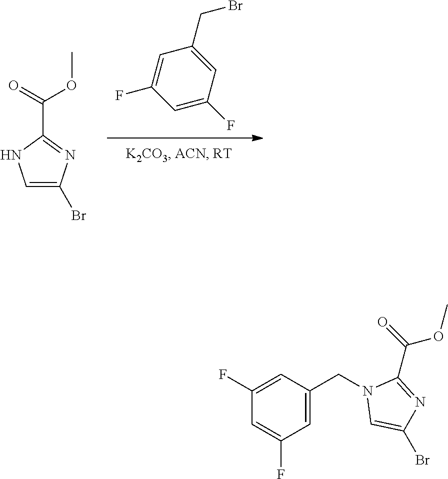

A solution of methyl 4-bromo-1H-imidazole-2-carboxylate (2.00 g, 9.8 mmol), 1-(bromomethyl)-3,5-difluorobenzene (2.40 g, 11.7 mmol) and K₂CO₃ (4.00 g, 29.3 mmol) in ACN (20 mL) was stirred at room temperature for 16 hours under nitrogen atmosphere. Upon completion, the reaction was quenched with water. The resulting mixture was extracted with EtOAc (3×60 mL). The combined organic layers were dried over anhydrous Na₂SO₄, filtered and concentrated under reduced pressure. The crude residue was purified by silica gel column chromatography, eluted with PE/EA (1:1) to afford methyl 4-bromo-1-(3,5-difluorobenzyl)-1H-imidazole-2-carboxylate (2.30 g, 71% yield) as a yellow solid. LCMS (ESI) [M+H]⁺: 331

### Step 2: 4-bromo-1-(3,5-difluorobenzyl)-1H-imidazole-2-carbaldehyde

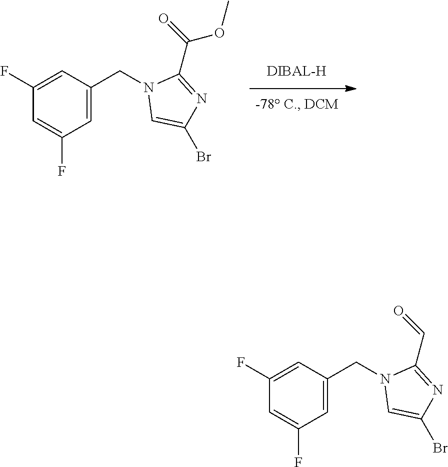 [SMILES: COC(=O)c1nc(Br)cn1Cc1cc(F)cc(F)c1 ; O=Cc1nc(Br)cn1Cc1cc(F)cc(F)c1]

To a mixture of methyl 4-bromo-1-(3,5-difluorobenzyl)-1H-imidazole-2-carboxylate (2.26 g, 6.8 mmol) in DCM (25 mL) was added DIBAL-H (13.5 mL, 13.6 mmol, 1M in toluene) dropwise at −78° C. under nitrogen atmosphere. The resulting mixture was stirred at −78° C. for 1 hour. Upon completion, the reaction was quenched with sat. NH₄Cl (aq.) at 0° C. The resulting mixture was extracted with EtOAc (3×60 mL). The combined organic layers were washed with brine, dried over anhydrous Na₂SO₄, filtered and concentrated under reduced pressure. The crude residue was purified by silica gel column chromatography, eluted with PE/EA (1:1) to afford 4-bromo-1-(3,5-difluorobenzyl)-1H-imidazole-2-carbaldehyde (2.03 g, 98% yield) as a yellow solid. LCMS (ESI) [M+H]⁺: 301

### Step 3: 4-bromo-1-(3,5-difluorobenzyl)-2-(difluoromethyl)-1H-imidazole

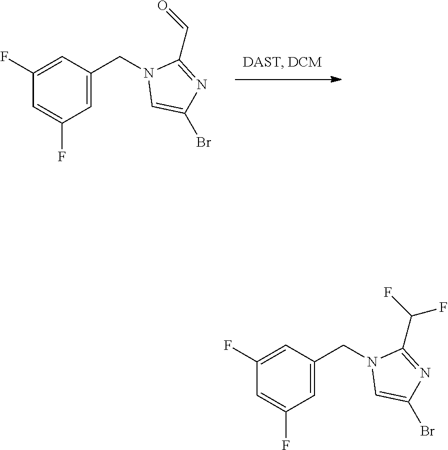 [SMILES: O=Cc1nc(Br)cn1Cc1cc(F)cc(F)c1 ; Fc1cc(F)cc(Cn2cc(Br)nc2C(F)F)c1]

To a mixture of 4-bromo-1-(3,5-difluorobenzyl)-1H-imidazole-2-carbaldehyde (2.03 g, 6.8 mmol) in DCM (40 mL) was added DAST (10.81 g, 67.3 mmol) dropwise at 0° C. under nitrogen atmosphere. The resulting mixture was stirred at room temperature for 16 hours. Upon completion, the reaction was quenched with NaHCO₃(aq.) at 0° C. The resulting mixture was extracted with CH₂Cl₂ (3×70 mL). The combined organic layers were washed with brine, dried over anhydrous MgSO₄, filtered and concentrated under reduced pressure. The crude residue was purified by silica gel column chromatography, eluted with PE/EA (2:1) to afford 4-bromo-1-(3,5-difluorobenzyl)-2-(difluoromethyl)-1H-imidazole (946 mg, 43% yield) as a yellow solid. LCMS (ESI) [M+H]⁺: 323.

### Step 4: (R)—N-(1-(3,5-difluorobenzyl)-2-(difluoromethyl)-1H-imidazol-4-yl)-2-hydroxypropanamide

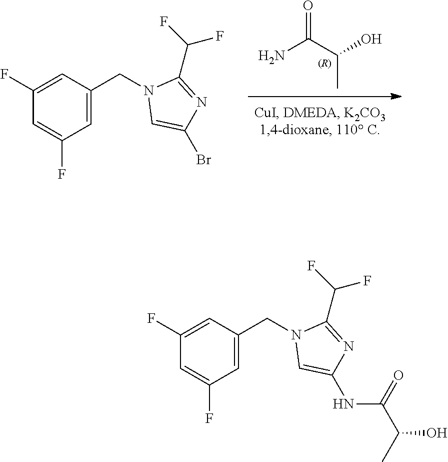

To a mixture of 4-bromo-1-(3,5-difluorobenzyl)-2-(difluoromethyl)-1H-imidazole (946 mg, 3.0 mmol) in 1,4-dioxane (12 mL) were added (R)-2-hydroxypropanamide (522 mg, 5.81 mmol), CuI (112 mg, 0.5 mmol), N,N-dimethyl-1,2-ethanediamine (51 mg, 0.5 mmol) and K₂CO₃ (810 mg, 5.8 mmol). The resulting mixture was stirred at 110° C. overnight under nitrogen atmosphere. Upon completion, the mixture was allowed to cool down to room temperature. The reaction was quenched with water at 0° C. The resulting mixture was extracted with EtOAc (3×20 mL). The combined organic layers were dried over anhydrous Na₂SO₄, filtered and concentrated under reduced pressure. The crude residue was purified by reversed-phase flash chromatography (conditions: column: C18 silica gel; mobile phase A: water (0.1% formic acid), Mobile phase B: MeCN; Gradient (B %): 5% to 100% in 20 minutes; detector: UV 254 nm and UV 220 nm) to afford (R)—N-(1-(3,5-difluorobenzyl)-2-(difluoromethyl)-1H-imidazol-4-yl)-2-hydroxypropanamide (473 mg, 49% yield) as a yellow solid. LCMS (ESI) [M+H]⁺: 332

### Step 5: (R)-1-((1-(3,5-difluorobenzyl)-2-(difluoromethyl)-1H-imidazol-4-yl)amino)-1-oxopropan-2-yl 2-nitrobenzenesulfonate

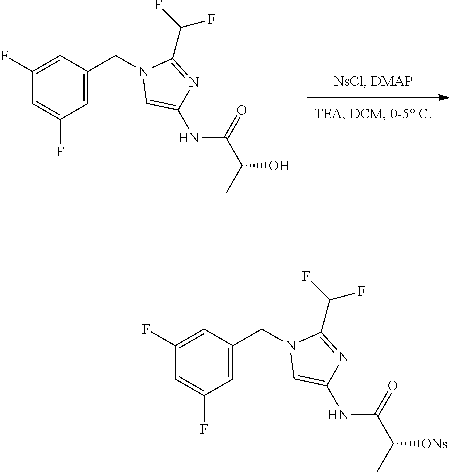

To a mixture of (R)—N-(1-(3,5-difluorobenzyl)-2-(difluoromethyl)-1H-imidazol-4-yl)-2-hydroxypropanamide (473 mg, 1.5 mmol) in DCM (10 mL) were added DMAP (70 mg, 0.5 mmol), TEA (434 mg, 4.3 mmol) and 2-nitrobenzene-1-sulfonyl chloride (475 mg, 2.2 mmol) at 0° C. under nitrogen atmosphere. The resulting mixture was stirred at 0° C. for 5 hours. Upon completion, the reaction was quenched with water at 0° C. The resulting mixture was extracted with EtOAc (3×20 mL). The combined organic layers were dried over anhydrous Na₂SO₄, filtered and concentrated under reduced pressure. The crude residue was purified by silica gel column chromatography, eluted with DCM/EA (1:1) to afford (R)-1-((1-(3,5-difluorobenzyl)- 2-(difluoromethyl)-1H-imidazol-4-yl)amino)-1-oxopropan-2-yl 2-nitrobenzenesulfonate (311 mg, 42% yield, 36% ee) as a yellow solid. LCMS (ESI) [M+H]⁺: 517.

### Step 6: tert-butyl (S)-4-(1-((1-(3,5-difluorobenzyl)-2-(difluoromethyl)-1H-imidazol-4-yl)amino)-1-oxopropan-2-yl)-2,2-dimethylpiperazine-1-carboxylate

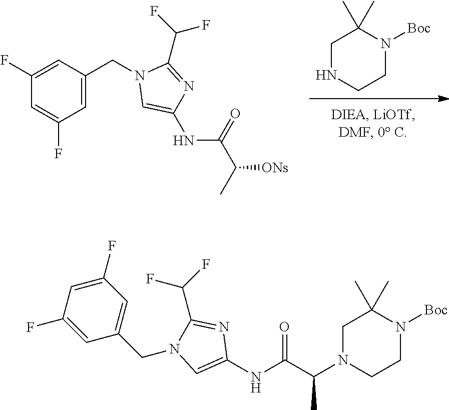

To a mixture of tert-butyl 2,2-dimethylpiperazine-1-carboxylate (311 mg, 1.5 mmol) in DMF (5 mL) was added DIEA (375 mg, 2.9 mmol), lithium trifluoromethanesulfonate (453 mg, 2.9 mmol) and (R)-1-((1-(3,5-difluorobenzyl)-2-(difluoromethyl)-1H-imidazol-4-yl)amino)-1-oxopropan-2-yl 2-nitrobenzenesulfonate (500 mg, 1.0 mmol, 36% ee) at 0° C. under nitrogen atmosphere. The resulting mixture was stirred at 0° C. overnight. Upon completion, the crude residue was directly purified by reversed-phase flash chromatography (conditions: column: C18 silica gel; mobile phase A: water (10 mmol/L NH₄HCO₃), Mobile phase B: MeCN; Gradient (B %): 0% to 100% in 20 min; detector: UV 254 nm) to afford tert-butyl (S)-4-(1-((1-(3,5-difluorobenzyl)-2-(difluoromethyl)-1H-imidazol-4-yl)amino)-1-oxopropan-2-yl)-2,2-dimethylpiperazine-1-carboxylate (450 mg, 88% yield, 36% ee) as a white solid. LCMS (ESI) [M+H]⁺: 528.

### Step 7: (S)—N-(1-(3,5-difluorobenzyl)-2-(difluoromethyl)-1H-imidazol-4-yl)-2-(3,3-dimethylpiperazin-1-yl)propanamide

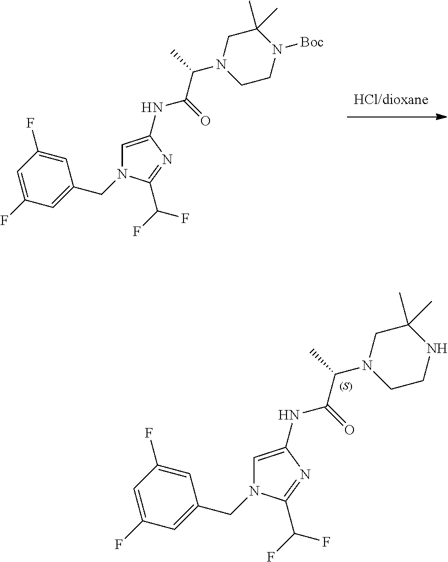

To a mixture of tert-butyl (S)-4-(1-((1-(3,5-difluorobenzyl)-2-(difluoromethyl)-1H-imidazol-4-yl)amino)-1-oxopropan-2-yl)-2,2-dimethylpiperazine-1-carboxylate (450 mg, 1.0 mmol, 36% ee) in 1,4-dioxane (2 mL) was added 4 M HCl in 1,4-dioxane (10 mL) dropwise at 0° C. The resulting mixture was warmed to room temperature and stirred for 2 hours. Upon completion, the reaction mixture was concentrated under reduced pressure. This resulted in (S)—N-(1-(3,5-difluorobenzyl)-2-(difluoromethyl)-1H-imidazol-4-yl)-2-(3,3-dimethylpiperazin-1 -yl)propanamide (Intermediate D) (400 mg, crude, 36% ee) as a white solid, which was used directly in the next step without further purification. LCMS (ESI) [M+H]⁺: 428 Example 22: (S)-4-(4-(1-((1-(3,5-difluorobenzyl)-2-(difluoromethyl)-1H-imidazol-4-yl)amino)-1-oxopropan-2-yl)-2,2-dimethylpiperazine-1-carbonyl)pyridine 1-oxide

### Step 1: (S)-4-(4-(1-((1-(3,5-difluorobenzyl)-2-(difluoromethyl)-1H-imidazol-4-yl)amino)-1-oxopropan-2-yl)-2,2-dimethylpiperazine-1-carbonyl)pyridine 1-oxide

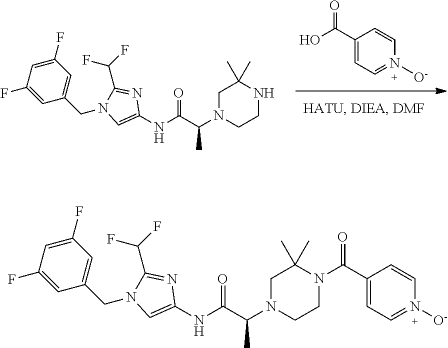

A solution of (S)—N-(1-(3,5-difluorobenzyl)-2-(difluoromethyl)-1H-imidazol-4-yl)-2-(3,3-dimethylpiperazin-1-yl)propanamide (50 mg, 0.1 mmol, 36% ee, Intermediate D), 4-carboxypyridine 1-oxide (32 mg, 0.2 mmol), HATU (89 mg, 0.2 mmol) and DIEA (75 mg, 0.6 mmol) in DMF (2.5 mL) was stirred at room temperature overnight under nitrogen atmosphere. Upon completion, the resulting solution was directly purified by Prep-HPLC (conditions: Column: Xselect CSH Prep C18, 30*150 mm 5 μm; Mobile Phase A: Water (0.1% FA), Mobile Phase B: ACN; Flow rate: 60 mL/min; Gradient (B %): 20% to 38% B in 10 minutes; Wave Length: 254 nm/220 nm; RT1 (min): 10.99) to afford (S)-4-(4-(1-((1-(3,5-difluorobenzyl)-2-(difluoromethyl)-1H-imidazol-4-yl)amino)-1-oxopropan-2-yl)-2,2-dimethylpiperazine-1-carbonyl)pyridine 1-oxide (Compound 22, 17.8 mg, 33% ee; Method J, 1.17 min, Peak 1 (major enantiomer); 1.27 min, Peak 2, (minor enantiomer)) as a white solid.

| Compound# | Characterization Data |
|---|---|
| 22 | LCMS (ESI) [M + H]⁺: 549.3 |
| | ¹H NMR (400 MHz, DMSO-d₆) δ 10.34 (s, 1H), 8.20 (d, J = 7.2 Hz, 2H), 7.53 (s, 1H), |
| | 7.40 (d, J = 7.2 Hz, 2H), 7.29-7.17 (m, 2H), 6.97-6.91 (m, 2H), 5.36 (s, 2H), 3.41-3.39 |
| | (m, 1H), 3.28-3.26 (m, 2H), 2.67-2.66 (m, 1H), 2.56-2.51 (m, 1H), 2.42 (s, 2H), 1.42 |
| | (d, J = 5.2 Hz, 6H), 1.16 (d, J = 7.2 Hz, 3H) |

### Example 23: (S)—N-(1-(3,5-difluorobenzyl)-2-(difluoromethyl)-1H-imidazol-4-yl)-2-(3,3-dimethyl-4-(6-oxo-1,6-dihydropyridine-3-carbonyl)piperazin-1-yl)propanamide

### Step 1: (S)—N-(1-(3,5-difluorobenzyl)-2-(difluoromethyl)-1H-imidazol-4-yl)-2-(3,3-dimethyl-4-(6-oxo-1,6-dihydropyridine-3-carbonyl)piperazin-1-yl)propanamide

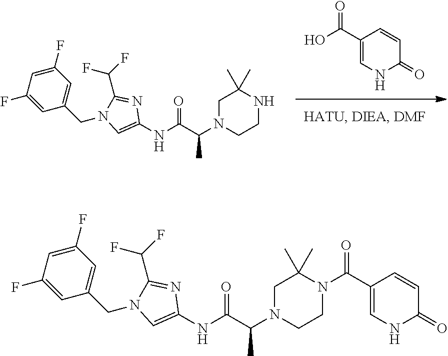 [SMILES: C[C@@H](C(=O)Nc1cn(Cc2cc(F)cc(F)c2)c(C(F)F)n1)N1CCNC(C)(C)C1 ; O=C(O)c1ccc(=O)[nH]c1 ; C[C@@H](C(=O)Nc1cn(Cc2cc(F)cc(F)c2)c(C(F)F)n1)N1CCN(C(=O)c2ccc(=O)[nH]c2)C(C)(C)C1]

A solution of (S)—N-(1-(3,5-difluorobenzyl)-2-(difluoromethyl)-1H-imidazol-4-yl)-2-(3,3-dimethylpiperazin-1-yl)propanamide (50 mg, 0.1 mmol, 36% ee, Intermediate D), 6-oxo-1,6-dihydropyridine-3-carboxylic acid (32 mg, 0.2 mmol), HATU (89 mg, 0.2 mmol) and DIEA (75 mg, 0.6 mmol) in DMF (2.5 mL) was stirred at room temperature overnight under nitrogen atmosphere. Upon completion, the resulting solution was purified by Prep-HPLC (conditions: Column: Xselect CSH Prep C18, 30*150 mm 5 μm; Mobile Phase A: Water (0.1% FA), Mobile Phase B: ACN; Flow rate: 60 mL/min; Gradient (B %): 17% to 35% B in 12 min; Wave Length: 254 nm/220 nm; RT1 (min): 12.82) to afford (S)—N-(1-(3,5-difluorobenzyl)-2-(difluoromethyl)-1H-imidazol-4-yl)-2-(3,3-dimethyl-4-(6-oxo-1,6-dihydropyridine-3-carbonyl)piperazin-1-yl)propanamide (Compound 23, 8.6 mg, 33% ee; Method J, 1.09 min, Peak 1 (major enantiomer); 1.15 min, Peak 2, (minor enantiomer)) as an off-white solid.

| Compound# | Characterization Data |
|---|---|
| 23 | LCMS (ESI) [M + H]⁺: 549.3 |
| | ¹H NMR (400 MHz, DMSO-d₆) δ 12.21-9.88 (m, 1H), 7.78-7.15 (m, 5H), 6.97-6.91 |
| | (m, 2H), 6.48-6.35 (m, 1H), 5.50-5.36 (m, 2H), 4.21-2.35 (m, 8H), 1.69-1.04 (m, 9H) |

### Example 24: (S)—N-(1-(3,5-difluorobenzyl)-2-(difluoromethyl)-1H-imidazol-4-yl)-2-(3,3-dimethyl-4-(5-oxo-4,5-dihydropyrazine-2-carbonyl)piperazin-1-yl)propanamide

### Step 1: (S)—N-(1-(3,5-difluorobenzyl)-2-(difluoromethyl)-1H-imidazol-4-yl)-2-(3,3-dimethyl-4-(5-oxo-4,5-dihydropyrazine-2-carbonyl)piperazin-1-yl)propanamide

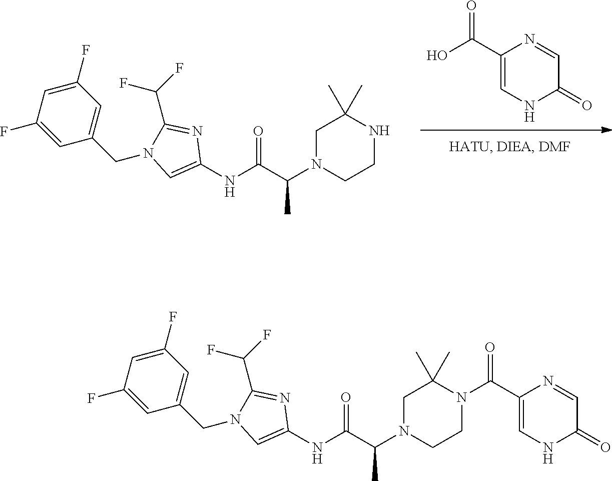 [SMILES: C[C@@H](C(=O)Nc1cn(Cc2cc(F)cc(F)c2)c(C(F)F)n1)N1CCNC(C)(C)C1 ; O=C(O)c1c[nH]c(=O)cn1 ; C[C@@H](C(=O)Nc1cn(Cc2cc(F)cc(F)c2)c(C(F)F)n1)N1CCN(C(=O)c2c[nH]c(=O)cn2)C(C)(C)C1]

A solution of(S)—N-(1-(3,5-difluorobenzyl)-2-(difluoromethyl)-1H-imidazol-4-yl)-2-(3,3-dimethylpiperazin-1-yl)propanamide (50 mg, 0.1 mmol, 36% ee, Intermediate D), 5-oxo-4,5-dihydropyrazine-2-carboxylic acid (32 mg, 0.2 mmol), HATU (88 mg, 0.2 mmol) and DIEA (75 mg, 0.6 mmol) in DMF (2.5 mL) was stirred at room temperature overnight under nitrogen atmosphere. Upon completion, the resulting solution was purified by Prep-HPLC (conditions: Column: Xselect CSH Prep C18, 30*150 mm 5 μm; Mobile Phase A: Water (0.1% FA), Mobile Phase B: ACN; Flow rate: 60 mL/min; Gradient (B %): 15% to 33% B in 12 min; Wave Length: 254 nm/220 nm; RT1 (min): 13.07) to afford (S)—N-(1-(3,5-difluorobenzyl)-2-(difluoromethyl)-1H-imidazol-4-yl)-2-(3,3-dimethyl-4-(5-oxo-4,5-dihydropyrazine-2-carbonyl)piperazin-1-yl)propanamide (Compound 24, 22.0 mg, 34% ee; Method K, 4.51 min, Peak 1 (major enantiomer); 4.78 min, Peak 2, (minor enantiomer)) as an off-white solid.

| Compound# | Characterization Data |
|---|---|
| 24 | LCMS (ESI) [M + H]⁺: 550.3 |
| | ¹H NMR (400 MHz, DMSO-d₆) δ 12.61-9.98 (m, 1H), 7.98-7.15 (m, 5H), |
| | 6.97-6.91 (m, 2H), 5.50-5.36 (m, 2H), 4.21-2.29 (m, 8H), 1.70-1.09 (m, 9H) |

### Example 25: (S)—N-(1-(3,5-difluorobenzyl)-2-(difluoromethyl)-1H-imidazol-4-yl)-2-(3,3-dimethyl-4-(4-methyl-5-oxo-4,5-dihydropyrazine-2-carbonyl)piperazin-1-yl)propenamide

### Step 1: (S)—N-(1-(3,5-difluorobenzyl)-2-(difluoromethyl)-1H-imidazol-4-yl)-2-(3,3-dimethyl-4-(4-methyl-5-oxo-4,5-dihydropyrazine-2-carbonyl)piperazin-1-yl)propanamide

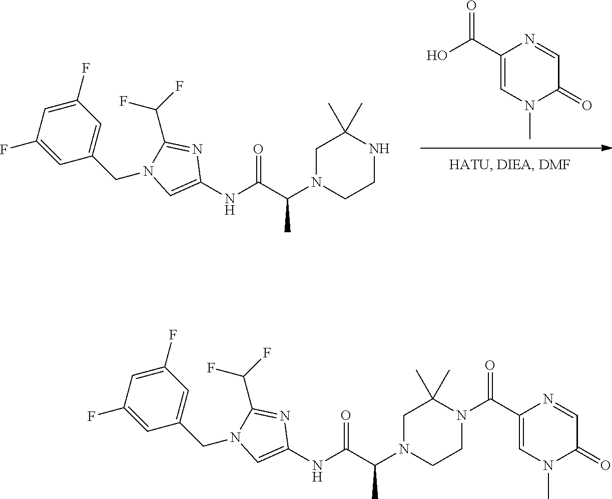 [SMILES: C[C@@H](C(=O)Nc1cn(Cc2cc(F)cc(F)c2)c(C(F)F)n1)N1CCNC(C)(C)C1 ; Cn1cc(C(=O)O)ncc1=O ; C[C@@H](C(=O)Nc1cn(Cc2cc(F)cc(F)c2)c(C(F)F)n1)N1CCN(C(=O)c2cn(C)c(=O)cn2)C(C)(C)C1]

A solution of (S)—N-(1-(3,5-difluorobenzyl)-2-(difluoromethyl)-1H-imidazol-4-yl)-2-(3,3-dimethylpiperazin-1-yl)propanamide (50 mg, 0.1 mmol, 36% ee, Intermediate D), 4-methyl-5-oxo-4,5-dihydropyrazine-2-carboxylic acid (36 mg, 0.2 mmol), HATU (88 mg, 0.2 mmol) and DIEA (75 mg, 0.6 mmol) in DMF (2 mL) was stirred at room temperature for 2 hours under nitrogen atmosphere. Upon completion, the resulting solution was purified by Prep-HPLC (conditions: Column: Xselect CSH Prep C18, 30*150 mm 5 μm; Mobile Phase A: Water (0.1% FA), Mobile Phase B: ACN; Flow rate: 60 mL/min; Gradient (B %): 21% to 37% B in 10 min; Wave Length: 254 nm/220 nm; RT1 (min): 11.57) to afford (S)—N-(1-(3,5-difluorobenzyl)-2-(difluoromethyl)-1H-imidazol-4-yl)-2-(3,3-dimethyl-4-(4-methyl-5-oxo-4,5-dihydropyrazine-2-carbonyl)piperazin-1-yl)propanamide (Compound 25, 27.2 mg, 32% ee; Method L, 1.20 min, Peak 1 (major enantiomer); 1.28 min, Peak 2, (minor enantiomer)) as a white solid.

| Compound# | Characterization Data |
|---|---|
| 25 | LCMS (ESI) [M + H]⁺: 564.2 |
| | ¹H NMR (400 MHz, DMSO-d₆) δ 11.46-9.60 (m, 1H), 8.23-8.13 (m, 1H), 7.92 (s, 1H), |
| | 7.60 (bs, 1H), 7.33-7.24 (m, 2H), 7.13-6.88 (m, 2H), 5.40 (s, 2H), 4.21-2.28 (m, 10H), |
| | 1.71-1.02 (m, 9H) |

### Example 26: (S)-4-(4-(1-((1-(3,5-difluorobenzyl)-2-methyl-1H-imidazol-4-yl)amino)-1-oxopropan-2-yl)-2,2-dimethylpiperazine-1-carbonyl)pyridine 1-oxide

### Step 1: 1-(3,5-difluorobenzyl)-2-methyl-4-nitro-1H-imidazole

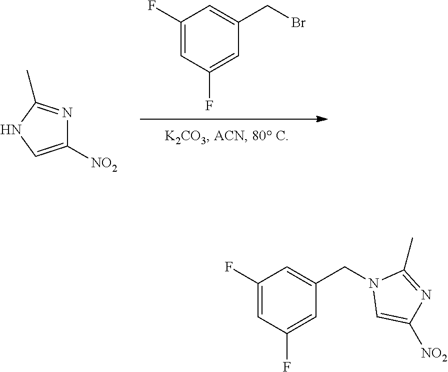

A solution of 2-methyl-4-nitro-1H-imidazole (5.00 g, 39.3 mmol), 1-(bromomethyl)-3,5-difluorobenzene (10.60 g, 51.1 mmol) and K₂CO₃ (10.80 g, 78.7 mmol) in ACN (60 mL) was stirred at 80° C. for 3 hours under nitrogen atmosphere. Upon completion, the mixture was allowed to cool down to room temperature was diluted with water (70 mL). The resulting mixture was extracted with EtOAc (3×80 mL). The combined organic layers were dried over anhydrous Na₂SO₄, filtered and concentrated under reduced pressure. The crude residue was purified by silica gel column chromatography, eluted with PE/EA (1:1) to afford 1-(3,5-difluorobenzyl)-2-methyl-4-nitro-1H-imidazole (5.00 g, 50% yield) as a white solid. LCMS (ESI) [M+H]⁺: 254.

### Step 2: 1-(3,5-difluorobenzyl)-2-methyl-1H-imidazol-4-amine

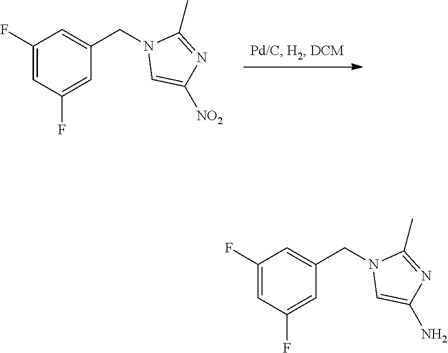 [SMILES: Cc1nc([N+](=O)[O-])cn1Cc1cc(F)cc(F)c1 ; Cc1nc(N)cn1Cc1cc(F)cc(F)c1]

To a mixture of 1-(3,5-difluorobenzyl)-2-methyl-4-nitro-1H-imidazole (4.90 g, 19.4 mmol) in DCM (50 mL) was added 10% Pd/C (490 mg, anhydrous) under nitrogen atmosphere. The resulting mixture was stirred at room temperature for 3 hours under hydrogen atmosphere. Upon completion, the resulting mixture was filtered over celite. The filter cake was washed with DCM (3×30 mL). The filtrate was concentrated under reduced pressure to afford 1-(3,5-difluorobenzyl)-2-methyl-1H-imidazol-4-amine (4.30 g, crude) as a yellow oil, which was used directly in the next step without further purification. LCMS (ESI) [M+H]⁺: 224.

### Step 3: (R)-1-((1-(3,5-difluorobenzyl)-2-methyl-1H-imidazol-4-yl)amino)-1-oxopropan-2-yl acetate

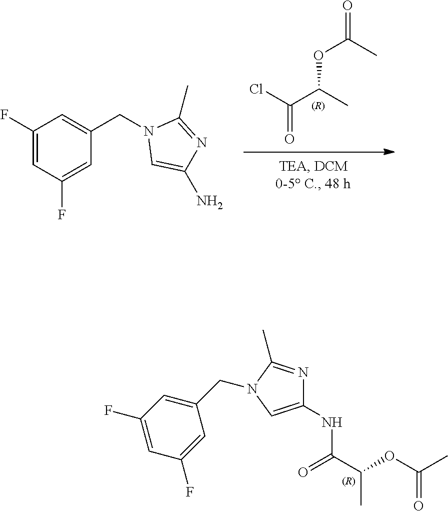

To a mixture of 1-(3,5-difluorobenzyl)-2-methyl-1H-imidazol-4-amine (4.30 g, 19.3 mmol) in DCM (40 mL) were added TEA (5.80 g, 57.8 mmol) and (R)-1-chloro-1-oxopropan-2-yl acetate (4.30 g, 28.9 mmol) dropwise at 0° C. under nitrogen atmosphere. The resulting mixture was stirred at 0° C. for 48 hours. Upon completion, the resulting mixture was concentrated under reduced pressure. The crude residue was directly purified by silica gel column chromatography, eluted with CH₂Cl₂/EA (1:1) to afford (R)-1-((1-(3,5-difluorobenzyl)-2-methyl-1H-imidazol-4-yl)amino)-1-oxopropan-2-yl acetate (1.70 g, 26% yield) as a white solid. LCMS (ESI) [M+H]⁺: 338.

### Step 4: (R)—N-(1-(3,5-difluorobenzyl)-2-methyl-1H-imidazol-4-yl)-2-hydroxypropanamide

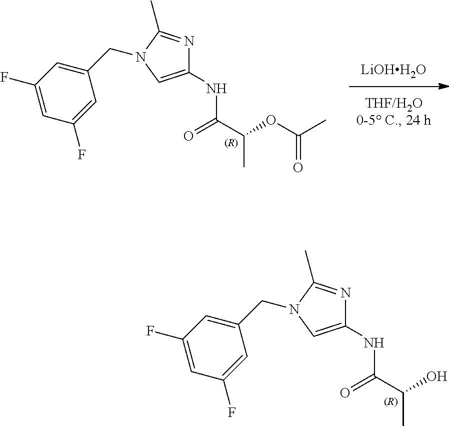

A solution of (R)-1-((1-(3,5-difluorobenzyl)-2-methyl-1H-imidazol-4-yl)amino)-1-oxopropan-2-yl acetate (820 mg, 2.4 mmol) and LiOH·H₂O (204 mg, 4.9 mmol) in THF (5 mL) and H₂O (5 mL) was stirred at 0° C. for 24 hours under nitrogen atmosphere. Upon completion, the mixture was acidified to pH 5 with HCl (1 M). The resulting mixture was concentrated under reduced pressure. The crude residue was purified by reversed-phase flash chromatography (conditions: column: C18 silica gel; mobile phase A: water (0.1% formic acid), Mobile Phase B: MeCN; Gradient (B %): 0% to 100% in 20 min; detector: UV 254 nm and UV 220 nm) to afford (R)—N-(1-(3,5-difluorobenzyl)-2-methyl-1H-imidazol-4-yl)-2-hydroxypropanamide (550 mg, 76% yield) as a white solid. LCMS (ESI) [M+H]⁺: 296.

### Step 5: (R)-1-((1-(3,5-difluorobenzyl)-2-methyl-1H-imidazol-4-yl)amino)-1-oxopropan-2-yl 2-nitrobenzenesulfonate

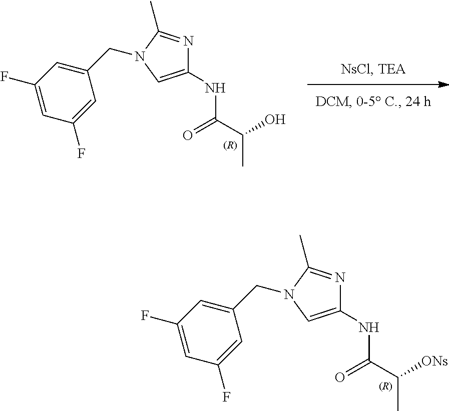

To a mixture of (R)—N-(1-(3,5-difluorobenzyl)-2-methyl-1H-imidazol-4-yl)-2-hydroxypropanamide (550 mg, 1.9 mmol) in DCM (10 mL) were added DMAP (22 mg, 0.2 mmol), TEA (376 mg, 3.7 mmol) and 2-nitrobenzene-1-sulfonyl chloride (619 mg, 2.8 mmol) dropwise at 0° C. under nitrogen atmosphere. The resulting mixture was stirred at 0° C. for 24 hours. Upon completion, the reaction was quenched with water at 0° C. The resulting mixture was extracted with EtOAc (3×12 mL). The combined organic layers were dried over anhydrous Na₂SO₄, filtered and concentrated under reduced pressure. The crude residue was purified by silica gel column chromatography, eluted with CH₂Cl₂/EA (1:1) to afford (R)-1-((1-(3,5-difluorobenzyl)-2-methyl-1H-imidazol-4-yl)amino)-1-oxopropan-2-yl 2-nitrobenzenesulfonate (690 mg, 77% yield) as a white solid. LCMS (ESI) [M+H]⁺: 481.

### Step 6: tert-butyl (S)-4-(1-((1-(3,5-difluorobenzyl)-2-methyl-1H-imidazol-4-yl)amino)-1-oxopropan-2-yl)-2,2-dimethylpiperazine-1-carboxylate

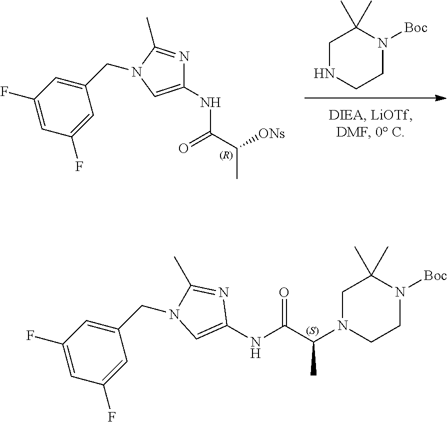

To a mixture of tert-butyl 2,2-dimethylpiperazine-1-carboxylate (448 mg, 2.1 mmol) in DMF (10 mL) were added DIEA (540 mg, 4.2 mmol), lithium trifluoromethanesulfonate (652 mg, 4.2 mmol) and (R)-1-((1-(3,5-difluorobenzyl)-2-methyl-1H-imidazol-4-yl)amino)-1-oxopropan-2-yl 2-nitrobenzenesulfonate (670 mg, 1.4 mmol) dropwise at 0° C. under nitrogen atmosphere. The resulting mixture was stirred at 0° C. overnight. Upon completion, the reaction was directly purified by reversed-phase flash chromatography (conditions: column: C18 silica gel; mobile phase A: water (10 mmol/L NH₄HCO₃), Mobile phase B: MeCN: Gradient (B %): 0% to 100% in 20 min; detector: UV 254 nm) to afford tert-butyl (S)-4-(1-((1-(3,5-difluorobenzyl)-2-methyl-1H-imidazol-4-yl)amino)-1-oxopropan-2-yl)-2,2-dimethylpiperazine-1-carboxylate (526 mg, 76% yield) as a white solid. LCMS (ESI) [M+H]⁺: 492.

### Step 7: (S)—N-(1-(3,5-difluorobenzyl)-2-methyl-1H-imidazol-4-yl)-2-(3,3-dimethylpiperazin-1-yl)propanamide

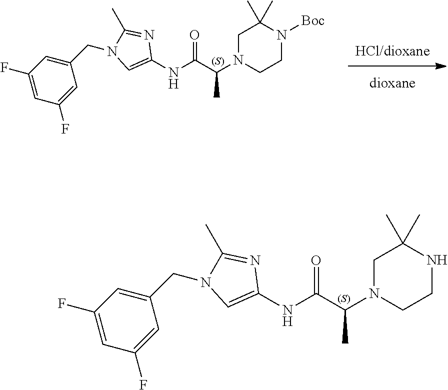

To a mixture of tert-butyl (S)-4-(1-((1-(3,5-difluorobenzyl)-2-methyl-1H-imidazol-4-yl)amino)-1-oxopropan-2-yl)-2,2-dimethylpiperazine-1-carboxylate (520 mg, 1.1 mmol) in 1,4-dioxane (2 mL) was added 4 M HCl in 1,4-dioxane (10 mL) dropwise at 0° C. The resulting mixture was warmed to room temperature and stirred for 2 hours. Upon completion, the resulting mixture was concentrated under reduced pressure to afford (S)—N-(1-(3,5 -difluorobenzyl)-2-methyl-1H-imidazol-4-yl)-2-(3,3-dimethylpiperazin-1-yl)propanamide (400 mg, crude) as a white solid, which was used directly in the next step without further purification. LCMS (ESI) [M+H]⁺: 392

### Step 8: (S)-4-(4-(1-((1-(3,5-difluorobenzyl)-2-methyl-1H-imidazol-4-yl)amino)-1-oxopropan-2-yl)-2,2-dimethylpiperazine-1-carbonyl)pyridine 1-oxide

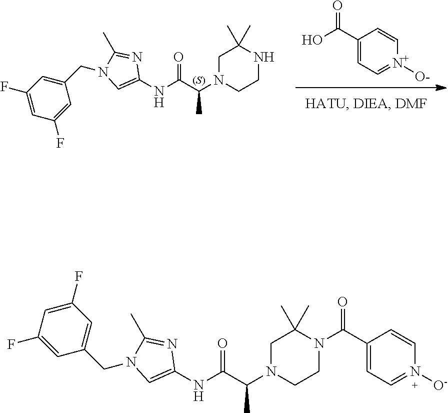

A solution of (S)—N-(1-(3,5-difluorobenzyl)-2-methyl-1H-imidazol-4-yl)-2-(3,3-dimethylpiperazin-1-yl)propanamide (50 mg, 0.1 mmol), 4-carboxypyridine 1-oxide (26 mg, 0.2 mmol), HATU (97 mg, 0.3 mmol) and DIEA (82 mg, 0.6 mmol) in DMF (2.5 mL) was stirred at room temperature for 3 hours under nitrogen atmosphere. Upon completion, the resulting solution was directly purified by Prep-HPLC (conditions: Column: Xselect CSH Prep C18, 30*150 mm 5 μm; Mobile Phase A: Water (0.1% FA), Mobile Phase B: ACN; Flow rate: 60 mL/min; Gradient (B %): 9% to 29% B in 10 minutes; Wave Length: 254 nm/220 nm; RT1 (min): 11.68) to afford (S)-4-(4-(1-((1-(3,5-difluorobenzyl)-2-methyl-1H-imidazol-4-yl)amino)-1-oxopropan-2-yl)-2,2-dimethylpiperazine-1-carbonyl)pyridine 1-oxide (Compound 26, 34.5 mg) as a yellow solid.

| Compound# | Characterization Data |
|---|---|
| 26 | LCMS (ESI) [M + H]⁺: 513.3 |
| | ¹H NMR (400 MHz, DMSO-d₆) δ 8.29 (d, J = 6.6 Hz, 2H), 7.54 (d, J = 6.4 Hz, 2H), |
| | 7.36 (s, 1H), 7.21 (t, J = 2.4 Hz, 1H), 6.98-6.96 (m, 2H), 5.25 (s, 2H), 3.89 (s, 1H), |
| | 3.70-3.55 (m, 2H), 3.29-2.94 (m, 4H), 2.39 (s, 3H), 1.53 (d, J = 4.4 Hz, 6H), 1.45 |
| | (d, J = 6.8 Hz, 3H) |

### Example 27: (S)—N—((R)-5-(3,5-difluorophenyl)-6,7-dihydro-5H-pyrrolo[1,2-a]imidazol-2-yl)-2-(4-(6-(hydroxymethyl)-5-oxo-4,5-dihydropyrazine-2-carbonyl)-3,3-dimethylpiperazin-1-yl)propenamide

### Step 1: methyl 6-bromo-3-methoxypyrazine-2-carboxylate

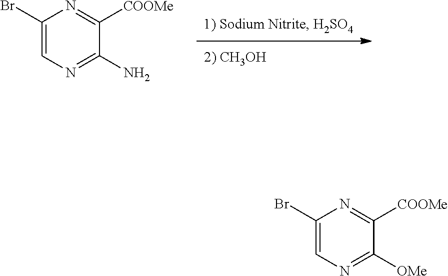 [SMILES: COC(=O)c1nc(Br)cnc1N ; COC(=O)c1nc(Br)cnc1OC]

To a mixture of methyl 3-amino-6-bromopyrazine-2-carboxylate (4.00 g, 17.2 mmol) in H₂SO₄ (40 mL) were added sodium nitrite (2.40 g, 34.8 mmol) in portions at 0° C. under nitrogen atmosphere. The resulting mixture was stirred at 0° C. for 30 minutes. Then, MeOH (80 mL) was added dropwise over 15 min at 0° C. The resulting mixture was stirred at 80° C. for additional 4 hours. Upon completion, the mixture was allowed to cool down to room temperature. The reaction was quenched with water at 0° C. The resulting mixture was extracted with EtOAc (3×200 mL). The combined organic layers were dried over anhydrous Na₂SO₄, filtered and concentrated under reduced pressure. The crude residue was purified by silica gel column chromatography, eluted with PE/EA (3:7) to afford methyl 6-bromo-3-methoxypyrazine-2-carboxylate (3.00 g, 70% yield) as a white solid. LCMS (ESI) [M+H]⁺: 247.

### Step 2: (6-bromo-3-methoxypyrazin-2-yl)methanol

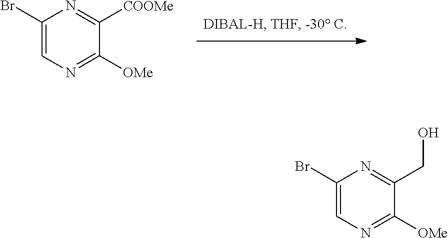 [SMILES: COC(=O)c1nc(Br)cnc1OC ; COc1ncc(Br)nc1CO]

To a mixture of methyl 6-bromo-3-methoxypyrazine-2-carboxylate (3.00 g, 12.1 mmol) in THF (30 mL) was added DIBAL-H (24 mL, 24.2 mmol, 1M in toluene) dropwise at −30° C. under nitrogen atmosphere. The resulting mixture was stirred at −30° C. for 1.5 hours. Upon completion, the reaction was quenched with sat. NH₄Cl (aq.) at 0° C. The resulting mixture was extracted with EtOAc (3×40 mL). The combined organic layers were washed with brine, dried over anhydrous Na₂SO₄, filtered and concentrated under reduced pressure. The crude residue was purified by silica gel column chromatography, eluted with PE/EA (1:1) to afford (6-bromo-3-methoxypyrazin-2-yl)methanol (1.40 g, 52% yield) as a white solid. LCMS (ESI) [M+H]⁺: 219.

### Step 3: methyl 6-(hydroxymethyl)-5-methoxypyrazine-2-carboxylate

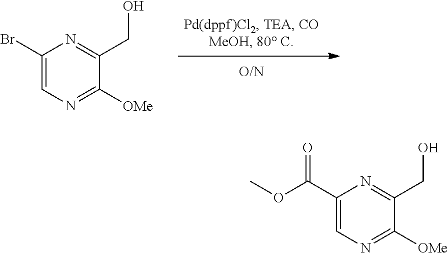

A solution of (6-bromo-3-methoxypyrazin-2-yl)methanol (500 mg, 2.3 mmol), Pd(dppf)Cl₂ (334 mg, 0.5 mmol) and TEA (692 mg, 6.8 mmol) in MeOH (10 mL) was stirred at 80° C. overnight under CO atmosphere (20 atm). Upon completion, the mixture was allowed to cool down to room temperature. The resulting mixture was concentrated under reduced pressure. The crude residue was purified by reversed-phase flash chromatography (conditions: column: C18 silica gel; mobile phase A: water (0.1% formic acid), Mobile phase B: MeCN; Gradient (B %): 0% to 100% in 20 minutes; detector: UV 254 nm) to afford methyl 6-(hydroxymethyl)-5-methoxypyrazine-2-carboxylate (400 mg, 88% yield) as a white solid. LCMS (ESI) [M+H]⁺: 199.

### Step 4: 6-(hydroxymethyl)-5-methoxypyrazine-2-carboxylic acid

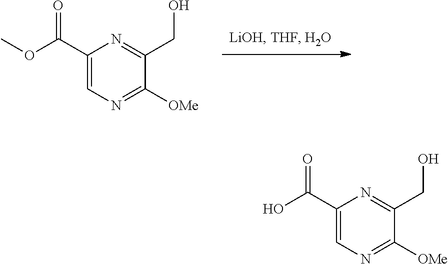

A solution of methyl 6-(hydroxymethyl)-5-methoxypyrazine-2-carboxylate (250 mg, 1.3 mmol) and LiOH·H₂O (151 mg, 6.3 mmol) in THF (4 mL) and H₂O (4 mL) was stirred at room temperature for 1 hour under nitrogen atmosphere. Upon completion, the mixture was acidified to pH 5 with HCl (1 M). The resulting mixture was concentrated under reduced pressure to afford 6-(hydroxymethyl)-5-methoxypyrazine-2-carboxylic acid (210 mg, crude) as a white solid, which was used directly in the next step without further purification. LCMS (ESI) [M+H]⁺: 185

### Step 5: (S)—N—((R)-5-(3,5-difluorophenyl)-6,7-dihydro-5H-pyrrolo[1,2-a]imidazol-2-yl)-2-(4-(6-(hydroxymethyl)-5-methoxypyrazine-2-carbonyl)-3,3-dimethylpiperazin-1-yl)propanamide

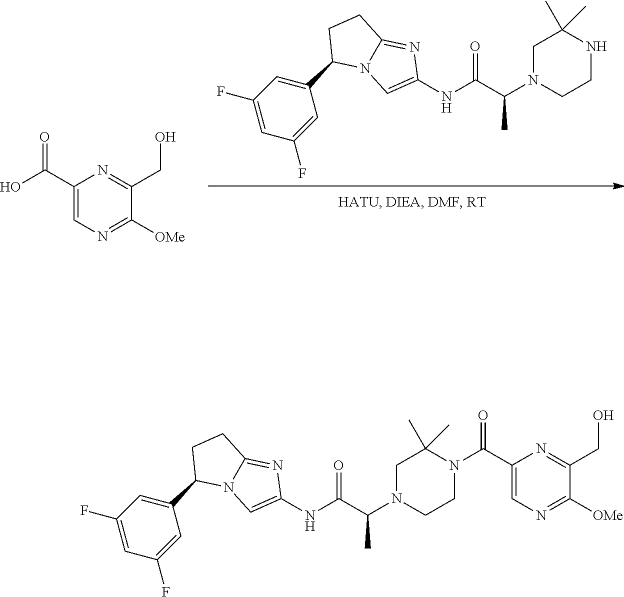 [SMILES: COc1ncc(C(=O)O)nc1CO ; C[C@@H](C(=O)Nc1cn2c(n1)CC[C@@H]2c1cc(F)cc(F)c1)N1CCNC(C)(C)C1 ; COc1ncc(C(=O)N2CCN([C@@H](C)C(=O)Nc3cn4c(n3)CC[C@@H]4c3cc(F)cc(F)c3)CC2(C)C)nc1CO]

A solution of (S)—N—((R)-5-(3,5-difluorophenyl)-6,7-dihydro-5H-pyrrolo[1,2-a]imidazol-2-yl)-2-(3,3-dimethylpiperazin-1-yl)propanamide (150 mg, 0.4 mmol), 6-(hydroxymethyl)-5-methoxypyrazine-2-carboxylic acid (137 mg, 0.7 mmol), HATU (282 mg, 0.7 mmol) and DIEA (240 mg, 1.9 mmol) in DMF (6 mL) was stirred at room temperature for 16 hours under nitrogen atmosphere. Upon completion, the resulting solution was directly purified by reversed-phase flash chromatography (conditions: column: C18 silica gel; mobile phase A: water (0.1% formic acid), Mobile Phase B: MeCN; Gradient (B %): 0% to 100% in 20 min; detector: UV 254 nm) to afford (S)—N—((R)-5-(3,5-difluorophenyl)-6,7-dihydro-5H-pyrrolo[1,2-a]imidazol-2-yl)-2-(4-(6-(hydroxymethyl)-5-methoxypyrazine-2-carbonyl)-3,3-dimethylpiperazin-1-yl)propanamide (95 mg, 44% yield) as a white solid. LCMS (ESI) [M+H]⁺: 570.

### Step 6: (S)—N—((R)-5-(3,5-difluorophenyl)-6,7-dihydro-5H-pyrrolo[1,2-a]imidazol-2-yl)-2-(4-(6-(hydroxymethyl)-5-oxo-4,5-dihydropyrazine-2-carbonyl)-3,3-dimethylpiperazin-1-yl)propanamide

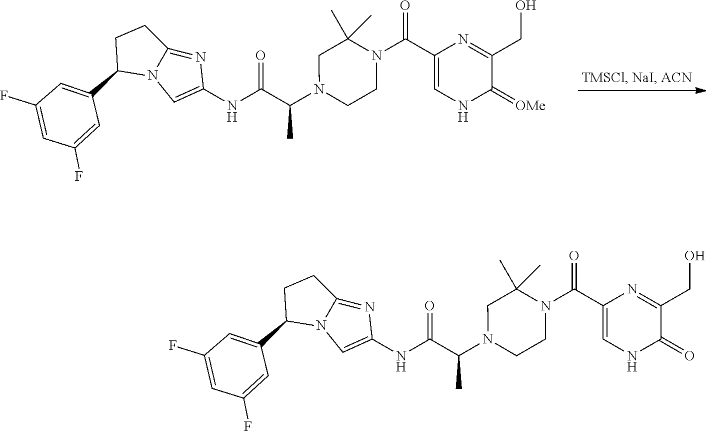

A solution of (S)—N—((R)-5-(3,5-difluorophenyl)-6,7-dihydro-5H-pyrrolo[1,2-a]imidazol-2-yl)-2-(4-(6-(hydroxymethyl)-5-methoxypyrazine-2-carbonyl)-3,3-dimethylpiperazin-1-yl)propanamide (85 mg, 0.2 mmol), TMSCl (102 mg, 0.9 mmol) and NaI (140 mg, 0.9 mmol) in MeCN (3 mL) was stirred at room temperature overnight under nitrogen atmosphere. Upon completion, the resulting mixture was concentrated under reduced pressure. The crude residue was purified by Prep-HPLC (conditions: Column: Xselect CSH Prep C18, 30*150 mm 5 μm; Mobile Phase A: Water (0.1% FA), Mobile Phase B: MeOH; Flow rate: 60 mL/min; Gradient (B %): 30% to 47% B in 9 minutes; Wave Length: 254 nm/220 nm; RT1 (min): 9.38) to afford (S)—N—((R)-5-(3,5-difluorophenyl)-6,7-dihydro-5H-pyrrolo[1,2-a]imidazol-2-yl)-2-(4-(6-(hydroxymethyl)-5-oxo-4,5-dihydropyrazine-2-carbonyl)-3,3-dimethylpiperazin-1-yl)propanamide (Compound 27, 12.1 mg) as a white solid.

| Compound# | Characterization Data |
|---|---|
| 27 | LCMS (ESI) [M + H]⁺: 556.2 |
| | ¹H NMR (400 MHz, DMSO-d₆) δ 12.48 (bs, 1H), 9.90 (s, 1H), 7.63 (s, 1H), 7.24 |
| | (t, J = 4.8 Hz, 1H), 6.99-6.92 (m, 3H), 5.38 (t, J = 6.4 Hz, 1H), 4.83 (bs, 1H), 4.43 |
| | (s, 2H), 3.58-3.46 (m, 2H), 3.40-3.34 (m, 1H), 3.00-2.89 (m, 2H), 2.84-2.76 (m, 1H), |
| | 2.73-2.66 (m, 1H), 2.60-2.53 (m, 1H), 2.43-2.26 (m, 3H), 1.42 (d, J = 2.2 Hz, 6H), |
| | 1.15 (d, J = 6.9 Hz, 3H) |

### Example 28: (S)—N—((R)-5-(3,5-difluorophenyl)-6,7-dihydro-5H-pyrrolo[1,2-a]imidazol-2-yl)-2-(4-isonicotinoyl-3,3-dimethylpiperazin-1-yl)propenamide

### Step 1: (S)—N—((R)-5-(3,5-difluorophenyl)-6,7-dihydro-5H-pyrrolo[1,2-a]imidazol-2-yl)-2-(4-isonicotinoyl-3,3-dimethylpiperazin-1-yl)propanamide

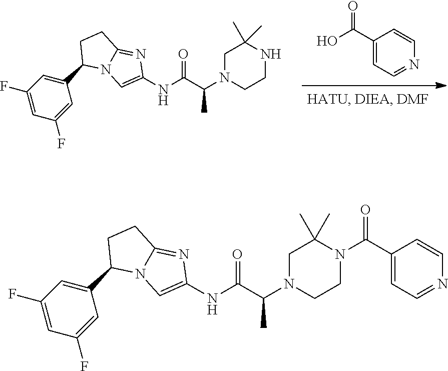

A solution of (S)—N—((R)-5-(3,5-difluorophenyl)-6,7-dihydro-5H-pyrrolo[1,2-a]imidazol-2-yl)-2-(3,3-dimethylpiperazin-1-yl)propanamide (50 mg, 0.1 mmol), isonicotinic acid (22 mg, 0.2 mmol), HATU (94 mg, 0.2 mmol) and DIEA (80 mg, 0.6 mmol) in DMF (2.5 mL) was stirred at room temperature overnight under nitrogen atmosphere. Upon completion, the resulting solution was directly purified by Prep-HPLC (conditions: Column: Xselect CSH Prep C18, 30*150 mm 5 μm; Mobile Phase A: Water (0.1% FA), Mobile Phase B: ACN; Flow rate: 60 mL/min; Gradient (B %): 16% to 34% B in 10 minutes; Wave Length: 254 nm/220 nm; RT1 (min): 11.35) to afford (S)—N—((R)-5-(3,5-difluorophenyl)-6,7-dihydro-5H-pyrrolo[1,2-a]imidazol-2-yl)-2-(4-isonicotinoyl-3,3-dimethylpiperazin-1-yl)propanamide (Compound 28, 21.1 mg) as a yellow solid.

| Compound# | Characterization Data |
|---|---|
| 28 | LCMS (ESI) [M + H]⁺: 509.3 |
| | ¹H NMR (400 MHz, DMSO-d₆) δ 8.69-8.68 (m, 2H), 7.43 (bs, 2H), 7.25 (t, J = 4.6 Hz, |
| | 1H), 7.05 (s, 1H), 7.04-6.92 (m, 2H), 5.45 (bs, 1H), 4.09-2.79 (m, 7H), 2.56-2.30 |
| | (m, 4H), 1.61-1.22 (m, 9H) |

### Example 29: 4-(4-((S)-1-(((R)-5-(2,5-difluorophenyl)-6,7-dihydro-5H-pyrrolo[1,2-a]imidazol-2-yl)amino)-1-oxopropan-2-yl)-2,2-dimethylpiperazine-1-carbonyl)pyridine 1-oxide (29a) and 4-(4-((S)-1-(((S)-5-(2,5-difluorophenyl)-6,7-dihydro-5H-pyrrolo[1,2-a]imidazol-2-yl)amino)-1-oxopropan-2-yl)-2,2-dimethylpiperazine-1-carbonyl)pyridine 1-oxide (29b)

### Step 1: 5-(2,5-difluorophenyl)pyrrolidin-2-one

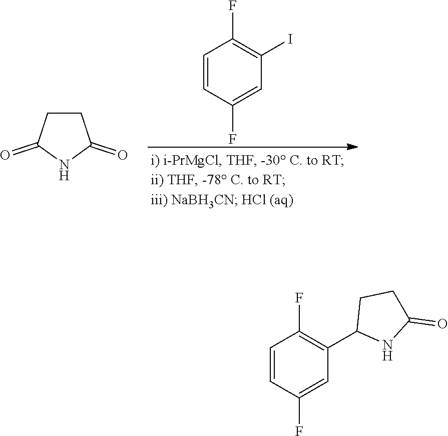 [SMILES: O=C1CCC(=O)N1 ; Fc1ccc(F)c(I)c1 ; O=C1CCC(c2cc(F)ccc2F)N1]

To a mixture of 1,4-difluoro-2-iodobenzene (26.6 g, 111.0 mmol) in THF (250 mL) was added isopropylmagnesium chloride-lithium chloride complex (1.3 M in THF, 85.5 mL) dropwise over 12 minutes at −30° C. under nitrogen atmosphere. The resulting mixture was stirred at room temperature for 1 hour. Then, the reaction was cooled to −78° C. and a solution of succinimide (5.00 g, 50.5 mmol, in 10 mL of THF) was added dropwise over 15 minutes. The resulting mixture was warmed to room temperature and was allowed to stir for an additional 2 hours. Then, the reaction mixture was cooled to 0° C. and NaBH₃CN (3.8 g, 60.6 mmol) was added in portions over 12 minutes. The resulting mixture was stirred at room temperature for an additional 30 minutes. Upon completion, the mixture was acidified to pH 3 with aqueous HCl (1 M) at 0° C. The resulting mixture was stirred at room temperature for 1.5 hours, at which point the mixture was neutralized to pH 7 with ammonium hydroxide. The resulting mixture was extracted with EtOAc (3×300 mL). The combined organic layers were dried over anhydrous Na₂SO₄, filtered and concentrated under reduced pressure. The crude residue was purified by silica gel column chromatography, eluted with CH₂Cl₂/MeOH (10:1) to afford 5-(2,5-difluorophenyl)pyrrolidin-2-one (3.30 g, 33% yield) as a yellow solid. LCMS (ESI) [M+H]⁺: 198.

### Step 2: 2-(2-(2,5-difluorophenyl)-5-oxopyrrolidin-1-yl)acetamide

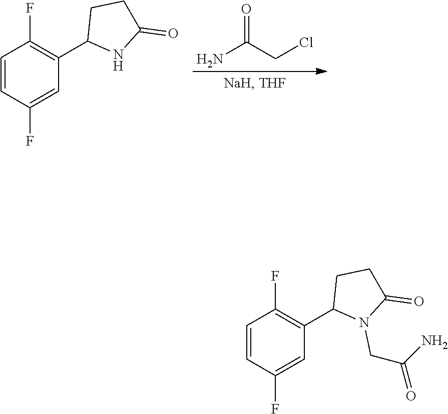 [SMILES: O=C1CCC(c2cc(F)ccc2F)N1 ; NC(=O)CCl ; NC(=O)CN1C(=O)CCC1c1cc(F)ccc1F]

To a mixture of 5-(2,5-difluorophenyl)pyrrolidin-2-one (3.30 g, 16.7 mmol) in THF (35 mL) was added NaH (60% in mineral oil, 1.20 g, 50.2 mmol) in portions at 0° C. under nitrogen atmosphere. The resulting mixture was stirred at room temperature for 30 minutes. Then, the reaction was cooled to 0° C. and 2-chloroacetamide (1.72 g, 18.4 mmol) was added dropwise over 5 minutes. The resulting mixture was stirred at room temperature for additional 3 hours. Upon completion, the reaction was quenched with water at 0° C. The resulting mixture was extracted with EtOAc (3×40 mL). The combined organic layers were dried over anhydrous Na₂SO₄, filtered and concentrated under reduced pressure. The crude residue was purified by silica gel column chromatography, eluted with CH₂Cl₂/MeOH (15:1) to afford 2-(2-(2,5-difluorophenyl)-5-oxopyrrolidin-1-yl)acetamide (2.10 g, 67% yield) as a yellow oil. LCMS (ESI) [M+H]⁺: 255

### Step 3: 2-bromo-5-(2,5-difluorophenyl)-6,7-dihydro-5H-pyrrolo[1,2-a]imidazole

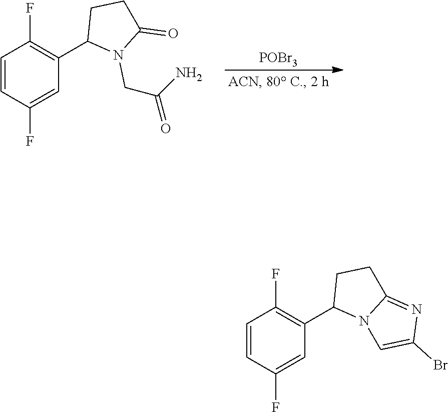 [SMILES: NC(=O)CN1C(=O)CCC1c1cc(F)ccc1F ; "POBr3" ; Fc1ccc(F)c(C2CCc3nc(Br)cn32)c1]

To a mixture of 2-(2-(2,5-difluorophenyl)-5-oxopyrrolidin-1-yl)acetamide (2.00 g, 7.9 mmol) in ACN (40 mL) was added phosphoroyl tribromide (9.00 g, 31.5 mmol) at 0° C. under nitrogen atmosphere. The resulting mixture was stirred at 80° C. for 2 hours. Upon completion, the mixture was allowed to cool down to room temperature and was then neutralized to pH 7 with ammonium hydroxide solution. The resulting mixture was extracted with EtOAc (3×50 mL). The combined organic layers were dried over anhydrous Na₂SO₄, filtered and concentrated under reduced pressure. The crude residue was purified by silica gel column chromatography, eluted with CH₂Cl₂/MeOH (13:1) to afford 2-bromo-5-(2,5-difluorophenyl)-6,7-dihydro-5H-pyrrolo[1,2-a]imidazole (1.80 g, 76% yield) as a yellow solid. LCMS (ESI) [M+H]⁺: 299

### Step 4: (2R)—N-(5-(2,5-difluorophenyl)-6,7-dihydro-5H-pyrrolo[1,2-a]imidazol-2-yl)-2-hydroxypropanamide

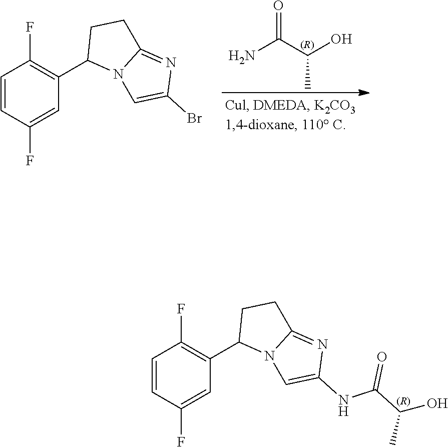

To a mixture of (R)-2-hydroxypropanamide (1.00 g, 12.0 mmol) in 1,4-dioxane (15 mL) were added CuI (229 mg, 1.2 mmol), K₂CO₃ (1.66 g, 12.0 mmol), 1,2-bis(methylamino)ethane (212 mg, 2.4 mmol) and 2-bromo-5-(2,5-difluorophenyl)-6,7-dihydro-5H-pyrrolo[1,2-a]imidazole (1.80 g, 6.0 mmol). The resulting mixture was stirred at 110° C. overnight under nitrogen atmosphere. Upon completion, the mixture was allowed to cool down to room temperature. The reaction was quenched with water at 0° C. The resulting mixture was extracted with EtOAc (3×20 mL). The combined organic layers were dried over anhydrous Na₂SO₄, filtered and concentrated under reduced pressure. The crude residue was purified by silica gel column chromatography, eluted with DCM/MeOH (18:1) to afford (2R)—N-(5-(2,5-difluorophenyl)-6,7-dihydro-5H-pyrrolo[1,2-a]imidazol-2-yl)-2-hydroxypropanamide (1.20 g, 64% yield) as a yellow oil. LCMS (ESI) [M+H]⁺: 308

### Step 5: (2R)-1-((5-(2,5-difluorophenyl)-6,7-dihydro-5H-pyrrolo[1,2-a]imidazol-2-yl)amino)-1-oxopropan-2-yl 2-nitrobenzenesulfonate

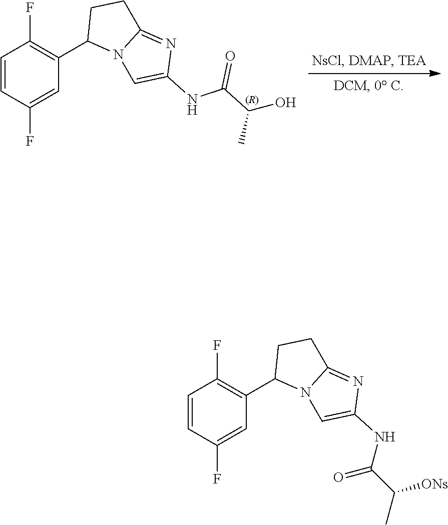

To a mixture of (2R)—N-(5-(2,5-difluorophenyl)-6,7-dihydro-5H-pyrrolo[1,2-a]imidazol-2-yl)-2-hydroxypropanamide (400 mg, 1.3 mmol) in DCM (6 mL) were added DMAP (31 mg, 0.3 mmol), TEA (395 mg, 3.9 mmol) and 2-nitrobenzene-1-sulfonyl chloride (317 mg, 1.4 mmol) in portions at 0° C. under nitrogen atmosphere. The resulting mixture was stirred at 0° C. for 4 hours. Upon completion, the reaction was quenched with water at 0° C. The resulting mixture was extracted with EtOAc (3×8 mL). The combined organic layers were dried over anhydrous Na₂SO₄, filtered and concentrated under reduced pressure. The crude residue was purified by silica gel column chromatography, eluted with DCM/EA (1:1) to afford (2R)-1-((5-(2,5-difluorophenyl)-6,7-dihydro-5H-pyrrolo[1,2-a]imidazol-2-yl)amino)-1-oxopropan-2-yl 2-nitrobenzenesulfonate (160 mg, 25% yield) as a yellow solid. LCMS (ESI) [M+H]⁺: 493

### Step 6: tert-butyl 4-((2S)-1-((5-(2,5-difluorophenyl)-6,7-dihydro-5H-pyrrolo[1,2-a]imidazol-2-yl)amino)-1-oxopropan-2-yl)-2,2-dimethylpiperazine-1-carboxylate

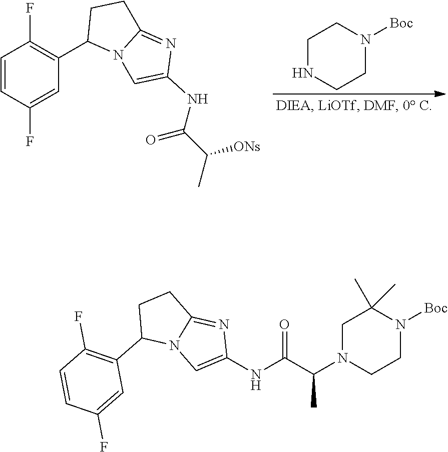

To a mixture of tert-butyl 2,2-dimethylpiperazine-1-carboxylate (164 mg, 0.8 mmol) in DMF (8 mL) were added DIEA (198 mg, 1.5 mmol), lithium trifluoromethanesulfonate (239 mg, 1.5 mmol) and (2R)-1-((5-(2,5-difluorophenyl)-6,7-dihydro-5H-pyrrolo[1,2-a]imidazol-2-yl)amino)-1-oxopropan-2-yl 2-nitrobenzenesulfonate (260 mg, 0.5 mmol) dropwise at 0° C. under nitrogen atmosphere. The resulting mixture was stirred at 0° C. overnight. Upon completion, the reaction mixture was directly purified by reversed-phase flash chromatography (conditions: column: C18 silica gel; mobile phase A: water (10 mmol/L NH₄HCO₃), Mobile phase B: MeCN; Gradient (B %): 0% to 100% in 20 min; detector: UV 254 nm) to afford tert-butyl 4-((2S)-1-((5-(2,5-difluorophenyl)-6,7-dihydro-5H-pyrrolo[1,2-a]imidazol-2-yl)amino)-1-oxopropan-2-yl)-2,2-dimethylpiperazine-1-carboxylate (240 mg, 92% yield) as a white solid. LCMS (ESI) [M+H]⁺: 504

### Step 7: (2S)—N-(5-(2,5-difluorophenyl)-6,7-dihydro-5H-pyrrolo[1,2-a]imidazol-2-yl)-2-(3,3-dimethylpiperazin-1-yl)propanamide

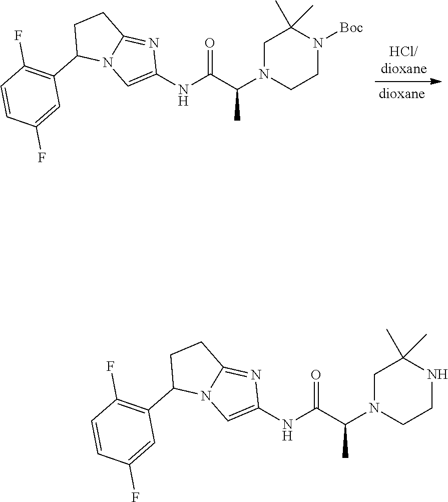 [SMILES: C[C@@H](C(=O)Nc1cn2c(n1)CCC2c1cc(F)ccc1F)N1CCN(C(=O)OC(C)(C)C)C(C)(C)C1 ; C[C@@H](C(=O)Nc1cn2c(n1)CCC2c1cc(F)ccc1F)N1CCNC(C)(C)C1]

To a mixture of tert-butyl 4-((2S)-1-((5-(2,5-difluorophenyl)-6,7-dihydro-5H-pyrrolo[1,2-a]imidazol-2-yl)amino)-1-oxopropan-2-yl)-2,2-dimethylpiperazine-1-carboxylate (230 mg, 0.5 mmol) in 1,4-dioxane (1 mL) was added 4 M HCl in 1,4-dioxane (5 mL) dropwise at 0° C. The resulting mixture was stirred at room temperature for 2 hours. Upon completion, the resulting mixture was concentrated under reduced pressure to afford (2S)—N-(5-(2,5-difluorophenyl)-6,7-dihydro-5H-pyrrolo[1,2-a]imidazol-2-yl)-2-(3,3-dimethylpiperazin-1-yl)propanamide (180 mg, crude) as a white solid, which was used directly in the next step without further purification. LCMS (ESI) [M+H]⁺: 404

### Step 8: 4-(4-((2S)-1-((5-(2,5-difluorophenyl)-6,7-dihydro-5H-pyrrolo[1,2-a]imidazol-2-yl)amino)-1-oxopropan-2-yl)-2,2-dimethylpiperazine-1-carbonyl)pyridine 1-oxide

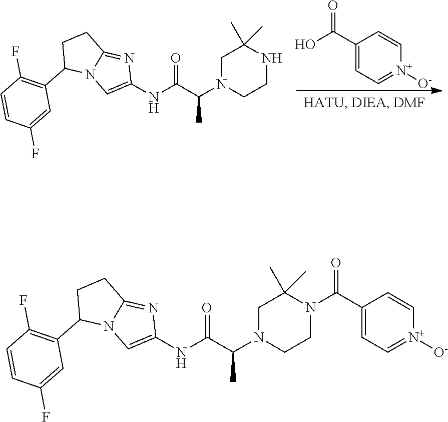 [SMILES: C[C@@H](C(=O)Nc1cn2c(n1)CCC2c1cc(F)ccc1F)N1CCNC(C)(C)C1 ; O=C(O)c1cc[n+]([O-])cc1 ; C[C@@H](C(=O)Nc1cn2c(n1)CCC2c1cc(F)ccc1F)N1CCN(C(=O)c2cc[n+]([O-])cc2)C(C)(C)C1]

A solution of (2S)—N-(5-(2,5-difluorophenyl)-6,7-dihydro-5H-pyrrolo[1,2-a]imidazol-2-yl)-2-(3,3-dimethylpiperazin-1-yl)propanamide (180 mg, 0.4 mmol), 4-carboxypyridine 1-oxide (124 mg, 0.9 mmol), HATU (339 mg, 0.9 mmol) and DIEA (288 mg, 2.2 mmol) in DMF (5 mL) was stirred at room temperature overnight under nitrogen atmosphere. Upon completion, the resulting solution was directly purified by Prep-HPLC (conditions: Column: XBridge Shield RP18 OBD Column 30*150 mm, 5 μm; Mobile Phase A: Water (10 mmol/L NH₄HCO₃), Mobile Phase B: ACN; Flow rate: 60 mL/min; Gradient (B %): 22% B to 37% B in 17 minutes; Wave Length: 254 nm/220 nm; RT1 (min): 11.35) to afford 4-(4-((2S)-1-((5-(2,5-difluorophenyl)-6,7-dihydro-5H-pyrrolo[1,2-a]imidazol-2-yl)amino)-1-oxopropan-2-yl)-2,2-dimethylpiperazine-1-carbonyl)pyridine 1-oxide (80 mg, 34% yield) as a white solid. LCMS (ESI) [M+H]⁺: 525

### Step 9: 4-(4-((S)-1-(((R)-5-(2,5-difluorophenyl)-6,7-dihydro-5H-pyrrolo[1,2-a]imidazol-2-yl)amino)-1-oxopropan-2-yl)-2,2-dimethylpiperazine-1-carbonyl)pyridine 1-oxide and 4-(4-((S)-1-(((S)-5-(2,5-difluorophenyl)-6,7-dihydro-5H-pyrrolo[1,2-a]imidazol-2-yl)amino)-1-oxopropan-2-yl)-2,2-dimethylpiperazine-1-carbonyl)pyridine 1-oxide

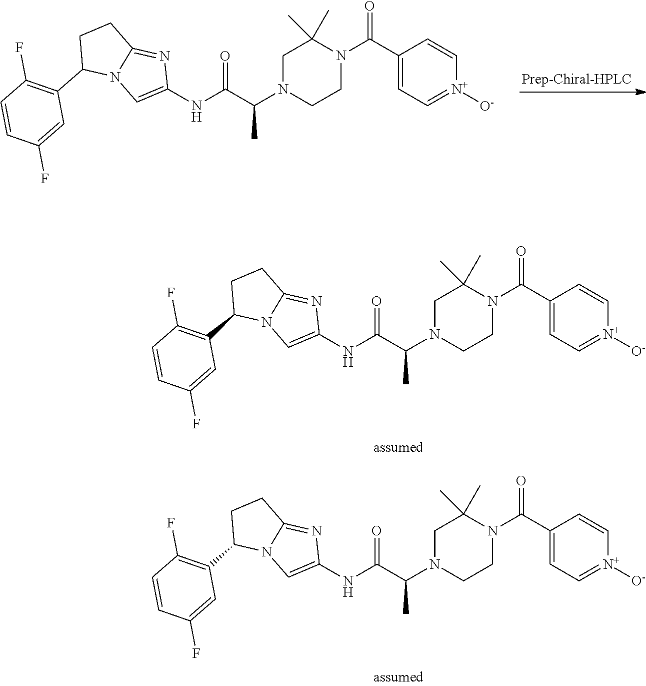 [SMILES: C[C@@H](C(=O)Nc1cn2c(n1)CCC2c1cc(F)ccc1F)N1CCN(C(=O)c2cc[n+]([O-])cc2)C(C)(C)C1 ; C[C@@H](C(=O)Nc1cn2c(n1)CC[C@@H]2c1cc(F)ccc1F)N1CCN(C(=O)c2cc[n+]([O-])cc2)C(C)(C)C1 ; C[C@@H](C(=O)Nc1cn2c(n1)CC[C@H]2c1cc(F)ccc1F)N1CCN(C(=O)c2cc[n+]([O-])cc2)C(C)(C)C1]

4-(4-((2S)-1-((5-(2,5-difluorophenyl)-6,7-dihydro-5H-pyrrolo[1,2-a]imidazol-2-yl)amino)-1-oxopropan-2-yl)-2,2-dimethylpiperazine-1-carbonyl)pyridine 1-oxide (80 mg) was resolved by Prep-Chiral HPLC (conditions: Column: CHIRAL ART Cellulose-SB, 3*25 cm, 5 m; Mobile Phase A: MTBE (0.5% 2M NH₃-MeOH), Mobile Phase B: EtOH; Flow rate: 40 mL/min; Gradient (B %): isocratic 30% B; Wave Length: 218/278 nm; RT1 (min): 6.92; RT2 (min): 11.33) to afford first peak 4-(4-((S)-1-(((R)-5-(2,5-difluorophenyl)-6,7-dihydro-5H-pyrrolo[1,2-a]imidazol-2-yl)amino)-1-oxopropan-2-yl)-2,2-dimethylpiperazine-1-carbonyl)pyridine 1-oxide (Compound 29a, 38.5 mg; Method M, 0.95 min, Peak 1) as a white solid and second peak 4-(4-((S)-1-(((S)-5-(2,5-difluorophenyl)-6,7-dihydro-5H-pyrrolo[1,2-a]imidazol-2-yl)amino)-1-oxopropan-2-yl)-2,2-dimethylpiperazine-1-carbonyl)pyridine 1-oxide (Compound 29b, 35.4 mg; Method M, 1.59 min, Peak 2) as a white solid.

| Compound# | Characterization Data |
|---|---|
| 29a | LCMS (ESI) [M + H]⁺: 525.3 |
| | ¹H NMR (400 MHz, DMSO-d₆) δ 9.96 (s, 1H), 8.20 (d, J = 7.2 Hz, 2H), 7.40 (d, J = 7.2 |
| | Hz, 2H), 7.36-7.33 (m, 1H), 7.32-7.25 (m, 1H), 7.00 (s, 1H), 6.90 (t, J = 7.4 Hz, 1H), |
| | 5.58 (t, J = 4.2 Hz, 1H), 3.38-3.36 (m, 1H), 3.29-3.27 (m, 2H), 3.05-2.86 (m, 2H), |
| | 2.86-2.77 (m, 1H), 2.67-2.56 (m, 1H), 2.55-2.53 (m, 1H), 2.46-2.34 (m, 3H), 1.43 |
| | (d, J = 4.6 Hz, 6H), 1.15 (d, J = 6.8 Hz, 3H) |
| 29b | LCMS (ESI) [M + H]⁺: 525.3 |
| | ¹H NMR (400 MHz, DMSO-d₆) δ 10.02 (s, 1H), 8.22 (d, J = 5.2 Hz, 2H), 7.41 (d, J = 7.2 |
| | Hz, 1H), 7.41-7.35 (m, 2H), 7.33-7.25 (m, 1H), 7.02 (s, 1H), 6.80 (t, J = 5.8 Hz, 1H), |
| | 5.58 (t, J = 4.8 Hz, 1H), 3.41-3.39 (m, 1H), 3.28 (t, J = 5.1 Hz, 2H), 3.06-2.95 (m, 1H), |
| | 2.94-2.77 (m, 2H), 2.66-2.64 (m, 1H), 2.56-2.51 (m, 1H), 2.45-2.34 (m, 3H), 1.43 |
| | (d, J = 4.2 Hz, 6H), 1.15 (d, J = 6.8 Hz, 3H) |

### General Procedure for the Preparation of Compounds 30-82

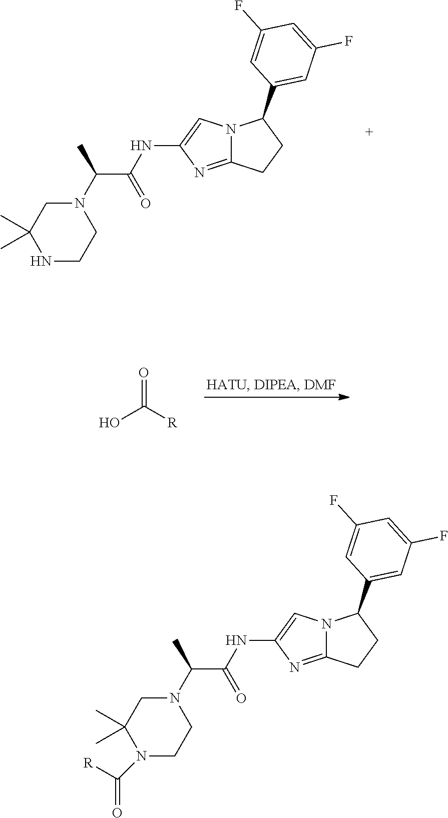

To a reaction vial was added (S)—N—((R)-5-(3,5-difluorophenyl)-6,7-dihydro-5H-pyrrolo[1,2-a]imidazol-2-yl)-2-(3,3-dimethylpiperazin-1-yl)propanamide·2HCl (42 mg, 0.07 mmol, 1.0 equiv.) and a carboxylic acid building block (1.5 equiv.) as shown in Table 2 below, followed by anhydrous DMF (1.0 mL) and DIPEA (5.0 equiv.). Then, HATU (1.5 equiv.) was added to the mixture while stirring. The mixture was stirred at ambient temperature for 10 hours. The progress of the reaction was analyzed by LCMS. Upon completion, the reaction mixture was filtered with a micropore filter and washed with DMF (1 mL). The filtrate was directly purified by prep-HPLC, using conditions shown in Table 2, to afford compounds 30-82. Structures and names of compounds 30-82 are shown in Table 1 and characterization data is shown in Table 2.

TABLE 2
Purification Processes and Characterization data for Compounds 30-82

| Cmp'd | Carboxylic Acid Building | Prep-HPLC Purification | 1H NMR; Mass Spectrum m/z [M + H]⁺ |
|---|---|---|---|
| # | Block Structure and Name | Conditions | (unless otherwise noted) |
| 30 | 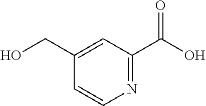 [SMILES: O=C(O)c1cc(CO)ccn1] 4-(hydroxymethyl)picolinic acid | Column: ASA-AZZOTA- C18-SB-10 μm-30*25 mm, Mobile phase: A: 0.1% FA/H2O B: ACN, Gradient (B%): 20% to 50% in 12 min, Flow rate: 35 mL/min, UV wavelength: 220 nm | ¹H NMR (400 MHz, DMSO-d₆) δ 11.52- 9.29 (m, 1H), 8.48 (d, J = 5.0 Hz, 1H), 7.59-7.32 (m, 2H), 7.24 (tt, J = 9.3, 2.4 Hz, 1H), 7.05 (s, 1H), 6.97 (bs, 2H), 5.45- 5.41 (m, 1H), 4.58 (s, 2H), 3.63-3.10 (m, 3H), 3.09-2.77 (m, 4H), 2.55-2.25 (m, 4H), 1.68-1.12 (m, 9H); 539.2 |
| 31 | 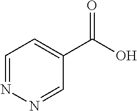 pyridazine-4-carboxylic acid | Column: Phenomenex- Luna-C18-10 μm-25*250 mm, Mobile phase: A: 0.1% FA/H2O B: ACN, Gradient (B%): 28% to 58% in 12 min, Flow rate: 30 mL/min, UV wavelength: 220 nm | ¹H NMR (400 MHz, DMSO-d₆) δ 11.46- 9.59 (m, 1H), 9.51-9.21 (m, 2H), 7.72 (bs, 1H), 7.27 (t, J = 2.4 Hz, 1H), 7.07 (s, 1H), 7.06-6.99 (bs, 2H), 5.46 (bs, 1H), 3.71-3.10 (m, 3H), 3.09-2.74 (m, 4H), 2.61-2.30 (m, 4H), 1.70-1.10 (m, 9H); 510.2 |
| 32 | 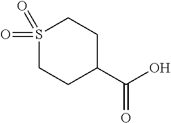 [SMILES: O=C(O)C1CCS(=O)(=O)CC1] tetrahydro-2H-thiopyran-4- carboxylic acid 1,1-dioxide | Column: Phenomenex- Luna-C18-10 μm-25*250 mm, Mobile phase: A: 0.1% FA/H2O B: ACN, Gradient (B%): 21% to 51% in 12 min, Flow rate: 30 mL/min, UV wavelength: 220 nm | ¹H NMR (400 MHz, DMSO-d₆) δ 11.38- 9.41 (m, 1H), 7.25 (tt, J = 9.3, 2.4 Hz, 1H), 7.08 (s, 1H), 7.07-6.90 (bs, 2H), 5.47 (bs, 1H), 4.09-3.30 (m, 6H), 3.29- 2.83 (m, 9H), 2.55-2.35 (m, 1H), 2.10- 1.85 (m, 4H), 1.70-1.30 (m, 9H); 564.2 |
| 33 | 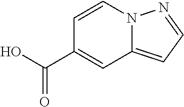 [SMILES: O=C(O)c1ccn2nccc2c1] pyrazolo[1,5-a]pyridine-5- carboxylic acid | Column: ASA-AZZOTA- C18-SB-10 μm-30*25 mm, Mobile phase: A: 0.1% FA/H2O B: ACN, Gradient (B%): 29% to 59% in 12 min, Flow rate: 35 mL/min, UV wavelength: 220 nm | ¹H NMR (400 MHz, DMSO-d₆) δ 9.97 (bs, 1H), 8.69 (d, J = 7.2 Hz, 1H), 8.06 (d, J = 2.3 Hz, 1H), 7.77 (s, 1H), 7.24 (tt, J = 9.3, 2.4 Hz, 1H), 7.01-6.95 (m, 3H), 6.85 (d, J = 7.2 Hz, 1H), 6.73 (d, J = 2.3 Hz, 1H), 5.40 (dd, J = 7.9, 5.3 Hz, 1H), 3.55-3.15 (m, 3H), 3.09-2.70 (m, 4H), 2.60-2.29 (m, 4H), 1.48 (bs, 6H), 1.16 (bs, 3H); 548.2 |
| 34 | 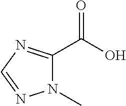 1-methyl-1H-1,2,4-triazole- 5-carboxylic acid | Column: ASA-AZZOTA- C18-SB-10 μm-30*25 mm, Mobile phase: A: 0.1% FA/H2O B: ACN, Gradient (B%): 28% to 58% in 12 min, Flow rate: 35 mL/min, UV wavelength: 220 nm | ¹H NMR (400 MHz, DMSO-d₆) δ 10.06 (bs, 1H), 8.01 (s, 1H), 7.25 (dt, J = 9.4, 2.4 Hz, 1H), 7.03-6.99 (m, 3H), 5.43 (t, J = 6.6 Hz, 1H), 3.87 (s, 3H), 3.60-3.20 (m, 3H), 3.09-2.78 (m, 4H), 2.65-2.31 (m, 4H), 1.48 (bs, 6H), 1.35-1.09 (m, 3H); 513.2 |
| 35 | 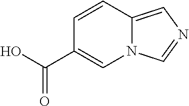 [SMILES: O=C(O)c1ccc2cncn2c1] imidazo[1,5-a]pyridine-6- carboxylic acid | Column: Phenomenex- Luna-C18-10 μm-25*250 mm, Mobile phase: A: 0.1% FA/H2O B: ACN, Gradient (B%): 20% to 50% in 12 min, Flow rate: 30 mL/min, UV wavelength: 220 nm | ¹H NMR (400 MHz, DMSO-d₆) δ 8.78 (bs, 1H), 8.65 (s, 1H), 7.77-7.58 (m, 2H), 7.25 (dd, J = 10.4, 7.9 Hz, 1H), 7.08 (s, 1H), 7.06-6.83 (m, 3H), 5.47 (s, 1H), 4.20-3.11 (m, 3H), 3.10-2.80 (m, 4H), 2.60-2.35 (m, 4H), 1.65-1.35 (m, 9H); 548.2 |
| 36 | 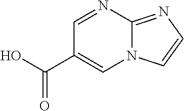 [SMILES: O=C(O)c1cnc2nccn2c1] imidazo[1,2-a]pyrimidine-6- carboxylic acid | Column: Phenomenex- Luna-C18-10 μm-25*250 mm, Mobile phase: A: 0.1% FA/H2O B: ACN, Gradient (B%): 19% to 49% in 12 min, Flow rate: 30 mL/min, UV wavelength: 220 nm | ¹H NMR (400 MHz, DMSO-d₆) δ 9.33 (d, J = 2.3 Hz, 1H), 8.83 (s, 1H), 8.28- 7.94 (m, 2H), 7.25 (tt, J = 9.4, 2.4 Hz, 1H), 7.09 (s, 1H), 6.99 (bs, 2H), 5.47 (bs, 1H), 3.53 (bs, 3H), 3.45-2.89 (m, 4H), 2.60-2.35 (m, 4H), 1.65-1.30 (m, 9H); 549.2 |
| 37 | 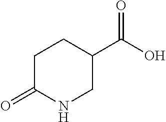 6-oxopiperidine-3- carboxylic acid | Column: ASA-AZZOTA- C18-SB-10 μm-30*25 mm, Mobile phase: A: 0.1% FA/H2O B: ACN, Gradient (B%): 22% to 52% in 12 min, Flow rate: 35 mL/min, UV wavelength: 220 nm | ¹H NMR (400 MHz, DMSO-d₆) δ 11.30- 9.20 (m, 1H), 7.45 (bs, 1H), 7.31-7.26 (m, 1H), 7.06 (s, 1H), 6.96 (bs, 2H), 5.46 (bs, 1H), 4.11-3.80 (m, 2H), 3.79-2.77 (m, 9H), 2.65-2.10 (m, 5H), 1.88-1.65 (m, 2H), 1.64-1.20 (m, 9H); 529.2 |
| 38 | 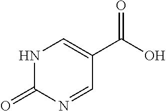 2-oxo-1,2- dihydropyrimidine-5- carboxylic acid | Column: ASA-AZZOTA- C18-SB-10 μm-30*25 mm, Mobile phase: A: 0.1% FA/H2O B: ACN, Gradient (B%): 21% to 51% in 12 min, Flow rate: 35 mL/min, UV wavelength: 220 nm | ¹H NMR (400 MHz, DMSO-d₆) δ 8.50 (bs, 2H), 7.25 (t, J = 9.4 Hz, 1H), 7.06 (s, 1H), 7.05-6.88 (m, 2H), 5.58-5.32 (m, 1H), 4.12-3.91-2.77 (m, 7H), 2.58- 2.20 (m, 4H), 1.65-1.10 (m, 9H); 526.2 |
| 39 | 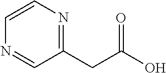 2-(pyrazin-2-yl)acetic acid | Column: ASA-AZZOTA- C18-SB-10 μm-30*25 mm, Mobile phase: A: 0.1% FA/H2O B: ACN, Gradient (B%): 24% to 54% in 12 min, Flow rate: 35 mL/min, UV wavelength: 220 nm | ¹H NMR (400 MHz, DMSO-d₆) δ 9.65 (bs, 1H), 8.73-8.39 (m, 3H), 7.30-7.21 (m, 1H), 7.08-6.90 (m, 3H), 5.52-5.39 (m, 1H), 3.92 (s, 2H), 3.65-3.15 (m, 3H), 3.14-2.72 (m, 4H), 2.70-2.25 (m, 4H), 1.37 (bs, 6H), 1.21-1.10 (m, 3H); 524.2 |
| 40 | 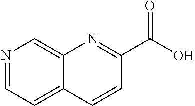 [SMILES: O=C(O)c1ccc2ccncc2n1] 1,7-naphthyridine-2- carboxylic acid | Column: ASA-AZZOTA- C18-SB-10 μm-30*25 mm, Mobile phase: A: 0.1% FA/H2O B: ACN, Gradient (B%): 33% to 63% in 12 min, Flow rate: 35 mL/min, UV wavelength: 220 nm | ¹H NMR (400 MHz, DMSO-d₆) δ 10.02 (bs, 1H), 9.41 (s, 1H), 8.67 (d, J = 5.5 Hz, 1H), 8.55 (d, J = 8.6 Hz, 1H), 7.97 (d, J = 5.6 Hz, 1H), 7.95-7.81 (m, 1H), 7.23 (tt, J = 9.3, 2.4 Hz, 1H), 7.06-6.87 (m, 3H), 5.41 (dd, J = 7.8, 5.3 Hz, 1H), 3.60-3.10 (m, 3H), 3.09-2.75 (m, 4H), 2.70-2.31 (m, 4H), 1.53 (bs, 6H), 1.20 (bs, 3H); 560.2 |
| 41 | 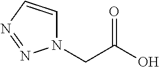 2-(1H-1,2,3-triazol-1- yl)acetic acid | Column: Phenomenex- Luna-C18-10 μm-25*250 mm, Mobile phase: A: 0.1% FA/H2O B: ACN, Gradient (B%): 21% to 51% in 12 min Flow rate: 30 mL/min, UV wavelength: 220 nm | ¹H NMR (400 MHz, DMSO-d₆) δ 7.96 (d, J = 1.0 Hz, 1H), 7.71 (s, 1H), 7.24 (tt, J = 9.4, 2.4 Hz, 1H), 7.04 (s, 1H), 7.03- 6.88 (m, 2H), 5.55-5.26 (m, 3H), 3.65- 3.16 (m, 3H), 3.12-2.80 (m, 4H), 2.60- 2.30 (m, 4H), 1.62-1.10 (m, 9H); 513.2 |
| 42 | 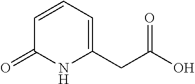 [SMILES: O=C(O)Cc1cccc(=O)[nH]1] 2-(6-oxo-1,6- dihydropyridin-2-yl)acetic acid | Column: Phenomenex- Luna-C18-10 μm-25*250 mm, Mobile phase: A: 0.1% FA/H2O B: ACN, Gradient (B%): 19% to 49% in 12 min, Flow rate: 30 mL/min, UV wavelength: 220 nm | ¹H NMR (400 MHz, DMSO-d₆) δ 11.36 (bs, 1H), 9.93 (s, 1H), 7.32 (dd, J = 9.2, 6.7 Hz, 1H), 7.26-7.18 (m, 1H), 7.05- 6.90 (m, 3H), 6.15 (d, J = 9.1 Hz, 1H), 5.94 (d, J = 6.7 Hz, 1H), 5.39 (dd, J = 7.8, 5.3 Hz, 1H), 3.54 (s, 2H), 3.45- 3.33 (m, 3H), 3.11-2.77 (m, 4H), 2.76- 2.21 (m, 4H), 1.37 (d, J = 4.4 Hz, 6H), 1.14 (d, J = 6.9 Hz, 3H); 539.2 |
| 43 | 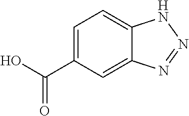 [SMILES: O=C(O)c1ccc2[nH]nnc2c1] 1H-benzo[d][1,2,3]triazole- 5-carboxylic acid | Column: ASA-AZZOTA- C18-SB-10 μm-30*25 mm, Mobile phase: A: 0.1% FA/H2O B: ACN, Gradient (B%): 28% to 58% in 12 min, Flow rate: 35 mL/min, UV wavelength: 220 nm | ¹H NMR (400 MHz, DMSO-d₆) δ 8.34- 7.88 (m, 2H), 7.66-7.48 (m, 1H), 7.29- 6.93 (m, 4H), 5.56-5.16 (m, 1H), 4.10- 2.79 (m, 7H), 2.60-2.31 (m, 4H), 1.80- 1.10 (m, 9H); 549.2 |
| 44 | 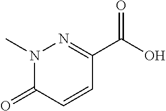 1-methyl-6-oxo-1,6- dihydropyridazine-3- carboxylic acid | Column: Phenomenex- Luna-C18-10 μm-25*250 mm, Mobile phase: A: 0.1% FA/H2O B: ACN, Gradient (B%): 29% to 59% in 12 min, Flow rate: 30 mL/min, UV wavelength: 220 nm | ¹H NMR (400 MHz, DMSO-d₆) δ 11.50- 9.46 (m, 1H), 7.54 (d, J = 9.6 Hz, 1H), 7.24 (tt, J = 9.4, 2.4 Hz, 1H), 7.04-6.87 (m, 4H), 5.44 (dt, J = 8.1, 3.2 Hz, 1H), 3.64 (s, 3H), 3.63-3.10 (m, 3H), 3.09- 2.79 (m, 4H), 2.60-2.30 (m, 4H), 1.70- 1.05 (m, 9H); 540.2 |
| 45 | 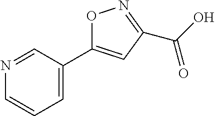 [SMILES: O=C(O)c1cc(-c2cccnc2)on1] 5-(pyridin-3-yl)isoxazole-3- carboxylic acid | Column: Phenomenex- Luna-C18-10 μm-25*250 mm, Mobile phase: A: 0.1% FA/H2O B: ACN, Gradient (B%): 31% to 61% in 12 min, Flow rate: 30 mL/min, UV wavelength: 220 nm | ¹H NMR (400 MHz, DMSO-d₆) δ 10.04 (bs, 1H), 9.12 (d, J = 2.3 Hz, 1H), 8.71 (dd, J = 4.8, 1.7 Hz, 1H), 8.29 (dt, J = 8.1, 2.0 Hz, 1H), 7.67-7.51 (m, 1H), 7.38 (s, 1H), 7.23 (tt, J = 9.3, 2.4 Hz, 1H), 7.03-6.92 (m, 3H), 5.41 (t, J = 6.5 Hz, 1H), 3.60-3.20 (m, 3H), 3.08-2.70 (m, 4H), 2.60-2.31 (m, 4H), 1.50 (bs, 6H), 1.17 (bs, 3H); 576.2 |
| 46 | 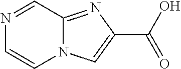 [SMILES: O=C(O)c1cn2ccncc2n1] imidazo[1,2-a]pyrazine-2- carboxylic acid | Column: ASA-AZZOTA- C18-SB-10 μm-30*25 mm, Mobile phase: A: 0.1% FA/H2O B: ACN, Gradient (B%): 24% to 54% in 12 min, Flow rate: 35 mL/min, UV wavelength: 220 nm | ¹H NMR (400 MHz, DMSO-d₆) δ 9.99 (bs, 1H), 9.10 (d, J = 1.6 Hz, 1H), 8.58 (d, J = 4.7 Hz, 1H), 8.38 (bs, 1H), 7.94 (d, J = 4.7 Hz, 1H), 7.35-7.14 (m, 1H), 7.09-6.83 (m, 3H), 5.41 (t, J = 6.6 Hz, 1H), 3.90-3.70 (m, 2H), 3.48-3.16 (m, 1H), 3.10-2.35 (m, 8H), 1.48 (bs, 6H), 1.20 (bs, 3H); 549.2 |
| 47 | 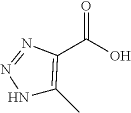 5-methyl-1H-1,2,3-triazole- 4-carboxylic acid | Column: ASA-AZZOTA- C18-SB-10 μm-30*25 mm, Mobile phase: A: 0.1% FA/H2O B: ACN, Gradient (B%): 24% to 54% in 12 min, Flow rate: 35 mL/min, UV wavelength: 220 nm | ¹H NMR (400 MHz, DMSO-d₆) δ 7.35- 7.13 (m, 1H), 7.10-6.81 (m, 3H), 5.43- 5.40 (m, 1H), 4.20-3.10 (m, 3H), 3.09- 2.67 (m, 4H), 2.55-2.25 (m, 7H), 1.47 (bs, 6H), 1.16 (bs, 3H); 513.2 |
| 48 | 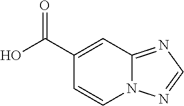 [SMILES: O=C(O)c1ccn2ncnc2c1] [1,2,4]triazolo[1,5- a]pyridine-7-carboxylic acid | Column: Phenomenex- Luna-C18-10 μm-25*250 mm, Mobile phase: A: 0.1% FA/H2O B: ACN, Gradient (B%): 23% to 53% in 12 min, Flow rate: 30 mL/min, UV wavelength: 220 nm | 1H NMR (400 MHz, DMSO-d₆) δ 9.02 (bs, 1H), 8.60 (s, 1H), 8.04-7.88 (m, 1H), 7.40-7.21 (m, 2H), 7.05-6.89 (m, 3H), 5.44 (dd, J = 8.3, 4.9 Hz, 1H), 4.20- 3.10 (m, 3H), 3.09-2.77 (m, 4H), 2.68- 2.32 (m, 4H), 1.78-1.08 (m, 9H); 549.2 |
| 49 | 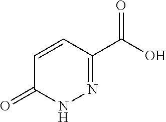 6-oxo-1,6- dihydropyridazine-3- carboxylic acid | Column: ASA-AZZOTA- C18-SB-10 μm-30*25 mm, Mobile phase: A: 0.1% FA/H2O B: ACN, Gradient (B%): 24% to 54% in 12 min, Flow rate: 35 mL/min, UV wavelength: 220 nm | ¹H NMR (400 MHz, DMSO-d₆) δ 13.16 (bs, 1H), 7.52 (d, J = 9.9 Hz, 1H), 7.24 (tt, J = 9.3, 2.4 Hz, 1H), 7.16-6.72 (m, 4H), 5.49-5.41 (m, 1H), 3.70-3.05 (m, 3H), 3.04-2.80 (m, 4H), 2.60-2.30 (m, 4H), 1.60-1.09 (m, 9H); 526.2 |
| 50 | 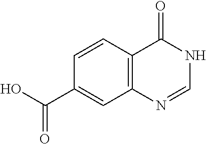 [SMILES: O=C(O)c1ccc2c(=O)[nH]cnc2c1] 4-oxo-3,4- dihydroquinazoline-7- carboxylic acid | Column: ASA-AZZOTA- C18-SB-10 μm-30*25 mm, Mobile phase: A: 0.1% FA/H2O B: ACN, Gradient (B%): 26% to 56% in 12 min, Flow rate: 35 mL/min, UV wavelength: 220 nm | ¹H NMR (400 MHz, DMSO-d₆) δ 12.39 (bs, 1H), 8.22-8.12 (m, 2H), 7.86-7.52 (m, 2H), 7.25 (t, J = 9.5 Hz, 1H), 7.06 (s, 1H), 7.05-6.95 (m, 2H), 5.49-5.41 (m, 1H), 3.71-3.10 (m, 3H), 3.09-2.80 (m, 4H), 2.56-2.35 (m, 4H), 1.70-1.08 (m, 9H); 576.2 |
| 51 | 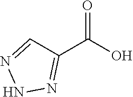 2H-1,2,3-triazole-4- carboxylic acid | Column: Phenomenex- Luna-C18-10 μm-25*250 mm, Mobile phase: A: 0.1% FA/H2O B: ACN, Gradient (B%): 19% to 49% in 12 min, Flow rate: 30 mL/min, UV wavelength: 220 nm | ¹H NMR (400 MHz, DMSO-d₆) δ 15.86- 15.09 (m, 1H), 10.11-9.82 (m, 1H), 8.32-8.02 (m, 1H), 7.24 (tt, J = 9.3, 2.4 Hz, 1H), 7.04 (s, 1H), 7.03-6.88 (m, 2H), 5.44 (dd, J = 8.2, 4.9 Hz, 1H), 3.90- 3.10 (m, 3H), 3.09-2.80 (m, 4H), 2.65- 2.32 (m, 4H), 1.72-1.09 (m, 9H); 499.2 |
| 52 | 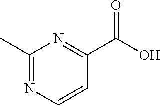 5-methylpyrimidine-4- carboxylic acid | Column: Phenomenex- Luna-C18-10 μm-25*250 mm, Mobile phase: A: 0.1% FA/H2O B: ACN, Gradient (B%): 25% to 55% in 12 min, Flow rate: 30 mL/min, UV wavelength: 220 nm | ¹H NMR (400 MHz, DMSO-d₆) δ 10.10 (bs, 1H), 9.00 (s, 1H), 8.76 (s, 1H), 7.24 (td, J = 9.3, 4.7 Hz, 1H), 7.10-6.87 (m, 3H), 5.51-5.33 (m, 1H), 3.63-2.80 (m, 7H), 2.70-2.30 (m, 4H), 2.15 (s, 3H), 1.56 (bs, 6H), 1.17 (bs, 3H); 524.2 |
| 53 | 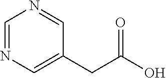 2-(pyrimidin-5-yl)acetic acid | Column: Phenomenex- Luna-C18-10 μm-25*250 mm, Mobile phase: A: 0.1% FA/H2O B: ACN, Gradient (B%): 24% to 54% 12 min, Flow rate: 30 mL/min, UV wavelength: 220 nm | ¹H NMR (400 MHz, DMSO-d₆) δ 9.05 (s, 1H), 8.59 (s, 2H), 7.25 (tt, J = 9.3, 2.4 Hz, 1H), 7.08 (s, 1H), 7.07-6.87 (m, 2H), 5.49-5.41 (m, 1H), 3.82 (s, 2H), 3.81-2.80 (m, 7H), 2.55-2.35 (m, 4H), 1.65-1.38 (m, 9H); 524.2 |
| 54 | 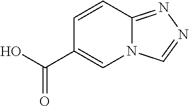 [SMILES: O=C(O)c1ccc2nncn2c1] [1,2,4]triazolo[4,3- a]pyridine-6-carboxylic acid | Column: ASA-AZZOTA- C18-SB-10 μm-30*25 mm, Mobile phase: A: 0.1% FA/H2O B: ACN, Gradient (B%): 29% to 59% in 12 min, Flow rate: 35 mL/min, UV wavelength: 220 nm | 1H NMR (400 MHz, Methanol-d4) δ 9.21 (s, 1H), 8.74 (s, 1H), 7.80 (d, J = 9.5 Hz, 1H), 7.54 (dd, J = 9.5, 1.5 Hz, 1H), 7.11 (s, 1H), 6.97 (tt, J = 9.0, 2.3 Hz, 1H), 6.84-6.92 (m, 2H), 5.51 (dd, J = 7.5, 6.1 Hz, 1H), 3.72-3.52 (m, 3H), 3.20-3.02 (m, 4H), 2.92 (bs, 1H), 2.77 (bs, 2H), 2.62-2.29 (m, 1H), 1.62 (d, J = 2.0 Hz, 6H), 1.43 (d, J = 6.8 Hz, 3H); 549.2 |
| 55 | 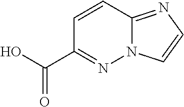 [SMILES: O=C(O)c1ccc2nccn2n1] imidazo[1,2-b]pyridazine-6- carboxylic acid | Column: Phenomenex- Luna-C18-10 μm-25*250 mm, Mobile phase: A: 0.1% FA/H2O B: ACN, Gradient (B%): 23% to 53% in 12 min, Flow rate: 30 mL/min, UV wavelength: 220 nm | ¹H NMR (400 MHz, DMSO-d₆) δ 8.35 (s, 1H), 8.25 (d, J = 9.4 Hz, 1H), 7.91 (d, J = 1.3 Hz, 1H), 7.34 (d, J = 9.4 Hz, 1H), 7.25 (tt, J = 9.4, 2.4 Hz, 1H), 7.05 (s, 1H), 7.04-6.88 (m, 2H), 5.48-5.40 (m, 1H), 3.80-3.10 (m, 3H), 3.09-2.81 (m, 4H), 2.55-2.35 (m, 4H), 1.70-1.10 (m, 9H); 549.2 |
| 56 | 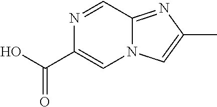 2-methylimidazo[1,2- a]pyrazine-6-carboxylic acid | Column: Phenomenex- Luna-C18-10 μm-25*250 mm, Mobile phase: A: 0.1% FA/H2O B: ACN, Gradient (B%): 25% to 55% in 12 min, Flow rate: 30 mL/min, UV wavelength: 220 nm | ¹H NMR (400 MHz, DMSO-d₆) δ 11.38- 9.88 (m, 1H), 8.99-8.87 (m, 2H), 7.99 (s, 1H), 7.30-7.20 (m, 1H), 7.07 (s, 1H), 7.02-6.92 (m, 2H), 5.48-5.40 (m, 1H), 4.21-2.77 (m, 7H), 2.55-2.45 (m, 7H), 1.68-1.10 (m, 9H); 563.2 |
| 57 | 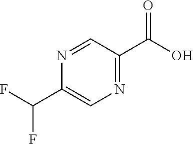 [SMILES: O=C(O)c1cnc(C(F)F)cn1] 5-(difluoromethyl)pyrazine- 2-carboxylic acid | Column: Phenomenex- Luna-C18-10 μm-25*250 mm, Mobile phase: A: 0.1% FA/H2O B: ACN, Gradient (B%): 38% to 68% in 12 min, Flow rate: 30 mL/min, UV wavelength: 220 nm | ¹H NMR (400 MHz, DMSO-d₆) δ 10.03 (s, 1H), 8.99-8.86 (m, 2H), 7.33-7.13 (m, 2H), 7.05-6.89 (m, 3H), 5.48-5.36 (m, 1H), 3.55-3.12 (m, 3H), 3.11-2.31 (m, 8H), 1.4 (bs, 6H), 1.15 (s, 3H); 560.2 |
| 58 | 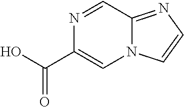 [SMILES: O=C(O)c1cn2ccnc2cn1] imidazo[1,2-a]pyrazine-6- carboxylic acid | Column: Phenomenex- Luna-C18-10 μm-25*250 mm, Mobile phase: A: 0.1% FA/H2O B: ACN, Gradient (B%): 20% to 50% in 12 min, Flow rate: 30 mL/min, UV wavelength: 220 nm | ¹H NMR (400 MHz, DMSO-d₆) δ 10.02 (bs, 1H), 9.03 (s, 1H), 8.93 (bs, 1H), 8.20 (s, 1H), 7.89 (s, 1H), 7.24 (tt, J = 9.6, 2.5 Hz, 1H), 7.14-6.81 (m, 3H), 5.42 (dd, J = 7.7, 5.2 Hz, 1H), 3.60-3.10 (m, 3H), 3.09-2.35 (m, 8H), 1.50 (bs, 6H), 1.17 (bs, 3H); 549.2 |
| 59 | 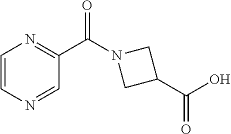 [SMILES: O=C(O)C1CN(C(=O)c2cnccn2)C1] 1-(pyrazine-2- carbonyl)azetidine-3- carboxylic acid | Column: Phenomenex- Luna-C18-10 μm-25*250 mm, Mobile phase: A: 0.1% FA/H2O B: ACN, Gradient (B%): 23% to 53% in 12 min, Flow rate: 30 mL/min, UV wavelength: 220 nm | ¹H NMR (400 MHz, Methanol-d₄): δ 9.16 (s, 1H), 8.72 (d, J = 2.4 Hz, 1H), 8.66 (d, J = 0.8 Hz, 1H), 7.10 (s, 1H), 6.99-6.94 (m, 1H), 6.89-6.83 (m, 2H), 5.52-5.46 (m, 1H), 4.98-4.77 (m, 2H), 4.40-4.28 (m, 2H), 3.87-3.78 (m, 1H), 3.65-3.39 (m, 3H), 3.17-2.90 (m, 4H), 2.89-2.76 (m, 1H), 2.62 (bs, 2H), 2.54- 2.42 (m, 1H), 1.54 (d, J = 3.6 Hz, 6H), 1.39 (d, J = 6.8 Hz, 3H); 593.2 |
| 60 | 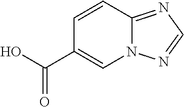 [SMILES: O=C(O)c1ccc2ncnn2c1] [1,2,4]triazolo[1,5- a]pyridine-6-carboxylic acid | Column: ASA-AZZOTA- C18-SB-10 μm-30*25 mm, Mobile phase: A: 0.1% FA/H2O B: ACN, Gradient (B%): 28% to 58% in 12 min, Flow rate: 35 mL/min, UV wavelength: 220 nm | ¹H NMR (400 MHz, Methanol-d₄): δ 8.99 (s, 1H), 8.49 (s, 1H), 7.84 (dd, J = 9.2, 0.8 Hz, 1H), 7.76 (dd, J = 9.2, 1.6 Hz, 1H), 7.09 (s, 1H), 6.99-6.87 (m, 1H), 6.86-6.80 (m, 2H), 5.46 (dd, J = 7.6, 5.6 Hz, 1H), 3.69-3.37 (m, 3H), 3.16-2.37 (m, 8H), 1.62 (d, J = 2.4 Hz, 6H), 1.36 (d, J = 7.2 Hz, 3H); 549.2 |
| 61 | 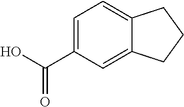 [SMILES: O=C(O)c1ccc2c(c1)CCC2] 1H-benzo[d]imidazole-5- carboxylic acid | Column: Phenomenex- Luna-C18-10 μm-25*250 mm, Mobile phase: A: 0.1% FA/H2O B: ACN, Gradient (B%): 20% to 50% in 12 min, Flow rate: 30 mL/min, UV wavelength: 220 nm | ¹H NMR (400 MHz, DMSO-d₆) δ 12.66- 12.52 (m, 1H), 9.92 (bs, 1H), 8.30 (s, 1H), 7.81-7.53 (m, 2H), 7.31-7.19 (m, 2H), 7.02-6.90 (m, 3H), 5.39 (dd, J = 7.8, 5.3 Hz, 1H), 3.41-3.25 (m, 3H), 3.05-2.65 (m, 4H), 2.55-2.32 (m, 4H), 1.47 (d, J = 3.2 Hz, 6H), 1.16 (d, J = 7.2 Hz, 3H); 548.2 |
| 62 | 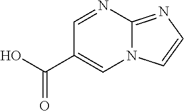 [SMILES: O=C(O)c1cnc2nccn2c1] imidazo[1,2-a]pyridine-6- carboxylic acid | Column: Phenomenex- Luna-C18-10 μm-25*250 mm, Mobile phase: A: 0.1% FA/H2O B: ACN, Gradient (B%): 15% to 45% in 12 min, Flow rate: 30 mL/min, UV wavelength: 220 nm | ¹H NMR (400 MHz, DMSO-d₆) δ 10.18 (bs, 1H), 9.03 (s, 1H), 8.25 (s, 1H), 8.13 (d, J = 6.2 Hz, 1H), 7.91 (d, J = 9.3 Hz, 1H), 7.82 (d, J = 9.3 Hz, 1H), 7.25 (tq, J = 7.6, 2.5 Hz, 1H), 7.11-6.84 (m, 3H), 5.44 (dd, J = 8.2, 5.1 Hz, 1H), 3.85- 3.05 (m, 3H), 3.04-2.78 (m, 4H), 2.55- 2.25 (m, 4H), 1.60-1.12 (m, 9H); 548.2 |
| 63 | 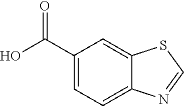 [SMILES: O=C(O)c1ccc2ncsc2c1] benzo[d]thiazole-6- carboxylic acid | Column: Phenomenex- Luna-C18-10 μm-25*250 mm, Mobile phase: A: 0.1% FA/H2O B: ACN, Gradient (B%): 29% to 59% in 12 min, Flow rate: 30 mL/min, UV wavelength: 220 nm | ¹H NMR (400 MHz, DMSO-d₆) δ 10.00 (bs, 1H), 9.49 (s, 1H), 8.27 (bs, 1H), 8.15- 8.08 (m, 1H), 7.56 (bs, 1H), 7.24 (t, J = 9.3 Hz, 1H), 7.09-6.88 (m, 3H), 5.49- 5.38 (m, 1H), 3.54-3.11 (m, 3H), 3.10- 2.69 (m, 4H), 2.60-2.29 (m, 4H), 1.50 (bs, 6H), 1.16 (bs, 3H); 565.2 |
| 64 | 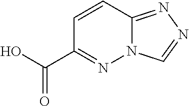 [SMILES: O=C(O)c1ccc2nncn2n1] [1,2,4]triazolo [4,3- b]pyridazine-6-carboxylic acid | Column: Phenomenex- Luna-C18-10 μm-25*250 mm, Mobile phase: A: 0.1% FA/H2O B: ACN, Gradient (B%): 23% to 53% in 12 min, Flow rate: 30 mL/min, UV wavelength: 220 nm | ¹H NMR (400 MHz, Methanol-d₄): δ 9.47 (s, 1H), 8.34 (d, J = 9.60 Hz, 1H), 7.49 (d, J = 9.6 Hz, 1H), 7.10 (s, 1H), 7.00-6.91 (m, 1H), 6.90-6.83 (m, 2H), 5.50 (dd, J = 7.6, 5.6 Hz, 1H), 3.73- 3.36 (m, 3H), 3.20-2.44 (m, 8H), 1.64 (d, J = 4.0 Hz, 6H), 1.38 (d, J = 6.8 Hz, 3H); 550.2 |
| 65 | 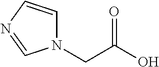 2-(1H-imidazol-1-yl)acetic acid | Column: Phenomenex- Luna-C18-10 μm-25*250 mm, Mobile phase: A: 0.1% FA/H2O B: ACN, Gradient (B%): 12% to 42% in 12 min, Flow rate: 30 mL/min, UV wavelength: 220 nm | ¹H NMR (400 MHz, DMSO-d₆) δ 10.08 (bs, 1H), 8.96 (d, J = 1.5 Hz, 1H), 7.65 (s, 1H), 7.58 (s, 1H), 7.24 (ddd, J = 9.4, 7.0, 2.4 Hz, 1H), 7.04-6.87 (m, 3H), 5.50-5.38 (m, 1H), 5.24 (s, 2H), 3.56- 3.12 (m, 3H), 3.11-2.68 (m, 4H), 2.52- 2.30 (m, 4H), 1.38 (bs, 6H), 1.28-1.10 (m, 3H); 512.2 |
| 66 | 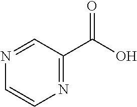 pyrazine-2-carboxylic acid | Column: Phenomenex- Luna-C18-10 μm-25*250 mm, Mobile phase: A: 0.1% FA/H2O B: ACN, Gradient (B%): 23% to 53% in 12 min, Flow rate: 30 mL/min, UV wavelength: 220 nm | ¹H NMR (400 MHz, DMSO-d₆) δ 8.85 (bs, 1H), 8.74 (s, 1H), 8.65 (s, 1H), 7.31- 7.18 (m, 1H), 7.06 (s, 1H), 7.05-6.89 (m, 2H), 5.46 (bs, 1H), 3.96-3.11 (m, 3H), 3.10-2.79 (m, 4H), 2.55-2.32 (m, 4H), 1.70-1.10 (m, 9H); 510.2 |
| 67 | 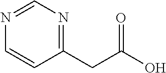 2-(pyrimidin-4-yl)acetic acid | Column: Phenomenex- Luna-C18-10 μm-25*250 mm, Mobile phase: A: 0.1% FA/H2O B: ACN, Gradient (B%): 19% to 49% in 12 min, Flow rate: 30 mL/min, UV wavelength: 220 nm | 1H NMR (400 MHz, DMSO-d₆) δ 9.08 (d, J = 1.4 Hz, 1H), 8.70 (d, J = 5.2 Hz, 1H), 7.39 (dd, J = 5.2, 1.4 Hz, 1H), 7.24 (tt, J = 9.3, 2.4 Hz, 1H), 7.03-6.85 (m, 3H), 5.43 (dd, J = 8.0, 5.1 Hz, 1H), 3.88 (bs, 2H), 3.70-3.05 (m, 3H), 3.04-2.78 (m, 4H), 2.60-2.18 (m, 4H), 1.68-1.02 (m, 9H); 524.2 |
| 68 | 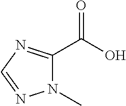 1-methyl-1H-1,2,3-triazole- 5-carboxylic acid | Column: Phenomenex- Luna-C18-10 μm-25*250 mm, Mobile phase: A: 0.1% FA/H2O B: ACN, Gradient (B%): 25% to 55% in 12 min, Flow rate: 30 mL/min, UV wavelength: 220 nm | ¹H NMR (400 MHz, DMSO-d₆) δ 11.66- 9.59 (m, 1H), 7.92 (bs, 1H), 7.25 (tt, J = 9.3, 2.4 Hz, 1H), 7.05 (s, 1H), 7.04- 6.77 (m, 2H), 5.45 (t, J = 6.5 Hz, 1H), 4.04 (s, 3H), 3.90-3.10 (m, 3H), 3.09- 2.81 (m, 4H), 2.52-2.32 (m, 4H), 1.70- 1.03 (m, 9H); 513.2 |
| 69 | 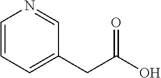 2-(pyridin-3-yl)acetic acid | Column: Phenomenex- Luna-C18-10 μm-25*250 mm, Mobile phase: A: 0.1% FA/H2O B: ACN, Gradient (B%): 13% to 43% in 12 min, Flow rate: 30 mL/min, UV wavelength: 220 nm | ¹H NMR (400 MHz, DMSO-d₆) δ 11.66- 9.36 (m, 1H), 8.73 (s, 1H), 8.64 (s, 1H), 8.17 (bs, 1H), 7.85 (bs, 1H), 7.25 (tt, J = 9.3, 2.3 Hz, 1H), 7.08 (s, 1H), 7.07- 6.91 (m, 2H), 5.48 (bs, 1H), 4.30-2.81 (m, 9H), 2.61-2.34 (m, 4H), 1.44 (bs, 9H); 523.2 |
| 70 | 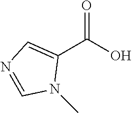 1-methyl-1H-imidazole-5- carboxylic acid | Column: Phenomenex- Luna-C18-10 μm-25*250 mm, Mobile phase: A: 0.1% FA/H2O B: ACN, Gradient (B%): 15% to 45% in 12 min, Flow rate: 30 mL/min, UV wavelength: 220 nm | ¹H NMR (400 MHz, DMSO-d₆) δ 8.97 (bs, 1H), 7.80 (bs, 1H), 7.25 (tt, J = 9.4, 2.4 Hz, 1H), 7.06 (s, 1H), 7.05-6.91 (m, 2H), 5.51-5.49 (m, 1H), 3.80 (s, 3H), 4.04-3.11 (m, 3H), 3.10-2.83 (m, 4H), 2.59-2.38 (m, 4H), 1.60-1.12 (m, 9H); 512.2 |
| 71 | 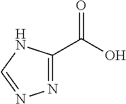 1H-1,2,4-triazole-5- carboxylic acid | Column: ASA-AZZOTA- C18-SB-10 μm-30*25 mm, Mobile phase: A: 0.1% FA/H2O B: ACN, Gradient (B%): 20% to 50% in 12 min, Flow rate: 35 mL/min, UV wavelength: 220 nm | ¹H NMR (400 MHz, Methanol-d₄) δ 8.45 (bs, 1H), 7.10 (s, 1H), 6.96 (tt, J = 9.2, 2.4 Hz, 1H), 6.91-6.79 (m, 2H), 5.48 (dd, J = 7.2, 5.2 Hz, 1H), 3.95-3.68 (m, 2H), 3.67-3.46 (m, 1H), 3.25-2.61 (m, 7H), 2.55-2.44 (m, 1H), 1.61 (d, J = 5.2 Hz, 6H), 1.41 (d, J = 6.4 Hz, 3H); 499.2 |
| 72 | 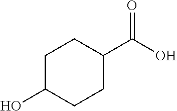 4-hydroxycyclohexane-1- carboxylic acid | Column: Phenomenex- Luna-C18-10 μm-25*250 mm, Mobile phase: A: 0.1% FA/H2O B: ACN, Gradient (B%): 26% to 56% in 12 min, Flow rate: 30 mL/min, UV wavelength: 220 nm | ¹H NMR (400 MHz, DMSO-d₆) δ 11.53- 9.49 (m, 1H), 7.24 (tt, J = 9.4, 2.4 Hz, 1H), 7.08 (s, 1H), 7.07-6.82 (m, 2H), 5.45 (bs, 1H), 4.32-2.73 (m, 11H), 2.61- 2.45 (m, 3H), 1.89-1.11 (m, 17H); 530.3 |
| 73 | 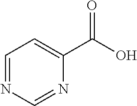 pyrimidine-4-carboxylic acid | Column: ASA-AZZOTA- C18-SB-10 μm-30*25 mm, Mobile phase: A: 0.1% FA/H2O B: ACN, Gradient (B%): 25% to 55% in 12 min, Flow rate: 35 mL/min, UV wavelength: 220 nm | ¹H NMR (400 MHz, Methanol-d₄) δ 9.19 (d, J = 1.4 Hz, 1H), 8.92 (d, J = 5.2 Hz, 1H), 7.60 (dd, J = 5.2, 1.2 Hz, 1H), 7.08 (s, 1H), 6.94 (tt, J = 9.2, 2.4 Hz, 1H), 6.86-6.80 (m, 2H), 5.47-5.42 (m, 1H), 3.56-3.29 (m, 3H), 3.20-2.89 (m, 3H), 2.85-2.74 (m, 1H), 2.73-2.53 (m, 3H), 2.50-2.41 (m, 1H), 1.62 (s, 6H), 1.32 (d, J = 7.2 Hz, 3H); 510.2 |
| 74 | 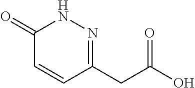 [SMILES: O=C(O)Cc1ccc(=O)[nH]n1] 2-(6-oxo-1,6- dihydropyridazin-3-yl)acetic acid | Column: ASA-AZZOTA- C18-SB-10 μm-30*25 mm, Mobile phase: A: 0.1% FA/H2O B: ACN, Gradient (B%): 20% to 50% in 12 min, Flow rate: 35 mL/min, UV wavelength: 220 nm | ¹H NMR (400 MHz, DMSO-d₆) δ 12.80 (bs, 1H), 9.96 (bs, 1H), 7.29-7.15 (m, 2H), 7.09-6.86 (m, 3H), 6.84-6.73 (m, 1H), 5.43-5.40 (m, 1H), 3.65 (s, 2H), 3.55-3.10 (m, 3H), 3.09-2.21 (m, 8H), 1.62-1.02 (m, 9H); 540.2 |
| 75 | 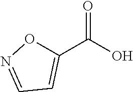 isoxazole-5-carboxylic acid | Column: Phenomenex- Luna-C18-10 μm-25*250 mm, Mobile phase: A: 0.1% FA/H2O B: ACN, Gradient (B%): 28% to 58% in 12 min, Flow rate: 30 mL/min, UV wavelength: 220 nm | ¹H NMR (400 MHz, DMSO-d₆) δ 10.02 (bs, 1H), 8.70 (s, 1H), 7.23 (t, J = 9.2 Hz, 1H), 7.02-6.80 (m, 4H), 5.48-5.39 (m, 1H), 3.50-3.19 (m, 3H), 3.05-2.78 (m, 4H), 2.62-2.30 (m, 4H), 1.49 (bs, 6H), 1.15 (bs, 3H); 499.2 |
| 76 | 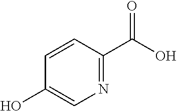 5-hydroxypicolinic acid | Column: Phenomenex- Luna-C18-10 μm-25*250 mm, Mobile phase: A: 0.1% FA/H2O B: ACN, Gradient (B%): 20% to 50% in 12 min, Flow rate: 30 mL/min, UV wavelength: 220 nm | ¹H NMR (400 MHz, DMSO-d₆) δ 10.40 (bs, 1H), 9.91 (s, 1H), 8.06 (d, J = 2.8 Hz, 1H), 7.41 (d, J = 8.4 Hz, 1H), 7.27- 7.13 (m, 2H), 7.00-6.91 (m, 3H), 5.48- 5.29 (m, 1H), 3.55-3.22 (m, 3H), 3.05- 2.32 (m, 8H), 1.43 (d, J = 3.2 Hz, 6H), 1.13 (d, J = 6.8 Hz, 3H); 525.2 |
| 77 | 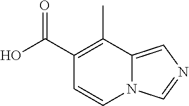 3-methylimidazo[1,5- a]pyridine-7-carboxylic acid | Column: Phenomenex- Luna-C18-10 μm-25*250 mm, Mobile phase: A: 0.1% FA/H2O B: ACN, Gradient (B%): 16% to 46% in 12 min, Flow rate: 30 mL/min, UV wavelength: 220 nm | ¹H NMR (400 MHz, DMSO-d₆) δ 10.03 (bs, 1H), 8.27-8.13 (m, 1H), 7.82-7.76 (m, 2H), 7.24 (t, J = 9.6 Hz, 1H), 7.05- 6.96 (m, 3H), 6.82-6.77 (m, 1H), 5.49- 5.38 (m, 1H), 3.56-3.11 (m, 3H), 3.15- 2.31 (m, 11H), 1.52-1.12 (m, 9H); 562.2 |
| 78 | 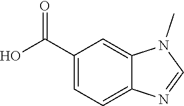 1-methyl-1H- benzo[d]imidazole-6- carboxylic acid | Column: ASA-AZZOTA- C18-SB-10 μm-30*25 mm, Mobile phase: A: 0.1% FA/H2O B: ACN, Gradient (B%): 22% to 52% in 12 min, Flow rate: 35 mL/min, UV wavelength: 220 nm | ¹H NMR (400 MHz, DMSO-d₆) δ 9.68 (bs, 1H), 8.25-7.76 (m, 2H), 7.60-7.42 (m, 1H), 7.29-6.86 (m, 4H), 5.45 (bs, 1H), 3.95 (s, 3H), 3.94-2.79 (m, 7H), 2.59-2.35 (m, 4H), 1.67-1.11 (m, 9H); 563.2 [M + 2H]⁺ |
| 79 | 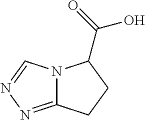 [SMILES: O=C(O)C1CCc2nncn21] 6,7-dihydro-5H-pyrrolo[2,1- c][1,2,4]triazole-5- carboxylic acid | Column: Phenomenex- Luna-C18-10 μm-25*250 mm, Mobile phase: A: H2O B: ACN, Gradient (B%): 18% to 48% in 12 min, Flow rate: 30 mL/min, UV wavelength: 220 nm | |
| 80 | 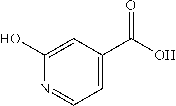 2-hydroxyisonicotinic acid | Column: Phenomenex- Luna-C18-10 μm-25*250 mm, Mobile phase: A: H2O B: ACN, Gradient (B%): 19% to 49% in 12 min, Flow rate: 30 mL/min, UV wavelength: 220 nm | ¹H NMR (400 MHz, DMSO-d₆) δ 11.69 (bs, 1H), 9.93 (s, 1H), 7.40 (d, J = 6.8 Hz, 1H), 7.27-7.20 (m, 1H), 7.02-6.91 (m, 3H), 6.18 (d, J = 1.6 Hz, 1H), 6.06 (dd, J = 6.4, 1.6 Hz, 1H), 5.38 (dd, J = 7.2, 5.2 Hz, 1H), 3.40-3.22 (m, 3H), 3.05-2.32 (m, 8H), 1.43 (d, J = 4.4 Hz, 6H), 1.13 (d, J = 6.8 Hz, 3H); 525.2 |
| 81 | 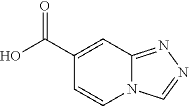 [SMILES: O=C(O)c1ccn2cnnc2c1] [1,2,4]triazolo[4,3- a]pyridine-7-carboxylic acid | Column: Phenomenex- Luna-C18-10 μm-25*250 mm, Mobile phase: A: 0.1% FA/H2O B: ACN, Gradient (B%): 20% to 50% in 12 min, Flow rate: 30 mL/min, UV wavelength: 220 nm | ¹H NMR (400 MHz, DMSO-d₆) δ 11.63- 9.56 (m, 1H), 9.32 (s, 1H), 8.60 (d, J = 6.8 Hz, 1H), 8.15-7.69 (m, 1H), 7.24- 6.83 (m, 5H), 5.46-5.40 (m, 1H), 4.15- 3.09 (m, 3H), 3.08-2.80 (m, 4H), 2.58- 2.32 (m, 4H), 1.71-1.09 (m, 9H); 549.2 |
| 82 | 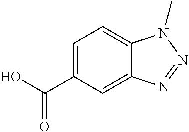 1-methyl-1H- benzo[d][1,2,3]triazole-5- carboxylic acid | Column: ASA-AZZOTA- C18-SB-10 μm-30*25 mm, Mobile phase: A: 0.1% FA/H2O B: ACN, Gradient (B%): 28% to 58% in 12 min, Flow rate: 35 mL/min, UV wavelength: 220 nm | ¹H NMR (400 MHz, DMSO-d₆) δ 9.93 (s, 1H), 8.05 (s, 1H), 7.88 (d, J = 8.6 Hz, 1H), 7.58 (d, J = 8.4 Hz, 1H), 7.29-7.19 (m, 1H), 7.03-6.90 (m, 3H), 5.49-5.26 (m, 1H), 4.33 (s, 3H), 3.56-3.22 (m, 3H), 3.05-2.71 (m, 4H), 2.61-2.35 (m, 4H), 1.49 (bs, 6H), 1.15 (d, J = 6.8 Hz, 3H); 563.2 |

### Example 83: 4-(4-((R)-1-(((R)-5-(3,5-difluorophenyl)-6,7-dihydro-5H-pyrrolo[1,2-a]imidazol-2-yl)amino)-1-oxopropan-2-yl)-2,2-dimethylpiperazine-1-carbonyl)pyridine 1-oxide

### Step 1: (S)—N—((R)-5-(3,5-difluorophenyl)-6,7-dihydro-5H-pyrrolo[1,2-a]imidazol-2-yl)-2-hydroxypropanamide

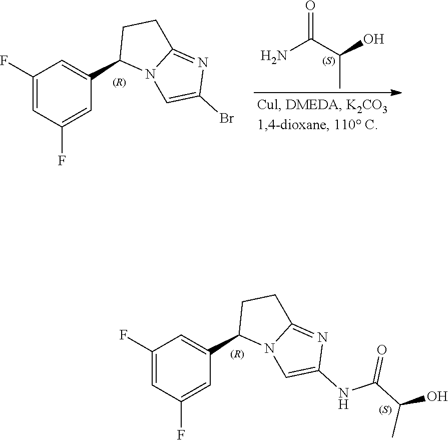

To a mixture of (R)-2-bromo-5-(3,5-difluorophenyl)-6,7-dihydro-5H-pyrrolo[1,2-a]imidazole (8.8 g, 29.4 mmol) in 1,4-dioxane (176 mL) were added (S′)-2-hydroxypropanamide (13.1 g, 147 mmol), CuI (2.24 g, 11.8 mmol), N,N-dimethyl-1,2-ethanediamine (3.34 g, 23.5 mmol) and K₃PO₄ (12.5 g, 58.8 mmol). The resulting mixture was stirred at 110° C. overnight under nitrogen atmosphere. Upon completion, the mixture was allowed to cool down to room temperature. The reaction was diluted with DCM (176 mL) and washed with water (88 mL). The aqueous layer was extracted DCM (88 mL×2). The combined organic layers were washed with brine (44 mL), dried over anhydrous Na₂SO₄, filtered, and concentrated under reduced pressure. The crude residue was purified by silica gel column chromatography, eluted with DCM/MeOH (19:1) to afford (S)—N—((R)-5-(3,5-difluorophenyl)-6,7-dihydro-5H-pyrrolo[1,2-a]imidazol-2-yl)-2-hydroxypropanamide (6.78 g, 75% yield) as a yellow solid. LCMS (ESI) [M+H]⁺: 307.9.

### Step 2: (S)-1-(((R)-5-(3,5-difluorophenyl)-6,7-dihydro-5H-pyrrolo[1,2-a]imidazol-2-yl)amino)-1-oxopropan-2-yl 2-nitrobenzenesulfonate

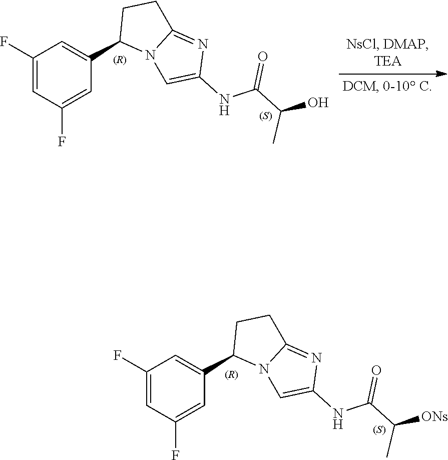

To a solution of (S)—N—((R)-5-(3,5-difluorophenyl)-6,7-dihydro-5H-pyrrolo[1,2-a]imidazol-2-yl)-2-hydroxypropanamide (4.0 g, 13.02 mmol) in DCM (120 ml) was added Et₃N (7.9 g, 78.10 mmol) at 0° C. After 10 minutes, DMAP (0.4 g, 3.25 mmol) and 2-NsCl (4.3 g, 19.53 mmol) were subsequently added. The resulting reaction mixture was warmed to 10° C. and stirred for 10 hours. Upon completion, the mixture was quenched with citric acid (40 mL, 10% aq.). The organic phase was washed with citric acid (40 mL×2, 10% aq.), water (40 mL×3), dried over Na₂SO₄, filtered, and concentrated under reduced pressure to afford (S)-1-(((R)-5-(3,5-difluorophenyl)-6,7-dihydro-5H-pyrrolo[1,2-a]imidazol-2-yl)amino)-1-oxopropan-2-yl 2-nitrobenzenesulfonate (6.0 g, 93% yield, crude), which was used in the next step without further purification. LCMS (ESI) [M+H]⁺: 493.1

### Step 3: tert-butyl 4-((R)-1-(((R)-5-(3,5-difluorophenyl)-6,7-dihydro-5H-pyrrolo[1,2-a]imidazol-2-yl)amino)-1-oxopropan-2-yl)-2,2-dimethylpiperazine-1-carboxylate

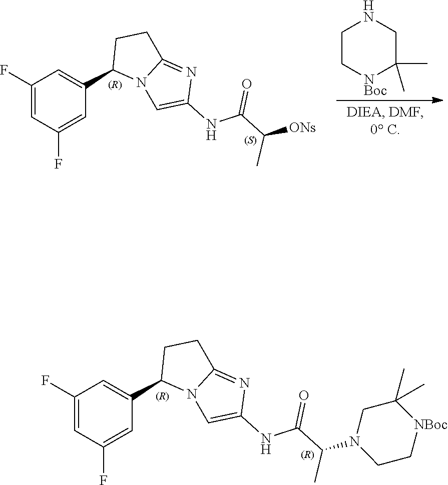

To a solution of (S)-1-(((R)-5-(3,5-difluorophenyl)-6,7-dihydro-5H-pyrrolo[1,2-a]imidazol-2-yl)amino)-1-oxopropan-2-yl 2-nitrobenzenesulfonate (6.0 g, 12.18 mmol) in DMF (60 mL) was added DIEA (4.7 g, 36.55 mmol) and tert-butyl 2,2-dimethylpiperazine-1-carboxylate (5.2 g, 24.37 mmol) at 0° C. The resulting reaction mixture was stirred at 0° C. for 16 hours. Upon completion, the reaction mixture was quenched with citric acid (60 mL, 10% aq.). The aqueous layer was extracted with DCM (60 mL×3). The combined organic layer was dried over Na₂SO₄, filtered, and concentrated under reduced pressure. The crude residue was purified by flash column chromatography (silica, eluent: DCM/MeOH=10/1) to give a brown solid. The brown solid was suspended in MTBE (60 mL) and stirred for 0.5 hour at room temperature. The resulting mixture was filtered to give tert-butyl 4-((R)-1-(((R)-5-(3,5-difluorophenyl)-6,7-dihydro-5H-pyrrolo[1,2-a]imidazol-2-yl)amino)-1-oxopropan-2-yl)-2,2-dimethylpiperazine-1-carboxylate as a white solid (4.5 g, 73% yield). LCMS (ESI) [M+H]⁺: 504.2

### Step 4: (R)—N—((R)-5-(3,5-difluorophenyl)-6,7-dihydro-5H-pyrrolo[1,2-a]imidazol-2-yl)-2-(3,3-dimethylpiperazin-1-yl)propanamide

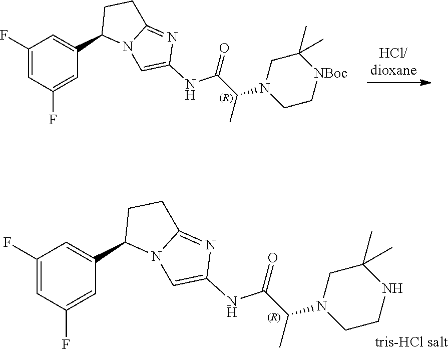

tert-Butyl 4-((R)-1-(((R)-5-(3,5-difluorophenyl)-6,7-dihydro-5H-pyrrolo[1,2-a]imidazol-2-yl)amino)-1-oxopropan-2-yl)-2,2-dimethylpiperazine-1-carboxylate (3.5 g, 6.95 mmol) was suspended in HCl (35 mL, 4 M in 1,4-dioxane) at 20° C. and the resulting mixture was stirred for 2 hours. Upon completion, the mixture was filtered. The precipitate was collected and dried in vacuo at 50° C. to afford (R)—N—((R)-5-(3,5-difluorophenyl)-6,7-dihydro-5H-pyrrolo[1,2-a]imidazol-2-yl)-2-(3,3-dimethylpiperazin-1-yl)propanamide as a white solid (3.36 g, 96% yield). LCMS (ESI) [M+H]⁺: 404.2

### Step 5: 4-(4-((R)-1-(((R)-5-(3,5-difluorophenyl)-6,7-dihydro-5H-pyrrolo[1,2-a]imidazol-2-yl)amino)-1-oxopropan-2-yl)-2,2-dimethylpiperazine-1-carbonyl)pyridine 1-oxide

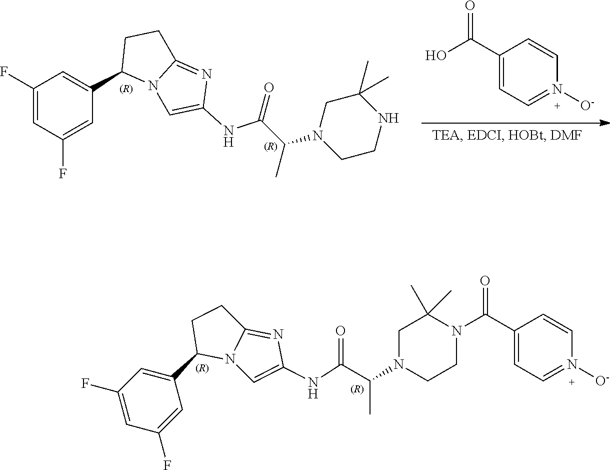

To a solution of 4-carboxypyridine 1-oxide (0.13 g, 0.94 mmol) in DMF (2 mL) was added Et₃N (0.38 g, 3.74 mmol), and (R)—N—((R)-5-(3,5-difluorophenyl)-6,7-dihydro-5H-pyrrolo[1,2-a]imidazol-2-yl)-2-(3,3-dimethylpiperazin-1-yl)propanamide (0.19 g, 0.47 mmol) at 10° C. EDCI (0.16 g, 0.84 mmol) and HOBt (0.11 g, 0.84 mmol) were subsequently added. The resulting mixture was stirred at 20° C. for 10 hours. Upon completion, the reaction mixture was quenched with water (6 ml) and extracted with DCM (4 mL×3). The combined organic layers were washed with Na₂CO₃ (1.3 mL, 10% aq.), brine (1.3 mL, 10% aq.), dried over Na₂SO₄, filtered, and concentrated under reduced pressure. The crude residue was triturated in DCM/MTBE (8 mL, v/v=1/3) to give 4-(4-((R)-1-(((R)-5-(3,5-difluorophenyl)-6,7-dihydro-5H-pyrrolo[1,2-a]imidazol-2-yl)amino)-1-oxopropan-2-yl)-2,2-dimethylpiperazine-1-carbonyl)pyridine 1-oxide (Compound 83, 120 mg, 62% yield) as an off-white solid.

| Compound# | Characterization Data |
|---|---|
| 83 | LCMS (ESI)[M + H]⁺: 525.3 |
| | ¹H NMR (400 MHz, DMSO-d₆) δ 10.0 (s, 1H), 8.20 (d, J = 6.6 Hz, 2H), 7.40 (d, J = 6.6 Hz, 2H), |
| | 7.21-7.19 (m, 1H), 6.99 (s, 1H), 6.92-6.90 (m, 2H), 5.41-5.37 (m, 1H), 3.40-3.28 (m, 3H), |
| | 3.07-2.64 (m, 5H), 2.40-2.36 (m, 3H), 1.43 (s, 3H), 1.42 (3H), 1.11 (d, J = 6.9 Hz, 3H). |

Chiral Analytical SFC Analysis of Compound 83 vs. Compound 1:

Compound 83 (Method L, 2.72 min, Peak 1); Compound 1 (Method L, 3.00 min, Peak 2).

### Example 84: (R)—N—((R)-5-(3,5-difluorophenyl)-6,7-dihydro-5H-pyrrolo[1,2-a]imidazol-2-yl)-2-(3,3-dimethyl-4-(5-oxo-4,5-dihydropyrazine-2-carbonyl)piperazin-1-yl)propanamide

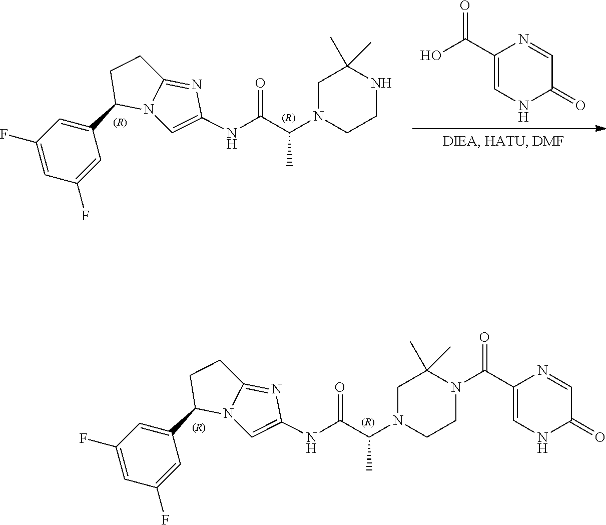

To a solution of 5-oxo-4,5-dihydropyrazine-2-carboxylic acid (0.13 g, 0.94 mmol) in DMF (1 mL) was added DIEA (0.18 g, 1.40 mmol) and HATU (0.32 g, 0.84 mmol) at 10° C. Then, a solution of (R)—N—((R)-5-(3,5-difluorophenyl)-6,7-dihydro-5H-pyrrolo[1,2-a]imidazol-2-yl)-2-(3,3-dimethylpiperazin-1-yl)propanamide (190 mg, 0.47 mmol) and DIEA (0.30 g, 2.34 mmol) in DMF were added. The reaction mixture was stirred at 10° C. for 16 hours. Upon completion, the reaction mixture was quenched with water (6 mL) and extracted with DCM (2 mL×3). The combined organic layers were washed with brine (2 mL, 10% aq.), dried over Na₂SO₄, filtered, and concentrated under reduced pressure. The crude residue was purified by flash column chromatography (silica, eluent: DCM/MeOH=10/1) to afford a brown solid, which was triturated in DCM (6 mL) to afford (R)—N—((R)-5-(3,5-difluorophenyl)-6,7-dihydro-5H-pyrrolo[1,2-a]imidazol-2-yl)-2-(3,3-dimethyl-4-(5-oxo-4,5-dihydropyrazine-2-carbonyl)piperazin-1-yl)propanamide (Compound 84, 120 mg, 61% yield) as a white solid.

| Compound# | Characterization Data |
|---|---|
| 84 | LCMS (ESI) [M + H]⁺: 526.2 |
| | ¹H NMR (400 MHz, DMSO-d₆) δ 12.56 (s, 1H, bs), 9.98 (s, 1H), 7.91 (s, 1H), 7.71 (s, 1H), |
| | 7.24-7.18 (m, 1H), 6.99 (s, 1H), 6.92-6.90 (m, 2H), 5.41-5.37 (m, 1H), 3.43-3.36 (m, 2H), |
| | 2.98-2.65 (m, 5H), 2.54-2.50 (m, 1H), 2.40-2.27 (m, 3H), 1.41 (s, 3H), 1.40 (s, 3H), 1.13 |
| | (d, J = 6.9 Hz, 3H). |

Chiral Analytical SFC Analysis of Compound 84 vs. Compound 3:

Compound 84 (Method N, 2.49 min, Peak 1); Compound 3 (Method N, 2.68 min, Peak 2).

### Example 85: 4-(4-((S)-1-(((S)-5-(3,5-difluorophenyl)-6,7-dihydro-5H-pyrrolo[1,2-a]imidazol-2-yl)amino)-1-oxopropan-2-yl)-2,2-dimethylpiperazine-1-carbonyl)pyridine 1-oxide

### Step 1: (R)—N—((S)-5-(3,5-difluorophenyl)-6,7-dihydro-5H-pyrrolo[1,2-a]imidazol-2-yl)-2-hydroxypropanamide

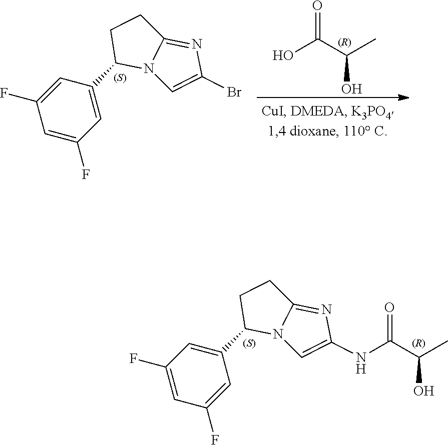

(S)-2-bromo-5-(3,5-difluorophenyl)-6,7-dihydro-5H-pyrrolo[1,2-a]imidazole was prepared following the same reaction sequence for the (R)-2-bromo-5-(3,5-difluorophenyl)-6,7-dihydro-5H-pyrrolo[1,2-a]imidazole employing (R)-tert-butylsulfinamide as the chiral auxiliary.

To a mixture of (S)-2-bromo-5-(3,5-difluorophenyl)-6,7-dihydro-5H-pyrrolo[1,2-a]imidazole (8.8 g, 29.4 mmol) in 1,4-dioxane (176 mL) were added (R)-2-hydroxypropanamide (13.1 g, 147 mmol), CuI (2.24 g, 11.8 mmol), N,N-dimethyl-1,2-ethanediamine (3.34 g, 23.5 mmol) and K₃PO₄ (12.5 g, 58.8 mmol). The resulting mixture was stirred at 110° C. overnight under nitrogen atmosphere. Upon completion, the mixture was allowed to cool down to room temperature. The reaction was diluted with DCM (176 ml) and was washed with water (88 ml). The aqueous layer was extracted DCM (88 mL×2). The combined organic layers were washed with brine (44 mL), dried over anhydrous Na₂SO₄, filtered, and concentrated under reduced pressure. The crude residue was purified by silica gel column chromatography, eluted with DCM/MeOH (19:1) to afford (R)—N—((S)-5-(3,5-difluorophenyl)-6,7-dihydro-5H-pyrrolo[1,2-a]imidazol-2-yl)-2-hydroxypropanamide (5.8 g, 70% yield) as a yellow solid. LCMS (ESI) [M+H]⁺: 308.0.

### Step 2: (R)-1-(((S)-5-(3,5-difluorophenyl)-6,7-dihydro-5H-pyrrolo[1,2-a]imidazol-2-yl)amino)-1-oxopropan-2-yl 2-nitrobenzenesulfonate

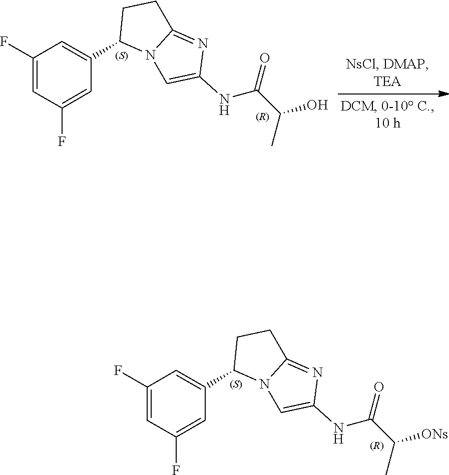

To a solution of (R)—N—((S)-5-(3,5-difluorophenyl)-6,7-dihydro-5H-pyrrolo[1,2-a]imidazol-2-yl)-2-hydroxypropanamide (5.0 g, 16.27 mmol) in DCM (150 ml) was added Et₃N (9.9 g, 97.63 mmol) at 0° C. After 10 minutes, DMAP (0.5 g, 4.07 mmol) and 2-NsCl (5.4 g, 24.41 mmol) were subsequently added. The resulting reaction mixture was warmed to 10° C. and stirred for 10 hours. Upon completion, the mixture was quenched with citric acid (450 mL, 10% aq.) The organic layer was washed with citric acid (250 mL×2, 10% aq.), water (450 mL×3), dried over Na₂SO₄, filtered, and concentrated under reduced pressure to afford (R)-1-(((S)-5-(3,5-difluorophenyl)-6,7-dihydro-5H-pyrrolo[1,2-a]imidazol-2-yl)amino)-1-oxopropan-2-yl 2-nitrobenzenesulfonate (8.0 g, 101% yield, crude), which was used in the next step without further purification. LCMS (ESI) [M+H]⁺: 493.1

### Step 3: tert-butyl 4-((S)-1-(((S)-5-(3,5-difluorophenyl)-6,7-dihydro-5H-pyrrolo[1,2-a]imidazol-2-yl)amino)-1-oxopropan-2-yl)-2,2-dimethylpiperazine-1-carboxylate

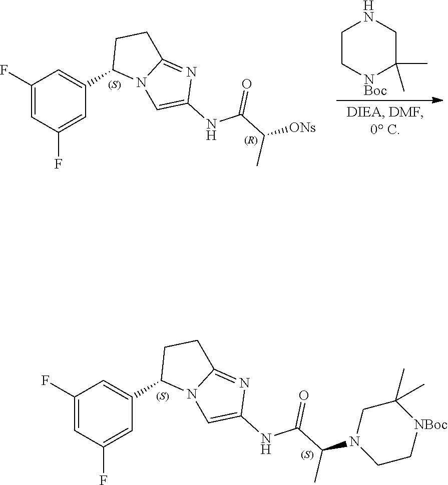

To a solution of (R)-1-(((S)-5-(3,5-difluorophenyl)-6,7-dihydro-5H-pyrrolo[1,2-a]imidazol-2-yl)amino)-1-oxopropan-2-yl 2-nitrobenzenesulfonate (8.0 g, 16.25 mmol) in DMF (80 mL) was added DIEA (6.3 g, 48.74 mmol) and tert-butyl 2,2-dimethylpiperazine-1-carboxylate (7.0 g, 32.49 mmol) at 0° C. The resulting mixture was stirred at 0° C. for 16 hours. Upon completion, the reaction mixture was quenched with citric acid (240 mL, 10% aq.) and extracted with DCM (240 mL×3). The combined organic layers were dried over Na₂SO₄, filtered, and concentrated under reduced pressure. The crude residue was purified by flash column chromatography (silica, eluent: DCM/MeOH=10/1) to give a brown solid. The brown solid was suspended in MTBE (30 mL) and stirred for 0.5 hour at room temperature. The resulting mixture was filtered to give tert-butyl 4-((S)-1-(((S)-5-(3,5-difluorophenyl)-6,7-dihydro-5H-pyrrolo[1,2-a]imidazol-2-yl)amino)-1-oxopropan-2-yl)-2,2-dimethylpiperazine-1-carboxylate as a white solid (4.5 g, 55% yield). LCMS (ESI) [M+H]⁺: 504.2

### Step 4: (S)—N—((S)-5-(3,5-difluorophenyl)-6,7-dihydro-5H-pyrrolo[1,2-a]imidazol-2-yl)-2-(3,3-dimethylpiperazin-1-yl)propanamide

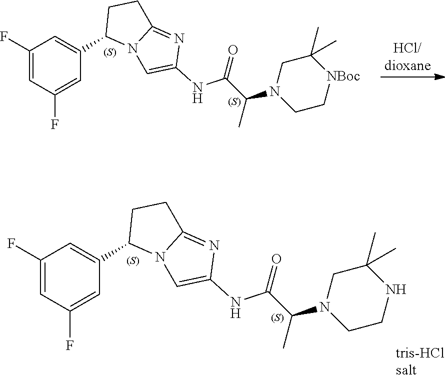

tert-butyl 4-((S)-1-(((S)-5-(3,5-difluorophenyl)-6,7-dihydro-5H-pyrrolo[1,2-a]imidazol-2-yl)amino)-1-oxopropan-2-yl)-2,2-dimethylpiperazine-1-carboxylate (4.0 g, 7.94 mmol) was suspended in HCl (40 mL, 4 M in 1,4-dioxane) at 20° C. and the resulting mixture was stirred for 2 hours. Upon completion, the mixture was filtered. The precipitate was collected and dried in vacuo at 50° C. to afford (S)—N—((S)-5-(3,5-difluorophenyl)-6,7-dihydro-5H-pyrrolo[1,2-a]imidazol-2-yl)-2-(3,3-dimethylpiperazin-1-yl)propanamide as a white tris-HCl salt (3.2 g, 63% yield). LCMS (ESI) [M+H]+. 404.2

### Step 5: 4-(4-((S)-1-(((S)-5-(3,5-difluorophenyl)-6,7-dihydro-5H-pyrrolo[1,2-a]imidazol-2-yl)amino)-1-oxopropan-2-yl)-2,2-dimethylpiperazine-1-carbonyl)pyridine 1-oxide

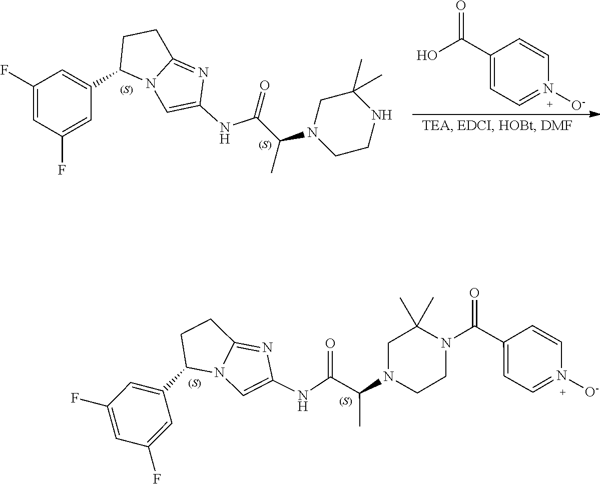

To a solution of 4-carboxypyridine 1-oxide (0.13 g, 0.94 mmol) in DMF (2 mL) was added Et₃N (0.38 g, 3.74 mmol), and (S)—N—((S)-5-(3,5-difluorophenyl)-6,7-dihydro-5H-pyrrolo[1,2-a]imidazol-2-yl)-2-(3,3-dimethylpiperazin-1-yl)propanamide (0.19 g, 0.47 mmol) at 10° C. EDCI (0.16 g, 0.84 mmol) and HOBt (0.11 g, 0.84 mmol) were subsequently added. The resulting mixture was warmed to 20° C. and stirred for 10 hours. Upon completion, the reaction mixture was quenched with water (6 mL) and extracted with DCM (4 mL×3). The combined organic layers were washed with Na₂CO₃ (1.3 mL, 10% aq.), brine (1.3 mL, 10% aq.), dried over Na₂SO₄, filtered, and concentrated under reduced pressure. The crude residue was triturated in DCM/MTBE (8 mL, v/v=1/3,) to give 4-(4-((S)-1-(((S)-5-(3,5-difluorophenyl)-6,7-dihydro-5H-pyrrolo[1,2-a]imidazol-2-yl)amino)-1-oxopropan-2-yl)-2,2-dimethylpiperazine-1-carbonyl)pyridine 1-oxide (Compound 85, 120 mg, 49% yield) as an off-white solid.

| Compound# | Characterization Data |
|---|---|
| 85 | LCMS (ESI) [M + H]⁺: 525.3 |
| | ¹H NMR (300 MHz, DMSO-d₆) δ 9.96(s, 1H), 8.20 (d, J = 6.6 Hz, 2H), 7.40 (d, J = 6.6 Hz, 2H), |
| | 7.25-7.18 (m, 1H), 6.99 (s, 1H), 6.92-6.90 (m, 2H), 5.40-5.36 (m, 1H), 3.41-3.28 (m, 3H), |
| | 3.08-2.65 (m, 5H), 2.39-2.36 (m, 3H), 1.44 (s, 3H), 1.42 (3H), 1.13 (d, J = 6.9 Hz, 3H). |

### Example 86: (S)—N—((S)-5-(3,5-difluorophenyl)-6,7-dihydro-5H-pyrrolo[1,2-a]imidazol-2-yl)-2-(3,3-dimethyl-4-(5-oxo-4,5-dihydropyrazine-2-carbonyl)piperazin-1-yl)propanamide

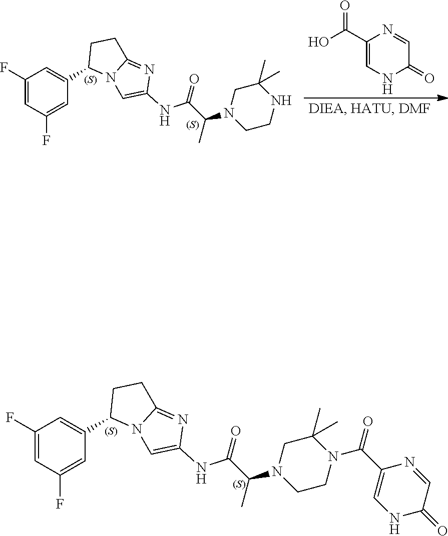

To a solution of 5-oxo-4,5-dihydropyrazine-2-carboxylic acid (0.13 g, 0.94 mmol) in DMF (2 mL) was added DIEA (0.18 g, 1.40 mmol) and HATU (0.32 g, 0.84 mmol) at 10° C. Then, a solution of (S)—N—((S)-5-(3,5-difluorophenyl)-6,7-dihydro-5H-pyrrolo[1,2-a]imidazol-2-yl)-2-(3,3-dimethylpiperazin-1-yl)propanamide (190 mg, 0.47 mmol) and DIEA (0.30 g, 2.34 mmol) in DMF (2 mL) were added. The reaction mixture was stirred at 10° C. for 16 hours. Upon completion, the reaction mixture was quenched with water (6 mL) and extracted with DCM (4 mL×3). The combined organic layers were washed with brine (4 mL, 10% aq.), dried over Na₂SO₄, filtered, and concentrated under reduced pressure. The crude residue was purified by flash column chromatography (silica, eluent: DCM/MeOH=10/1) to afford a brown solid, which was triturated in DCM (6 mL) to afford (S)—N—((S)-5-(3,5-difluorophenyl)-6,7-dihydro-5H-pyrrolo[1,2-a]imidazol-2-yl)-2-(3,3-dimethyl-4-(5-oxo-4,5-dihydropyrazine-2-carbonyl)piperazin-1-yl)propanamide (Compound 86, 35 mg, 14% yield) as a white solid.

| Compound# | Characterization Data |
|---|---|
| 86 | LCMS (ESI) [M + H]⁺: 526.2 |
| | ¹H NMR (400 MHz, DMSO-d₆) δ 12.60 (s, 1H, bs), 9.92 (s, 1H), 7.91 (s, 1H), 7.72 (s, 1H), |
| | 7.24-7.20 (m, 1H), 7.00 (s, 1H), 6.93-6.90 (m, 2H), 5.39-5.37 (m, 1H), 3.45-3.44 (m, 1H), |
| | 3.35-3.33 (m, 2H), 3.14-3.12 (m, 1H), 2.95-2.92 (m, 2H), 2.78-2.64 (m, 2H) 2.54-2.50 |
| | (m, 1H), 2.38-2.36 (m, 3H), 1.42 (s, 3H), 1.41 (s, 3H), 1.14 (d, J = 6.9 Hz, 3H). |

### Example 87: 4-(4-((R)-1-(((S)-5-(3,5-difluorophenyl)-6,7-dihydro-5H-pyrrolo[1,2-a]imidazol-2-yl)amino)-1-oxopropan-2-yl)-2,2-dimethylpiperazine-1-carbonyl)pyridine 1-oxide

### Step 1: (S)—N—((S)-5-(3,5-difluorophenyl)-6,7-dihydro-5H-pyrrolo[1,2-a]imidazol-2-yl)-2-hydroxypropanamide

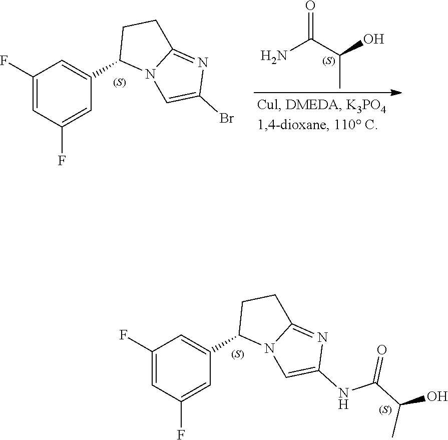

To a mixture of (S)-2-bromo-5-(3,5-difluorophenyl)-6,7-dihydro-5H-pyrrolo[1,2-a]imidazole (8.8 g, 29.4 mmol) in 1,4-dioxane (176 mL) were added (S)-2-hydroxypropanamide (13.1 g, 147 mmol), CuI (2.24 g, 11.8 mmol), N,N-dimethyl-1,2-ethanediamine (3.34 g, 23.5 mmol) and K₃PO₄ (12.5 g, 58.8 mmol). The resulting mixture was stirred at 110° C. overnight under nitrogen atmosphere. Upon completion, the mixture was allowed to cool down to room temperature. The reaction was diluted with DCM (176 ml) and washed with water (88 ml). The aqueous layer was extracted DCM (88 mL×2). The combined organic layers were washed with brine (44 ml), dried over anhydrous Na₂SO₄, filtered, and concentrated under reduced pressure. The crude residue was purified by silica gel column chromatography, eluted with DCM/MeOH (19:1) to afford (S)—N—((S)-5-(3,5-difluorophenyl)-6,7-dihydro-5H-pyrrolo[1,2-a]imidazol-2-yl)-2-hydroxypropanamide (5.5 g, 75% yield) as a yellow solid. LCMS (ESI) [M+H]⁺: 307.9.

### Step 2: (S)-1-(((S)-5-(3,5-difluorophenyl)-6,7-dihydro-5H-pyrrolo[1,2-a]imidazol-2-yl)amino)-1-oxopropan-2-yl 2-nitrobenzenesulfonate

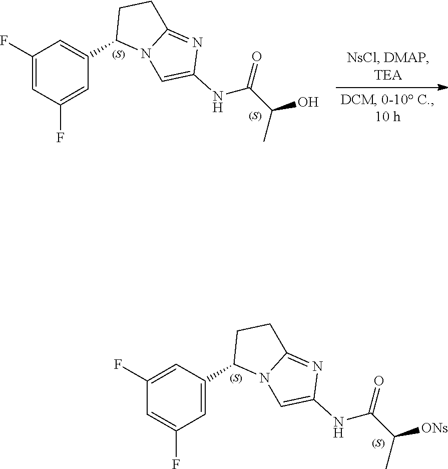

To a solution of (S)—N—((S)-5-(3,5-difluorophenyl)-6,7-dihydro-5H-pyrrolo[1,2-a]imidazol-2-yl)-2-hydroxypropanamide (4.5 g, 14.64 mmol) in DCM (135 ml) was added Et₃N (8.9 g, 87.86 mmol) at 0° C. After 10 minutes, DMAP (0.45 g, 3.66 mmol) and 2-NsCl (4.87 g, 21.97 mmol) were subsequently added. The resulting reaction mixture was warmed to 10° C. and stirred for 10 hours. Upon completion, the mixture was quenched with citric acid (400 mL, 10% aq.). The organic phase was washed with citric acid (200 mL×2, 10% aq.), water (200 mL×3), dried over Na₂SO₄, filtered, and concentrated under reduced pressure to afford (S)-1-(((S)-5-(3,5-difluorophenyl)-6,7-dihydro-5H-pyrrolo[1,2-a]imidazol-2-yl)amino)-1-oxopropan-2-yl 2-nitrobenzenesulfonate (7.5 g, 104% yield, crude), which was used in the next step without further purification. LCMS (ESI) [M+H]⁺: 493.1

### Step 3: tert-butyl 4-((R)-1-(((S)-5-(3,5-difluorophenyl)-6,7-dihydro-5H-pyrrolo[1,2-a]imidazol-2-yl)amino)-1-oxopropan-2-yl)-2,2-dimethylpiperazine-1-carboxylate

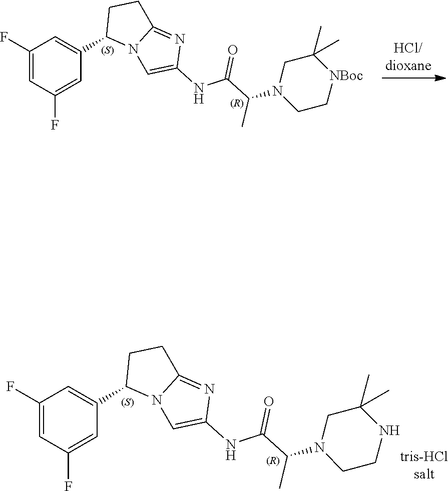

To a solution of (S)-1-(((S)-5-(3,5-difluorophenyl)-6,7-dihydro-5H-pyrrolo[1,2-a]imidazol-2-yl)amino)-1-oxopropan-2-yl 2-nitrobenzenesulfonate (7.5 g, 15.22 mmol) in DMF (75 mL) was added DIEA (5.9 g, 45.69 mmol) and tert-butyl 2,2-dimethylpiperazine-1-carboxylate (6.5 g, 30.46 mmol) at 0° C. The resulting reaction mixture was stirred at 0° C. for 16 hours. Upon completion, the reaction mixture was quenched with citric acid (60 mL, 10% aq.) and extracted with DCM (225 mL×3). The combined organic layers were dried over Na₂SO₄, filtered, and concentrated under reduced pressure. The crude residue was purified by flash column chromatography (silica, eluent: DCM/MeOH=10/1) to give a brown solid. The brown solid was suspended in MTBE (40 mL) and stirred for 0.5 hour at room temperature. The resulting mixture was filtered to give tert-butyl 4-((R)-1-(((S)-5-(3,5-difluorophenyl)-6,7-dihydro-5H-pyrrolo[1,2-a]imidazol-2-yl)amino)-1-oxopropan-2-yl)-2,2-dimethylpiperazine-1-carboxylate as a white solid (4.2 g, 93% purity). LCMS (ESI) [M+H]⁺: 504.2

### Step 4: (R)—N—((S)-5-(3,5-difluorophenyl)-6,7-dihydro-5H-pyrrolo[1,2-a]imidazol-2-yl)-2-(3,3-dimethylpiperazin-1-yl)propanamide

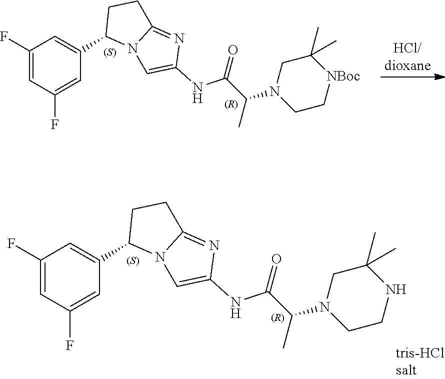

tert-Butyl 4-((R)-1-(((S)-5-(3,5-difluorophenyl)-6,7-dihydro-5H-pyrrolo[1,2-a]imidazol-2-yl)amino)-1-oxopropan-2-yl)-2,2-dimethylpiperazine-1-carboxylate (3.5 g, 6.95 mmol) was suspended in HCl (35 mL, 4 M in 1,4-dioxane) at 20° C. The resulting reaction mixture was stirred for 2 hours at 20° C. Upon completion, the mixture was filtered. The precipitate was collected and dried in vacuo at 50° C. to afford (R)—N—((S)-5-(3,5-difluorophenyl)-6,7-dihydro-5H-pyrrolo[1,2-a]imidazol-2-yl)-2-(3,3-dimethylpiperazin-1-yl)propanamide as a white tris-HCl salt (2.9 g, 65% yield). LCMS (ESI) [M+H]⁺: 404.2

### Step 5: 4-(4-((R)-1-(((S)-5-(3,5-difluorophenyl)-6,7-dihydro-5H-pyrrolo[1,2-a]imidazol-2-yl)amino)-1-oxopropan-2-yl)-2,2-dimethylpiperazine-1-carbonyl)pyridine 1-oxide (Compound 87)

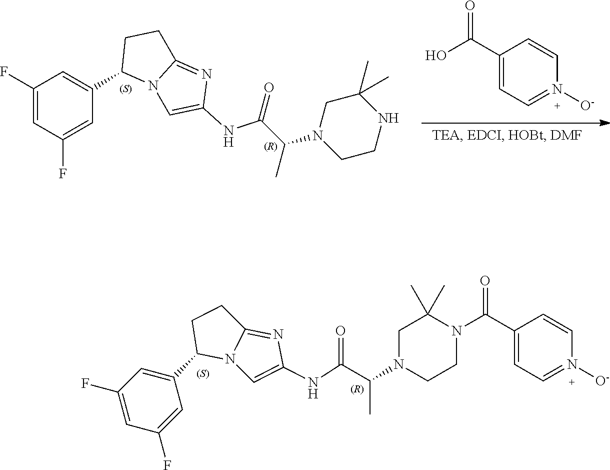

To a solution of 4-carboxypyridine 1-oxide (0.13 g, 0.94 mmol) in DMF (2 mL) were added Et₃N (0.38 g, 3.74 mmol), and (R)—N—((S)-5-(3,5-difluorophenyl)-6,7-dihydro-5H-pyrrolo[1,2-a]imidazol-2-yl)-2-(3,3-dimethylpiperazin-1-yl)propanamide (0.19 g, 0.47 mmol) at 10° C. EDCI (0.16 g, 0.84 mmol) and HOBt (0.11 g, 0.84 mmol) were subsequently added. The resulting mixture was warmed to 20° C. and stirred for 10 hours. Upon completion, the reaction mixture was quenched with water (6 mL) and extracted with DCM (4 mL×3). The combined organic layers were washed with Na₂CO₃ (1.3 mL, 10% aq.), brine (1.3 mL, 10% aq.), dried over Na₂SO₄, filtered, and concentrated under reduced pressure. The crude residue was triturated in DCM/MTBE (8 mL, v/v=1/3) to give 4-(4-((R)-1-(((S)-5-(3,5-difluorophenyl)-6,7-dihydro-5H-pyrrolo[1,2-a]imidazol-2-yl)amino)-1-oxopropan-2-yl)-2,2-dimethylpiperazine-1-carbonyl)pyridine 1-oxide (Compound 87, 125 mg, 51% yield) as an off-white solid.

| Compound# | Characterization Data |
|---|---|
| 87 | LCMS (ESI) [M + H]⁺: 525.3 |
| | ¹H NMR (400 MHz, DMSO-d₆) δ 9.96 (s, 1H), 8.20 (d, J = 6.6 Hz, 2H), 7.41 (d, J = 6.6 Hz, |
| | 2H), 7.25-7.20 (m, 1H), 6.96-6.95 (m, 3H), 5.40-5.36 (m, 1H), 3.41-3.28 (m, 3H), 3.08-2.38 |
| | (m, 8H), 1.43 (s, 3H), 1.42 (s, 3H), 1.13 (d, J = 6.9 Hz, 3H). |

### Example 88: (R)—N—((S)-5-(3,5-difluorophenyl)-6,7-dihydro-5H-pyrrolo[1,2-a]imidazol-2-yl)-2-(3,3-dimethyl-4-(5-oxo-4,5-dihydropyrazine-2-carbonyl)piperazin-1-yl)propanamide

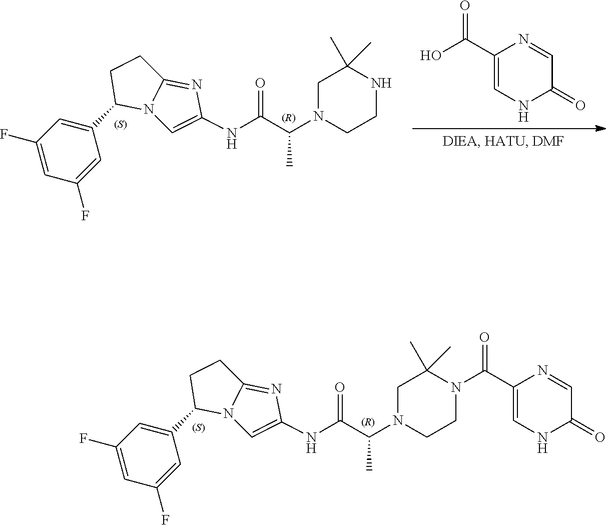

To a solution of 5-oxo-4,5-dihydropyrazine-2-carboxylic acid (0.13 g, 0.94 mmol) in DMF (2 mL) was added DIEA (0.18 g, 1.40 mmol) and HATU (0.32 g, 0.84 mmol) at 10° C. To the resulting solution was added a solution of (R)—N—((S)-5-(3,5-difluorophenyl)-6,7-dihydro-5H-pyrrolo[1,2-a]imidazol-2-yl)-2-(3,3-dimethylpiperazin-1-yl)propanamide (190 mg, 0.47 mmol) and DIEA (0.30 g, 2.34 mmol) in DMF (2 mL) at 10° C. The reaction mixture was stirred at 10° C. for 16 hours. Upon completion, the reaction mixture was quenched with water (6 mL) and extracted with DCM (4 mL×3). The combined organic layers were washed with brine (4 mL, 10% aq.), dried over Na₂SO₄, filtered, and concentrated under reduced pressure. The crude residue was purified by flash column chromatography (silica, eluent: DCM/MeOH=10/1) to afford a brown solid, which was triturated in DCM (6 mL) to afford (R)—N—((S)-5-(3,5-difluorophenyl)-6,7-dihydro-5H-pyrrolo[1,2-a]imidazol-2-yl)-2-(3,3-dimethyl-4-(5-oxo-4,5 -dihydropyrazine-2-carbonyl)piperazin-1-yl)propanamide (Compound 88, 35 mg, 14% yield) as a white solid.

| Compound# | Characterization Data |
|---|---|
| 88 | LCMS (ESI) [M + H]⁺: 526.2 |
| | ¹H NMR (400 MHz, DMSO-d₆) δ 12.56 (s, 1H, bs), 9.98 (s, 1H), 7.91 (s, 1H), 7.71 (s, 1H), |
| | 7.24-7.18 (m, 1H), 6.99 (s, 1H), 6.92-6.90 (m, 2H), 5.41-5.37 (m, 1H), 3.63-3.35 (m, 2H), |
| | 3.33-2.98 (m, 1H), 2.98-2.65 (m, 4H), 2.54-2.50 (m, 1H), 2.40-2.27 (m, 3H), 1.41 (s, 3H), |
| | 1.40 (s, 3H), 1.13 (d, J = 6.9 Hz, 3H). |

### Example 89: Salt Formation, Crystallization and Characterization of Compound 3

### Solubility of Compound 3 Free Base.

The solubility of Compound 3 was estimated by visual observation in 24 single solvents. Approximately 10 mg of solids were weighed into an 8 mL glass vial, and then solvent was added stepwise until solids were dissolved completely or a total of solvent was reached 5 mL. The results are summarized below.

| | Solubility |
|---|---|
| Solvent | (mg/ml) |
| MeOH | 11.1 < s < 12.5 |
| EtOH | 2.8 < s < 3.3 |
| IPA | ≈2.0 |
| Acetone | <2.0 |
| MIBK | <2.0 |
| MEK | ≈2.0 |
| NMP | 25.0 < s < 33.3 |
| EA | <2.0 |
| IPAc | <2.0 |
| MTBE | <2.0 |
| Anisole | <2.0 |
| CPME | <2.0 |
| ACN | <2.0 |
| THF | 8.3 < s < 9.1 |
| 2-MeTHF | <2.0 |
| DMF | 12.5 < s < 14.3 |
| DMAC | 8.3 < s < 10.0 |
| DCM | <2.0 |
| CYH | <2.0 |
| Heptane | <2.0 |
| Toluene | <2.0 |
| DMSO | 20.0 < s < 25.0 |
| H₂O | <2.0 |
| 1,4-Dioxane | 5.0 < s < 6.7 |

### Polymorph Screen of Compound 3 Free Base

A polymorph screening was conducted for the free base of Compound 3. The slurry conversion process included adding 20 mg of Compound 3 into different solvents to make suspensions, which were kept stirring at room temperature for 7 days and 50° C. for 3 days. Solid samples were collected by filtration and analyzed by X-Ray Powder Diffraction (XRPD).

A total of 10 polymorphic Forms were found in this polymorph screening campaign, including Forms I-X. Form I and Form V were selected and evaluated based on hygroscopicity, Solid stability, and solubility.

Hygroscopicity evaluation was performed by Dynamic Vapor Sorption (DVS). Free base Form I, with water uptake of 0.4808%, was in the category of slightly hygroscopic. Free base Form V, with water uptake of 0.6261%, was in the category of slightly hygroscopic. No form changes were observed after the DVS test.

Solid stability showed free base Form I and free base Form V were physically stable with no XRPD change after 10 days.

HPLC showed that the purity of Form I decreased by 0.2% at 4500 lux, and the purity of Form V decreased by 0.11% at 60° C. and 0.60% at 4500 lux, which indicated that Form I was more chemically stable than Form V.

The solubility of Form I and Form V were performed in biorelevant media (water, Fasted State Simulated Intestinal Fluid (FaSSIF) at pH 6.5, FeSSIF at pH 5.0, and SGF at pH 1.2) at 37° C. The result showed that the solubility of Free base Form I was relatively higher in FaSSIF and FeSSIF compared with Form V, and the XPRD pattern of Form I and Form V didn't change after 24 h suspension in biorelevant media (water, FaSSIF at pH 6.5, FeSSIF at pH 5.0, and SGF at pH 1.2) at 37° C.

Form I showed high crystallinity and a good thermal profile though Form I can convert to Form V spontaneously in the MeOH solvent system.

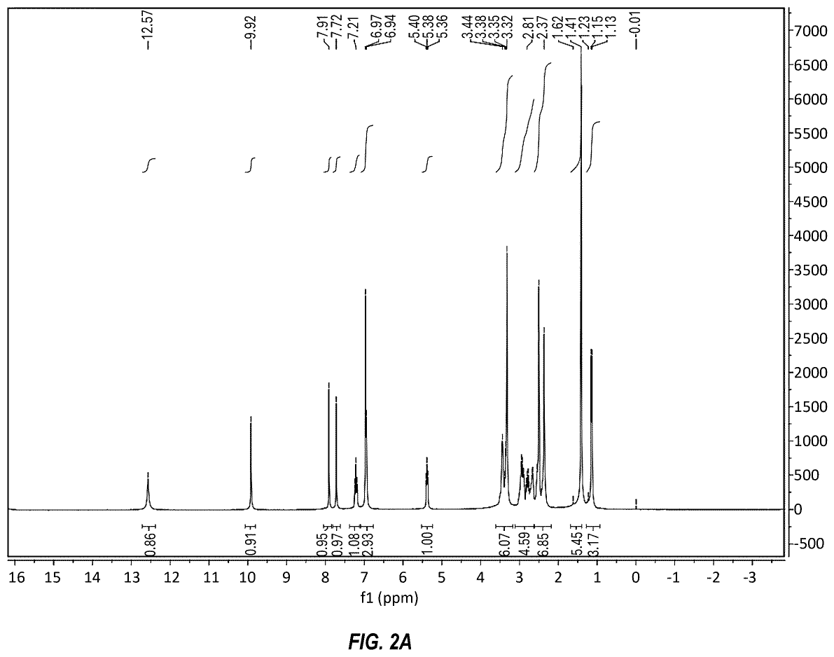

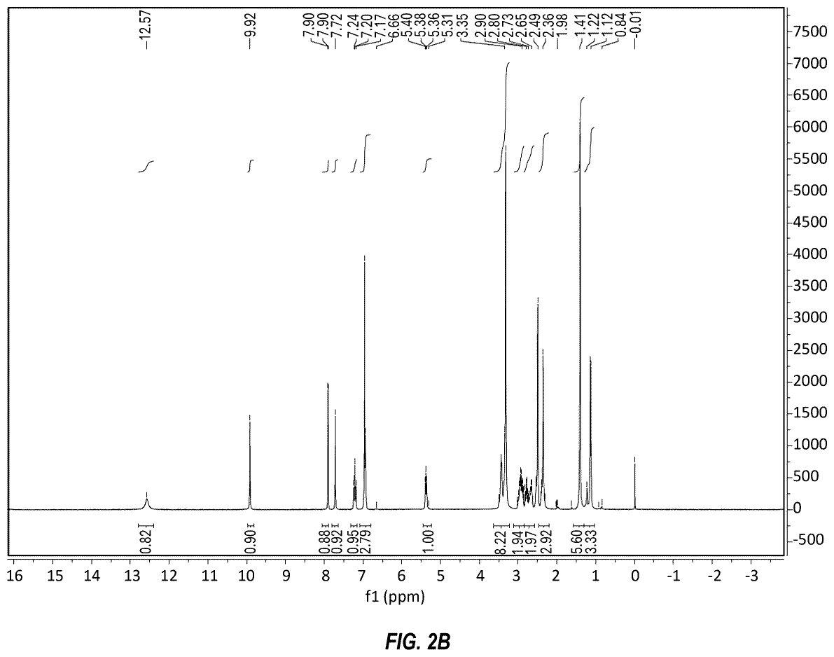

FIGS. 2A and 2B show ¹H-NMR spectra for Compound 3 Forms I and V, respectively. Spectra were collected on a Bruker 300 MHz instrument. Samples were prepared in DMSO-d₆ solvent and measured with the parameters below. The data was analyzed using MestReNova.
Number of scans: 8
Probe: PA BBO 300S1 BBF-H-D-05 Z
Temperature: 297.6K

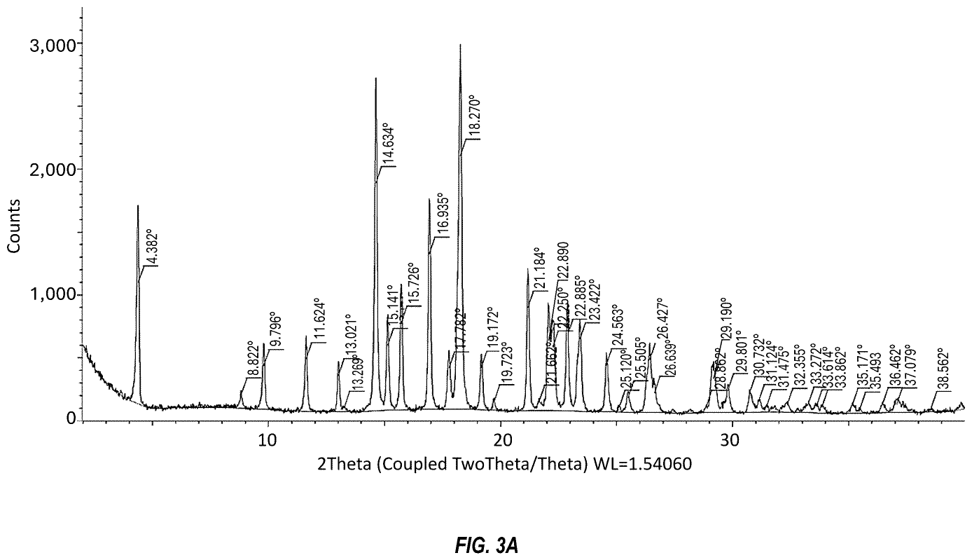

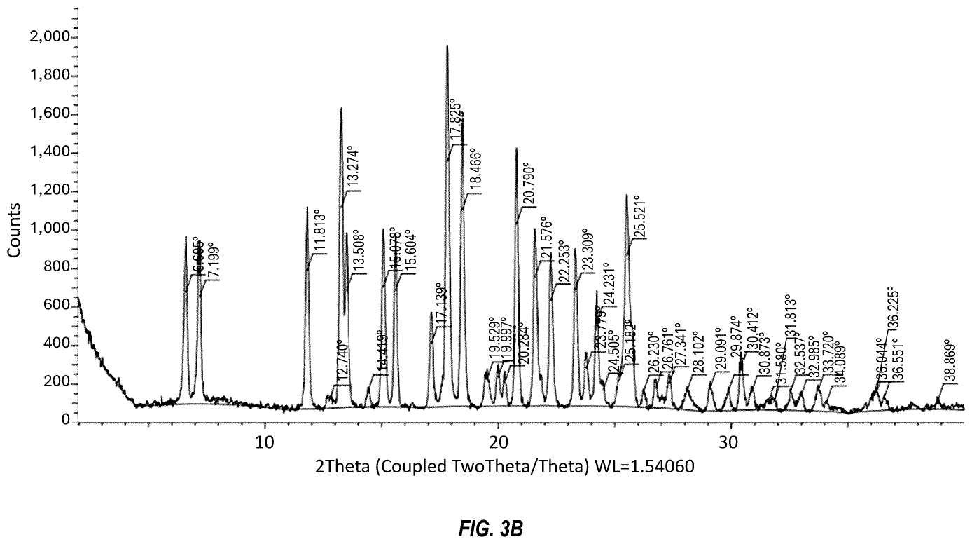

FIGS. 3A and 3B show XRPD results for Forms I and V of Compound 3, respectively. XRPD diffractograms were collected with an X-ray diffractometer. The sample was prepared on a zero-background silicon wafer by gently pressing it onto the flat surface. The parameters of XRPD diffraction are shown below.
Instrument: Bruker D8 Advance
Radiation: Cu, Kαl (λ=1.54056 A)
Detector: LynxEye
Scan angle: 3°-50°
Scan step: 0.02°
Scan speed: 0.2 s
Scan mode: Continuous
Tube voltage/current: 40 KV/40 mA
Divergence slit: 0.6 mm
Sample rotation speed: 15 rpm

| Compound 3 Form I | | | Compound 3 Form V | | |
|---|---|---|---|---|---|
| | | Rel. | | | Rel. |
| Name | Angle | Intensity | Name | Angle | Intensity |
| Peak #1 | 4.382° | 0.48 | Peak #1 | 6.605° | 45.9% |
| Peak #2 | 8.822° | 0.05 | Peak #2 | 7.199° | 43.7% |
| Peak #3 | 9.796° | 0.17 | Peak #3 | 11.813° | 56.4% |
| Peak #4 | 11.624° | 0.21 | Peak #5 | 13.274° | 81.6% |
| Peak #5 | 13.021° | 0.14 | Peak #6 | 13.508° | 47.7% |
| Peak #6 | 13.269° | 0.02 | Peak #8 | 15.078° | 48.9% |
| Peak #7 | 14.634° | 0.90 | Peak #9 | 15.604° | 47.5% |
| Peak #8 | 15.141° | 0.26 | Peak #11 | 17.825° | 100.0% |
| Peak #9 | 15.726° | 0.34 | Peak #12 | 18.466° | 80.2% |
| Peak #10 | 16.935° | 0.61 | Peak #16 | 20.790° | 74.2% |
| Peak #11 | 17.782° | 0.16 | Peak #17 | 21.576° | 52.3% |
| Peak #12 | 18.270° | 1.00 | Peak #18 | 22.253° | 42.9% |
| Peak #13 | 19.172° | 0.17 | Peak #19 | 23.309° | 47.3% |
| Peak #14 | 19.723° | 0.03 | Peak #21 | 24.231° | 33.8% |
| Peak #15 | 21.184° | 0.41 | Peak #24 | 25.521° | 61.6% |

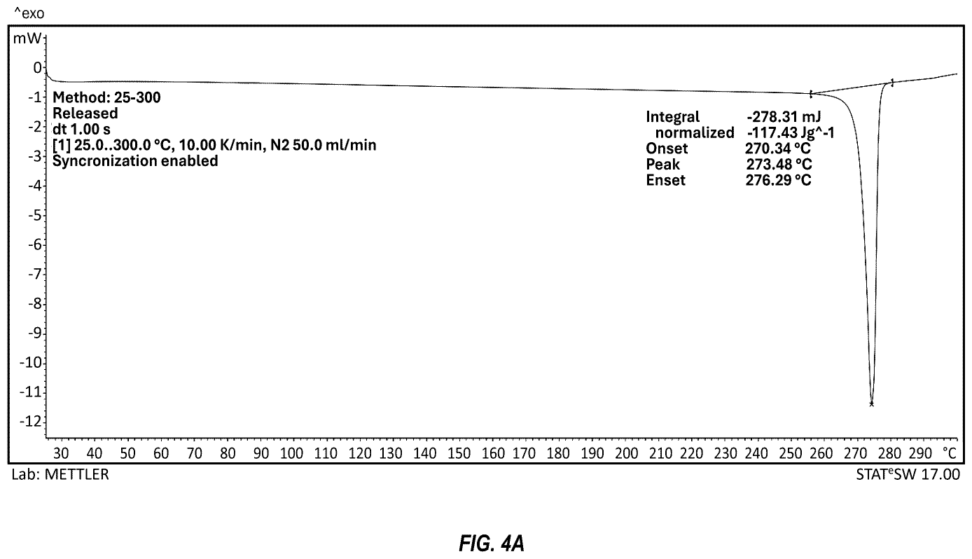

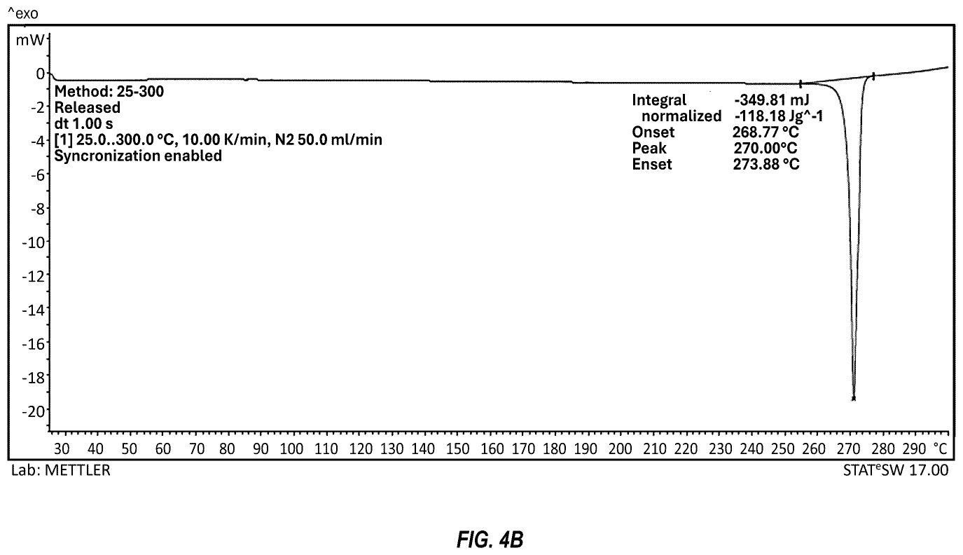

FIGS. 4A and 3B show Differential Scanning Calorimetry (DSC) results for Forms I and V, respectively. DSC analysis was performed with DSC 3 from Mettler Toledo Instrument. About 1-3 mg of a sample was placed into an aluminum pan with a pinhole and heated with the parameters shown below. The data was analyzed using STARe.
Instrument: DSC 3
Sample pan: Aluminum, pin-holed
Temperature range: 25~250° C.
Heating rate: 10 k/min
Purge gas: N2
Flow rate: 50 ml/min.

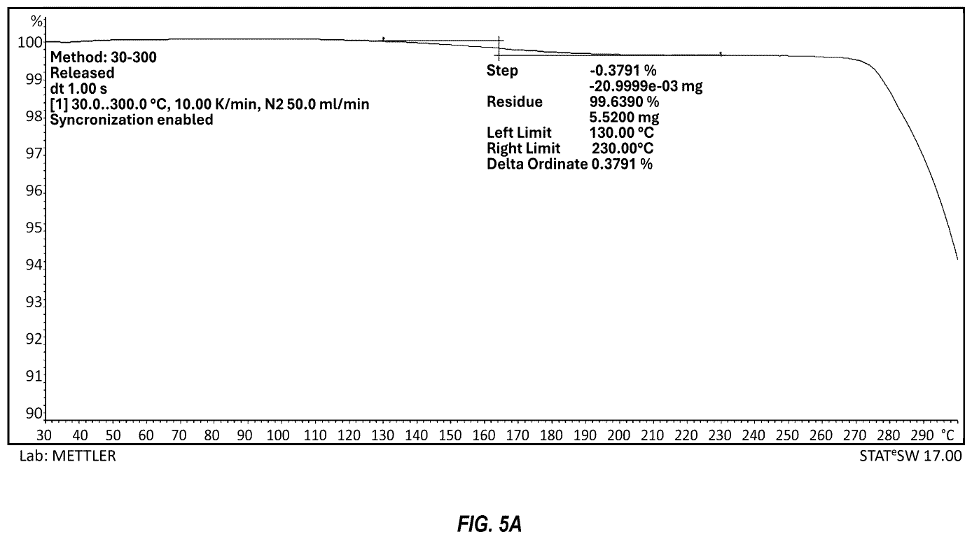

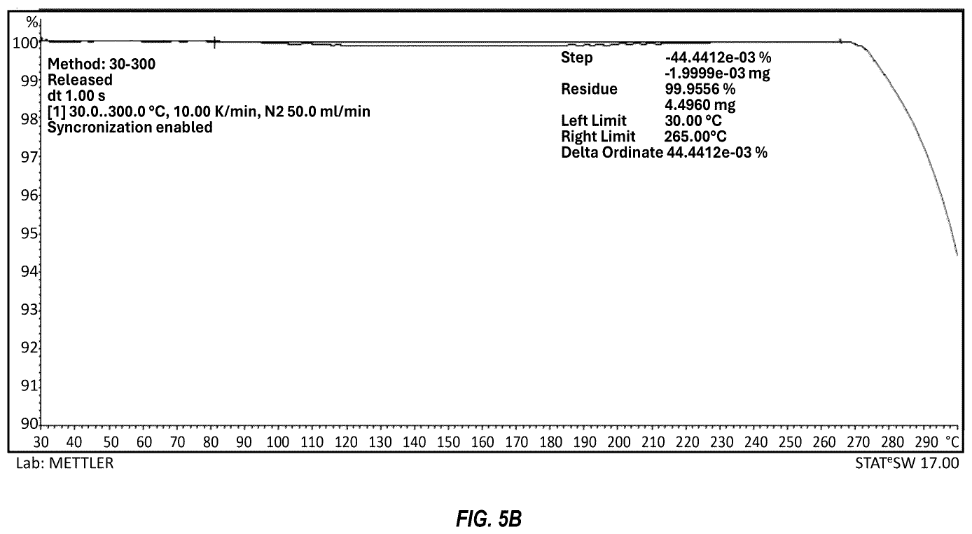

FIGS. 5A and 5B show Thermogravimetric Analysis (TGA) results for Forms I and V, respectively. TGA analysis was performed with TGA 2 from Mettler Toledo Instrument. About 1-5 mg of a sample was loaded onto a pre-tared aluminum oxide pan and heated with the parameters below. The data was analyzed using STARe.
Sample pan: Aluminum oxide, open
Temperature range: 30~300° C.
Heating rate: 10 k/mmn
b Purge gas: N2
Flow rate: 50 mN/min

### Salt Screen

According to the approximate solubility and pKa of the free base, salt screening was performed with 9 acids in 5 solvent systems at room temperature. The scale of salt preparation was 50 mg. The summary of the salt screening is presented in the table below.

Salts of Compound 3 with 5 crystal forms were found by this salt screening experiment, which are shown in bold text in the table below and included Sulfate Pastern I, Mesylate Pattern I and Pattern II, Tosylate Pattern I, and Oxalate Pattern I.

| Lot# | Acid | EtOH | Acetone | EtOAc | MTBE | ACN |
|---|---|---|---|---|---|---|
| 3-1-1~5 | HCl 1.0 eq | Off-white | off-white | off-white | Off-white | N/A |
| | (4M/IPA) | solid; Free | solid; Free | solid; Free | solid; Free | |
| | | base Form I | base Form I | base Form I | base Form I | |
| 3-2-1~5 | HCl 2.0 eq | N/A | yellow solid; | Off-white | Off-white | N/A |
| | (4M/IPA) | | Amorphous | solid; | solid; | |
| | | | | Amorphous | Amorphous | |
| 3-3-1~5 | H₂SO₄ 1.0 eq | Off-white | off-white | off-white | off-white | off-white |
| | | solid; | solid; Sulfate | solid; | solid; Free | solid; |
| | | Sulfate | Pattern | Sulfate | base Form I | Sulfate |
| | | Pattern I | I + Free base | Pattern I | | Pattern I |
| | | | Form I | | | |
| 3-4-1~5 | H₂SO₄ 2.0 eq | N/A | Yellow solid; | off-white | off-white | Pale yellow |
| | | | Amorphous | solid; | solid; | solid; |
| | | | | Amorphous | Amorphous | Amorphous |
| 3-5-1~5 | Methane sulfonic | N/A | N/A | off-white | off-white | off-white |
| | acid 1.0 eq | | | solid; | solid; Free | solid; |
| | | | | Mesylate | base Form I | Mesylate |
| | | | | Pattern I | | Pattern II |
| 3-6-1~5 | Methane sulfonic | N/A | N/A | off-white | off-white | N/A |
| | acid 2.0 eq | | | solid; | solid; | |
| | | | | Amorphous | Amorphous | |
| 3-7-1~5 | L-tartaric acid | off-white | off-white | off-white | off-white | off-white |
| | 1.0 eq | solid; Free | solid; Free | solid; Free | solid; Free | solid; Free |
| | | base form I | base form I | base form I | base form I | base form I |
| 3-8-1~5 | Fumaric acid | off-white | off-white | off-white | Precipitation | off-white |
| | 1.0 eq | solid; Free | solid; Free | solid; Free | off-white | solid; Free |
| | | base form I | base form I | base form I | solid; Free | base form I |
| | | | | | base form I | |
| 3-9-1~5 | Phosphoric acid | Precipitation | Precipitation | Precipitation | Precipitation | Precipitation |
| | 1.0 eq | off-white | off-white | off-white | off-white | off-white |
| | | solid; Free | solid; Free | solid; Free | solid; Free | solid; Free |
| | | base form I | base form I | base form I | base form I | base form I |
| 3-10-1~5 | Citric acid 1.0 eq | off-white | off-white | off-white | off-white | off-white |
| | | solid; Free | solid; Free | solid; Free | solid; Free | solid; Free |
| | | base form I | base form I | base form I | base form I | base form I |
| 3-11-1~5 | p-Toluenesulfonic | Pale yellow | off-white | off-white | off-white | Pale yellow |
| | acid 1.0 eq | solid; Free | solid; | solid; | solid; Free | solid; Free |
| | | base form I | Tosylate | Tosylate | base Form I | base Form I |
| | | | Pattern | Pattern I | | |
| | | | I + Free base | | | |
| | | | Form I | | | |
| 3-12-1~5 | p-Toluenesulfonic | N/A | N/A | Pale yellow | off-white | N/A |
| | acid 2.0 eq | | | solid; Free | solid; Free | |
| | | | | base Form I | base Form | |
| | | | | | I + Amorphous | |
| 3-13-1~5 | Oxalic acid 1.0 eq | off-white | off-white | off-white | off-white | off-white |
| | | solid; Free | solid; Free | solid; | solid; Free | solid; Free |
| | | base Form I | base Form | Oxalate | base Form I | base form I |
| | | | I + 2 peaks | Pattern I | | |

Further characterization data for the different polymorph forms are detailed in the table below.

| | | | | Residual | | |
|---|---|---|---|---|---|---|
| | Experiment | Counter- | Crystallinity | solvent by | | |
| Salt | ID | ion class | by XRPD | 1H-NMR | DSC | TGA |
| Sulfate | 3-3-ACN | I | High | 0.2% | Dehydration | 4.5688%@30° |
| Pattern I | | | | ACN | Tₒₙₛₑₜ: 92.52° C.; Melting | C.-140° C.; |
| | | | | | Tₒₙₛₑₜ: 189.78° C. | |
| Mesylate | 3-5-EA | II | Medium | 5.23% EA | Dehydration or | 4.1591%@30° |
| Pattern I | | | | | Desolvation | C.-130° C.; |
| | | | | | Tₒₙₛₑₜ: 27.36° C.; Melting | 2.6591%@130° |
| | | | | | Tₒₙₛₑₜ: 149.87° C. | C.-175° C. |
| Mesylate | 3-5-ACN | II | High | 0.26% | Dehydration | 5.2743%@30° |
| Pattern II | | | | ACN | Tₒₙₛₑₜ: 43.03° C.; Melting | C.-160° C.; |
| | | | | | Tₒₙₛₑₜ: 168.38° C. | |
| P-tosylate | 3-11-EA | II | High | 6.49% EA | Dehydration or | 5.4102%@30° |
| Pattern I | | | | | Desolvation | C.-140° C.; |
| | | | | | Tₒₙₛₑₜ: 84.22° C.; Melting | |
| | | | | | Tₒₙₛₑₜ: 154.36° C. | |
| Oxalate | 3-13-EA | II | Medium | 12.52% EA | Two Dehydration or | 7.2289@30° |
| Pattern I | | | | | Desolvation | C.-170° C.; |
| | | | | | Tₒₙₛₑₜ: 28.76° C.; | |
| | | | | | Tₒₙₛₑₜ: 104.50° C. | |
| | | | | | Melting Tₒₙₛₑₜ: 161.01° C. | |

### Example 90: Salt Formation, Crystallization and Characterization of the Tartrate Salt of Compound 1

Dissolve 2.0 g Compound 1 freebase in 10 vol EtOH at 25° C. Dissolve 1.1 eq L-tartaric acid in 4 vol EtOH at ambient temperature (25-30° C.). Charge 1 vol acid solution (0.28 eq acid) into R1. Load 0.2% seed (Pattern I, small particle size). Stir for 1.0 h at 25° C. Charge the remaining acid into R1 in 0.5 h (XRPD: Pattern I). Hold for 1.0 h at 25° C. and cooled to 15° C. in 1.0 h and held for 3 h. Filter the suspension and wash with 2 vol EtOH (pre-cooled). Dry the wet cake at 50° C. for 20 h. Obtain 2.43 g of white solid. Yield by solid: 940%; Purity: 100.00%; Residual solvent: EtOH: 2580 ppm; Particle Size D90: 11.7 μm; Tartaric acid content by IC: 20.5%.

Crystallization in pure EtOH (single solvent) delivered a qualified product with Pattern I, 100.0% purity, 93-94% yield, and low residual solvents, which is a recommended crystallization system. Based on the acid equivalent investigation, the recommended L-tartaric acid range was 1.05 eq-1.30 eq.

For the particle size control by process, 0.2%-1% milled seeds were applied to the process, and a small particle size (D90: 27.3-29.1 μm) was delivered

The solubility of Compound 1/L-tartrate salt was measured in single solvents at different temperatures by the gravimetric/visual method after equilibrium for overnight (~20 h). The supernatant was collected for solubility, and the residual solid was measured by XRPD. The results are presented below. The data indicated that Compound 1/L-tartrate had high solubility in DMSO, some solubility in MeOH, while poor solubility in all other studied solvents.

| | 50° C. | | 25° C. | |
|---|---|---|---|---|
| | Solubility | | Solubility | |
| Solvents | (mg/mL) | XRPD | (mg/mL) | XRPD |
| MeOH | 64 | Pattern I | 27 | Pattern I |
| EtOH | 15 | Pattern I | 5 | Pattern I |
| IPA | 7 | Pattern I | 2 | Pattern I |
| Acetone | 3 | Pattern I | 1 | Pattern I |
| MEK | 1 | Pattern I | 1 | Pattern I |
| MIBK | 1 | Pattern I | 1 | Pattern I |
| EA | 2 | Pattern I | 1 | Pattern I |
| IPAc | 2 | Pattern I | 1 | Pattern I |
| THF | 5 | Pattern I | 4 | Pattern I |
| 2-MeTHF | 2 | Pattern I | 2 | Pattern I |
| MtBE | 2 | Pattern I | 1 | Pattern I |
| n-heptane | ~0 | Pattern I | ~0 | Pattern I |
| Water | 10 | Freebase | 9 | Freebase |
| | | Form II | | Form II |
| | | (dissociation) | | |
| DMSO* | >200 | N/A | 100-125 | Type VI |
| | | | | (dissociation) |

To evaluate the solution stability of Compound 1/L-tartrate, three experiments in MeOH, EtOH, and DMSO at 55° C. were conducted, and the purity at different times was tracked. The data indicated that the Compound 1/L-tartrate was stable in MeOH and EtOH at 55° C. for 24 h and basically stable in DMSO and water at 55° C. for 6 h; it degraded at 55° C. after 24 h.

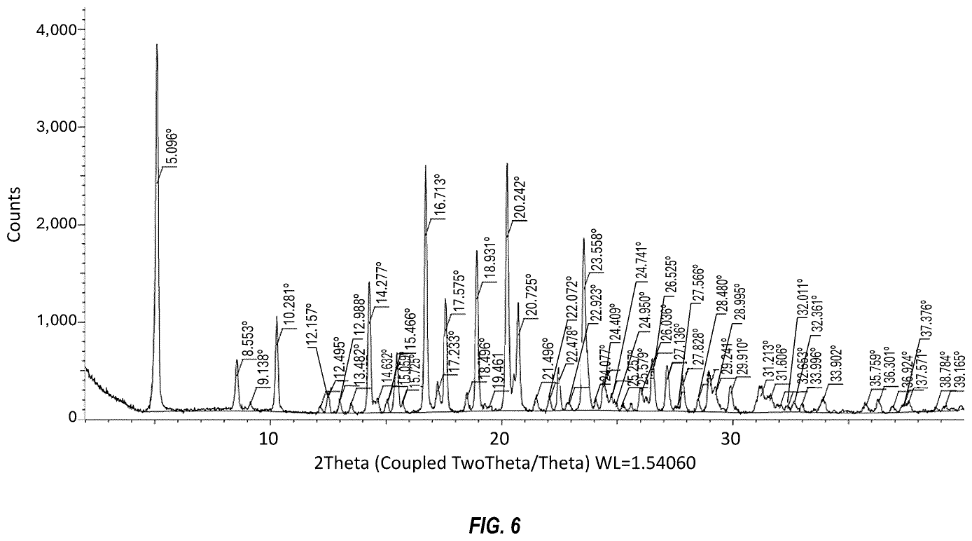

A drying stability study at 60° C. under vacuum was conducted to evaluate the stability of Compound 1/L-tartrate during the drying process. The data indicate that Compound 1/L-tartrate is both chemically and physically (XRPD) stable during drying under vacuum at 60° C. for 6 days. To evaluate the stability of Compound 1/L-tartrate at ambient conditions (25-30° C./40-70 RH %), the stability of the wet solid at ambient conditions was carried out. The data indicates that the compound is both chemically and physically (XRPD) stable at ambient conditions for 6 days. FIG. 6 shows the XRPD pattern for Compound 1/L-tartrate. The table below provides exemplary XRPD peaks.

Compound 1 L-Tartrate salt

| | Angle | Rel. |
|---|---|---|
| Name | (2θ) | Intensity |
| Peak #1 | 5.096° | 100.0% |
| Peak #2 | 8.553° | 15.3% |
| Peak #4 | 10.281° | 29.2% |
| Peak #6 | 12.495° | 6.7% |
| Peak #9 | 14.277° | 39.3% |
| Peak #12 | 15.466° | 18.7% |
| Peak #14 | 16.713° | 77.6% |
| Peak #15 | 17.233° | 9.1% |
| Peak #16 | 17.575° | 35.6% |
| Peak #17 | 18.496° | 5.6% |
| Peak #18 | 18.931° | 49.3% |
| Peak #20 | 20.242° | 76.0% |
| Peak #21 | 20.725° | 33.1% |
| Peak #24 | 22.478° | 12.5% |
| Peak #26 | 23.558° | 53.0% |

### Biological Examples

The utility of the compounds of the present invention and the salts of such compounds as medical agents in the treatment of the above described disease/conditions in mammals (e.g. humans, male or female) is demonstrated by the activity of the compounds of the present invention in one or more of the conventional assays and in vivo assays described and noted below. The in vivo assays (with appropriate modifications within the skill in the art) can be used to determine the activity of other agents as well as the compounds of the present invention. Thus, the protocols described below can also be used to demonstrate the utility of the combinations of the compounds of the present invention. The assays and models may also demonstrate particular other property advantages e.g., side effect profile; half-life. In addition, such assays provide a means whereby the activities of the compounds of the present invention and the salts of such compounds (or the other agents described herein) can be compared to each other and with the activities of other known compounds. The results of these comparisons are useful for determining dosage levels in mammals, including humans, for the treatment of such diseases.

Absorption, Distribution, Metabolism and Excretion (ADME) and pharmacokinetics (PK) of compounds and exemplary assays are discussed in the on-line publication by Thomas D. Y. Chung, David B. Terry and Layton H. Smith “In Vitro and In Vivo Assessment of ADME and PK Properties During Lead Selection and Lead Optimization—Guidelines, Benchmarks and Rules of Thumb—(https://www.ncbi.nlm.nih.gov/books/NBK326710/) Exemplary in vivo animal models of atopic dermatitis are included in J. Invest Dermal. 2009 January: 129(1): 31-40. Exemplary pruritus mouse models are described in Front Med (Lausanne) 2021 Feb. 23; 8:630237 “Comparative Study on Different Skin Pruritus Mouse Models.” Exemplary in vivo animal models of inflammation are included in Int. J. Mol. Si 2019 September; 20(18): 4367. Exemplary in vivo models of autoimmune disease/conditions are included in Autoimmun Rev. 2018 May; 17(5): 473-479. Exemplary in vitro assays of cancer are described in Front Bioeng Biotechnol 2016; 4:12 “In Vitro Tumore Models: Advantages, Disadvantages, Variables, and Selecting the Right Platform.” Exemplary in vivo animal models of cancer are described in Front Oncol, 2019; 8: 429 “Next-Generation in vivo Modeling of Human Cancers. Exemplary in vivo animal models of pain are described in Journal of Neuroscience Methods Volume 348, January 2021, 108997, “Animal models of pain: Diversity and benefits” and J. Pain. 2013 November: 14(11): 10.1016/j.jpain.2013.06.008” An overview of animal models of pain: disease models and outcome measures”.

The following protocols may be varied by those skilled in the art.

### MRGPRX2 Activity Assay:

FlpIn TRex-MRGPRX2-HEK293 cells (herein TRex-MRPGRX2-HEK) stably transfected to express human MRGPRX2 were maintained at 37° C. with 5% CO2 and grown in DMEM supplemented with 10% FBS, 1% L-Glutamate, 1% penicillin/streptomycin, 100 μg/mL Hygromycin B, and 15 μg/mL Blasticidin.

GOI (MRGPRX2) expression was induced with 100 ng/mL of Doxycycline 18-24 hours prior to experiment, in flask. On the day of experiment, cells were lifted with TripLE Express and seeded in 384-well white walled opaque bottomed, assay plates, at 8,000 cells per well in 5 μL freshly made assay buffer (HBSS, 20 mM HEPES, 0.1% BSA and 10 mM LiCl). For dose-response assays, compounds solubilized in 100% DMSO at 30 mM were serially diluted (1:4) first in 100% DMSO, and an intermediate plate of assay buffer was used to establish a final top concentration of 30 μM (in assay plate). For single point assays, dilution of DMSO stocks to 250 nM (in assay plate) was performed in the same manner as described for dose-response assays. Final DMSO concentration was kept constant at 1% across assay plates. Cortistatin-14, at 30 mM stock concentration was solubilized in HBSS+0.1% BSA, stored at −20° C. in 5 μL aliquots and discarded after one freeze-thaw cycle. Final concentration of agonist used in antagonist mode assay was 1 μM, estimated EC80 (EC50 of Cortistatin-14~2.5e-7 M).

Antagonist equilibrium was established with 30 minutes preincubation, followed by no wash, direct addition, and stimulation of agonist for 1 hour at 37° C./5% CO2. IP-1 standards and HTRF detection reagents were added according to the IP-one Gq kit protocol purchased from CisBio (part number 62IPAPEJ). The plate was read on an EnVision XCite plate reader. The HTRF ratio was then calculated from raw data (channel 1/channel 2×10,000) and analyzed using either GraphPad Prism or a custom Python script to calculate IC50 using Log[Antagonist] vs. response (4 parameter) nonlinear regression (dose-response assays) or % control IP1 concentration.

Activity data for selected MRGPRX2 antagonist (versus 1 μM Cortistatin-14 agonist) are displayed in the table below.

To characterize antagonist activity of candidate compounds on human MRGPRX2, HEK293 cells stably expressing the receptor were incubated with DAT compounds and stimulated with MRGPRX2 peptide agonists. Receptor activation was quantified by assay of the second messenger molecule inositol monophosphate (IP1). MRGPRX2-expressing cells responded to agonist stimulation, including cortistatin-14, with expected potencies. Compounds prepared and tested within produced potent dose-dependent inhibition of agonist-stimulated MRGPRX2 activity.

### LAD2 Degranulation Assay:

LAD-2 cells were maintained at 37° C. with 5% CO₂ and grown in complete StemPro-34 media supplemented with 1% L-Glutamate, 1% penicillin/streptomycin, and 250 ng/mL human SCF.

18-24 hours prior to experiment, LAD-2 cells were collected and transferred to growth media without SCF. On the day of experiment, cells were collected by centrifugation and seeded in 384-well assay plates, at 4,000 cells per well in 17 ml freshly made assay buffer (10 mM HEPES, 137 mM NaCl, 2.7 mM KCl, 1.8 mM CaCl₂), 1.0 mM MgCl₂, 5.6 mM glucose, 0.4 mM NaH₂PO₄, 0.04% BSA, pH=7.40). For dose-response assays, compounds solubilized in 100% DMSO at 30 mM were serially diluted (1:3) first in 100% DMSO, and 2 intermediate dilutions with assay buffer were used to establish a final top concentration of 3 mM (in assay plate). For single point assays, dilution of DMSO stocks to 250 nM (in assay plate) was performed in the same manner as described for dose-response assays. Final DMSO concentration was kept constant at 0.1% across plates for all assays. Cortistatin-14 at 30 mM stock concentration was solubilized in HBSS+0.1% BSA, stored at −20° C. in 5 mL aliquots and discarded after one freeze-thaw cycle. Final concentration of agonist used in both dose-response and single point assays was 0.5 mM, prepared in assay buffer.

Antagonist equilibrium was established with 2-hour preincubation, followed by no wash, direct addition, and stimulation of agonist for 30 minutes at 37° C. For detection of 0-hexosaminidase activity, assay plates were centrifuged and 5 mL of cell-free supernatant from each assay well was transferred to a black-walled black-bottomed 384-well plate. Substrate solution (1 mM 4-methylumbelliferyl N-acetyl-β-D-glucosaminide in citrate buffer, pH=4.5, 15 ml) was added to each well and plates were incubated for 1 hour at 37° C. The plate was read on an EnVision XCite plate reader for umbelliferone fluorescence intensity (λex=355 nm, λem=460 nm). Background-subtracted data was analyzed using either GraphPad Prism or a custom Python script to calculate IC₅₀ using either Log[Antagonist] vs. response (4 parameter) nonlinear regression (dose-response assays).

To characterize antagonist activity of candidate compounds on human mast cell degranulation, immortalized human mast cells (LAD2) were incubated with DAT compounds and stimulated with MRGPRX2 peptide agonists. MRGPRX2-stimulated degranulation was quantified by assay of secreted beta-hexosaminidase activity. LAD2 mast cells responded to agonist stimulation, including cortistatin-14, with expected potencies. Compounds prepared and tested within produced potent dose-dependent inhibition of LAD2 degranulation.

TABLE 3
Results of MRGPRX2 Activity Assay and LAD2 Degranulation Assay

| | | | LAD2 |
|---|---|---|---|
| | MRGPRX2 | MRGPRX2 | Degranulation |
| | Activity Assay | Activity Assay | Assay |
| Compound | IC₅₀ | % Control | IC₅₀ |
| # | (nM) | @ 250 nM | (nM) |
| 1 | 4.9 | 9.7 | 3.0 |
| 2 | 3.1 | | 3.6 |
| 3 | 3.1 | 7.1 | 0.9 |
| 4 | 3.9 | 8.7 | 0.7 |
| 5a | 1.2 | | 1.9 |
| 5b | >750 | | |
| 6a | 2.2 | | 1.5 |
| 6b | >750 | | >750 |
| 7a | 1.2 | 6.9 | 1.1 |
| 7b | 327 | | |
| 8a | | | >750 |
| 8b | | 7.8 | 0.3 |
| 9a | | 8.1 | 0.5 |
| 9b | | | >750 |
| 10a | | | >750 |
| 10b | | | >750 |
| 11a | 6.6 | | 84 |
| 11b | >6000 | | >750 |
| 12 | | | 193 |
| 13 | 1.1 | 7.0 | 0.5 |
| 14a | 1.6 | | 1.0 |
| 14b | >750 | | |
| 15a | 1.3 | 7.5 | 0.1 |
| 15b | 166 | | |
| 16 | 280 | | |
| 17 | >750 | | |
| 18 | >750 | | |
| 19 | 5.7 | 8.0 | 3.8 |
| 20 | 108 | | |
| 21 | 110 | | |
| 30 | | 36 | |
| 31 | | 10 | 5.8 |
| 32 | | 9.4 | 3.4 |
| 33 | | 8.8 | 4.9 |
| 34 | | 13 | |
| 35 | | 51 | |
| 36 | | 14 | |
| 37 | | 9.3 | 2.4 |
| 38 | | 13 | 9.7 |
| 39 | | 85 | |
| 40 | | 79 | |
| 41 | | 71 | |
| 42 | | 55 | |
| 43 | | 9.2 | 6.8 |
| 44 | | 9.0 | 14 |
| 45 | | 37 | |
| 46 | | 9.0 | 5.5 |
| 47 | | 14 | |
| 48 | | 8.4 | 7.0 |
| 49 | | 7.7 | 1.0 |
| 50 | | 12 | |
| 51 | | 27 | |
| 52 | | 13 | |
| 53 | | 18 | |
| 54 | | 8.7 | 3.3 |
| 55 | | 10 | 14 |
| 56 | | 9.3 | 3.7 |
| 57 | | 56 | |
| 58 | | 9.9 | 1.5 |
| 59 | | 60 | |
| 60 | | 9.1 | 3.7 |
| 22 | 12.5 | | 10.4 |
| 23 | 18.8 | | 9.1 |
| 24 | 4.5 | | 3.0 |
| 27 | 158 | | 220 |
| 61 | | 9.1 | 1.6 |
| 62 | | 9.6 | 0.3 |
| 63 | | 12 | |
| 64 | | 9.4 | 9.9 |
| 65 | | 38 | |
| 66 | | 17 | |
| 67 | | 86 | |
| 68 | | 14 | |
| 69 | | 41 | |
| 70 | | 52 | |
| 26 | 3.7 | | 4.9 |
| 28 | 6.3 | | |
| 29a | 13.5 | | |
| 29b | >750 | | |
| 25 | 1.7 | | 2.9 |
| 71 | | 26 | |
| 72 | | 8.9 | 8.1 |
| 73 | | 10 | 6.4 |
| 74 | | 77 | |
| 75 | | 35 | |
| 76 | | 11 | |
| 77 | | 40 | |
| 78 | | 9.7 | 9.4 |
| 79 | | 35 | |
| 80 | | 37 | |
| 81 | | 53 | |
| 82 | | 72 | |
| 83 | 70 | | |
| 84 | 2.9 | | |
| 85 | >750 | | |
| 86 | >750 | | |
| 87 | >750 | | |
| 88 | >750 | | |

### Mast Cell Degranulation—In Vivo

Validation of MRGPRX2-KI mice

MRGPRX2 knock-in mice (X2-KI) were generated and licensed from Shanghai Model Organisms (SMOC) and bread at Taconic. For validation, 8- to 10-week-old MRGPRX2 transgenic MRGPRX2 KI mice were sacrificed and subject to peritoneal lavage with 5 mL of cold PBS (Phosphate-Buffered Saline). Cells were washed twice with FACS Staining Buffer, incubated with mouse TruStain FcX anti-mouse CD16/32 blocking solution, anti-mouse CD117-APC (Biolegend, Cat #17-1171-83, clone 2B8), anti-mouse Fcε1a BV421 (Biolegend Cat #750852, clone mar-1) and anti-human MRGPRX2-PE Pecy7 (Biolegend Cat #359008, clone K125H4) or isotype control (Biolegend 400326, clone MPC-11) for 30 min on ice and protected from light. Samples were washed 3 times in FACS staining buffer, resuspended in 200 μL buffer and run on an AttuneNxT flow cytometer. Live cells were detected with Live/Dead Fixable Dead Cell Stain Aqua, and peritoneal mast cells were identified and gated based on the expression of CD177 and FcεR1a.

### In Vivo Vascular Permeability Assay

For the vascular permeability assays approximately 8- to 10-week-old X2-KI mice were injected intravenously (IV) with 100 μL of 1% Evans Blue in 0.9% saline, before being placed under isoflurane anesthesia. Fifteen minutes after IV injections, animals were injected intradermally with 30 μL of vehicle PBS or cortistatin-14 (C-14) (10 mg/mL; Tocris) in the ear.

Fifteen minutes later, animals were sacrificed. The entire ear was excised, weighed, and placed in 1.5 mL Eppendorf tubes. Evans Blue dye was extracted from the tissue by adding 1 mL of formamide, vortexing, and incubating the tissue at 55° C. overnight. Extracted dye in formamide was placed into a 96-well plate in duplicate along with a standard curve of known Evans Blue concentration standards. The plate was read at 620 nM on a plate reader, and the results were expressed as ng of dye per mg tissue. Compound 1 was dosed PO (by mouth) at 3, 6 and 15 mg/kg (mpk).

### CONCLUSIONS

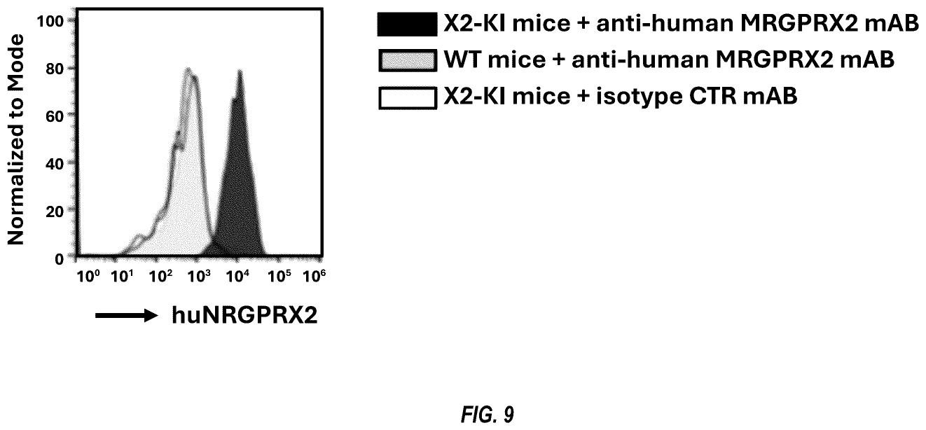

Murine orthologs to human MRGPRX2 have moderate to low sequence identity compared to human (50-69%) and while they share some agonists, the potencies are largely shifted. Therefore, mice that express human MRGPRX2 (X2-KI) were generated and it was confirmed that human MRGPRX2 in expressed in murine mast cells (FIG. 9).

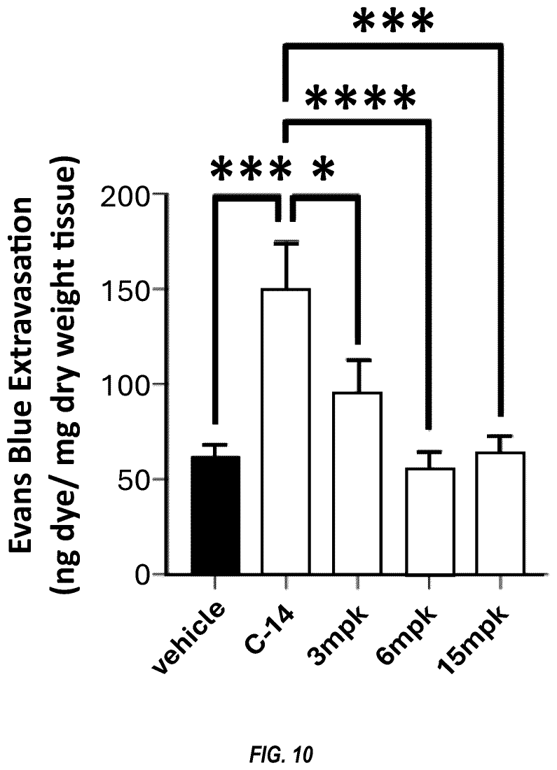

To investigate whether Compound 1 inhibits mast cell degranulation in vivo, a vascular permeability assay using Evans Blue dye was performed. Evans blue leakage from blood vessels into ear tissue is a process that is triggered by mast cell degranulation. Intradermal injection of cortistatin-14 into the ear of X2-KI mice induced mast cell degranulation in the skin, as measured by extravasation of Evans Blue dye into the ear. Oral treatment of X2-KI mice with Compound 1 showed a dose-dependent inhibition of Evans Blue extravasation in the ear (FIG. 10), consistent with inhibition of mast cell degranulation.

All publications, including but not limited to, issued patents, patent applications, and journal articles, cited in this application are each herein incorporated by reference in their entirety.

Although the invention has been described above with reference to the disclosed embodiments, those skilled in the art will readily appreciate that the specific experiments detailed are only illustrative of the invention. It should be understood that various modifications can be made without departing from the spirit of the invention. Accordingly, the invention is limited only by the following claims.

1. A compound of Formula (I) or a pharmaceutically acceptable salt thereof: 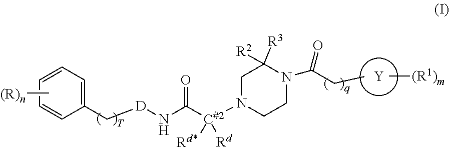
wherein
T is 1 or 0; provided that when T is 1, D is a 5-membered heteroaryl group having one or two heteroatoms selected from nitrogen and oxygen, and D is unsubstituted or is mono-substituted with (C₁-C₄)alkyl, mono-, di-, or tri-fluoromethyl; and
when T is 0, D is 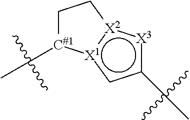
and;
X¹ and X² are each independently N or C;
X³ is N or CH;
C^{#1} is a first stereocenter;
C^{#2} is a second stereocenter;
n is 1, 2 or 3;
each R is independently H or halo;
either R^{d*} is H and R^{d} is methyl or R^{d*} is methyl and R^{d} is H;
Y is absent or Y is selected from
a 5- or 6-membered heteroaryl group having one to three heteroatoms independently selected from nitrogen, oxygen, and sulfur;
a 4- to 6-membered heterocycloalkyl group having 1 to 2 heteroatoms independently selected from nitrogen, oxygen, and sulfur;
a phenyl ring fused to a 5- or 6-membered heteroaryl group to form a bicyclic group having one to three heteroatoms independently selected from nitrogen, oxygen, and sulfur;
a 5- or 6-membered heteroaryl group fused to a 5- or 6-membered heteroaryl group to form a bicyclic group having one to five heteroatoms independently selected from nitrogen, oxygen and sulfur;
a 5- to 7-membered heterocycloalkyl group fused to a 5- or 6-membered heteroaryl group to form a bicyclic group having one to four heteroatoms independently selected from nitrogen, oxygen and sulfur;
a C₅-C₇)cycloalkyl group fused to a 5- or 6-membered heteroaryl group to form a bicyclic group, said 5- or 6-membered heteroaryl group having one to three heteroatoms independently selected from nitrogen, oxygen and sulfur;
a phenyl group;
a (C₄-C₇)cycloalkyl group; or
a phenyl group fused to a 5- to 7-membered heterocycloalkyl group to form a bicyclic group, said heterocycloalkyl group having one or two heteroatoms independently selected from nitrogen, oxygen and sulfur;
m is 0, 1 or 2;
each R¹ is independently H, oxo, amino, cyano, halo, hydroxy, (C₁-C₄)alkyl, (C₃-C₄)cycloalkyl, (C₂-C₄)alkenyl, (C₁-C₄)alkoxy, mono-, di- or tri-fluoromethyl, hydroxy(C₁-C₄)alkyl, aminosulfonyl, methylsulfonylamino, (C₁-C₄)alkylsulfonyl, mono-N, or di-N,N—(C₁-C₄)amino, piperazinylcarbonyl, pyridinyl, pyrazinylcarbonyl, or R¹ forms an N-oxide when bonded to a ring nitrogen;
R² and R³ are H or R² and R³ are methyl; and
q is 0, 1 or 2.

2.-7. (canceled)

8. The compound of claim 1, wherein D is

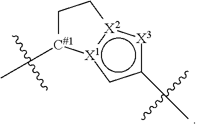

9. The compound of claim 1, wherein R is halo.

10. The compound of claim 9, wherein R is fluoro.

11. The compound of claim 1, wherein n is 2.

12. The compound of claim 1, wherein, X¹ and X³ are N and X² is C.

13. The compound of claim 1, wherein X² and X³ are N and X¹ is C.

14. The compound of claim 1, wherein the first stereocenter is in an R configuration.

15. The compound of claim 1, wherein the first stereocenter is in an S configuration.

16. The compound of claim 1, wherein the second stereocenter is in an R configuration.

17. The compound of claim 1, wherein the second stereocenter is in an S configuration.

18. (canceled)

19. The compound of claim 1, wherein R² and R³ are methyl.

20. The compound of claim 1, wherein q is 0.

21. (canceled)

22. (canceled)

23. The compound of claim 1, wherein Y is a 5- or 6-membered heteroaryl group having one to three heteroatoms independently selected from nitrogen, oxygen, and sulfur.

24. The compound of claim 1, wherein Y is a 4- to 6-membered heterocycloalkyl group having one or two heteroatoms independently selected from nitrogen, oxygen and sulfur.

25.-31. (canceled)

32. The compound of claim 1, wherein R¹ is oxo or R¹ forms an N-oxide when bonded to a ring nitrogen.

33. The compound of claim 1, wherein R¹ is oxo or R¹ forms an N-oxide when bonded to a ring nitrogen; and m is 1.

34. (canceled)

35. The compound of claim 1, wherein Y is

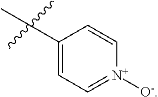

36. (canceled)

37. The compound of claim 1, wherein Y is

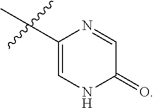 [SMILES: *c1c[nH]c(=O)cn1]

38.-45. (canceled)

46. The compound of claim 1, wherein D is 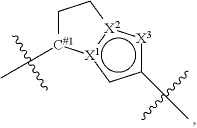
R is halo, n is 2, X¹ and X³ are N, X² is C, and q is 0 or 1.

47. (canceled)

48. The compound of claim 1, wherein D is 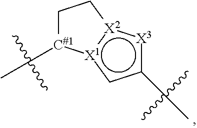
R is halo, n is 2, X² and X³ are N, X¹ is C and q is 0 or 1.

49. (canceled)

50. The compound of claim 1, wherein Y is a 5- or 6-membered heteroaryl group having one or two nitrogen, R¹ is oxo or R¹ forms an N-oxide when bonded to a ring nitrogen, and m is 1.

51. (canceled)

52. A compound chosen from
(S)—N—((R)-5-(3,5-difluorophenyl)-6,7-dihydro-5H-pyrrolo[1,2-a]imidazol-2-yl)-2-(3,3-dimethyl-4-(4-methyl-5-oxo-4,5-dihydropyrazine-2-carbonyl)piperazin-1-yl)propanamide; and
(S)—N—((R)-5-(3,5-difluorophenyl)-6,7-dihydro-5H-pyrrolo[1,2-a]imidazol-2-yl)-2-(3,3-dimethyl-4-((R)-5,6,7,8-tetrahydro-[1,2,4]triazolo[4,3-a]pyridine-6-carbonyl)piperazin-1-yl)propanamide,
or a pharmaceutically acceptable salt thereof.

53.-57. (canceled)

58. A pharmaceutical composition comprising a compound and/or a pharmaceutically acceptable salt of claim 1 and a pharmaceutically acceptable excipient.

59. A method of modulating MRGPRX2 activity, the method comprising administering an effective amount of a compound of claim 1 or a pharmaceutically acceptable salt thereof.

60. A method of modulating mast cell degranulation comprising administering an effective amount of a compound of claim 1 or a pharmaceutically acceptable salt thereof.

61. A method of treating, preventing, or ameliorating an MRGPRX2-mediated disease or disorder in a subject in need thereof comprising administering to the subject an effective amount of a compound of claim 1 or a pharmaceutically acceptable salt thereof.

62. The method of claim 61, wherein the disease is chosen from chronic spontaneous urticaria, mastocytosis, cold urticaria, atopic dermatitis, Asian atopic dermatitis, European atopic dermatitis, rosacea, autoimmune diseases, Crohn's disease, ulcerative colitis, irritable bowel syndrome, rheumatoid arthritis, fibromyalgia, endometriosis, nasal polyps, neuropathic pain, inflammatory pain, pseudo-allergic drug reactions, chronic itch, drug-induced anaphylactoid reactions, metabolic syndrome, esophagus reflux, asthma, cough, migraine, sinusitis, urticaria, chronic inducible urticaria, chronic pruritus, acute pruritus, prurigo nodularis, osteoarthritis, pseudo anaphylaxis, contact urticaria, lupus erythematosus (SLE), psoriasis, psoriatic arthritis, bronchial asthma, systemic mastocytosis, cutaneous mastocytosis, mastocytic enterocolitis, mast cell activation syndrome (MCAS), interstitial cystitis, food allergy, allergic rhinitis, microbial infection, eosinophilic esophagitis (EOE) and chronic pain.

63. A method of treating a condition chosen from chronic spontaneous urticaria, mastocytosis, cold urticaria, atopic dermatitis, Asian atopic dermatitis, European atopic dermatitis, rosacea, autoimmune diseases, Crohn's disease, ulcerative colitis, irritable bowel syndrome, rheumatoid arthritis, fibromyalgia, endometriosis, nasal polyps, neuropathic pain, inflammatory pain, pseudo-allergic drug reactions, chronic itch, drug-induced anaphylactoid reactions, metabolic syndrome, esophagus reflux, asthma, cough, migraine, sinusitis, urticaria, chronic inducible urticaria, chronic pruritus, acute pruritus, prurigo nodularis, osteoarthritis, pseudo anaphylaxis, contact urticaria, lupus erythematosus (SLE), psoriasis, psoriatic arthritis, bronchial asthma, systemic mastocytosis, cutaneous mastocytosis, mastocytic enterocolitis, mast cell activation syndrome (MCAS), interstitial cystitis, food allergy, allergic rhinitis, microbial infection, eosinophilic esophagitis (EOE) and chronic pain comprising administering to the subject an effective amount of a compound of claim 1 or a pharmaceutically acceptable salt thereof.

64. The method of claim 63, wherein the condition is atopic dermatitis.

65.-77. (canceled)
